SEQUENCE LISTING (391 sequences)

SEQ ID NO: 1 - RNA, 22 nt, Homo sapiens
uagcagcacauaaugguuugug

SEQ ID NO: 2 - RNA, 22 nt, Homo sapiens
uagcagcacaucaugguuuaca

SEQ ID NO: 3 - RNA, 22 nt, Homo sapiens
uagcagcacguaaauauuggcg

SEQ ID NO: 4 - RNA, 21 nt, Homo sapiens
uagcagcacagaaauauuggc

SEQ ID NO: 5 - RNA, 22 nt, Ateles geoffroyi
uagcagcacauaaugguuugug

SEQ ID NO: 6 - RNA, 22 nt, Ateles geoffroyi
uagcagcacaucaugguuuaca

SEQ ID NO: 7 - RNA, 22 nt, Ateles geoffroyi
uagcagcacguaaauauuggcg

SEQ ID NO: 8 - RNA, 22 nt, Bos taurus
uagcagcacaucaugguuuaca

SEQ ID NO: 9 - RNA, 21 nt, Bos taurus
uagcagcacguaaauauuggc

SEQ ID NO: 10 - RNA, 22 nt, Danio rerio
uagcagcacagaaugguuugug

SEQ ID NO: 11 - RNA, 22 nt, Danio rerio
caggccguacugugcugcggca

SEQ ID NO: 12 - RNA, 22 nt, Danio rerio
uagcagcacaucaugguuugua

SEQ ID NO: 13 - RNA, 21 nt, Danio rerio
aagcagcgcgucaugguuuuc

SEQ ID NO: 14 - RNA, 22 nt, Danio rerio
uagcagcacguaaauauuggug

SEQ ID NO: 15 - RNA, 22 nt, Danio rerio
uagcagcacguaaauauuggag

SEQ ID NO: 16 - RNA, 22 nt, Danio rerio
uagcagcauguaaauauuggag

SEQ ID NO: 17 - RNA, 22 nt, Danio rerio
aagcagcacaucaauauuggca

SEQ ID NO: 18 - RNA, 22 nt, Danio rerio
aagcagcacauaaauacuggag

SEQ ID NO: 19 - RNA, 22 nt, Fugu rubripes
uagcagcacggaaugguuugug

SEQ ID NO: 20 - RNA, 22 nt, Fugu rubripes
uagcagcgcaucaugguuugua

SEQ ID NO: 21 - RNA, 22 nt, Fugu rubripes
uagcagcacguaaauauuggag

SEQ ID NO: 22 - RNA, 21 nt, Gallus gallus
uagcagcacauaaugguuugu

SEQ ID NO: 23 - RNA, 22 nt, Gallus gallus
uagcagcacaucaugguuugca

SEQ ID NO: 24 - RNA, 22 nt, Gallus gallus
uagcagcacguaaauauuggug

SEQ ID NO: 25 - RNA, 22 nt, Gorilla gorilla
uagcagcacauaaugguuugug

SEQ ID NO: 26 - RNA, 22 nt, Gorilla gorilla
uagcagcacaucaugguuuaca

SEQ ID NO: 27 - RNA, 22 nt, Gorilla gorilla
uagcagcacguaaauauuggcg

SEQ ID NO: 28 - RNA, 21 nt, Gorilla gorilla
uagcagcacagaaauauuggc

SEQ ID NO: 29 - RNA, 22 nt, Lemur catta
uagcagcacauaaugguuugug

SEQ ID NO: 30 - RNA, 22 nt, Lemur catta
uagcagcacguaaauauuggug

SEQ ID NO: 31 - RNA, 22 nt, Lagothrix lagotricha
uagcagcacauaaugguuugug

SEQ ID NO: 32 - RNA, 22 nt, Lagothrix lagotricha
uagcagcacaucaugguuuaca

SEQ ID NO: 33 - RNA, 22 nt, Lagothrix lagotricha
uagcagcacguaaauauuggcg

SEQ ID NO: 34 - RNA, 22 nt, Monodelphis domestica
uagcagcacauaaugguuuguu

SEQ ID NO: 35 - RNA, 22 nt, Monodelphis domestica
uagcagcacguaaauauuggcg

SEQ ID NO: 36 - RNA, 22 nt, Macaca mulatta
uagcagcacauaaugguuugug

SEQ ID NO: 37 - RNA, 22 nt, Macaca mulatta
uagcagcacaucaugguuuaca

SEQ ID NO: 38 - RNA, 22 nt, Macaca mulatta
uagcagcacguaaauauuggcg

SEQ ID NO: 39 - RNA, 22 nt, Mus musculus
uagcagcacauaaugguuugug

SEQ ID NO: 40 - RNA, 22 nt, Mus musculus
uagcagcacaucaugguuuaca

SEQ ID NO: 41 - RNA, 22 nt, Mus musculus
uagcagcacguaaauauuggcg

SEQ ID NO: 42 - RNA, 21 nt, Mus musculus
uagcagcacagaaauauuggc

SEQ ID NO: 43 - RNA, 22 nt, Macaca nemestrina
uagcagcacauaaugguuugug

SEQ ID NO: 44 - RNA, 22 nt, Macaca nemestrina
uagcagcacaucaugguuuaca

SEQ ID NO: 45 - RNA, 22 nt, Macaca nemestrina
uagcagcacguaaauauuggcg

SEQ ID NO: 46 - RNA, 22 nt, Pan paniscus
uagcagcacauaaugguuugug

SEQ ID NO: 47 - RNA, 22 nt, Pan paniscus
uagcagcacaucaugguuuaca

SEQ ID NO: 48 - RNA, 22 nt, Pan paniscus
uagcagcacguaaauauuggcg

SEQ ID NO: 49 - RNA, 21 nt, Pan paniscus
uagcagcacagaaauauuggc

SEQ ID NO: 50 - RNA, 22 nt, Pongo pygmaeus
uagcagcacauaaugguuugug

SEQ ID NO: 51 - RNA, 22 nt, Pongo pygmaeus
uagcagcacaucaugguuuaca

SEQ ID NO: 52 - RNA, 22 nt, Pongo pygmaeus
uagcagcacguaaauauuggcg

SEQ ID NO: 53 - RNA, 22 nt, Pan troglodytes
uagcagcacauaaugguuugug

SEQ ID NO: 54 - RNA, 22 nt, Pan troglodytes
uagcagcacaucaugguuuaca

SEQ ID NO: 55 - RNA, 22 nt, Pan troglodytes
uagcagcacguaaauauuggcg

SEQ ID NO: 56 - RNA, 22 nt, Rattus norvegicus
uagcagcacaucaugguuuaca

SEQ ID NO: 57 - RNA, 22 nt, Rattus norvegicus
uagcagcacguaaauauuggcg

SEQ ID NO: 58 - RNA, 21 nt, Rattus norvegicus
uagcagcacagaaauauuggc

SEQ ID NO: 59 - RNA, 22 nt, Sagyubys labiatus
uagcagcacauaaugguuugug

SEQ ID NO: 60 - RNA, 22 nt, Sagyubys labiatus
uagcagcacguaaauauuggcg

SEQ ID NO: 61 - RNA, 22 nt, Sus scrofa
ccgcagcacaucaugguuuaca

SEQ ID NO: 62 - RNA, 22 nt, Tetraodon nigroviridis
uagcagcacggaaugguuugug

SEQ ID NO: 63 - RNA, 22 nt, Tetraodon nigroviridis
uagcagcgcaucaugguuugua

SEQ ID NO: 64 - RNA, 22 nt, Tetraodon nigroviridis
uagcagcacguaaauauuggag

SEQ ID NO: 65 - RNA, 22 nt, Xenopus tropicalis
uagcagcacauaaugguuugug

SEQ ID NO: 66 - RNA, 22 nt, Xenopus tropicalis
uagcagcacaucaugauuugca

SEQ ID NO: 67 - RNA, 22 nt, Xenopus tropicalis
uagcagcacaucaugguuugua

SEQ ID NO: 68 - RNA, 22 nt, Xenopus tropicalis
uagcagcacguaaauauuggug

SEQ ID NO: 69 - RNA, 22 nt, Xenopus tropicalis
uagcagcacguaaauauugggu

SEQ ID NO: 70 - RNA, 22 nt, Xenopus tropicalis
uagcagcacguaaauacuggag

SEQ ID NO: 71 - RNA, 21 nt, Homo sapiens
uucaaguaauccaggauaggc

SEQ ID NO: 72 - RNA, 22 nt, Homo sapiens
uucaaguaauucaggauagguu

SEQ ID NO: 73 - RNA, 22 nt, Bos taurus
uucaaguaauccaggauaggcu

SEQ ID NO: 74 - RNA, 22 nt, Bos taurus
uucaaguaauucaggauagguu

SEQ ID NO: 75 - RNA, 22 nt, Danio rerio
uucaaguaauccaggauaggcu

SEQ ID NO: 76 - RNA, 22 nt, Danio rerio
uucaaguaauccaggauagguu

SEQ ID NO: 77 - RNA, 22 nt, Fugu rubripes
uucaaguaauccaggauaggcu

SEQ ID NO: 78 - RNA, 21 nt, Gallus gallus
uucaaguaauccaggauaggc

SEQ ID NO: 79 - RNA, 22 nt, Gorilla gorilla
uucaaguaauccaggauaggcu

SEQ ID NO: 80 - RNA, 22 nt, Lagothrix lagotricha
uucaaguaauccaggauaggcu

SEQ ID NO: 81 - RNA, 22 nt, Macaca mulatta
uucaaguaauccaggauaggcu

SEQ ID NO: 82 - RNA, 21 nt, Mus musculus
uucaaguaauccaggauaggc

SEQ ID NO: 83 - RNA, 22 nt, Mus musculus
uucaaguaauucaggauagguu

SEQ ID NO: 84 - RNA, 22 nt, Macaca nemestrina
uucaaguaauccaggauaggcu

SEQ ID NO: 85 - RNA, 22 nt, Pan paniscus
uucaaguaauccaggauaggcu

SEQ ID NO: 86 - RNA, 22 nt, Pongo pygmaeus
uucaaguaauccaggauaggcu

SEQ ID NO: 87 - RNA, 22 nt, Pan troglodytes
uucaaguaauccaggauaggcu

SEQ ID NO: 88 - RNA, 21 nt, Rattus norvegicus
uucaaguaauccaggauaggc

SEQ ID NO: 89 - RNA, 22 nt, Rattus norvegicus
uucaaguaauucaggauagguu

SEQ ID NO: 90 - RNA, 22 nt, Sus scrofa
uucaaguaauccaggauaggcu

SEQ ID NO: 91 - RNA, 22 nt, Tetraodon nigroviridis
uucaaguaauccaggauaggcu

SEQ ID NO: 92 - RNA, 21 nt, Xenopus tropicalis
uucaaguaauccaggauaggc

SEQ ID NO: 93 - RNA, 21 nt, Homo sapiens
ggcaagaugcuggcauagcug

SEQ ID NO: 94 - RNA, 21 nt, Bombyx mori
ggcaagaagucggcauagcug

SEQ ID NO: 95 - RNA, 21 nt, Bos taurus
aggcaagaugcuggcauagcu

SEQ ID NO: 96 - RNA, 23 nt, Drosophila melanogaster
uggcaagaugucggcauagcuga

SEQ ID NO: 97 - RNA, 22 nt, Drosophila melanogaster
uggcaagaugucggaauagcug

SEQ ID NO: 98 - RNA, 23 nt, Drosophila pseudoobscura
uggcaagaugucggcauagcuga

SEQ ID NO: 99 - RNA, 23 nt, Drosophila pseudoobscura
uggcaagaugucggaauagcuga

SEQ ID NO: 100 - RNA, 21 nt, Danio rerio
ggcaagauguuggcauagcug

SEQ ID NO: 101 - RNA, 22 nt, Gallus gallus
aggcaagauguuggcauagcug

SEQ ID NO: 102 - RNA, 21 nt, Gorilla gorilla
ggcaagaugcuggcauagcug

SEQ ID NO: 103 - RNA, 24 nt, Monodelphis domestica
ggaggcaagauguuggcauagcug

SEQ ID NO: 104 - RNA, 21 nt, Macaca mulatta
ggcaagaugcuggcauagcug

SEQ ID NO: 105 - RNA, 22 nt, Mus musculus
aggcaagaugcuggcauagcug

SEQ ID NO: 106 - RNA, 21 nt, Macaca nemestrina
ggcaagaugcuggcauagcug

SEQ ID NO: 107 - RNA, 21 nt, Pan paniscus
ggcaagaugcuggcauagcug

SEQ ID NO: 108 - RNA, 21 nt, Pongo pygmaeus
ggcaagaugcuggcauagcug

SEQ ID NO: 109 - RNA, 21 nt, Pan troglodytes
ggcaagaugcuggcauagcug

SEQ ID NO: 110 - RNA, 22 nt, Rattus norvegicus
aggcaagaugcuggcauagcug

SEQ ID NO: 111 - RNA, 23 nt, Schmidtea mediterranea
aggcaagaugcuggcauagcuga

SEQ ID NO: 112 - RNA, 22 nt, Xenopus tropicalis
aggcaagauguuggcauagcug

SEQ ID NO: 113 - RNA, 24 nt, Homo sapiens
guccaguuuucccaggaaucccuu

SEQ ID NO: 114 - RNA, 20 nt, Homo sapiens
guguguggaaaugcuucugc

SEQ ID NO: 115 - RNA, 22 nt, Homo sapiens
caucccuugcaugguggagggu

SEQ ID NO: 116 - RNA, 22 nt, Homo sapiens
caugccuugaguguaggaccgu

SEQ ID NO: 117 - RNA, 22 nt, Bos taurus
caugccuugaguguaggaccgu

SEQ ID NO: 118 - RNA, 22 nt, Homo sapiens
uacccauugcauaucggaguug

SEQ ID NO: 119 - RNA, 22 nt, Macaca mulatta
caucccuugcaugguggagggu

SEQ ID NO: 120 - RNA, 22 nt, Mus musculus
caucccuugcaugguggagggu

SEQ ID NO: 121 - RNA, 22 nt, Mus musculus
caugccuugaguguaggaccgu

SEQ ID NO: 122 - RNA, 22 nt, Macaca nemestrina
caucccuugcaugguggagggu

SEQ ID NO: 123 - RNA, 22 nt, Pan paniscus
caucccuugcaugguggagggu

SEQ ID NO: 124 - RNA, 22 nt, Pongo pygmaeus
caucccuugcaugguggagggu

SEQ ID NO: 125 - RNA, 22 nt, Pan troglodytes
caucccuugcaugguggagggu

SEQ ID NO: 126 - RNA, 21 nt, Homo sapiens
augaccuaugaauugacagac

SEQ ID NO: 127 - RNA, 21 nt, Homo sapiens
cugaccuaugaauugacagcc

SEQ ID NO: 128 - RNA, 23 nt, Bos taurus
cugaccuaugaauugacagccag

SEQ ID NO: 129 - RNA, 22 nt, Bos taurus
augaccuaugaauugacagaca

SEQ ID NO: 130 - RNA, 21 nt, Danio rerio
augaccuaugaauugacagcc

SEQ ID NO: 131 - RNA, 21 nt, Fugu rubripes
augaccuaugaauugacagcc

SEQ ID NO: 132 - RNA, 21 nt, Gallus gallus
augaccuaugaauugacagac

SEQ ID NO: 133 - RNA, 21 nt, Gorilla gorilla
augaccuaugaauugacagac

SEQ ID NO: 134 - RNA, 21 nt, Macaca mulatta
augaccuaugaauugacagac

SEQ ID NO: 135 - RNA, 18 nt, Mus musculus
cugaccuaugaauugaca

SEQ ID NO: 136 - RNA, 21 nt, Mus musculus
augaccuaugauuugacagac

SEQ ID NO: 137 - RNA, 21 nt, Macaca nemestrina
augaccuaugaauugacagac

SEQ ID NO: 138 - RNA, 21 nt, Pongo pygmaeus
augaccuaugaauugacagac

SEQ ID NO: 139 - RNA, 21 nt, Pan troglodytes
augaccuaugaauugacagac

SEQ ID NO: 140 - RNA, 21 nt, Rattus norvegicus
cugaccuaugaauugacagcc

SEQ ID NO: 141 - RNA, 21 nt, Rattus norvegicus
augaccuaugauuugacagac

SEQ ID NO: 142 - RNA, 21 nt, Tetraodon nigroviridis
augaccuaugaauugacagcc

SEQ ID NO: 143 - RNA, 21 nt, Xenopus tropicalis
augaccuaugaauugacagcc

SEQ ID NO: 144 - RNA, 21 nt, Xenopus tropicalis
augaccuaugaaaugacagcc

SEQ ID NO: 145 - RNA, 21 nt, Homo sapiens
uaaucucagcuggcaacugug

SEQ ID NO: 146 - RNA, 22 nt, Danio rerio
uaaucucagcuggcaacuguga

SEQ ID NO: 147 - RNA, 22 nt, Danio rerio
uaaucucugcaggcaacuguga

SEQ ID NO: 148 - RNA, 22 nt, Fugu rubripes
aaaucucagcuggcaacuguga

SEQ ID NO: 149 - RNA, 22 nt, Fugu rubripes
uaaucucugcaggcaacuguga

SEQ ID NO: 150 - RNA, 21 nt, Gallus gallus
uaaucucagcuggcaacugug

SEQ ID NO: 151 - RNA, 21 nt, Gorilla gorilla
uaaucucagcuggcaacugug

SEQ ID NO: 152 - RNA, 21 nt, Lemur catta
uaaucucagcuggcaacugug

SEQ ID NO: 153 - RNA, 21 nt, Monodelphis domestica
uaaucucagcuggcaacugug

SEQ ID NO: 154 - RNA, 21 nt, Mus musculus
uaaucucagcuggcaacugug

SEQ ID NO: 155 - RNA, 25 nt, Mus musculus
gggaaaucucugcaggcaaauguga

SEQ ID NO: 156 - RNA, 21 nt, Pan paniscus
uaaucucagcuggcaacugug

SEQ ID NO: 157 - RNA, 21 nt, Pongo pygmaeus
uaaucucagcuggcaacugug

SEQ ID NO: 158 - RNA, 21 nt, Pan troglodytes
uuaucucagcuggcaacugug

SEQ ID NO: 159 - RNA, 21 nt, Rattus norvegicus
uaaucucagcuggcaacugug

SEQ ID NO: 160 - RNA, 21 nt, Sus scrofa
uaaucucagcuggcaacugug

SEQ ID NO: 161 - RNA, 22 nt, Tetraodon nigroviridis
aaaucucagcuggcaacuguga

SEQ ID NO: 162 - RNA, 22 nt, Tetraodon nigroviridis
uaaucucugcaggcaacuguga

SEQ ID NO: 163 - RNA, 21 nt, Xenopus tropicalis
uaaucucagcuggcaacugug

SEQ ID NO: 164 - RNA, 21 nt, Homo sapiens
gccccugggccuauccuagaa

SEQ ID NO: 165 - RNA, 23 nt, Mus musculus
aagugccgccagguuuugagugu

SEQ ID NO: 166 - RNA, 21 nt, Homo sapiens
gugccgccaucuuuugagugu

SEQ ID NO: 167 - RNA, 23 nt, Homo sapiens
aaagugcugcgacauuugagcgu

SEQ ID NO: 168 - RNA, 23 nt, Mus musculus
cucaaacuaugggggcacuuuuu

SEQ ID NO: 169 - RNA, 23 nt, Mus musculus
aaagugcuuccacuuugugugcc

SEQ ID NO: 170 - RNA, 22 nt, Mus musculus
caucaaaguggaggcccucucu

SEQ ID NO: 171 - RNA, 23 nt, Mus musculus
aaagugcauccauuuuguuuguc

SEQ ID NO: 172 - RNA, 21 nt, Mus musculus
gaucaaaguggaggcccucuc

SEQ ID NO: 173 - RNA, 22 nt, Mus musculus
acucaaacugggggcucuuuug

SEQ ID NO: 174 - RNA, 22 nt, Mus musculus
agugccgcagaguuuguagugu

SEQ ID NO: 175 - RNA, 22 nt, Mus musculus
aaagugcuucccuuuugugugu

SEQ ID NO: 176 - RNA, 23 nt, Mus musculus
aaagugcuacuacuuuugagucu

SEQ ID NO: 177 - RNA, 23 nt, Rattus norvegicus
cucaaacuaugggggcacuuuuu

SEQ ID NO: 178 - RNA, 23 nt, Rattus norvegicus
aaagugcuuccacuuugugugcc

SEQ ID NO: 179 - RNA, 22 nt, Rattus norvegicus
caucaaaguggaggcccucucu

SEQ ID NO: 180 - RNA, 23 nt, Rattus norvegicus
aagugccgccagguuuugagugu

SEQ ID NO: 181 - RNA, 22 nt, Rattus norvegicus
acucaaacugggggcucuuuug

SEQ ID NO: 182 - RNA, 83 nt, Homo sapiens
ccuuggaguaaaguagcagcacauaaugguuuguggauuuugaaaaggugcaggccauauugugcugccucaaaaauacaagg

SEQ ID NO: 183 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 184 - RNA, 89 nt, Homo sapiens
gucagcagugccuuagcagcacguaaauauuggcguuaagauucuaaaauuaucuccaguauuaacugugcugcugaaguaagguugac

SEQ ID NO: 185 - RNA, 81 nt, Homo sapiens
guuccacucuagcagcacguaaauauuggcguagugaaauauauauuaaacaccaauauuacugugcugcuuuagugugac

SEQ ID NO: 186 - RNA, 87 nt, Homo sapiens
agcuucccuggcucuagcagcacagaaauauuggcacagggaagcgagucugccaauauuggcugugcugcuccaggcaggguggug

SEQ ID NO: 187 - RNA, 83 nt, Ateles geoffroyi; identical to SEQ ID NO: 182

SEQ ID NO: 188 - RNA, 98 nt, Ateles geoffroyi
uugaggccuuaaaguacuguagcagcacaucaugguuuacauacuacagucaagaugcgaaucauuauuugcugcucuagaaauuuaaggaaauucau

SEQ ID NO: 189 - RNA, 89 nt, Ateles geoffroyi; identical to SEQ ID NO: 184

SEQ ID NO: 190 - RNA, 83 nt, Bos taurus; identical to SEQ ID NO: 182

SEQ ID NO: 191 - RNA, 98 nt, Bos taurus
uugagaccuuaaaguacuguagcagcacaucaugguuuacauacuacagucaagaugcgaaucauuauuugcugcucuagaaauuuaaggaaauucau

SEQ ID NO: 192 - RNA, 87 nt, Bos taurus
agcuccccuggcucuagcagcacagaaauauuggcacugggaagaaagccugccaauauuggcugugcugcuccaggcaggguggug

SEQ ID NO: 193 - RNA, 84 nt, Danio rerio
ccugucgguacuguagcagcacagaaugguuugugaguuauaacgggggugcaggccguacugugcugcggcaacaacgacagg

SEQ ID NO: 194 - RNA, 102 nt, Danio rerio
gccgaggcucucuaggugaugguguagcagcacagaaugguuuguggugauacagagaugcaggccaugaugugcugcagcaucaauuccugggaccuacgc

SEQ ID NO: 195 - RNA, 134 nt, Danio rerio
gucugucgucaucuuuuuauuuagcccugagugcccuguagcagcacaucaugguuuguaaguuauaagggcaaauuccgaaucaugaugugcugucacugggagccugggaguuucuccauuaacaugacagc

SEQ ID NO: 196 - RNA, 83 nt, Danio rerio
ccuuagaccgcuaaagcagcgcgucaugguuuucaacauuagagaaggugcaagccaucauuugcugcucuagaguuuuaagg

SEQ ID NO: 197 - RNA, 87 nt, Danio rerio
ccuuccucgcuuuagcagcacguaaauauugguguguuauagucaaggccaaccccaauauuaugugugcugcuucaguaaggcagg

SEQ ID NO: 198 - RNA, 140 nt, Danio rerio
ccugaacuuggccgugugacagacuggcugccuggcuguagcagcacguaaauauuggagucaaagcacuugcgaauccuccaguauugaccgugcugcuggaguuaggcgggccguuuaccgucugcgggggccucggg

SEQ ID NO: 199 - RNA, 136 nt, Danio rerio
gagguugugugugugugcguguguugucuugcuuuagcagcauguaaauauuggaguuacuccuuggccaaugccuccaauauugcucgugcugcugaagcaagaagucaccaagcagcacaugcacgucauccuu

SEQ ID NO: 200 - RNA, 83 nt, Danio rerio
ugccugacagaagcagcacaucaauauuggcagcugcccucucucuggguugccaguaugguuugugcugcucccgucagaca

SEQ ID NO: 201 - RNA, 78 nt, Danio rerio
gaauguacuaaagcagcacauaaauacuggaggugauugugguguuauccaguauugcuguucugcuguaguaagacc

SEQ ID NO: 202 - RNA, 71 nt, Fugu rubripes
cuggugaugcuguagcagcacggaaugguuuguggguuacacugagauacaggccauacugugcugccgca

SEQ ID NO: 203 - RNA, 95 nt, Fugu rubripes
ugagucccuuagacugcuauagcagcgcaucaugguuuguaacgauguagaaaagggugcaagccauaaucugcugcuuuagaauuuuaaggaaa

SEQ ID NO: 204 - RNA, 89 nt, Fugu rubripes
gccacugugcuguagcagcacguaaauauuggaguuaaggcucucugugauaccuccaguauugaucgugcugcugaagcaaagaugac

SEQ ID NO: 205 - RNA, 83 nt, Gallus gallus
ccuuggcauaacguagcagcacauaaugguuuguggguuuugaaaaggugcaggccauauugugcugccucaaaaauacaagg

SEQ ID NO: 206 - RNA, 91 nt, Gallus gallus
ugaggccuuaaaguacucuagcagcacaucaugguuugcaugcuguagugaagaugcgaaucauuauuugcugcuuuagaaauuuaaggaa

SEQ ID NO: 207 - RNA, 84 nt, Gallus gallus
gucugucauacucuagcagcacguaaauauugguguuaaaacuguaaauaucuccaguauuaacugugcugcugaaguaaggcu

SEQ ID NO: 208 - RNA, 94 nt, Gallus gallus
ccuacuuguuccgcccuagcagcacguaaauauugguguaguaaaauaaaccuuaaaccccaauauuauugugcugcuuaagcguggcagagau

SEQ ID NO: 209 - RNA, 83 nt, Gorilla gorilla; identical to SEQ ID NO: 182

SEQ ID NO: 210 - RNA, 98 nt, Gorilla gorilla; identical to SEQ ID NO: 183

SEQ ID NO: 211 - RNA, 89 nt, Gorilla gorilla; identical to SEQ ID NO: 184

SEQ ID NO: 212 - RNA, 93 nt, Bos taurus
cauacuuguuccgcuguagcagcacguaaauauuggcguaguaaaauaaauauuaaacaccaauauuauugugcugcuuuagcgugacaggga

SEQ ID NO: 213 - RNA, 87 nt, Gorilla gorilla
agcuuccugggcucuagcagcacagaaauauuggcacagggaagcgagucugccaauauuggcugugcugcuccaggcaggguggug

SEQ ID NO: 214 - RNA, 83 nt, Lemur catta; identical to SEQ ID NO: 182

SEQ ID NO: 215 - RNA, 74 nt, Lemur catta
gucagcagugccuuagcagcacguaaauauugguguuaagauucuaaaauuaucucuaaguauuaacugugccg

SEQ ID NO: 216 - RNA, 83 nt, Lagothrix lagotricha; identical to SEQ ID NO: 182

SEQ ID NO: 217 - RNA, 98 nt, Lagothrix lagotricha; identical to SEQ ID NO: 188

SEQ ID NO: 218 - RNA, 89 nt, Lagothrix lagotricha
gucagcagugccuuagcagcacguaaauauuggcgcuaagauucuaaaauuaucuccaguauuaacugugcugcugaaguaagguuggc

SEQ ID NO: 219 - RNA, 83 nt, Monodelphis domestica
ccuugggguaaaguagcagcacauaaugguuuguugguuuugaaaaggugcaggccauauugugcugccucaaaaauacaagg

SEQ ID NO: 220 - RNA, 90 nt, Monodelphis domestica
gucaacagugccuuagcagcacguaaauauuggcguuaagauuuuaaaaguaucuccaguauuaacugugcugcugaaguaagguuggcc

SEQ ID NO: 221 - RNA, 83 nt, Macaca mulatta; identical to SEQ ID NO: 182

SEQ ID NO: 222 - RNA, 98 nt, Macaca mulatta; identical to SEQ ID NO: 188

SEQ ID NO: 223 - RNA, 89 nt, Macaca mulatta; identical to SEQ ID NO: 184

SEQ ID NO: 224 - RNA, 84 nt, Mus musculus
cccuuggaguaaaguagcagcacauaaugguuuguggauguugaaaaggugcaggccauacugugcugccucaaaauacaagga

SEQ ID NO: 225 - RNA, 64 nt, Mus musculus
cuguagcagcacaucaugguuuacauacuacagucaagaugcgaaucauuauuugcugcucuag

SEQ ID NO: 226 - RNA, 93 nt, Mus musculus
augucagcggugccuuagcagcacguaaauauuggcguuaagauucugaaauuaccuccaguauugacugugcugcugaaguaagguuggcaa

SEQ ID NO: 227 - RNA, 95 nt, Mus musculus
caugcuuguuccacucuagcagcacguaaauauuggcguagugaaauaaauauuaaacaccaauauuauugugcugcuuuagugugacagggaua

SEQ ID NO: 228 - RNA, 94 nt, Mus musculus
acacccaacucuccuggcucuagcagcacagaaauauuggcauggggaagugagucugccaauauuggcugugcugcuccaggcagggugguga

SEQ ID NO: 229 - RNA, 83 nt, Macaca nemestrina; identical to SEQ ID NO: 182

SEQ ID NO: 230 - RNA, 98 nt, Macaca nemestrina; identical to SEQ ID NO: 188

SEQ ID NO: 231 - RNA, 89 nt, Macaca nemestrina; identical to SEQ ID NO: 184

SEQ ID NO: 232 - RNA, 83 nt, Pan paniscus; identical to SEQ ID NO: 182

SEQ ID NO: 233 - RNA, 98 nt, Pan paniscus; identical to SEQ ID NO: 183

SEQ ID NO: 234 - RNA, 89 nt, Pan paniscus; identical to SEQ ID NO: 184

SEQ ID NO: 235 - RNA, 87 nt, Pan paniscus; identical to SEQ ID NO: 186

SEQ ID NO: 236 - RNA, 83 nt, Pongo pygmaeus; identical to SEQ ID NO: 182

SEQ ID NO: 237 - RNA, 98 nt, Pongo pygmaeus; identical to SEQ ID NO: 183

SEQ ID NO: 238 - RNA, 89 nt, Pongo pygmaeus; identical to SEQ ID NO: 184

SEQ ID NO: 239 - RNA, 83 nt, Pan troglodytes; identical to SEQ ID NO: 182

SEQ ID NO: 240 - RNA, 98 nt, Pan troglodytes; identical to SEQ ID NO: 183

SEQ ID NO: 241 - RNA, 89 nt, Pan troglodytes; identical to SEQ ID NO: 184

SEQ ID NO: 242 - RNA, 98 nt, Rattus norvegicus
uuggaaccuuaaaguacuguagcagcacaucaugguuuacauacuacagucaagaugcgaaucauuauuugcugcucuagaaauuuaaggaaauucau

SEQ ID NO: 243 - RNA, 95 nt, Rattus norvegicus
cauacuuguuccgcucuagcagcacguaaauauuggcguagugaaauaaauauuaaacaccaauauuauugugcugcuuuagugugacagggaua

SEQ ID NO: 244 - RNA, 87 nt, Rattus norvegicus
aacucuccuggcucuagcagcacagaaauauuggcacggguaagugagucugccaauauuggcugugcugcuccaggcaggguggug

SEQ ID NO: 245 - RNA, 83 nt, Sagyubys labiatus; identical to SEQ ID NO: 182

SEQ ID NO: 246 - RNA, 89 nt, Sagyubys labiatus; identical to SEQ ID NO: 184

SEQ ID NO: 247 - RNA, 98 nt, Sus scrofa
uugaggccuuaaaguacugccgcagcacaucaugguuuacauacuacaaucaagaugcgaaucauuauuugcugcucuagaaauuuaaggaaauucau

SEQ ID NO: 248 - RNA, 71 nt, Tetraodon nigroviridis
cuggugaugcuguagcagcacggaaugguuugugaguuacacugagauacaagccaugcugugcugccgca

SEQ ID NO: 249 - RNA, 91 nt, Tetraodon nigroviridis
gcccuuagacugcuuuagcagcgcaucaugguuuguaaugauguggaaaaaaggugcaaaccauaauuugcugcuuuagaauuuuaaggaa

SEQ ID NO: 250 - RNA, 72 nt, Tetraodon nigroviridis
uagcagcacguaaauauuggaguuaaggcucucugugauaccuccaguauugaucgugcugcugaagcaaag

SEQ ID NO: 251 - RNA, 83 nt, Xenopus tropicalis
ccuugacguaaaguagcagcacauaaugguuuguggguuacacagaggugcaggccauacugugcugccgccaaaacacaagg

SEQ ID NO: 252 - RNA, 88 nt, Xenopus tropicalis
uguccuaaagaaguguagcagcacaucaugauuugcaugcuguauuauagauucuaaucauuuuuugcugcuucaugauauugggaaa

SEQ ID NO: 253 - RNA, 74 nt, Xenopus tropicalis
cuuugaggugaucuagcagcacaucaugguuuguagaaacaaggagauacagaccauucugagcugccucuuga

SEQ ID NO: 254 - RNA, 82 nt, Xenopus tropicalis
gccagcaguccuuuagcagcacguaaauauugguguuaaaauggucccaauauuaacugugcugcuagaguaagguuggccu

SEQ ID NO: 255 - RNA, 84 nt, Xenopus tropicalis
aauugcuccgcauuagcagcacguaaauauugggugauaugauauggagccccaguauuauuguacugcuuaaguguggcaagg

SEQ ID NO: 256 - RNA, 70 nt, Xenopus tropicalis
uuuagcagcacguaaauacuggaguucaugaccauaucugcacucuccaguauuacuuugcugcuauauu

SEQ ID NO: 257 - RNA, 77 nt, Homo sapiens
guggccucguucaaguaauccaggauaggcugugcaggucccaaugggccuauucuugguuacuugcacggggacgc

SEQ ID NO: 258 - RNA, 84 nt, Homo sapiens
ggcuguggcuggauucaaguaauccaggauaggcuguuuccaucugugaggccuauucuugauuacuuguuucuggaggcagcu

SEQ ID NO: 259 - RNA, 77 nt, Homo sapiens
ccgggacccaguucaaguaauucaggauagguugugugcuguccagccuguucuccauuacuuggcucggggaccgg

SEQ ID NO: 260 - RNA, 84 nt, Bos taurus; identical to SEQ ID NO: 258

SEQ ID NO: 261 - RNA, 85 nt, Bos taurus
ugcccgggacccaguucaaguaauucaggauagguugugugcuguccagccuguucuccauuacuuggcucgggggccggugccc

SEQ ID NO: 262 - RNA, 78 nt, Danio rerio
uuuggccugguucaaguaauccaggauaggcuugugauguccggaaagccuauucgggaugacuugguucaggaauga

SEQ ID NO: 263 - RNA, 128 nt, Danio rerio
guguggacuugagugcuggaagugguuguucccuuguucaaguaauccaggauaggcugucuguccuggaggccuauucaugauuacuugcacuagguggcagccguugcccuucauggaacucaugc

SEQ ID NO: 264 - RNA, 137 nt, Danio rerio
cuaagcugauacugagucaguguguggcugcaaccugguucaaguaauccaggauaggcuuuguggacuaggguuggccuguucuugguuacuugcacuggguugcagcuacuaaacaacuaagaagaucagaagag

SEQ ID NO: 265 - RNA, 88 nt, Fugu rubripes
aggccucggccugguucaaguaauccaggauaggcugguuaacccugcacggccuauucuugauuacuugugucaggaaguggccgug

SEQ ID NO: 266 - RNA, 77 nt, Gallus gallus
gucaccugguucaaguaauccaggauaggcuguauccauuccugcuggccuauucuugguuacuugcacugggaggc

SEQ ID NO: 267 - RNA, 77 nt, Gorilla gorilla; identical to SEQ ID NO: 257

SEQ ID NO: 268 - RNA, 77 nt, Lagothrix lagotricha; identical to SEQ ID NO: 257

SEQ ID NO: 269 - RNA, 77 nt, Macaca mulatta; identical to SEQ ID NO: 257

SEQ ID NO: 270 - RNA, 90 nt, Mus musculus
aaggccguggccucguucaaguaauccaggauaggcugugcaggucccaaggggccuauucuugguuacuugcacggggacgcgggccug

SEQ ID NO: 271 - RNA, 84 nt, Mus musculus
ggcugcggcuggauucaaguaauccaggauaggcuguguccguccaugaggccuguucuugauuacuuguuucuggaggcagcg

SEQ ID NO: 272 - RNA, 85 nt, Mus musculus
ugcccgggacccaguucaaguaauucaggauagguuguggugcugaccagccuguucuccauuacuuggcucgggggccggugcc

SEQ ID NO: 273 - RNA, 77 nt, Macaca nemestrina
guggccucguucaaguaauccaggauaggcugugcaggucccaaugggccuauucuugauuacuugcacggggacgc

SEQ ID NO: 274 - RNA, 77 nt, Pan paniscus; identical to SEQ ID NO: 257

SEQ ID NO: 275 - RNA, 77 nt, Pan troglodytes; identical to SEQ ID NO: 257

SEQ ID NO: 276 - RNA, 90 nt, Rattus norvegicus
aaggccguggccuuguucaaguaauccaggauaggcugugcaggucccaaggggccuauucuugguuacuugcacggggacgcgggccug

SEQ ID NO: 277 - RNA, 85 nt, Rattus norvegicus
ugcccgggacccaguucaaguaauucaggauagguuguggugcuggccagccuguucuccauuacuuggcucgggggccggugcc

SEQ ID NO: 278 - RNA, 84 nt, Sus scrofa; identical to SEQ ID NO: 258

SEQ ID NO: 279 - RNA, 96 nt, Tetraodon nigroviridis
gcguuaggccucggccugguucaaguaauccaggauaggcugguuaacccugcacggccuauucuugauuacuugugucaggaaguggccgccagc

SEQ ID NO: 280 - RNA, 85 nt, Xenopus tropicalis
ggcugcugccugguucaaguaauccaggauaggcuguuuccucaaagcacggccuacucuugauuacuuguuucaggaaguagcu

SEQ ID NO: 281 - RNA, 18 nt, Xenopus tropicalis
ugggcgcucgcuucaagu

SEQ ID NO: 282 - RNA, 71 nt, Homo sapiens
ggagaggaggcaagaugcuggcauagcuguugaacugggaaccugcuaugccaacauauugccaucuuucc

SEQ ID NO: 283 - RNA, 100 nt, Apis mellifera
aucacgauucuaacugggcgccucgaaggcaagaugucggcauagcugaugcgauuuuaaaauucggcugugucacauccagccaaccgaacgcucagac

SEQ ID NO: 284 - RNA, 121 nt, Bombyx mori
gucgagccgguggcugggaaggcaagaagucggcauagcuguuugaauaagauacacggcugugucacuucgagccagcucaauccgccggcuuucuucaauuucaagauuugcggaugcu

SEQ ID NO: 285 - RNA, 94 nt, Bos taurus
uccuguaacuuggaacuggagaggaggcaagaugcuggcauagcuguugaacugcgaaccugcuaugccaacauauugccaucucucuuguccg

SEQ ID NO: 286 - RNA, 92 nt, Drosophila melanogaster
uccguugguaaauuggcaagaugucggcauagcugacguugaaaagcgauuuugaagagcgcuaugcugcaucuagucaguuguucaaugga

SEQ ID NO: 287 - RNA, 127 nt, Drosophila melanogaster
caaauaaugaauuuggcaagaugucggaauagcugagagcacagcggaucgaacauuuuaucguccgaaaaaaugugauuauuuuugaaaagcggcuaugccucaucuagucaauugcauuacuuug

SEQ ID NO: 288 - RNA, 92 nt, Drosophila pseudoobscura
ucuguugguaaauuggcaagaugucggcauagcugaaguugaaaagcgaucuuugagaacgcuaugcugcaucuagucaguuauucaaugga

SEQ ID NO: 289 - RNA, 89 nt, Drosophila pseudoobscura
aauuuggcaagaugucggaauagcugagagcaaaaagaagaugauuugaaaugcggcuaugccucaucuagucaauugcauucauuuga

SEQ ID NO: 290 - RNA, 72 nt, Danio rerio
gaagagauggcaagauguuggcauagcuguuaauguuuaugggccugcuaugccuccauauugccauuucug

SEQ ID NO: 291 - RNA, 94 nt, Gallus gallus
uucuuucaugcagagcuggaggggaggcaagauguuggcauagcuguuaaccuaaaaaccugcuaugccaacauauugucaucuuuccugucug

SEQ ID NO: 292 - RNA, 71 nt, Gorilla gorilla; identical to SEQ ID NO: 282

SEQ ID NO: 293 - RNA, 87 nt, Monodelphis domestica
agcuggagaggaggcaagauguuggcauagcuguugaacugagaaccugcuaugccaacauauugccaucuuucuugucuaucagca

SEQ ID NO: 294 - RNA, 71 nt, Macaca mulatta; identical to SEQ ID NO: 282

SEQ ID NO: 295 - RNA, 106 nt, Mus musculus
ugcuccuguaacucggaacuggagaggaggcaagaugcuggcauagcuguugaacugagaaccugcuaugccaacauauugccaucuuuccugucugacagcagcu

SEQ ID NO: 296 - RNA, 71 nt, Macaca nemestrina; identical to SEQ ID NO: 282

SEQ ID NO: 297 - RNA, 71 nt, Pan paniscus; identical to SEQ ID NO: 282

SEQ ID NO: 298 - RNA, 71 nt, Pongo pygmaeus; identical to SEQ ID NO: 282

SEQ ID NO: 299 - RNA, 71 nt, Pan troglodytes; identical to SEQ ID NO: 282

SEQ ID NO: 300 - RNA, 106 nt, Rattus norvegicus
ugcuccugaaacuuggaacuggagaggaggcaagaugcuggcauagcuguugaacugagaaccugcuaugccaacauauugccaucuuuccugucugacagcagcu

SEQ ID NO: 301 - RNA, 85 nt, Schmidtea mediterranea
auugauaaugacaaggcaagaugcuggcauagcugauaaacuauuuauuaccagcuauucaggaucuuucccugaauauaucaau

SEQ ID NO: 302 - RNA, 88 nt, Xenopus tropicalis
ccuaguucuagagaggaggcaagauguuggcauagcuguugcaucugaaaccaguugugccaaccuauugccaucuuucuugucuacc

SEQ ID NO: 303 - RNA, 88 nt, Homo sapiens
caccuuguccucacgguccaguuuucccaggaaucccuuagaugcuaagauggggauuccuggaaauacuguucuugaggucaugguu

SEQ ID NO: 304 - RNA, 88 nt, Bos taurus; identical to SEQ ID NO: 303

SEQ ID NO: 305 - RNA, 113 nt, Danio rerio
ucagucuucaucauuuccucauccccgggguccaguuuucccaggaaucccuugggcaaucgaaagggggauuccuggaaauacuguucuugggguuggggguggacuacuga

SEQ ID NO: 306 - RNA, 88 nt, Gorilla gorilla; identical to SEQ ID NO: 303

SEQ ID NO: 307 - RNA, 70 nt, Monodelphis domestica
cucaggguccaguuuucccaggaaucccuuagaugcuaagauggggauuccuggaaauacuguucuugag

SEQ ID NO: 308 - RNA, 88 nt, Macaca mulatta
caccuuguccucacgguccaguuuucccaggaaucccuuaaaugcuaagauggggauuccuggaaauacuguucuugaggucaugguu

SEQ ID NO: 309 - RNA, 70 nt, Mus musculus
cucacgguccaguuuucccaggaaucccuuggaugcuaagauggggauuccuggaaauacuguucuugag

SEQ ID NO: 310 - RNA, 88 nt, Macaca nemestrina; identical to SEQ ID NO: 308

SEQ ID NO: 311 - RNA, 88 nt, Pongo pygmaeus; identical to SEQ ID NO: 303

SEQ ID NO: 312 - RNA, 88 nt, Pan troglodytes; identical to SEQ ID NO: 303

SEQ ID NO: 313 - RNA, 88 nt, Rattus norvegicus
caccuuguccucacgguccaguuuucccaggaaucccuuggaugcuaagauggggauuccuggaaauacuguucuugaggucauggcu

SEQ ID NO: 314 - RNA, 86 nt, Sus scrofa
caccuuguccucacgguccaguuuucccaggaaucccuuagaugcugagauggggauuccuguaaauacuguucuugaggucaugg

SEQ ID NO: 315 - RNA, 86 nt, Xenopus tropicalis
accuauuccucaagguccaguuuucccaggaaucccuugggugcugugguggggauuccuggaaauacuguucuugggguguaggc

SEQ ID NO: 316 - RNA, 72 nt, Homo sapiens
aaucuaaagacaacauuucugcacacacaccagacuauggaagccaguguguggaaaugcuucugcuagauu

SEQ ID NO: 317 - RNA, 70 nt, Gallus gallus
aaucuaguggaaucacuucugcacaaacuugacuacugaaaucagugugcggaaaugcuucugcuacauu

SEQ ID NO: 318 - RNA, 70 nt, Gallus gallus; identical to SEQ ID NO: 317

SEQ ID NO: 319 - RNA, 72 nt, Macaca nemestrina
aaucuaaagaaaacauuucugcacacacaccagacuauugaagccaguguguggaaaugcuucugcuacauu

SEQ ID NO: 320 - RNA, 72 nt, Pan paniscus
aaucuaaagaaaacauuucugcacacacaccagacuauggaagccaguguguggaaaugcuucugcuagauu

SEQ ID NO: 321 - RNA, 72 nt, Pongo pygmaeus; identical to SEQ ID NO: 320

SEQ ID NO: 322 - RNA, 72 nt, Pan troglodytes; identical to SEQ ID NO: 320

SEQ ID NO: 323 - RNA, 72 nt, Sagyubys labiatus
aaucuaaagaaaacauuucugcacacacaccagacuauugaagccaguguguggaaaugcuucugccacauu

SEQ ID NO: 324 - RNA, 86 nt, Homo sapiens
ugcucccucucucacaucccuugcaugguggagggugagcuuucugaaaaccccucccacaugcaggguuugcaggauggcgagcc

SEQ ID NO: 325 - RNA, 91 nt, Homo sapiens
cgacuugcuuucucuccuccaugccuugaguguaggaccguuggcaucuuaauuacccucccacacccaaggcuugcaaaaaagcgagccu

SEQ ID NO: 326 - RNA, 97 nt, Homo sapiens
cugcuccuucucccauacccauugcauaucggaguugugaauucucaaaacaccuccugugugcauggauuacaggagggugagccuugucaucgug

SEQ ID NO: 327 - RNA, 86 nt, Bos taurus
gacuugcuuucucucuuacaugccuugaguguaggaccguuggcaucuuaauuacccucccacacccaaggcuugcaggagagcca

SEQ ID NO: 328 - RNA, 80 nt, Bos taurus
cugcuccuucucccguacccauugcauaucggagcugugaauucucaaagcaccuccuaugugcauggauuacaggaggg

SEQ ID NO: 329 - RNA, 86 nt, Macaca mulatta
ugcucccucucucacaucccuugcaugguggagggugagcuuuaugaaaaccccucccacaugcaggguuugcaggauggugagcc

SEQ ID NO: 330 - RNA, 68 nt, Mus musculus
ucucacaucccuugcaugguggagggugagcucucugaaaaccccucccacaugcaggguuugcagga

SEQ ID NO: 331 - RNA, 96 nt, Mus musculus
cagauuugcuuuuucucuuccaugccuugaguguaggaccguugacaucuuaauuacccucccacacccaaggcuugcaggagagcaagccuucuc

SEQ ID NO: 332 - RNA, 86 nt, Macaca nemestrina; identical to SEQ ID NO: 329

SEQ ID NO: 333 - RNA, 86 nt, Pan paniscus; identical to SEQ ID NO: 324

SEQ ID NO: 334 - RNA, 86 nt, Pongo pygmaeus; identical to SEQ ID NO: 324

SEQ ID NO: 335 - RNA, 86 nt, Pan troglodytes
ugcucccucucucacaucccuugcaugguggagggugaacuuucugaaaaccccucccacaugcaggguuugcaggauggcgagcc

SEQ ID NO: 336 - RNA, 110 nt, Homo sapiens
aucauucagaaaugguauacaggaaaaugaccuaugaauugacagacaauauagcugaguuugucugucauuucuuuaggccaauauucuguaugacugugcuacuucaa

SEQ ID NO: 337 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 338 - RNA, 106 nt, Bos taurus
agaccgagugcacagggcucugaccuaugaauugacagccagugcucuuguguccccucuggcugccaauuccauaggucacagguauguucgccucaaugccagc

SEQ ID NO: 339 - RNA, 79 nt, Bos taurus
uguacaggaaaaugaccuaugaauugacagacaacgugacuaagucugucugucauuucuguaggccaauguucuguau

SEQ ID NO: 340 - RNA, 85 nt, Danio rerio
cuaggacacagggugaugaccuaugaauugacagccaguguuugcaguccagcugccugucaguucuguaggccacugcccuguu

SEQ ID NO: 341 - RNA, 82 nt, Fugu rubripes
ugggacgugaggugaugaccuaugaauugacagccaguaacuggagccucugccugucaguucuguaggccacugcuacguu

SEQ ID NO: 342 - RNA, 105 nt, Gallus gallus
ucaguaagaacugguguccaggaaaaugaccuaugaauugacagacugcuuucaaaaugugccugucauuucuauaggccaauauucugugcacuuuuccuacuu

SEQ ID NO: 343 - RNA, 110 nt, Gorilla gorilla
aucauucagaaaugguauacgggaaaaugaccuaugaauugacagacaauauagcugaguuugucugucauuucuuuagaccaauauucuguaugacugugcuacuucaa

SEQ ID NO: 344 - RNA, 110 nt, Macaca mulatta
aucauuaagaaaugguauacaggaaaaugaccuaugaauugacagacacuauagcugaguuugucugucauuucuuuaggccaauauucuguaugacugugcuacuucaa

SEQ ID NO: 345 - RNA, 89 nt, Mus musculus
cgugcacagggcucugaccuaugaauugacagccaguacucuuuucucuccucuggcugccaauuccauaggucacagguauguucacc

SEQ ID NO: 346 - RNA, 112 nt, Mus musculus
agcucucagcaucaacgguguacaggagaaugaccuaugauuugacagaccgugcagcuguguaugucugucauucuguaggccaauauucuguaugucacugcuacuuaaa

SEQ ID NO: 347 - RNA, 110 nt, Macaca nemestrina; identical to SEQ ID NO: 344

SEQ ID NO: 348 - RNA, 110 nt, Pongo pygmaeus
aucauucagaaaugguauacaggaaaaugaccuaugaauugacagacaauacagcugaguuugucugucauuucuuuaggccaauauucuguacaacugugcuacuucaa

SEQ ID NO: 349 - RNA, 110 nt, Pan troglodytes
aucauucagaaaugguauacgggaaaaugaccuaugaauugacagacaauauagcugaguuugucugucauuucuuuaggccaauauucuguaugacugugcuacuucaa

SEQ ID NO: 350 - RNA, 110 nt, Rattus norvegicus
gucaagauggagugcacagggcucugaccuaugaauugacagccaguacucugaucucgccucuggcugccaguuccauaggucacagguauguucgccucaaugccagc

SEQ ID NO: 351 - RNA, 97 nt, Rattus norvegicus
gguguacaggacaaugaccuaugauuugacagacaguguggcugcgugugucugucauucuguaggccaauauucuguaugucucuccuccuuacaa

SEQ ID NO: 352 - RNA, 83 nt, Tetraodon nigroviridis
cacgaggugaugaccuaugaauugacagccaguaacuggagccucugccugucaguucuguaggccacugcugcguccguccc

SEQ ID NO: 353 - RNA, 87 nt, Xenopus tropicalis
gaguguacgggccuaugaccuaugaauugacagccaguggaugugaagucugccugucaauucuguaggccacagguucguccaccu

SEQ ID NO: 354 - RNA, 97 nt, Xenopus tropicalis
aacugguaaccaggaggaugaccuaugaaaugacagccacuuccauaccaaacaugucugucauuucuguaggccaauauucugauugcuuuguuga

SEQ ID NO: 355 - RNA, 110 nt, Homo sapiens
gauggcugugaguuggcuuaaucucagcuggcaacugugagauguucauacaaucccucacaguggucucugggauuaugcuaaacagagcaauuuccuagcccucacga

SEQ ID NO: 356 - RNA, 89 nt, Danio rerio
gcugauuuuuggcauaaucucagcuggcaacugugaguaguguuuucaucccucucacaggcgcugcugggguucugucacacacagca

SEQ ID NO: 357 - RNA, 89 nt, Danio rerio; identical to SEQ ID NO: 356

SEQ ID NO: 358 - RNA, 77 nt, Danio rerio
acugacuggguaaucucugcaggcaacugugaugugauuacagucucacauugaccugaagagguugagcagucugu

SEQ ID NO: 359 - RNA, 76 nt, Danio rerio
cugacuggguaaucucugcaggcaacugugaugugauuacagucucacauugaccugaagagguugugcagucugu

SEQ ID NO: 360 - RNA, 73 nt, Fugu rubripes
uugguaaaaucucagcuggcaacugugagucguucacuagcugcucucacaauggccucugggauuaugcuaa

SEQ ID NO: 361 - RNA, 77 nt, Fugu rubripes
ugacuguuuaaucucugcaggcaacugugaugguguuuuauauucucacaaucaccuggagagauucugcaguuuau

SEQ ID NO: 362 - RNA, 106 nt, Gallus gallus
gauggcugugaauuggcuuaaucucagcuggcaacugugagcaguuaauaauucucacagugguaucugggauuaugcuaaacacagcaauuucuuugcucuaaug

SEQ ID NO: 363 - RNA, 110 nt, Gorilla gorilla; identical to SEQ ID NO: 355

SEQ ID NO: 364 - RNA, 110 nt, Lemur catta; identical to SEQ ID NO: 355

SEQ ID NO: 365 - RNA, 97 nt, Monodelphis domestica
gauggcugugaauuggcuuaaucucagcuggcaacugugagauguuaauaaauucccucacaguggucucugggauuaugcuaaacagagcaauuuc

SEQ ID NO: 366 - RNA, 72 nt, Mus musculus
uugguuuaaucucagcuggcaacugugagaugucccuaucauuccucacaguggucucugggauuaugcuaa

SEQ ID NO: 367 - RNA, 86 nt, Mus musculus
uuggcagacugggaaaucucugcaggcaaaugugaugucacugaagaaaccacacacuuaccuguagagauucuucagucugacaa

SEQ ID NO: 368 - RNA, 110 nt, Pan paniscus; identical to SEQ ID NO: 355

SEQ ID NO: 369 - RNA, 110 nt, Pongo pygmaeus
gauggcugugaguuggcuuaaucucagcuggcaacugugagauguucauacaaucccucacaguggucucugggauuaugcuaaacagagcaauuuccuugcccucacga

SEQ ID NO: 370 - RNA, 109 nt, Pan troglodytes
gauggcugugaguuggcuuaucucagcuggcaacugugagauguucauacaaucccucacaguggucucugggauuaaacuaaacagagcaauuuccuagcccucacga

SEQ ID NO: 371 - RNA, 106 nt, Rattus norvegicus
guuagcuaugaguuaguuuaaucucagcuggcaacugugagaugucccuaucauuccucacaguggucucugggauuaugcuaaacagagcaauuuccuugaccuc

SEQ ID NO: 372 - RNA, 105 nt, Sus scrofa
gauggcugugaguuggcuuaaucucagcuggcaacugugagauguucauacaaucccccacaguggucucugggauuaugcuaaacagagcaauuuccuugcccu

SEQ ID NO: 373 - RNA, 73 nt, Tetraodon nigroviridis
uuggugaaaucucagcuggcaacugugagucguucacuagcugcucucacaauggccucugggauuaugcuaa

SEQ ID NO: 374 - RNA, 77 nt, Tetraodon nigroviridis
ugacuguuuaaucucugcaggcaacugugauggugauuuuuauucucacaaucaccuggagagauucugcaguuuau

SEQ ID NO: 375 - RNA, 91 nt, Xenopus tropicalis
uggcugugaauuggcuuaaucucagcuggcaacugugagcaguuaauaaauuaucucacaguggucucugggauuauacuaaacacagcaa

SEQ ID NO: 376 - RNA, 94 nt, Homo sapiens
gaguuugguuuuguuuggguuuguucuagguauggucccagggaucccagaucaaaccaggccccugggccuauccuagaaccaaccuaagcuc

SEQ ID NO: 377 - RNA, 90 nt, Bos taurus
gaguuugguuuuguuuggguuuguucuagguauggucccagggaucccagaucaaaccaggccccugggccuauccuagaaccaaccuaa

SEQ ID NO: 378 - RNA, 96 nt, Mus musculus
gagucugguuuuguuuggguuuguucuagguauggucccagggaucccagaucaaaccaggccccugggccuauccuagaaccaaccuaaacccgu

SEQ ID NO: 379 - RNA, 96 nt, Rattus norvegicus
gagucuggucuuguuuggguuuguucuagguauggucccagggaucccagaucaaaccaggccccugggccuauccuagaaccaaccuaaacccau

SEQ ID NO: 380 - RNA, 82 nt, Mus musculus
cagccugugauacucaaacugggggcucuuuuggauuuucaucggaagaaaagugccgccagguuuugagugucaccgguug

SEQ ID NO: 381 - RNA, 67 nt, Homo sapiens
guggcacucaaacugugggggcacuuucugcucucuggugaaagugccgccaucuuuugaguguuac

SEQ ID NO: 382 - RNA, 67 nt, Homo sapiens
gugggccucaaauguggagcacuauucugauguccaaguggaaagugcugcgacauuugagcgucac

SEQ ID NO: 383 - RNA, 83 nt, Mus musculus
cucaucuugcgguacucaaacuaugggggcacuuuuuuuuuucuuuaaaaagugccgccuaguuuuaagccccgccgguugag

SEQ ID NO: 384 - RNA, 82 nt, Mus musculus
ccuauguagcggccaucaaaguggaggcccucucuugagccugaaugagaaagugcuuccacuuugugugccacugcauggg

SEQ ID NO: 385 - RNA, 79 nt, Mus musculus
acauacagugucgaucaaaguggaggcccucuccgcggcuuggcgggaaagugcauccauuuuguuugucucugugugu

SEQ ID NO: 386 - RNA, 80 nt, Mus musculus
uucaaucugugguacucaaacugugugacauuuuguucuuuguaagaagugccgcagaguuuguaguguugccgauugag

SEQ ID NO: 387 - RNA, 84 nt, Mus musculus
uuccauauagccauacucaaaauggaggcccuaucuaagcuuuuaaguggaaagugcuucccuuuuguguguugccauguggag

SEQ ID NO: 388 - RNA, 69 nt, Mus musculus
ggugagacucaaauguggggcacacuucuggacuguacauagaaagugcuacuacuuuugagucucucc

SEQ ID NO: 389 - RNA, 81 nt, Rattus norvegicus
ucaucuugcgguucucaaacuaugggggcacuuuuuuuuucuuuaaaaagugccgccagguuuuagggccugccgguugag

SEQ ID NO: 390 - RNA, 82 nt, Rattus norvegicus
ccgguguaguagccaucaaaguggaggcccucucuugggcccgagcuagaaagugcuuccacuuugugugccacugcauggg

SEQ ID NO: 391 - RNA, 82 nt, Rattus norvegicus
caaccugugauacucaaacugggggcucuuuuggguuuucuuuggaagaaaagugccgccagguuuugaguguuaccgauug

(19) United States
(12) Patent Application Publication
(10) Pub. No.: US 2009/0131356 A1
(43) Pub. Date: May 21, 2009

(54) miR-15, miR-26, miR-31, miR-145, miR-147, miR-188, miR-215, miR-216, miR-331, mmu-miR-292-3P REGULATED GENES AND PATHWAYS AS TARGETS FOR THERAPEUTIC INTERVENTION

(75) Inventors: Andreas G. Bader, Austin, TX (US); Mike W. Byrom, Austin, TX (US); Lubna Patrawala, Austin, TX (US); Charles D. Johnson, Austin, TX (US); David Brown, Austin, TX (US)

Correspondence Address: Fullbright & Jaworski L.L.P., 600 Congress Avenue, Suite 2400, Austin, TX, 78701, US

(73) Assignee: ASURAGEN, INC., Austin, TX (US)

(21) Appl. No.: 12/167,492

(22) Filed: Jul. 3, 2008

Related U.S. Application Data
(63) Continuation-in-part of application No. PCT/US2007/078952, filed on Sep. 19, 2007.
(60) Provisional application No. 60/948,350, filed on Jul. 6, 2007.
(60) Provisional application No. 60/826,173, filed on Sep. 19, 2006.

(51) Int. Cl. A61K 31/7105; C12N 5/06; C12Q 1/68

(52) U.S. Cl. 514/44; 435/375; 435/6

(57) ABSTRACT

The present invention concerns methods and compositions for identifying genes or genetic pathways modulated by miR-15, miR-26, miR-31, miR-145, miR-147, miR-188, miR-215, miR-216, miR-331, mmu-miR-292-3p, and using nucleic acid comprising all or part of the miR-15, miR-26, miR-31, miR-145, miR-147, miR-188, miR-215, miR-216, miR-331, mmu-miR-292-3p sequences to modulate a gene or gene pathway, using this profile in assessing the condition of a patient and/or treating the patient with an appropriate miRNA.

This application claims the benefit of priority to U.S. Provisional Patent Application Ser. No. 60/948,350 filed Jul. 6, 2007; U.S. Provisional Patent Application Ser. No. 60/826,173 filed Sep. 19, 2006; International Application PCT/US2007/078952 filed Sep. 19, 2007; all of which are hereby incorporated by reference in their entirety.

### BACKGROUND OF THE INVENTION

I. Field of the Invention

The present invention relates to the fields of molecular biology and medicine. More specifically, the invention relates to methods and compositions for the treatment of diseases or conditions that are affected by microRNA (miRNA) miR-15, miR-26, miR-31, miR-145, miR-147, miR-188, miR-215, miR-216, miR-331, or mmu-miR-292-3p expression or lack thereof, and genes and cellular pathways directly and indirectly modulated by such.

II. Background

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are distinct.

miRNAs thus far observed have been approximately 21-22 nucleotides in length, and they arise from longer precursors, which are transcribed from non-protein-encoding genes. See review of Carrington and Ambros (2003). The precursors form structures that fold back on themselves in self-complementary regions; they are then processed by the nuclease Dicer (in animals) or DCL1 (in plants) to generate the short double-stranded miRNA. One of the miRNA strands is incorporated into a complex of proteins and miRNA called the RNA-induced silencing complex (RISC). The miRNA guides the RISC complex to a target mRNA, which is then cleaved or translationally silenced, depending on the degree of sequence complementarity of the miRNA to its target mRNA. Currently, it is believed that perfect or nearly perfect complementarity leads to mRNA degradation, as is most commonly observed in plants. In contrast, imperfect base pairing, as is primarily found in animals, leads to translational silencing. However, recent data suggest additional complexity (Bagga et al., 2005; Lim et al., 2005), and mechanisms of gene silencing by miRNAs remain under intense study.

Recent studies have shown that changes in the expression levels of numerous miRNAs are associated with various cancers (reviewed in Esquela-Kerscher and Slack, 2006; Calin and Croce, 2006). miRNAs have also been implicated in regulating cell growth and cell and tissue differentiation—cellular processes that are associated with the development of cancer.

The inventors previously demonstrated that the microRNAs described in this application are involved with the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005, each of which is incorporated herein by reference in its entirety). For example, cell proliferation, cell division, and cell survival are frequently altered in human cancers. Overexpression of hsa-miR-147, -215 or mmu-miR-292-3p decreases the proliferation and/or viability of certain normal or cancerous cell lines. Overexpression of hsa-miR-216 increases the proliferation of normal skin and lung cancer cells. Overexpression of hsa-miR-15a, -26a, -145, -188 or -331 can inhibit or stimulate proliferation or viability of certain normal or cancerous cell lines, depending on the individual cell type. Similarly, the inventors previously observed that miRNA inhibitors of hsa-miR-215, -216, and -331 reduce proliferation of certain cell lines, and miRNA inhibitors of hsa-miR-15a increase proliferation of skin basal cell carcinoma cells. Apoptosis, programmed cell death, is frequently disrupted in cancers. Insufficient apoptosis results in uncontrolled cell proliferation, a hallmark of cancer. The inventors observed that overexpression of hsa-miR-31, -15a, -147, -215, -331 increase apoptosis; overexpression of hsa-miR-145, hsa-miR-216, or mmu-miR-292-3p decrease apoptosis in various cancer cell lines. Overexpression of hsa-miR-26a or -188 induces or suppresses apoptosis, depending on the cell type.

More than 90% of human cancer samples have active telomerase (Dong et al., 2005); whereas most terminally-differentiated cells lack telomerase. The hTert gene encodes the catalytic domain of telomerase. The inventors previously observed that hsa-miR-15a, hsa-26a, and hsa-147 activate the hTert gene in normal human fibroblasts. Such activity might contribute to cancer by activating telomerase.

These data suggest that expression or lack of expression of a specific miRNA in certain cells could likely contribute to cancer and other diseases. The inventors have also previously observed associations between miRNA expression and certain human cancers. For example, hsa-miR-145, -188, and -331 are expressed at significantly lower levels in the tumors of most lung cancer patients than in lung tissues from patients without disease. Hsa-mir-145 and -331 are also expressed at lower levels in colon tumors, but hsa-miR-31 is expressed at higher levels in colon tumors than in normal colon tissues. Hsa-mir-15a is expressed at higher levels in cancerous breast, prostate, and thyroid tissues than in corresponding normal tissues. Hsa-miR-145 is expressed at lower levels in colon, breast, and bladder cancers than in corresponding normal tissues. microRNAs described in this application were also previously observed by the inventors to be differentially expressed in tissues from patients with prion disease, lupus, multiple sclerosis, or Alzheimer's disease.

Bioinformatics analyses suggest that any given miRNA may bind to and alter the expression of up to several hundred different genes. In addition, a single gene may be regulated by several miRNAs. Thus, each miRNA may regulate a complex interaction among genes, gene pathways, and gene networks. Mis-regulation or alteration of these regulatory pathways and networks, involving miRNAs, are likely to contribute to the development of disorders and diseases such as cancer. Although bioinformatics tools are helpful in predicting miRNA binding targets, all have limitations. Because of the imperfect complementarity with their target binding sites, it is difficult to accurately predict the mRNA targets of miRNAs with bioinformatics tools alone. Furthermore, the complicated interactive regulatory networks among miRNAs and target genes make it difficult to accurately predict which genes will actually be mis-regulated in response to a given miRNA.

Correcting gene expression errors by manipulating miRNA expression or by repairing miRNA mis-regulation represent promising methods to repair genetic disorders and cure diseases like cancer. A current, disabling limitation of this approach is that, as mentioned above, the details of the regulatory pathways and gene networks that are affected by any given miRNA, have been largely unknown. This represents a significant limitation for treatment of cancers in which a specific miRNA may play a role. A need exists to identify the genes, genetic pathways, and genetic networks that are regulated by or that may regulate expression of miRNAs.

### SUMMARY OF THE INVENTION

The present invention provides additional compositions and methods by identifying genes that are direct targets for miR-15, miR-26, miR-31, miR-145, miR-147, miR-188, miR-215, miR-216, miR-331, or mmu-miR-292-3p regulation or that are indirect or downstream targets of regulation following the miR-15-, miR-26-, miR-31-, miR-145-, miR-147-, miR-188-, miR-25-, miR-26-, miR-331-, or mmu-miR-292-3p-mediated modification of another gene(s) expression. Furthermore, the invention describes genes, diseases, and/or physiologic pathways and networks that are influenced by miR-15, miR-26, miR-31, miR-145, miR-147, miR-188, miR-215, miR-216, miR-331, or mmu-miR-292-3p and their family members. In certain aspects, compositions of the invention are administered to a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

In particular aspects, a subject or patient may be selected for treatment based on expression and/or aberrant expression of one or more miRNA or mRNA. In a further aspect, a subject or patient may be selected for treatment based on aberrations in one or more biologic or physiologic pathway(s), including aberrant expression of one or more gene associated with a pathway, or the aberrant expression of one or more protein encoded by one or more gene associated with a pathway. In still a further aspect, a subject or patient may be selected based on aberrations in miRNA expression, or biologic and/or physiologic pathway(s). A subject may be assessed for sensitivity, resistance, and/or efficacy of a therapy or treatment regime based on the evaluation and/or analysis of miRNA or mRNA expression or lack thereof. A subject may be evaluated for amenability to certain therapy prior to, during, or after administration of one or therapy to a subject or patient. Typically, evaluation or assessment may be done by analysis of miRNA and/or mRNA, as well as combination of other assessment methods that include but are not limited to histology, immunohistochemistry, blood work, etc.

In some embodiments, an infectious disease or condition includes a bacterial, viral, parasite, or fungal infection. Many of these genes and pathways are associated with various cancers and other diseases. Cancerous conditions include, but are not limited to astrocytoma, acute myeloid leukemia, anaplastic large cell lymphoma, acute lymphoblastic leukemia, angiosarcoma, B-cell lymphoma, Burkitt's lymphoma, breast carcinoma, bladder carcinoma, carcinoma of the head and neck, cervical carcinoma, chronic lymphoblastic leukemia, chronic myeloid leukemia, colorectal carcinoma, endometrial carcinoma, esophageal squamous cell carcinoma, Ewing's sarcoma, fibrosarcoma, glioma, glioblastoma, gastric carcinoma, hepatoblastoma, hepatocellular carcinoma, Kaposi's sarcoma, Hodgkin lymphoma, laryngeal squamous cell carcinoma, larynx carcinoma, leukemia, leiomyosarcoma, lipoma, liposarcoma, melanoma, mantle cell lymphoma, medulloblastoma, mesothelioma, myxofibrosarcoma, myeloid leukemia, myeloma, mucosa-associated lymphoid tissue B cell lymphoma, multiple myeloma, nasopharyngeal carcinoma, neuroblastoma, neurofibroma, high-grade non-Hodgkin lymphoma, non-Hodgkin lymphoma, lung carcinoma, non-small cell lung carcinoma, ovarian carcinoma, oesophageal carcinoma, oligodendroglioma, osteosarcoma, pancreatic carcinoma, pheochromocytoma, prostate carcinoma, renal cell carcinoma, retinoblastoma, rhabdomyosarcoma, salivary gland tumor, Schwanomma, small cell lung cancer, squamous cell carcinoma of the head and neck, testicular tumor, thyroid carcinoma, urothelial carcinoma, and Wilm's tumor, wherein the modulation of one or more gene is sufficient for a therapeutic response. Typically a cancerous condition is an aberrant hyperproliferative condition associated with the uncontrolled growth or inability to undergo cell death, including apoptosis.

The present invention provides methods and compositions for identifying genes that are direct targets for miR-15, miR-26, miR-31, miR-145, miR-147, miR-188, miR-215, miR-216, miR-331, or mmu-miR-292-3p regulation or that are downstream targets of regulation following the miR-15-, miR-26-, miR-31-, miR-145-, miR-147-, miR-188-, miR-25-, miR-26-, miR-331-, or mmu-miR-292-3p-mediated modification of upstream gene expression. Furthermore, the invention describes gene pathways and networks that are influenced by miR-15, miR-26, miR-31, miR-145, miR-147, miR-188, miR-215, miR-216, miR-331, or mmu-miR-292-3p expression. Many of these genes and pathways are associated with various cancers and other diseases. The altered expression or function of miR-15, miR-26, miR-31, miR-145, miR-147, miR-188, miR-215, miR-216, miR-331, or mmu-miR-292-3p in cells would lead to changes in the expression of these key genes and contribute to the development of disease or other conditions. Introducing miR-15, miR-26, miR-31, miR-145, miR-147, miR-188, miR-215, miR-216, miR-331, or mmu-miR-292-3p (for diseases where the miRNA is down-regulated) or a miR-15, miR-26, miR-31, miR-145, miR-147, miR-188, miR-215, miR-216, miR-331, or mmu-miR-292-3p inhibitor (for diseases where the miRNA is up-regulated) into diseased or abnormal cells or tissues or subjects would result in a therapeutic response. The identities of key genes that are regulated directly or indirectly by miR-15, miR-26, miR-31, miR-145, miR-147, miR-188, miR-215, miR-216, miR-331, or mmu-miR-292-3p and the disease with which they are associated are provided herein. In certain aspects a cell may be an epithelial, an endothelial, a mesothelial, a glial, a stromal, or a mucosal cell. The cell can be, but is not limited to a brain, a neuronal, a blood, an endometrial, an oligodendrocyte, a meninges, an esophageal, a lung, a cardiovascular, a leukemic, a liver, a lymphoid, a breast, a bone, a connective tissue, a fat, a retinal, a thyroid, a glandular, an adrenal, a pancreatic, a stomach, an intestinal, a kidney, a bladder, a colon, a prostate, a uterine, an ovarian, a cervical, a testicular, a splenic, a skin, a smooth muscle, a cardiac muscle, or a striated muscle cell.

In certain aspects, the cell, tissue, or target may not be defective in miRNA expression yet may still respond therapeutically to expression or over expression of a miRNA. miR-15, miR-26, miR-31, miR-145, miR-147, miR-188, miR-215, miR-216, miR-331, or mmu-miR-292-3p could be used as a therapeutic target for any of these diseases. In certain embodiments miR-15, miR-26, miR-31, miR-145, miR-147, miR-188, miR-215, miR-216, miR-331, or mmu-miR-292-3p can be used to modulate the activity of miR-15, miR-26, miR-31, miR-145, miR-147, miR-188, miR-215, miR-216, miR-331, or mmu-miR-292-3p in a subject, organ, tissue, or cell. A cell, tissue, or subject may be a cancer cell, a cancerous tissue, harbor cancerous tissue, or be a subject or patient diagnosed or at risk of developing a disease or condition. In certain aspects a cell may be an epithelial, an endothelial, a mesothelial, a glial, a stromal, or a mucosal cell. The cell can be, but is not limited to a brain, a neuronal, a blood, an endometrial, an oligodendrocyte, a meninges, an esophageal, a lung, a cardiovascular, a liver, a lymphoid, a breast, a bone, a connective tissue, a fat, a retinal, a thyroid, a glandular, an adrenal, a pancreatic, a stomach, an intestinal, a kidney, a bladder, a colon, a prostate, a uterine, an ovarian, a cervical, a testicular, a splenic, a skin, a smooth muscle, a cardiac muscle, or a striated muscle cell. In still a further aspect cancer includes, but is not limited to astrocytoma, acute myeloid leukemia, anaplastic large cell lymphoma, acute lymphoblastic leukemia, angiosarcoma, B-cell lymphoma, Burkitt's lymphoma, breast carcinoma, bladder carcinoma, carcinoma of the head and neck, cervical carcinoma, chronic lymphoblastic leukemia, chronic myeloid leukemia, colorectal carcinoma, endometrial carcinoma, esophageal squamous cell carcinoma, Ewing's sarcoma, fibrosarcoma, glioma, glioblastoma, gastric carcinoma, hepatoblastoma, hepatocellular carcinoma, Kaposi's sarcoma, Hodgkin lymphoma, laryngeal squamous cell carcinoma, larynx carcinoma, leukemia, leiomyosarcoma, lipoma, liposarcoma, melanoma, mantle cell lymphoma, medulloblastoma, mesothelioma, myxofibrosarcoma, myeloid leukemia, mucosa-associated lymphoid tissue B cell lymphoma, multiple myeloma, myeloma, nasopharyngeal carcinoma, neuroblastoma, neurofibroma, high-grade non-Hodgkin lymphoma, non-Hodgkin lymphoma, lung carcinoma, non-small cell lung carcinoma, ovarian carcinoma, oesophageal carcinoma, oligodendroglioma, osteosarcoma, pancreatic carcinoma, pheochromocytoma, prostate carcinoma, renal cell carcinoma, retinoblastoma, rhabdomyosarcoma, salivary gland tumor, Schwanomma, small cell lung cancer, squamous cell carcinoma of the head and neck, testicular tumor, thyroid carcinoma, urothelial carcinoma, and Wilm's tumor.

Embodiments of the invention include methods of modulating gene expression, or biologic or physiologic pathways in a cell, a tissue, or a subject comprising administering to the cell, tissue, or subject an amount of an isolated nucleic acid or mimetic thereof comprising a miR-15, miR-26, miR-31, miR-145, miR-147, miR-188, miR-215, miR-216, miR-331, or mmu-miR-292-3p nucleic acid, mimetic, or inhibitor sequence in an amount sufficient to modulate the expression of a gene positively or negatively modulated by a miR-15, miR-26, miR-31, miR-145, miR-147, miR-188, miR-215, miR-216, miR-331, or mmu-miR-292-3p miRNA. A “miR-15, miR-26, miR-31, miR-145, miR-147, miR-188, miR-215, miR-216, miR-331, or mmu-miR-292-3p nucleic acid sequence” or “miR-15, miR-26, miR-31, miR-145, miR-147, miR-188, miR-215, miR-216, miR-331, or mmu-miR-292-3p inhibitor” includes the full length precursor of miR-15, miR-26, miR-31, miR-145, miR-147, miR-188, miR-215, miR-216, miR-331, or mmu-miR-292-3p, or complement thereof or processed (i.e., mature) sequence of miR-15, miR-26, miR-31, miR-145, miR-147, miR-188, miR-215, miR-216, miR-331, or mmu-miR-292-3p and related sequences set forth herein, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the miR-15, miR-26, miR-31, miR-145, miR-147, miR-188, miR-215, miR-216, miR-331, or mmu-miR-292-3p nucleic acid sequence or miR-15, miR-26, miR-31, miR-145, miR-147, miR-188, miR-215, miR-216, miR-331, or mmu-miR-292-3p inhibitor contains the full-length processed miRNA sequence or complement thereof and is referred to as the “miR-15, miR-26, miR-31, miR-145, miR-147, miR-188, miR-215, miR-216, miR-331, or mmu-miR-292-3p full-length processed nucleic acid sequence” or “miR-15, miR-26, miR-31, miR-145, miR-147, miR-188, miR-215, miR-216, miR-331, or mmu-miR-292-3p full-length processed inhibitor sequence.” In still further aspects, the miR-15, miR-26, miR-31, miR-145, miR-147, miR-188, miR-215, miR-216, miR-331, or mmu-miR-292-3p nucleic acid comprises at least 5, 6, 7, 8, 9, 10, 11, 12, 13, 14, 15, 16, 17, 18, 19, 20, 21, 22, 23, 24, 25, 50 nucleotide (including all ranges and integers there between) segment or complementary segment of a miR-15, miR-26, miR-31, miR-145, miR-147, miR-188, miR-215, miR-216, miR-331, or mmu-miR-292-3p that is at least 75, 80, 85, 90, 95, 98, 99 or 100% identical to SEQ ID NO:1 to SEQ ID NO:391. The general terms miR-15, miR-26, miR-31, miR-145, miR-147, miR-188, miR-215, miR-216, miR-331, or mmu-miR-292-3p includes all members of the miR-15, miR-26, miR-31, miR-145, miR-147, miR-188, miR-215, miR-216, miR-331, or mmu-miR-292-3p family that share at least part of a mature miRNA sequence.

Mature miR-15 sequences include: hsa-miR-15a, UAGCAGCACAUAAUGGUUUGUG, MIMAT0000068, SEQ ID NO:1); hsa-miR-15b, UAGCAGCACAUCAUGGUUUACA (MIMAT0000417, SEQ ID NO:2); hsa-miR-16, UAGCAGCACGUAAAUAUUGGCG (MIMAT0000069, SEQ ID NO:3); hsa-miR-195, UAGCAGCACAGAAAUAUUGGC (MIMAT0000461, SEQ ID NO:4); age-miR-15a, UAGCAGCACAUAAUGGUUUGUG (MIMAT0002638, SEQ ID NO:5); age-miR-15b, UAGCAGCACAUCAUGGUUUACA (MIMAT0002203, SEQ ID NO:6); age-miR-16, UAGCAGCACGUAAAUAUUGGCG (MIMAT0002639, SEQ ID NO:7); bta-miR-15b, UAGCAGCACAUCAUGGUUUACA (MIMAT0003792, SEQ ID NO:8); bta-miR-16, UAGCAGCACGUAAAUAUUGGC (MIMAT0003525, SEQ ID NO:9); dre-miR-15a, UAGCAGCACAGAAUGGUUUGUG (MIMAT0001772, SEQ ID NO:10); dre-miR-15a*, CAGGCCGUACUGUGCUGCGGCA (MIMAT0003395, SEQ ID NO:11); dre-miR-15b, UAGCAGCACAUCAUGGUUUGUA (MIMAT0001773, SEQ ID NO:12); dre-miR-15c, AAGCAGCGCGUCAUGGUUUUC (MIMAT0003764, SEQ ID NO:13); dre-miR-16a, UAGCAGCACGUAAAUAUUGGUG (MIMAT0001774, SEQ ID NO:14); dre-miR-16b, UAGCAGCACGUAAAUAUUGGAG (MIMAT0001775, SEQ ID NO:15); dre-miR-16c, UAGCAGCAUGUAAAUAUUGGAG (MIMAT0001776, SEQ ID NO:16); dre-miR-457a, AAGCAGCACAUCAAUAUUGGCA (MIMAT0001883, SEQ ID NO:17); dre-miR-457b, AAGCAGCACAUAAAUACUGGAG (MIMAT0001884, SEQ ID NO:18); fru-miR-15a, UAGCAGCACGGAAUGGUUUGUG (MIMAT0003105, SEQ ID NO:19); fru-miR-15b, UAGCAGCGCAUCAUGGUUUGUA (MIMAT0003085, SEQ ID NO:20); fru-miR-16, UAGCAGCACGUAAAUAUUGGAG (MIMAT0003107, SEQ ID NO:21); gga-miR-15a, UAGCAGCACAUAAUGGUUUGU (MIMAT0001117, SEQ ID NO:22); gga-miR-15b, UAGCAGCACAUCAUGGUUUGCA (MIMAT0001154, SEQ ID NO:23); gga-miR-16, UAGCAGCACGUAAAUAUUGGUG (MIMAT0001116, SEQ ID NO:24); ggo-miR-15a, UAGCAGCACAUAAUGGUUUGUG (MIMAT0002640, SEQ ID NO:25); ggo-miR-15b, UAGCAGCACAUCAUGGUUUACA (MIMAT0002202, SEQ ID NO:26); ggo-miR-16, UAGCAGCACGUAAAUAUUGGCG (MIMAT0002641, SEQ ID NO:27); ggo-miR-195, UAGCAGCACAGAAAUAUUGGC (MIMAT0002316, SEQ ID NO:28); lca-miR-15a, UAGCAGCACAUAAUGGUUUGUG (MIMAT0002648, SEQ ID NO:29); lca-miR-16, UAGCAGCACGUAAAUAUUGGUG (MIMAT0002649, SEQ ID NO:30); lla-miR-15a, UAGCAGCACAUAAUGGUUUGUG (MIMAT0002656, SEQ ID NO:31); lla-miR-15b, UAGCAGCACAUCAUGGUUUACA (MIMAT0002208, SEQ ID NO:32); lla-miR-16, UAGCAGCACGUAAAUAUUGGCG (MIMAT0002657, SEQ ID NO:33); mdo-miR-15a, UAGCAGCACAUAAUGGUUUGUU (MIMAT0004144, SEQ ID NO:34); mdo-miR-16, UAGCAGCACGUAAAUAUUGGCG (MIMAT0004145, SEQ ID NO:35); mml-miR-15a, UAGCAGCACAUAAUGGUUUGUG (MIMAT0002650, SEQ ID NO:36); mml-miR-15b, UAGCAGCACAUCAUGGUUUACA (MIMAT0002207, SEQ ID NO:37); mml-miR-16, UAGCAGCACGUAAAUAUUGGCG (MIMAT0002651, SEQ ID NO:38); mmu-miR-15a, UAGCAGCACAUAAUGGUUUGUG (MIMAT0000526, SEQ ID NO:39); mmu-miR-15b, UAGCAGCACAUCAUGGUUUACA (MIMAT0000124, SEQ ID NO:40); mmu-miR-16, UAGCAGCACGUAAAUAUUGGCG (MIMAT0000527, SEQ ID NO:41); mmu-miR-195, UAGCAGCACAGAAAUAUUGGC (MIMAT0000225, SEQ ID NO:42); mne-miR-15a, UAGCAGCACAUAAUGGUUUGUG (MIMAT0002642, SEQ ID NO:43); mne-miR-15b, UAGCAGCACAUCAUGGUUUACA (MIMAT0002209, SEQ ID NO:44); mne-miR-16, UAGCAGCACGUAAAUAUUGGCG (MIMAT0002643, SEQ ID NO:45); ppa-miR-15a, UAGCAGCACAUAAUGGUUUGUG (MIMAT0002646, SEQ ID NO:46); ppa-miR-15b, UAGCAGCACAUCAUGGUUUACA (MIMAT0002204, SEQ ID NO:47); ppa-miR-16, UAGCAGCACGUAAAUAUUGGCG (MIMAT0002647, SEQ ID NO:48); ppa-miR-195, UAGCAGCACAGAAAUAUUGGC (MIMAT0002317, SEQ ID NO:49); ppy-miR-15a, UAGCAGCACAUAAUGGUUUGUG (MIMAT0002652, SEQ ID NO:50); ppy-miR-15b, UAGCAGCACAUCAUGGUUUACA (MIMAT0002205, SEQ ID NO:51); ppy-miR-16, UAGCAGCACGUAAAUAUUGGCG (MIMAT0002653, SEQ ID NO:52); ptr-miR-15a, UAGCAGCACAUAAUGGUUUGUG (MIMAT0002654, SEQ ID NO:53); ptr-miR-15b, UAGCAGCACAUCAUGGUUUACA (MIMAT0002206, SEQ ID NO:54); ptr-miR-16, UAGCAGCACGUAAAUAUUGGCG (MIMAT0002655, SEQ ID NO:55); rno-miR-15b, UAGCAGCACAUCAUGGUUUACA (MIMAT0000784, SEQ ID NO:56); rno-miR-16, UAGCAGCACGUAAAUAUUGGCG (MIMAT0000785, SEQ ID NO:57); rno-miR-195, UAGCAGCACAGAAAUAUUGGC (MIMAT0000870, SEQ ID NO:58); sla-miR-15a, UAGCAGCACAUAAUGGUUUGUG (MIMAT0002644, SEQ ID NO:59); sla-miR-16, UAGCAGCACGUAAAUAUUGGCG (MIMAT0002645, SEQ ID NO:60); ssc-miR-15b, CCGCAGCACAUCAUGGUUUACA (MIMAT0002125, SEQ ID NO:61); tni-miR-15a, UAGCAGCACGGAAUGGUUUGUG (MIMAT0003106, SEQ ID NO:62); tni-miR-15b, UAGCAGCGCAUCAUGGUUUGUA (MIMAT0003086, SEQ ID NO:63); tni-miR-16, UAGCAGCACGUAAAUAUUGGAG (MIMAT0003108, SEQ ID NO:64); xtr-miR-15a, UAGCAGCACAUAAUGGUUUGUG (MIMAT0003560, SEQ ID NO:65); xtr-miR-15b, UAGCAGCACAUCAUGAUUUGCA (MIMAT0003561, SEQ ID NO:66); xtr-miR-15c, UAGCAGCACAUCAUGGUUUGUA (MIMAT0003651, SEQ ID NO:67); xtr-miR-16a, UAGCAGCACGUAAAUAUUGGUG (MIMAT0003563, SEQ ID NO:68); xtr-miR-16b, UAGCAGCACGUAAAUAUUGGGU (MIMAT0003668, SEQ ID NO:69); xtr-miR-16c, UAGCAGCACGUAAAUACUGGAG (MIMAT0003562, SEQ ID NO:70); or a complement thereof.

Mature miR-26 sequences include: hsa-miR-26a, UUCAAGUAAUCCAGGAUAGGC (MIMAT0000082, SEQ ID NO:71); hsa-miR-26b, UUCAAGUAAUUCAGGAUAGGUU (MIMAT0000083, SEQ ID NO:72); bta-miR-26a, UUCAAGUAAUCCAGGAUAGGCU (MIMAT0003516, SEQ ID NO:73); bta-miR-26b, UUCAAGUAAUUCAGGAUAGGUU (MIMAT0003531, SEQ ID NO:74); dre-miR-26a, UUCAAGUAAUCCAGGAUAGGCU (MIMAT0001794, SEQ ID NO:75); dre-miR-26b, UUCAAGUAAUCCAGGAUAGGUU (MIMAT0001795, SEQ ID NO:76); fru-miR-26, UUCAAGUAAUCCAGGAUAGGCU (MIMAT0003037, SEQ ID NO:77); gga-miR-26a, UUCAAGUAAUCCAGGAUAGGC (MIMAT0001118, SEQ ID NO:78); ggo-miR-26a, UUCAAGUAAUCCAGGAUAGGCU (MIMAT0002345, SEQ ID NO:79); lla-miR-26a, UUCAAGUAAUCCAGGAUAGGCU (MIMAT0002347, SEQ ID NO:80); mml-miR-26a, UUCAAGUAAUCCAGGAUAGGCU (MIMAT0002349, SEQ ID NO:81); mmu-miR-26a, UUCAAGUAAUCCAGGAUAGGC (MIMAT0000533, SEQ ID NO:82); mmu-miR-26b, UUCAAGUAAUUCAGGAUAGGUU (MIMAT0000534, SEQ ID NO:83); mne-miR-26a, UUCAAGUAAUCCAGGAUAGGCU (MIMAT0002348, SEQ ID NO:84); ppa-miR-26a, UUCAAGUAAUCCAGGAUAGGCU (MIMAT0002350, SEQ ID NO:85); ppy-miR-26a, UUCAAGUAAUCCAGGAUAGGCU (MIMAT0002346, SEQ ID NO:86); ptr-miR-26a, UUCAAGUAAUCCAGGAUAGGCU (MIMAT0002344, SEQ ID NO:87); rno-miR-26a, UUCAAGUAAUCCAGGAUAGGC (MIMAT0000796, SEQ ID NO:88); rno-miR-26b, UUCAAGUAAUUCAGGAUAGGUU (MIMAT0000797, SEQ ID NO:89); ssc-miR-26a, UUCAAGUAAUCCAGGAUAGGCU (MIMAT0002135, SEQ ID NO:90); tni-miR-26, UUCAAGUAAUCCAGGAUAGGCU (MIMAT0003038, SEQ ID NO:91); xtr-miR-26, UUCAAGUAAUCCAGGAUAGGC (MIMAT0003569, SEQ ID NO:92), or a complement thereof.

Mature miR-31 sequences include: hsa-miR-31, GGCAAGAUGCUGGCAUAGCUG, (MIMAT0000089, SEQ ID NO:93); bmo-miR-31, GGCAAGAAGUCGGCAUAGCUG, (MIMAT0004213, SEQ ID NO:94); bta-miR-31, AGGCAAGAUGCUGGCAUAGCU, (MIMAT0003548, SEQ ID NO:95); dme-miR-31a, UGGCAAGAUGUCGGCAUAGCUGA, (MIMAT0000400, SEQ ID NO:96); dme-miR-31b, UGGCAAGAUGUCGGAAUAGCUG, (MIMAT0000389, SEQ ID NO:97); dps-miR-31a, UGGCAAGAUGUCGGCAUAGCUGA, (MIMAT0001220, SEQ ID NO:98); dps-miR-31b, UGGCAAGAUGUCGGAAUAGCUGA, (MIMAT0001221, SEQ ID NO:99); dre-miR-31, GGCAAGAUGUUGGCAUAGCUG, (MIMAT0003347, SEQ ID NO:100); gga-miR-31, AGGCAAGAUGUUGGCAUAGCUG, (MIMAT0001189, SEQ ID NO:101); ggo-miR-31, GGCAAGAUGCUGGCAUAGCUG, (MIMAT0002381, SEQ ID NO:102); mdo-miR-31, GGAGGCAAGAUGUUGGCAUAGCUG, (MIMAT0004094, SEQ ID NO:103); mml-miR-31, GGCAAGAUGCUGGCAUAGCUG, (MIMAT0002379, SEQ ID NO:104); mmu-miR-31, AGGCAAGAUGCUGGCAUAGCUG, (MIMAT0000538, SEQ ID NO:105); mne-miR-31, GGCAAGAUGCUGGCAUAGCUG, (MIMAT0002383, SEQ ID NO:106); ppa-miR-31, GGCAAGAUGCUGGCAUAGCUG, (MIMAT0002384, SEQ ID NO:107); ppy-miR-31, GGCAAGAUGCUGGCAUAGCUG, (MIMAT0002382, SEQ ID NO:108); ptr-miR-31, GGCAAGAUGCUGGCAUAGCUG, (MIMAT0002380, SEQ ID NO:109); rno-miR-31, AGGCAAGAUGCUGGCAUAGCUG, (MIMAT0000810, SEQ ID NO:110); sme-miR-31b, AGGCAAGAUGCUGGCAUAGCUGA, (MIMAT0003980, SEQ ID NO: 111); xtr-miR-31, AGGCAAGAUGUUGGCAUAGCUG, (MIMAT0003679, SEQ ID NO: 112) or a complement thereof.

Mature miR-145 sequences include: hsa-miR-145 GUCCAGUUUUCCCAGGAAUCCCUU (MIMAT0000437, SEQ ID NO:113), or a complement thereof.

Mature miR-147 sequences include: hsa-miR-147 GUGUGUGGAAAUGCUUCUGC (MIMAT0000251, SEQ ID NO:114), or a complement thereof.

Mature miR-188 sequences include: hsa-miR-188, CAUCCCUUGCAUGGUGGAGGGU (MIMAT0000457, SEQ ID NO:115); hsa-miR-532, CAUGCCUUGAGUGUAGGACCGU (MIMAT0002888, SEQ ID NO:116); bta-miR-532, CAUGCCUUGAGUGUAGGACCGU (MIMAT0003848, SEQ ID NO:117); hsa-miR-660, UACCCAUUGCAUAUCGGAGUUG (MIMAT0003338, SEQ ID NO:118); mml-miR-188, CAUCCCUUGCAUGGUGGAGGGU (MIMAT0002307, SEQ ID NO:119); mmu-miR-188, CAUCCCUUGCAUGGUGGAGGGU (MIMAT0000217, SEQ ID NO:120); mmu-miR-532, CAUGCCUUGAGUGUAGGACCGU (MIMAT0002889, SEQ ID NO:121); mne-miR-188, CAUCCCUUGCAUGGUGGAGGGU (MIMAT0002310, SEQ ID NO:122); ppa-miR-188, CAUCCCUUGCAUGGUGGAGGGU (MIMAT0002311, SEQ ID NO:123); ppy-miR-188, CAUCCCUUGCAUGGUGGAGGGU (MIMAT0002309, SEQ ID NO:124); or ptr-miR-188, CAUCCCUUGCAUGGUGGAGGGU (MIMAT0002308, SEQ ID NO: 125), or a complement thereof.

Mature miR-215 sequences include: hsa-miR-215, AUGACCUAUGAAUUGACAGAC (MIMAT0000272, SEQ ID NO:126); hsa-miR-192, CUGACCUAUGAAUUGACAGCC (MIMAT0000222, SEQ ID NO:127); bta-miR-192, CUGACCUAUGAAUUGACAGCCAG (MIMAT0003820, SEQ ID NO:128); bta-miR-215, AUGACCUAUGAAUUGACAGACA (MIMAT0003797, SEQ ID NO:129); dre-miR-192, AUGACCUAUGAAUUGACAGCC (MIMAT0001275, SEQ ID NO:130); fru-miR-192, AUGACCUAUGAAUUGACAGCC (MIMAT0002941, SEQ ID NO:131); gga-miR-215, AUGACCUAUGAAUUGACAGAC (MIMAT0001134, SEQ ID NO:132); ggo-miR-215, AUGACCUAUGAAUUGACAGAC (MIMAT0002734, SEQ ID NO:133); mml-miR-215, AUGACCUAUGAAUUGACAGAC (MIMAT0002728, SEQ ID NO:134); mmu-miR-192, CUGACCUAUGAAUUGACA (MIMAT0000517, SEQ ID NO:135); mmu-miR-215, AUGACCUAUGAUUUGACAGAC (MIMAT0000904, SEQ ID NO:136); mne-miR-215, AUGACCUAUGAAUUGACAGAC (MIMAT0002736, SEQ ID NO:137); ppy-miR-215, AUGACCUAUGAAUUGACAGAC (MIMAT0002732, SEQ ID NO: 138); ptr-miR-215, AUGACCUAUGAAUUGACAGAC (MIMAT0002730, SEQ ID NO:139); mo-miR-192, CUGACCUAUGAAUUGACAGCC (MIMAT0000867, SEQ ID NO:140); mo-miR-215, AUGACCUAUGAUUUGACAGAC (MIMAT0003118, SEQ ID NO: 141); tni-miR-192, AUGACCUAUGAAUUGACAGCC (MIMAT0002942, SEQ ID NO:142); xtr-miR-192, AUGACCUAUGAAUUGACAGCC (MIMAT0003615, SEQ ID NO:143); or xtr-miR-215, AUGACCUAUGAAAUGACAGCC (MIMAT0003628, SEQ ID NO:144), or a complement thereof.

Mature miR-216 sequences include: hsa-miR-216, UAAUCUCAGCUGGCAACUGUG, (MIMAT0000273, SEQ ID NO:145); dre-miR-216a, UAAUCUCAGCUGGCAACUGUGA, (MIMAT0001284, SEQ ID NO:146); dre-miR-216b, UAAUCUCUGCAGGCAACUGUGA, (MIMAT0001867, SEQ ID NO:147); fru-miR-216a, AAAUCUCAGCUGGCAACUGUGA, (MIMAT0002973, SEQ ID NO:148); fru-miR-216b, UAAUCUCUGCAGGCAACUGUGA, (MIMAT0002975, SEQ ID NO:149); gga-miR-216, UAAUCUCAGCUGGCAACUGUG, (MIMAT0001131, SEQ ID NO:150); ggo-miR-216, UAAUCUCAGCUGGCAACUGUG, (MIMAT0002560, SEQ ID NO:151); lca-miR-216, UAAUCUCAGCUGGCAACUGUG, (MIMAT0002558, SEQ ID NO:152); mdo-miR-216, UAAUCUCAGCUGGCAACUGUG, (MIMAT0004131, SEQ ID NO:153); mmu-miR-216a, UAAUCUCAGCUGGCAACUGUG, (MIMAT0000662, SEQ ID NO:154); mmu-miR-216b, GGGAAAUCUCUGCAGGCAAAUGUGA, (MIMAT0003729, SEQ ID NO:155); ppa-miR-216, UAAUCUCAGCUGGCAACUGUG, (MIMAT0002562, SEQ ID NO:156); ppy-miR-216, UAAUCUCAGCUGGCAACUGUG, (MIMAT0002561, SEQ ID NO:157); ptr-miR-216, UUAUCUCAGCUGGCAACUGUG, (MIMAT0002559, SEQ ID NO: 158); rno-miR-216, UAAUCUCAGCUGGCAACUGUG, (MIMAT0000886, SEQ ID NO:159); ssc-miR-216, UAAUCUCAGCUGGCAACUGUG, (MIMAT0002130, SEQ ID NO:160); tni-miR-216a, AAAUCUCAGCUGGCAACUGUGA, (MIMAT0002974, SEQ ID NO:161); tni-miR-216b, UAAUCUCUGCAGGCAACUGUGA, (MIMAT0002976, SEQ ID NO:162); or xtr-miR-216, UAAUCUCAGCUGGCAACUGUG, (MIMAT0003629, SEQ ID NO: 163).

Mature miR-331 sequences include hsa-miR-331 GCCCCUGGGCCUAUCCUAGAA (MIMAT0000760, SEQ ID NO:164), or a complement thereof.

Mature mmu-miR-292-3p sequences include mmu-miR-292-3p, AAGUGCCGCCAGGUUUUGAGUGU, (MIMAT00000370, SEQ ID NO:165); hsa-miR-371, GUGCCGCCAUCUUUUGAGUGU, (MIMAT0000723, SEQ ID NO:166); hsa-miR-372, AAAGUGCUGCGACAUUUGAGCGU, (MIMAT0000724, SEQ ID NO:167); mmu-miR-290, CUCAAACUAUGGGGGCACUUUUU, (MIMAT0000366, SEQ ID NO: 168); mmu-miR-291a-3p, AAAGUGCUUCCACUUUGUGUGCC, (MIMAT0000368, SEQ ID NO:169); mmu-miR-291a-5p, CAUCAAAGUGGAGGCCCUCUCU, (MIMAT0000367, SEQ ID NO:170); mmu-miR-291b-3p, AAAGUGCAUCCAUUUUGUUUGUC, (MIMAT0003190, SEQ ID NO:171); mmu-miR-291b-5p, GAUCAAAGUGGAGGCCCUCUC, (MIMAT0003189, SEQ ID NO:172); mmu-miR-292-5p, ACUCAAACUGGGGGCUCUUUUG, (MIMAT0000369, SEQ ID NO:173); mmu-miR-293, AGUGCCGCAGAGUUUGUAGUGU, (MIMAT0000371, SEQ ID NO:174); mmu-miR-294, AAAGUGCUUCCCUUUUGUGUGU, (MIMAT0000372, SEQ ID NO:175); mmu-miR-295, AAAGUGCUACUACUUUUGAGUCU, (MIMAT0000373, SEQ ID NO:176); mo-miR-290, CUCAAACUAUGGGGGCACUUUUU, (MIMAT0000893, SEQ ID NO:177); rno-miR-291-3p, AAAGUGCUUCCACUUUGUGUGCC, (MIMAT0000895, SEQ ID NO:178); mo-miR-291-5p, CAUCAAAGUGGAGGCCCUCUCU, (MIMAT0000894, SEQ ID NO:179); mo-miR-292-3p, AAGUGCCGCCAGGUUUUGAGUGU, (MIMAT0000897, SEQ ID NO:180); or mo-miR-292-5p, ACUCAAACUGGGGGCUCUUUUG, (MIMAT0000896, SEQ ID NO:181), or a complement thereof.

In certain aspects, a subset of these miRNAs will be used that include some but not all of the listed miR-15, miR-26, miR-31, miR-145, miR-147, miR-188, miR-215, miR-216, miR-331, or mmu-miR-292-3p family members.

In one aspect, miR-15, miR-26, miR-31, miR-145, miR-147, miR-188, miR-215, miR-216, miR-331, or mmu-miR-292-3p sequences have a consensus sequence that can be determined by alignment of all miR family members or the alignment of miR family members from one or more species of origin. In certain embodiments one or more miR family member may be excluded from a claimed subset of miR family members.

The term miR-15, miR-26, miR-31, miR-145, miR-147, miR-188, miR-215, miR-216, miR-331, or mmu-miR-292-3p includes all members of the miR-15, miR-26, miR-31, miR-145, miR-147, miR-188, miR-215, miR-216, miR-331, or mmu-miR-292-3p or complements thereof. The mature sequences of miR-15, miR-26, miR-31, miR-145, miR-147, miR-188, miR-215, miR-216, miR-331, or mmu-miR-292-3p family includes hsa-miR-15a, hsa-miR-26a, hsa-miR-31, hsa-miR-145, hsa-miR-147, hsa-miR-188, hsa-miR-215, hsa-miR-216, hsa-miR-331, or mmu-miR-292-3p. Stem-loop sequences of miR-15, family members include hsa-mir-15a, CUUGGAGUAAAGUAGCAGCACAUAAUGGUUUGUGGAUUUUGAAAAGGUGC AGGCCAUAUUGUGCUGCCUCAAAAAUACAAGG (MI0000069, SEQ ID NO:182); hsa-mir-15b, UUGAGGCCUUAAAGUACUGUAGCAGCACAUCAUGGUUU ACAUGCUACAGUCAAGAUGCGAAUCAUUAUUUGCUGCUCUAGAAAUUUAA GGAAAUUCAU (MI0000438, SEQ ID NO:183); hsa-mir-16-1, GUCAGCAGUGCCUUAGCAGCACGUAAAUAUUGGCGUUAAGAUUCUAAAAU UAUCUCCAGUAUUAACUGUGCUGCUGAAGUAAGGUUGAC (MI0000070, SEQ ID NO:184); hsa-mir-16-2, GUUCCACUCUAGCAGCACGUAAAUAUUGGCGU AGUGAAAUAUAUAUUAAACACCAAUAUUACUGUGCUGCUUUAGUGUGAC (MI0000115, SEQ ID NO:185); hsa-mir-195, AGCUUCCCUGGCU CUAGCAGCACAGAAAUAUUGGCACAGGGAAGCGAGUCUGCCAAUAUUGGC UGUGCUGCUCCAGGCAGGGUGGUG (MI0000489, SEQ ID NO:186); age-mir-15a, CCUUGGAGUAAAGUAGCAGCACAUAAUGGUUUGUGGAUUUUGAAAAGGUG CAGGCCAUAUUGUGCUGCCUCAAAAAUACAAGG (MI0002945, SEQ ID NO:187); age-mir-15b, UUGAGGCCUUAAAGUACUGUAGCAGCACAUCAUGG UUUACAUACUACAGUCAAGAUGCGAAUCAUUAUUUGCUGCUCUAGAAAUU UAAGGAAAUUCAU (MI0002492, SEQ ID NO:188); age-mir-16, GUCAGCAGUGCCUUAGCAGCACGUAAAUAUUGGCGUUAAGAUUCUAAAAU UAUCUCCAGUAUUAACUGUGCUGCUGAAGUAAGGUUGAC (MI0002946, SEQ ID NO:189); bta-mir-15a, CCUUGGAGUAAAGUAGCAGCACAU AAUGGUUUGUGGAUUUUGAAAAGGUGCAGGCCAUAUUGUGCUGCCUCAAA AAUACAAGG (MI0005458, SEQ ID NO:190); bta-mir-15b, UUGAGACCUUAAAGUACUGUAGCAGCACAUCAUGGUUUACAUACUACAGU CAAGAUGCGAAUCAUUAUUUGCUGCUCUAGAAAUUUAAGGAAAUUCAU (MI0005012, SEQ ID NO:191); bta-mir-195, AGCUCCCC UGGCUCUAGCAGCACAGAAAUAUUGGCACUGGGAAGAAAGCCUGCCAAUA UUGGCUGUGCUGCUCCAGGCAGGGUGGUG (MI0005459, SEQ ID NO:192); dre-mir-15a-1, CCUGUCGGUACUGUAGCAGCACAGAAUGGUUUGUGAGUUAUAA CGGGGGUGCAGGCCGUACUGUGCUGCGGCAACAACGACAGG (MI0001891, SEQ ID NO:193); dre-mir-15a-2, GCCGAGGCUCUCUAGGUGAUGGUGUAG CAGCACAGAAUGGUUUGUGGUGAUACAGAGAUGCAGGCCAUGAUGUGCUG CAGCAUCAAUUCCUGGGACCUACGC (MI0001892, SEQ ID NO:194); dre-mir-15b, GUCUGUCGUCAUCUUUUUAUUUAGCCCUGAGUGCCCUGUAGCAGCACAUC AUGGUUUGUAAGUUAUAAGGGCAAAUUCCGAAUCAUGAUGUGCUGUCACU GGGAGCCUGGGAGUUUCUCCAUUAACAUGACAGC (MI0001893, SEQ ID NO:195); dre-mir-15c, CCUUAGACCGCUAAAGCAGCGCGUCAUGGUUUUC AACAUUAGAGAAGGUGCAAGCCAUCAUUUGCUGCUCUAGAGUUUUAAGG (MI0004779, SEQ ID NO:196); dre-mir-16a, CCUUCCUCGCUU UAGCAGCACGUAAAUAUUGGUGUGUUAUAGUCAAGGCCAACCCCAAUAUU AUGUGUGCUGCUUCAGUAAGGCAGG (MI0001894, SEQ ID NO:197); dre-mir-16b, CCUGAACUUGGCCGUGUGACAGACUGGCUGCCUGGCUGUAGCAGC ACGUAAAUAUUGGAGUCAAAGCACUUGCGAAUCCUCCAGUAUUGACCGUG CUGCUGGAGUUAGGCGGGCCGUUUACCGUCUGCGGGGGCCUCGGG (MI0001895, SEQ ID NO:198); dre-mir-16c, GAGGUUG UGUGUGUGUGCGUGUGUUGUCUUGCUUUAGCAGCAUGUAAAUAUUGGAGU UACUCCUUGGCCAAUGCCUCCAAUAUUGCUCGUGCUGCUGAAGCAAGAAG UCACCAAGCAGCACAUGCACGUCAUCCUU (MI0001896, SEQ ID NO:199); dre-mir-457a, UGCCUGACAGAAGCAGCACAUCAAUAUUGGCAGCUGCCCUCUCUC UGGGUUGCCAGUAUGGUUUGUGCUGCUCCCGUCAGACA (MI0002177, SEQ ID NO:200); dre-mir-457b, GAAUGUACUAAAGCAGCACAUAAAUACUGGAGG UGAUUGUGGUGUUAUCCAGUAUUGCUGUUCUGCUGUAGUAAGACC (MI0002178, SEQ ID NO:201); fru-mir-15a, CUGGUGAUGCUGUA GCAGCACGGAAUGGUUUGUGGGUUACACUGAGAUACAGGCCAUACUGUGC UGCCGCA (MI0003469, SEQ ID NO:202); fru-mir-15b, UGAGUCCCUUAGACUGCUAUAGCAGCGCAUCAUGGUUUGUAACGAUGUAG AAAAGGGUGCAAGCCAUAAUCUGCUGCUUUAGAAUUUUAAGGAAA (MI0003447, SEQ ID NO:203); fru-mir-16, GCCACUG UGCUGUAGCAGCACGUAAAUAUUGGAGUUAAGGCUCUCUGUGAUACCUCC AGUAUUGAUCGUGCUGCUGAAGCAAAGAUGAC (MI0003471, SEQ ID NO:204); gga-mir-15a, CCUUGGCAUAACGUAGCAGCACAUAAUGGUUUGUGGGU UUUGAAAAGGUGCAGGCCAUAUUGUGCUGCCUCAAAAAUACAAGG (MI0001186, SEQ ID NO:205); gga-mir-15b, UGAGGCCUU AAAGUACUCUAGCAGCACAUCAUGGUUUGCAUGCUGUAGUGAAGAUGCGA AUCAUUAUUUGCUGCUUUAGAAAUUUAAGGAA (MI0001223, SEQ ID NO:206); gga-mir-16-1, GUCUGUCAUACUCUAGCAGCACGUAAAUAUUGGUGUUA AAACUGUAAAUAUCUCCAGUAUUAACUGUGCUGCUGAAGUAAGGCU (MI0001185, SEQ ID NO:207); gga-mir-16-2, CCUACUUGUU CCGCCCUAGCAGCACGUAAAUAUUGGUGUAGUAAAAUAAACCUUAAACCC CAAUAUUAUUGUGCUGCUUAAGCGUGGCAGAGAU (MI0001222, SEQ ID NO:208); ggo-mir-15a, CCUUGGAGUAAAGUAGCAGCACAUAAUGGUUUGUG GAUUUUGAAAAGGUGCAGGCCAUAUUGUGCUGCCUCAAAAAUACAAGG (MI0002947, SEQ ID NO:209); ggo-mir-15b, UUGAGGC CUUAAAGUACUGUAGCAGCACAUCAUGGUUUACAUGCUACAGUCAAGAUG CGAAUCAUUAUUUGCUGCUCUAGAAAUUUAAGGAAAUUCAU (MI0002491, SEQ ID NO:210); ggo-mir-16, GUCAGCAGUGCCUUAGCAGCA CGUAAAUAUUGGCGUUAAGAUUCUAAAAUUAUCUCCAGUAUUAACUGUGC UGCUGAAGUAAGGUUGAC (MI0002948, SEQ ID NO:211); bta-mir-16, CAUACUUGUUCCGCUGUAGCAGCACGUAAAUAUUGGCGUAGUAAAAUAAA UAUUAAACACCAAUAUUAUUGUGCUGCUUUAGCGUGACAGGGA (MI0004739, SEQ ID NO:212); ggo-mir-195, AGCUUCCUGGGCUCUAGCAGCACAGAAAUAUUGGCACAGGGAAGCGAGUC UGCCAAUAUUGGCUGUGCUGCUCCAGGCAGGGUGGUG (MI0002617, SEQ ID NO:213); lca-mir-15a, CCUUGGAGUAAAGUAGCAGCACAUAAUG GUUUGUGGAUUUUGAAAAGGUGCAGGCCAUAUUGUGCUGCCUCAAAAAUA CAAGG (MI0002955, SEQ ID NO:214); lca-mir-16, GUCAGCAGUGC CUUAGCAGCACGUAAAUAUUGGUGUUAAGAUUCUAAAAUUAUCUCUAAGU AUUAACUGUGCCG (MI0002956, SEQ ID NO:215); lla-mir-15a, CCUUGGAGUAAAGUAGCAGCACAUAAUGGUUUGUGGAUUUUGAAAAGGUG CAGGCCAUAUUGUGCUGCCUCAAAAAUACAAGG (MI0002963, SEQ ID NO:216); lla-mir-15b, UUGAGGCCUUAAAGUACUGUAGCAGCACAU CAUGGUUUACAUACUACAGUCAAGAUGCGAAUCAUUAUUUGCUGCUCUAG AAAUUUAAGGAAAUUCAU (MI0002497, SEQ ID NO:217); lla-mir-16, GUCAGCAGUGCCUUAGCAGCACGUAAAUAUUGGCGCUAAGAUUCUAAAAU UAUCUCCAGUAUUAACUGUGCUGCUGAAGUAAGGUUGGC (MI0002964, SEQ ID NO:218); mdo-mir-15a, CCUUGGGGUAAAGUAGCAGCACAUA AUGGUUUGUUGGUUUUGAAAAGGUGCAGGCCAUAUUGUGCUGCCUCAAAA AUACAAGG (MI0005333, SEQ ID NO:219); mdo-mir-16, GUCAACAG UGCCUUAGCAGCACGUAAAUAUUGGCGUUAAGAUUUUAAAAGUAUCUCCA GUAUUAACUGUGCUGCUGAAGUAAGGUUGGCC (MI0005334, SEQ ID NO:220); mml-mir-15a, CCUUGGAGUAAAGUAGCAGCACAUAAUGGUUUGUGGAU UUUGAAAAGGUGCAGGCCAUAUUGUGCUGCCUCAAAAAUACAAGG (MI0002957, SEQ ID NO:221); mml-mir-15b, UUGAGGCCUUAAA GUACUGUAGCAGCACAUCAUGGUUUACAUACUACAGUCAAGAUGCGAAUC AUUAUUUGCUGCUCUAGAAAUUUAAGGAAAUUCAU (MI0002496, SEQ ID NO:222); mml-mir-16, GUCAGCAGUGCCUUAGCAGCACGUAAAUAUUGGCG UUAAGAUUCUAAAAUUAUCUCCAGUAUUAACUGUGCUGCUGAAGUAAGGU UGAC (MI0002958, SEQ ID NO:223); mmu-mir-15a, CCCUUGGAGUAAAGUAGCAGCACAUAAUGGUUUGUGGAUGUUGAAAAGGU GCAGGCCAUACUGUGCUGCCUCAAAAUACAAGGA (MI0000564, SEQ ID NO:224); mmu-mir-15b, CUGUAGCAGCACAUCAUGGUUUACAUACUAC AGUCAAGAUGCGAAUCAUUAUUUGCUGCUCUAG (MI0000140, SEQ ID NO:225); mmu-mir-16-1, AUGUCAGCGGUGCCUUAGCAGCACG UAAAUAUUGGCGUUAAGAUUCUGAAAUUACCUCCAGUAUUGACUGUGCUG CUGAAGUAAGGUUGGCAA (MI0000565, SEQ ID NO:226); mmu-mir-16-2, CAUGCUUGUUCCACUCUAGCAGCACGUAAAUAUUGGCGUAGUGAAAUAAA UAUUAAACACCAAUAUUAUUGUGCUGCUUUAGUGUGACAGGGAUA (MI0000566, SEQ ID NO:227); mmu-mir-195, ACACCCAACUC UCCUGGCUCUAGCAGCACAGAAAUAUUGGCAUGGGGAAGUGAGUCUGCCA AUAUUGGCUGUGCUGCUCCAGGCAGGGUGGUGA (MI0000237, SEQ ID NO:228); mne-mir-15a, CCUUGGAGUAAAGUAGCAGCACAUAAUG GUUUGUGGAUUUUGAAAAGGUGCAGGCCAUAUUGUGCUGCCUCAAAAAUA CAAGG (MI0002949, SEQ ID NO:229); mne-mir-15b, UUGAGGCCU UAAAGUACUGUAGCAGCACAUCAUGGUUUACAUACUACAGUCAAGAUGCG AAUCAUUAUUUGCUGCUCUAGAAAUUUAAGGAAAUUCAU (MI0002498, SEQ ID NO:230); mne-mir-16, GUCAGCAGUGCCUUAGCAGCACGUAAA UAUUGGCGUUAAGAUUCUAAAAUUAUCUCCAGUAUUAACUGUGCUGCUGA AGUAAGGUUGAC (MI0002950, SEQ ID NO:231); ppa-mir-15a, CCUUGGAGU AAAGUAGCAGCACAUAAUGGUUUGUGGAUUUUGAAAAGGUGCAGGCCAUA UUGUGCUGCCUCAAAAAUACAAGG (MI0002953, SEQ ID NO:232); ppa-mir-15b, UUGAGGCCUUAAAGUACUGUAGCAGCACAUCAUGGUUUACAUGCUACAGU CAAGAUGCGAAUCAUUAUUUGCUGCUCUAGAAAUUUAAGGAAAUUCAU (MI0002493, SEQ ID NO:233); ppa-mir-16, GUCAGCAGUGCCUUAGCAGCAC GUAAAUAUUGGCGUUAAGAUUCUAAAAUUAUCUCCAGUAUUAACUGUGCU GCUGAAGUAAGGUUGAC (MI0002954, SEQ ID NO:234); ppa-mir-195, AGCUUCCCUGGCUCUAGCAGCACAGAAAUAUUGGCACAGGGAAGCGAGUC UGCCAAUAUUGGCUGUGCUGCUCCAGGCAGGGUGGUG (MI0002618, SEQ ID NO:235); ppy-mir-15a, CCUUGGAGUAAAGUAGCAGCACAUAAUGGUUU GUGGAUUUUGAAAAGGUGCAGGCCAUAUUGUGCUGCCUCAAAAAUACAAG G (MI0002959, SEQ ID NO:236); ppy-mir-15b, UUGAGGCCUUAAAGU ACUGUAGCAGCACAUCAUGGUUUACAUGCUACAGUCAAGAUGCGAAUCAU UAUUUGCUGCUCUAGAAAUUUAAGGAAAUUCAU (MI0002494, SEQ ID NO:237); ppy-mir-16, GUCAGCAGUGCCUUAGCAGCACGUAAAUAUUGGCG UUAAGAUUCUAAAAUUAUCUCCAGUAUUAACUGUGCUGCUGAAGUAAGGU UGAC (MI0002960, SEQ ID NO:238); ptr-mir-15a, CCUUGGAGU AAAGUAGCAGCACAUAAUGGUUUGUGGAUUUUGAAAAGGUGCAGGCCAUA UUGUGCUGCCUCAAAAAUACAAGG (MI0002961, SEQ ID NO:239); ptr-mir-15b, UUGAGGCCUUAAAGUACUGUAGCAGCACAUCAUGGUUUACAUGCUACAGU CAAGAUGCGAAUCAUUAUUUGCUGCUCUAGAAAUUUAAGGAAAUUCAU (MI0002495, SEQ ID NO:240); ptr-mir-16, GUCAGCAGUGCCUUAGCAGCAC GUAAAUAUUGGCGUUAAGAUUCUAAAAUUAUCUCCAGUAUUAACUGUGCU GCUGAAGUAAGGUUGAC (MI0002962, SEQ ID NO:241); mo-mir-15b, UUGGAACCUUAAAGUACUGUAGCAGCACAUCAUGGUUUACAUACUACAGU CAAGAUGCGAAUCAUUAUUUGCUGCUCUAGAAAUUUAAGGAAAUUCAU (MI0000843, SEQ ID NO:242); mo-mir-16, CAUACUUGUUCC GCUCUAGCAGCACGUAAAUAUUGGCGUAGUGAAAUAAAUAUUAAACACCA AUAUUAUUGUGCUGCUUUAGUGUGACAGGGAUA (MI0000844, SEQ ID NO:243); mo-mir-195, AACUCUCCUGGCUCUAGCAGCACAGAAAUAUU GGCACGGGUAAGUGAGUCUGCCAAUAUUGGCUGUGCUGCUCCAGGCAGGG UGGUG (MI0000939, SEQ ID NO:244); sla-mir-15a, CCUUGGAGUAAAGU AGCAGCACAUAAUGGUUUGUGGAUUUUGAAAAGGUGCAGGCCAUAUUGUG CUGCCUCAAAAAUACAAGG (MI0002951, SEQ ID NO:245); sla-mir-16, GUCAGCAGUGCCUUAGCAGCACGUAAAUAUUGGCGUUAAGAUUCUAAAAU UAUCUCCAGUAUUAACUGUGCUGCUGAAGUAAGGUUGAC (MI0002952, SEQ ID NO:246); ssc-mir-15b, UUGAGGCCUUAAAGUACUGCCGCAG CACAUCAUGGUUUACAUACUACAAUCAAGAUGCGAAUCAUUAUUUGCUGC UCUAGAAAUUUAAGGAAAUUCAU (MI0002419, SEQ ID NO:247); tni-mir-15a, CUGGUGAUGCUGUAGCAGCACGGAAUGGUUUGUGAGUUACACUGAGAUAC AAGCCAUGCUGUGCUGCCGCA (MI0003470, SEQ ID NO:248); tni-mir-15b, GCCCUUAGACUGCUUUAGCAGCGCAUCAUGGUUUGUAAUGAUGUGGAAAA AAGGUGCAAACCAUAAUUUGCUGCUUUAGAAUUUUAAGGAA (MI0003448, SEQ ID NO:249); tni-mir-16, UAGCAGCACGUAAAUAUUGGAGUU AAGGCUCUCUGUGAUACCUCCAGUAUUGAUCGUGCUGCUGAAGCAAAG (MI0003472, SEQ ID NO:250); xtr-mir-15a, CCUUGACGUAAAGUAGCAGCACAUA AUGGUUUGUGGGUUACACAGAGGUGCAGGCCAUACUGUGCUGCCGCCAAA ACACAAGG (MI0004799, SEQ ID NO:251); xtr-mir-15b, UGUCCUAAAGAAGUGUAGCAGCACAUCAUGAUUUGCAUGCUGUAUUAUAG AUUCUAAUCAUUUUUUGCUGCUUCAUGAUAUUGGGAAA (MI0004800, SEQ ID NO:252); xtr-mir-15c, CUUUGAGGUGAUCUAGCAGCACAUCAUG GUUUGUAGAAACAAGGAGAUACAGACCAUUCUGAGCUGCCUCUUGA, M10004892 (SEQ ID NO:253); xtr-mir-16a, GCCAGCAGUCCUUUAGCAGCACG UAAAUAUUGGUGUUAAAAUGGUCCCAAUAUUAACUGUGCUGCUAGAGUAA GGUUGGCCU (MI0004802, SEQ ID NO:254); xtr-mir-16b, AAUUGCUCCGCAUUAGCAGCACGUAAAUAUUGGGUGAUAUGAUAUGGAGC CCCAGUAUUAUUGUACUGCUUAAGUGUGGCAAGG (MI0004910, SEQ ID NO:255); and xtr-mir-16c, UUUAGCAGCACGUAAAUACUGGAGU UCAUGACCAUAUCUGCACUCUCCAGUAUUACUUUGCUGCUAUAUU (MI0004801, SEQ ID NO:256) or complements thereof. Stem-loop sequences of miR-26, family members include, hsa-mir-26a-1, GUGGCCUCGUUCAAGUAAUCCAGGAUAGGCUGUGCAGGUCCCAAUGGGCC UAUUCUUGGUUACUUGCACGGGGACGC (MI0000083, SEQ ID NO:257); hsa-mir-26a-2, GGCUGUGGCUGGAUUCAAGUAAUCCAGGAUAGGCUGUUUCCAU CUGUGAGGCCUAUUCUUGAUUACUUGUUUCUGGAGGCAGCU (MI0000750, SEQ ID NO:258); hsa-mir-26b, CCGGGACCCAGUUCAAGUAAUUCAGGAUA GGUUGUGUGCUGUCCAGCCUGUUCUCCAUUACUUGGCUCGGGGACCGG (MI0000084, SEQ ID NO:259); bta-mir-26a, GGCUGUGGCUGGAUU CAAGUAAUCCAGGAUAGGCUGUUUCCAUCUGUGAGGCCUAUUCUUGAUUA CUUGUUUCUGGAGGCAGCU (MI0004731, SEQ ID NO:260); bta-mir-26b, UGCCCGGGACCCAGUUCAAGUAAUUCAGGAUAGGUUGUGUGCUGUCCAGC CUGUUCUCCAUUACUUGGCUCGGGGGCCGGUGCCC (MI0004745, SEQ ID NO:261); dre-mir-26a-1, UUUGGCCUGGUUCAAGUAAUCCAGGAUAGGCU UGUGAUGUCCGGAAAGCCUAUUCGGGAUGACUUGGUUCAGGAAUGA (MI0001923, SEQ ID NO:262); dre-mir-26a-2, GUGUGGACUUGAGUGCUGG AAGUGGUUGUUCCCUUGUUCAAGUAAUCCAGGAUAGGCUGUCUGUCCUGG AGGCCUAUUCAUGAUUACUUGCACUAGGUGGCAGCCGUUGCCCUUCAUGG AACUCAUGC (MI0001925, SEQ ID NO:263); dre-mir-26a-3, CUAAGCUGAU ACUGAGUCAGUGUGUGGCUGCAACCUGGUUCAAGUAAUCCAGGAUAGGCU UUGUGGACUAGGGUUGGCCUGUUCUUGGUUACUUGCACUGGGUUGCAGCU ACUAAACAACUAAGAAGAUCAGAAGAG (MI0001926, SEQ ID NO:264); fru-mir-26, AGGCCUCGGCCUGGUUCAAGUAAUCCAGGAUAGGCUGGUUAACCCU GCACGGCCUAUUCUUGAUUACUUGUGUCAGGAAGUGGCCGUG (MI0003369, SEQ ID NO:265); gga-mir-26a, GUCACCUGGUUCAAGUAA UCCAGGAUAGGCUGUAUCCAUUCCUGCUGGCCUAUUCUUGGUUACUUGCA CUGGGAGGC (MI0001187, SEQ ID NO:266); ggo-mir-26a, GUGGCCUCGUUCA AGUAAUCCAGGAUAGGCUGUGCAGGUCCCAAUGGGCCUAUUCUUGGUUAC UUGCACGGGGACGC (MI0002642, SEQ ID NO:267); lla-mir-26a, GUGGCCUCGUUCAAGUAAUCCAGGAUAGGCUGUGCAGGUCCCAAUGGGCC UAUUCUUGGUUACUUGCACGGGGACGC (MI0002644, SEQ ID NO:268); mml-mir-26a, GUGGCCUCGUUCAAGUAAUCCAGGAUAGGCUGUGCAGGUCCC AAUGGGCCUAUUCUUGGUUACUUGCACGGGGACGC (MI0002646, SEQ ID NO:269); mmu-mir-26a-1, AAGGCCGUGGCCUCGUUCAAGUAAUCCAGG AUAGGCUGUGCAGGUCCCAAGGGGCCUAUUCUUGGUUACUUGCACGGGGA CGCGGGCCUG (MI0000573, SEQ ID NO:270); mmu-mir-26a-2, GGCUGCGGCUGGAUUCAAGUAAUCCAGGAUAGGCUGUGUCCGUCCAUGAG GCCUGUUCUUGAUUACUUGUUUCUGGAGGCAGCG (MI0000706, SEQ ID NO:271); mmu-mir-26b, UGCCCGGGACCCAGUUCAAGUAAUUCAGGAUAGGUU GUGGUGCUGACCAGCCUGUUCUCCAUUACUUGGCUCGGGGGCCGGUGCC (MI0000575, SEQ ID NO:272); mne-mir-26a, GUGGCCUCG UUCAAGUAAUCCAGGAUAGGCUGUGCAGGUCCCAAUGGGCCUAUUCUUGA UUACUUGCACGGGGACGC (MI0002645, SEQ ID NO:273); ppa-mir-26a, GUGGCCUCGUUCAAGUAAUCCAGGAUAGGCUGUGCAGGUCCCAAUGGGCC UAUUCUUGGUUACUUGCACGGGGACGC (MI0002647, SEQ ID NO:274); ptr-mir-26a, GUGGCCUCGUUCAAGUAAUCCAGGAUAGGCUGUGCAGGUCCCAA UGGGCCUAUUCUUGGUUACUUGCACGGGGACGC (MI0002641, SEQ ID NO:275); rno-mir-26a, AAGGCCGUGGCCUUGUUCAAGUAAUCCAGG AUAGGCUGUGCAGGUCCCAAGGGGCCUAUUCUUGGUUACUUGCACGGGGA CGCGGGCCUG (MI0000857, SEQ ID NO:276); rno-mir-26b, UGCCCGGGACCCAGUUCAAGUAAUUCAGGAUAGGUUGUGGUGCUGGCCAG CCUGUUCUCCAUUACUUGGCUCGGGGGCCGGUGCC (MI0000858, SEQ ID NO:277); ssc-mir-26a, GGCUGUGGCUGGAUUCAAGUAAUCCAGGAUAG GCUGUUUCCAUCUGUGAGGCCUAUUCUUGAUUACUUGUUUCUGGAGGCAG CU (MI0002429, SEQ ID NO:278); tni-mir-26, GCGUUAG GCCUCGGCCUGGUUCAAGUAAUCCAGGAUAGGCUGGUUAACCCUGCACGG CCUAUUCUUGAUUACUUGUGUCAGGAAGUGGCCGCCAGC (MI0003370, SEQ ID NO:279); xtr-mir-26-1, GGCUGCUGCCUGGUUCAAGUAAUCCAGG AUAGGCUGUUUCCUCAAAGCACGGCCUACUCUUGAUUACUUGUUUCAGGA AGUAGCU (MI0004807, SEQ ID NO:280); xtr-mir-26-2, UGGGCGCUCGCUUCAAGU, M10004808, SEQ ID NO:281) or complement thereof. Stem-loop sequences of miR-31, family members include Hsa-mir-31, GGAGAGGAGGCAAGAUGCUGGCAUAGCUGUUGAACUGGGAACCUGCUAUG CCAACAUAUUGCCAUCUUUCC (MI0000089, SEQ ID NO:282); Ame-mir-31a, AUCACGAUUCUAACUGGGCGCCUCGAAGGCAAGAUGUCGGCAUAGCUGAU GCGAUUUUAAAAUUCGGCUGUGUCACAUCCAGCCAACCGAACGCUCAGAC (MI0005737, SEQ ID NO:283); Bmo-mir-31, GUCGAGCCGGU GGCUGGGAAGGCAAGAAGUCGGCAUAGCUGUUUGAAUAAGAUACACGGCU GUGUCACUUCGAGCCAGCUCAAUCCGCCGGCUUUCUUCAAUUUCAAGAUU UGCGGAUGCU (MI0005377, SEQ ID NO:284); Bta-mir-31, UCCUGUAA CUUGGAACUGGAGAGGAGGCAAGAUGCUGGCAUAGCUGUUGAACUGCGAA CCUGCUAUGCCAACAUAUUGCCAUCUCUCUUGUCCG (MI0004762, SEQ ID NO:285); Dme-mir-31a, UCCGUUGGUAAAUUGGCAAGAUGUCGGCAUAGCUGA CGUUGAAAAGCGAUUUUGAAGAGCGCUAUGCUGCAUCUAGUCAGUUGUUC AAUGGA (MI0000420, SEQ ID NO:286); Dme-mir-31b, CAAAUAAU GAAUUUGGCAAGAUGUCGGAAUAGCUGAGAGCACAGCGGAUCGAACAUUU UAUCGUCCGAAAAAAUGUGAUUAUUUUUGAAAAGCGGCUAUGCCUCAUCU AGUCAAUUGCAUUACUUUG (MI0000410, SEQ ID NO:287); Dps-mir-31a, UCUGUUGGUAAAUUGGCAAGAUGUCGGCAUAGCUGAAGUUGAAAAGCGAU CUUUGAGAACGCUAUGCUGCAUCUAGUCAGUUAUUCAAUGGA (MI0001314, SEQ ID NO:288); Dps-mir-31b, AAUUUGGCAAGAUGUCGGAAUAGCUGAGAGC AAAAAGAAGAUGAUUUGAAAUGCGGCUAUGCCUCAUCUAGUCAAUUGCAU UCAUUUGA (MI0001315, SEQ ID NO:289); Dre-mir-31, GAAGAGAU GGCAAGAUGUUGGCAUAGCUGUUAAUGUUUAUGGGCCUGCUAUGCCUCCA UAUUGCCAUUUCUG (MI0003691, SEQ ID NO:290); Gga-mir-31, UUCUUUCAUGCAGAGCUGGAGGGGAGGCAAGAUGUUGGCAUAGCUGUUAA CCUAAAAACCUGCUAUGCCAACAUAUUGUCAUCUUUCCUGUCUG (MI0001276, SEQ ID NO:291); Ggo-mir-31, GGAGAGGAGGCAAGAUG CUGGCAUAGCUGUUGAACUGGGAACCUGCUAUGCCAACAUAUUGCCAUCU UUcc (MI0002673, SEQ ID NO:292); Mdo-mir-31, AGCUGGAGAGGAGGCAAGAUGUUGGCAUAGCUGUUGAACUGAGAACCUGC UAUGCCAACAUAUUGCCAUCUUUCUUGUCUAUCAGCA (MI0005278, SEQ ID NO:293); mml-mir-31, GGAGAGGAGGCAAGAUGCUGGCAUAGCUGUUGA ACUGGGAACCUGCUAUGCCAACAUAUUGCCAUCUUUCC (MI0002671, SEQ ID NO:294); Mmu-mir-31, UGCUCCUGUAACUCGGAACUGGAGAGGAGGCAAGA UGCUGGCAUAGCUGUUGAACUGAGAACCUGCUAUGCCAACAUAUUGCCAU CUUUCCUGUCUGACAGCAGCU (MI0000579, SEQ ID NO:295); Mne-mir-31, GGAGAGGAGGCAAGAUGCUGGCAUAGCUGUUGAACUGGGAACCUGCUAUG CCAACAUAUUGCCAUCUUUCC (MI0002675, SEQ ID NO:296); ppa-mir-31, GGAGAGGAGGCAAGAUGCUGGCAUAGCUGUUGAACUGGGAACCUGCUAUG CCAACAUAUUGCCAUCUUUCC (MI0002676, SEQ ID NO:297); ppy-mir-31, GGAGAGGAGGCAAGAUGCUGGCAUAGCUGUUGAACUGGGAACCUGCUAUG CCAACAUAUUGCCAUCUUUCC (MI0002674, SEQ ID NO:298); ptr-mir-31, GGAGAGGAGGCAAGAUGCUGGCAUAGCUGUUGAACUGGGAACCUGCUAUG CCAACAUAUUGCCAUCUUUCC (MI0002672, SEQ ID NO:299); rno-mir-31, UGCUCCUGAAACUUGGAACUGGAGAGGAGGCAAGAUGCUGGCAUAGCUGU UGAACUGAGAACCUGCUAUGCCAACAUAUUGCCAUCUUUCCUGUCUGACA GCAGCU (MI0000872, SEQ ID NO:300); sme-mir-31b, AUUGAUAA UGACAAGGCAAGAUGCUGGCAUAGCUGAUAAACUAUUUAUUACCAGCUAU UCAGGAUCUUUCCCUGAAUAUAUCAAU (MI0005146, SEQ ID NO:301); xtr-mir-31, CCUAGUUCUAGAGAGGAGGCAAGAUGUUGGCAUAGCUGUUGCAU CUGAAACCAGUUGUGCCAACCUAUUGCCAUCUUUCUUGUCUACC (MI0004921, SEQ ID NO:302) or complement thereof. Stem-loop sequences of miR-145, family members include hsa-mir-145, CACCUUGUCCUCACGGUCCAGUUUUCCCAGGAAUCCCUUAGAUGCUAAGAU GGGGAUUCCUGGAAAUACUGUUCUUGAGGUCAUGGUU (MI0000461, SEQ ID NO:303); bta-mir-145, CACCUUGUCCUCACGGUCCAGUUUUCCCAGGAAUCCCU UAGAUGCUAAGAUGGGGAUUCCUGGAAAUACUGUUCUUGAGGUCAUGGUU (MI0004756, SEQ ID NO:304); dre-mir-145, UCAGUCUUCAUCAU UUCCUCAUCCCCGGGGUCCAGUUUUCCCAGGAAUCCCUUGGGCAAUCGAAA GGGGGAUUCCUGGAAAUACUGUUCUUGGGGUUGGGGGUGGACUACUGA (MI0002010, SEQ ID NO:305); ggo-mir-145, CACCUUGUCCUCACG GUCCAGUUUUCCCAGGAAUCCCUUAGAUGCUAAGAUGGGGAUUCCUGGAA AUACUGUUCUUGAGGUCAUGGUU (MI0002560, SEQ ID NO:306); mdo-mir-145, CUCAGGGUCCAGUUUUCCCAGGAAUCCCUUAGAUGCUAAGAUGGGGAUUC CUGGAAAUACUGUUCUUGAG (MI0005305, SEQ ID NO:307); mml-mir-145, CACCUUGUCCUCACGGUCCAGUUUUCCCAGGAAUCCCUUAAAUGCUAAGAU GGGGAUUCCUGGAAAUACUGUUCUUGAGGUCAUGGUU (MI0002558, SEQ ID NO:308); mmu-mir-145, CUCACGGUCCAGUUUUCCCAGGAAUCCCU UGGAUGCUAAGAUGGGGAUUCCUGGAAAUACUGUUCUUGAG (MI0000169, SEQ ID NO:309); mne-mir-145, CACCUUGUCCUCACGGUCCAGU UUUCCCAGGAAUCCCUUAAAUGCUAAGAUGGGGAUUCCUGGAAAUACUGU UCUUGAGGUCAUGGUU (MI0002562, SEQ ID NO:310); ppy-mir-145, CACCUUGUCCUCACGGUCCAGUUUUCCCAGGAAUCCCUUAGAUGCUAAGAU GGGGAUUCCUGGAAAUACUGUUCUUGAGGUCAUGGUU (MI0002561, SEQ ID NO:311); ptr-mir-145, CACCUUGUCCUCACGGUCCAGUUUUCCCA GGAAUCCCUUAGAUGCUAAGAUGGGGAUUCCUGGAAAUACUGUUCUUGAG GUCAUGGUU (M10002559, SEQ ID NO:312); rno-mir-145, CACCUUGUCCUCACGGUCCAGUUUUCCCAGGAAUCCCUUGGAUGCUAAGAU GGGGAUUCCUGGAAAUACUGUUCUUGAGGUCAUGGCU (MI0000918, SEQ ID NO:313); ssc-mir-145, CACCUUGUCCUCACGGUCCAGUUUUCCCAGGAAUCCCU UAGAUGCUGAGAUGGGGAUUCCUGUAAAUACUGUUCUUGAGGUCAUGG (MI0002417, SEQ ID NO:314); xtr-mir-145, ACCUAUUCCUCA AGGUCCAGUUUUCCCAGGAAUCCCUUGGGUGCUGUGGUGGGGAUUCCUGG AAAUACUGUUCUUGGGGUGUAGGC (MI0004939, SEQ ID NO:315) or complements thereof.

Stem-loop sequences of miR-147, family members include hsa-mir-147, AAUCUAAAGACAACAUUUCUGCACACACACCAGACUAUGGAAGCCAGUGU GUGGAAAUGCUUCUGCUAGAUU (MI0000262, SEQ ID NO:316); gga-mir-147-1, AAUCUAGUGGAAUCACUUCUGCACAAACUUGACUACUGAAAUCAGUGUGC GGAAAUGCUUCUGCUACAUU (MI0003696, SEQ ID NO:317); gga-mir-147-2, AAUCUAGUGGAAUCACUUCUGCACAAACUUGACUACUGAAAUCAGUGUGC GGAAAUGCUUCUGCUACAUU (MI0003697, SEQ ID NO:318); mne-mir-147, AAUCUAAAGAAAACAUUUCUGCACACACACCAGACUAUUGAAGCCAGUGU GUGGAAAUGCUUCUGCUACAUU (MI0002773, SEQ ID NO:319); ppa-mir-147, AAUCUAAAGAAAACAUUUCUGCACACACACCAGACUAUGGAAGCCAGUGU GUGGAAAUGCUUCUGCUAGAUU (MI0002774, SEQ ID NO:320); ppy-mir-147, AAUCUAAAGAAAACAUUUCUGCACACACACCAGACUAUGGAAGCCAGUGU GUGGAAAUGCUUCUGCUAGAUU (MI0002771, SEQ ID NO:321); ptr-mir-147, AAUCUAAAGAAAACAUUUCUGCACACACACCAGACUAUGGAAGCCAGUGU GUGGAAAUGCUUCUGCUAGAUU (MI0002770, SEQ ID NO:322); sla-mir-147, AAUCUAAAGAAAACAUUUCUGCACACACACCAGACUAUUGAAGCCAGUGU GUGGAAAUGCUUCUGCCACAUU (MI0002772, SEQ ID NO:323) or a complement thereof.

Stem-loop sequences of miR-188, family members include hsa-mir-188, UGCUCCCUCUCUCACAUCCCUUGCAUGGUGGAGGGUGAGCUUUCUGAAAA CCCCUCCCACAUGCAGGGUUUGCAGGAUGGCGAGCC (MI0000484, SEQ ID NO:324); hsa-mir-532, CGACUUGCUUUCUCUCCUCCAUGCCUUGAGUGUAGG ACCGUUGGCAUCUUAAUUACCCUCCCACACCCAAGGCUUGCAAAAAAGCGA GCCU (MI0003205, SEQ ID NO:325); hsa-mir-660, CUGCUCCUUCUCCCAUACCCAUUGCAUAUCGGAGUUGUGAAUUCUCAAAAC ACCUCCUGUGUGCAUGGAUUACAGGAGGGUGAGCCUUGUCAUCGUG (MI0003684, SEQ ID NO:326); bta-mir-532, GACUUGCUUUCUCUCU UACAUGCCUUGAGUGUAGGACCGUUGGCAUCUUAAUUACCCUCCCACACCC AAGGCUUGCAGGAGAGCCA (MI0005061, SEQ ID NO:327); bta-mir-660, CUGCUCCUUCUCCCGUACCCAUUGCAUAUCGGAGCUGUGAAUUCUCAAAGC ACCUCCUAUGUGCAUGGAUUACAGGAGGG (MI0005468, SEQ ID NO:328); mml-mir-188, UGCUCCCUCUCUCACAUCCCUUGCAUGGUGGAGGGUGAG CUUUAUGAAAACCCCUCCCACAUGCAGGGUUUGCAGGAUGGUGAGCC (MI0002608, SEQ ID NO:329); mmu-mir-188, UCUCACAUCCCUUGCAUGGUGGAGGGUGAGCUCUCUGAAAACCCCUCCCAC AUGCAGGGUUUGCAGGA (MI0000230, SEQ ID NO:330); mmu-mir-532, CAGAUUUGCUUUUUCUCUUCCAUGCCUUGAGUGUAGGACCGUUGACAUCU UAAUUACCCUCCCACACCCAAGGCUUGCAGGAGAGCAAGCCUUCUC (MI0003206, SEQ ID NO:331); mne-mir-188, UGCUCCCUCUCU CACAUCCCUUGCAUGGUGGAGGGUGAGCUUUAUGAAAACCCCUCCCACAU GCAGGGUUUGCAGGAUGGUGAGCC (MI0002611, SEQ ID NO:332); ppa-mir-188, UGCUCCCUCUCUCACAUCCCUUGCAUGGUGGAGGGUGAGCUUUCUGAAAA CCCCUCCCACAUGCAGGGUUUGCAGGAUGGCGAGCC (MI0002612, SEQ ID NO:333); ppy-mir-188, UGCUCCCUCUCUCACAUCCCUUGCAUGGUGGAG GGUGAGCUUUCUGAAAACCCCUCCCACAUGCAGGGUUUGCAGGAUGGCGA GCC (MI0002610, SEQ ID NO:334); ptr-mir-188, UGCUCCCUCUCUCACA UCCCUUGCAUGGUGGAGGGUGAACUUUCUGAAAACCCCUCCCACAUGCAG GGUUUGCAGGAUGGCGAGCC (MI0002609, SEQ ID NO:335) or complements thereof.

Stem-loop sequences of miR-215, family members include hsa-mir-215, AUCAUUCAGAAAUGGUAUACAGGAAAAUGACCUAUGAAUUGACAGACAAU AUAGCUGAGUUUGUCUGUCAUUUCUUUAGGCCAAUAUUCUGUAUGACUGU GCUACUUCAA (MI0000291, SEQ ID NO:336); hsa-mir-192, GCCGAGA CCGAGUGCACAGGGCUCUGACCUAUGAAUUGACAGCCAGUGCUCUCGUCUC CCCUCUGGCUGCCAAUUCCAUAGGUCACAGGUAUGUUCGCCUCAAUGCCAG C (MI0000234, SEQ ID NO:337); bta-mir-192, AGACCGAGUGCACAG GGCUCUGACCUAUGAAUUGACAGCCAGUGCUCUUGUGUCCCCUCUGGCUGC CAAUUCCAUAGGUCACAGGUAUGUUCGCCUCAAUGCCAGC (MI0005035, SEQ ID NO:338); bta-mir-215, UGUACAGGAAAAUGACCUAUGAAUUGACAG ACAACGUGACUAAGUCUGUCUGUCAUUUCUGUAGGCCAAUGUUCUGUAU (MI0005016, SEQ ID NO:339); dre-mir-192, CUAGGACACAGGGU GAUGACCUAUGAAUUGACAGCCAGUGUUUGCAGUCCAGCUGCCUGUCAGU UCUGUAGGCCACUGCCCUGUU (MI0001371, SEQ ID NO:340); fru-mir-192, UGGGACGUGAGGUGAUGACCUAUGAAUUGACAGCCAGUAACUGGAGCCUC UGCCUGUCAGUUCUGUAGGCCACUGCUACGUU (MI0003257, SEQ ID NO:341); gga-mir-215, UCAGUAAGAACUGGUGUCCAGGAAAAUGACCUAUGAAUUGA CAGACUGCUUUCAAAAUGUGCCUGUCAUUUCUAUAGGCCAAUAUUCUGUG CACUUUUCCUACUU (MI0001203, SEQ ID NO:342); ggo-mir-215, AUCAUUCAGAAAUGGUAUACGGGAAAAUGACCUAUGAAUUGACAGACAAU AUAGCUGAGUUUGUCUGUCAUUUCUUUAGACCAAUAUUCUGUAUGACUGU GCUACUUCAA (MI0003031, SEQ ID NO:343); mml-mir-215, AUCAUUAAGAAAUGGUAUACAGGAAAAUGACCUAUGAAUUGACAGACACU AUAGCUGAGUUUGUCUGUCAUUUCUUUAGGCCAAUAUUCUGUAUGACUGU GCUACUUCAA (MI0003025, SEQ ID NO:344); mmu-mir-192, CGUGCACAGGGCUCUGACCUAUGAAUUGACAGCCAGUACUCUUUUCUCUCC UCUGGCUGCCAAUUCCAUAGGUCACAGGUAUGUUCACC (MI0000551, SEQ ID NO:345); mmu-mir-215, AGCUCUCAGCAUCAACGGUGUACAGGAGAAUGA CCUAUGAUUUGACAGACCGUGCAGCUGUGUAUGUCUGUCAUUCUGUAGGC CAAUAUUCUGUAUGUCACUGCUACUUAAA (MI0000974, SEQ ID NO:346); mne-mir-215, AUCAUUAAGAAAUGGUAUACAGGAAAAUGACCUAUGAAUUGACA GACACUAUAGCUGAGUUUGUCUGUCAUUUCUUUAGGCCAAUAUUCUGUAU GACUGUGCUACUUCAA (MI0003033, SEQ ID NO:347); ppy-mir-215, AUCAUUCAGAAAUGGUAUACAGGAAAAUGACCUAUGAAUUGACAGACAAU ACAGCUGAGUUUGUCUGUCAUUUCUUUAGGCCAAUAUUCUGUACAACUGU GCUACUUCAA (MI0003029, SEQ ID NO:348); ptr-mir-215, AUCAUUCAGAAAUGGUAUACGGGAAAAUGACCUAUGAAUUGACAGACAAU AUAGCUGAGUUUGUCUGUCAUUUCUUUAGGCCAAUAUUCUGUAUGACUGU GCUACUUCAA (MI0003027, SEQ ID NO:349); rno-mir-192, GUCAAGAUGGAGUGCACAGGGCUCUGACCUAUGAAUUGACAGCCAGUACU CUGAUCUCGCCUCUGGCUGCCAGUUCCAUAGGUCACAGGUAUGUUCGCCUC AAUGCCAGC (MI0000935, SEQ ID NO:350); rno-mir-215, GGUGUACA GGACAAUGACCUAUGAUUUGACAGACAGUGUGGCUGCGUGUGUCUGUCAU UCUGUAGGCCAAUAUUCUGUAUGUCUCUCCUCCUUACAA (MI0003482, SEQ ID NO:351); tni-mir-192, CACGAGGUGAUGACCUAUGAAUUGACAGCCAGUAA CUGGAGCCUCUGCCUGUCAGUUCUGUAGGCCACUGCUGCGUCCGUCCC (MI0003258, SEQ ID NO:352); xtr-mir-192, GAGUGUACGGGCCUA UGACCUAUGAAUUGACAGCCAGUGGAUGUGAAGUCUGCCUGUCAAUUCUG UAGGCCACAGGUUCGUCCACCU (MI0004855, SEQ ID NO:353); xtr-mir-215, AACUGGUAACCAGGAGGAUGACCUAUGAAAUGACAGCCACUUCCAUACCA AACAUGUCUGUCAUUUCUGUAGGCCAAUAUUCUGAUUGCUUUGUUGA (MI0004868, SEQ ID NO:354) or complements thereof. Stem-loop sequences of miR-216, family members include hsa-mir-216, GAUGGCUGUGAGUUGGCUUAAUCUCAGCUGGCAACUGUGAGAUGUUCAUA CAAUCCCUCACAGUGGUCUCUGGGAUUAUGCUAAACAGAGCAAUUUCCUA GCCCUCACGA (MI0000292, SEQ ID NO:355); dre-mir-216a-1, GCUGAUUUUUGGCAUAAUCUCAGCUGGCAACUGUGAGUAGUGUUUUCAUC CCUCUCACAGGCGCUGCUGGGGUUCUGUCACACACAGCA (MI0001382, SEQ ID NO:356); dre-mir-216a-2, GCUGAUUUUUGGCAUAAUCUCAGCUGGCAA CUGUGAGUAGUGUUUUCAUCCCUCUCACAGGCGCUGCUGGGGUUCUGUCA CACACAGCA (MI0002047, SEQ ID NO:357); dre-mir-216b-1, ACUGACUGG GUAAUCUCUGCAGGCAACUGUGAUGUGAUUACAGUCUCACAUUGACCUGA AGAGGUUGAGCAGUCUGU (MI0002048, SEQ ID NO:358); dre-mir-216b-2, CUGACUGGGUAAUCUCUGCAGGCAACUGUGAUGUGAUUACAGUCUCACAU UGACCUGAAGAGGUUGUGCAGUCUGU (MI0002049, SEQ ID NO:359); fru-mir-216a, UUGGUAAAAUCUCAGCUGGCAACUGUGAGUCGUUCACUAGCUGCU CUCACAAUGGCCUCUGGGAUUAUGCUAA (MI0003291, SEQ ID NO:360); fru-mir-216b, UGACUGUUUAAUCUCUGCAGGCAACUGUGAUGGUGUUUUAUAU UCUCACAAUCACCUGGAGAGAUUCUGCAGUUUAU (MI0003293, SEQ ID NO:361); gga-mir-216, GAUGGCUGUGAAUUGGCUUAAUCUCAGCUGGCAAC UGUGAGCAGUUAAUAAUUCUCACAGUGGUAUCUGGGAUUAUGCUAAACAC AGCAAUUUCUUUGCUCUAAUG (MI0001200, SEQ ID NO:362); ggo-mir-216, GAUGGCUGUGAGUUGGCUUAAUCUCAGCUGGCAACUGUGAGAUGUUCAUA CAAUCCCUCACAGUGGUCUCUGGGAUUAUGCUAAACAGAGCAAUUUCCUA GCCCUCACGA (MI0002863, SEQ ID NO:363); lca-mir-216, GAUGGCUGUGAGUUGGCUUAAUCUCAGCUGGCAACUGUGAGAUGUUCAUA CAAUCCCUCACAGUGGUCUCUGGGAUUAUGCUAAACAGAGCAAUUUCCUA GCCCUCACGA (MI0002861, SEQ ID NO:364); mdo-mir-216, GAUGGCUGUGAAUUGGCUUAAUCUCAGCUGGCAACUGUGAGAUGUUAAUA AAUUCCCUCACAGUGGUCUCUGGGAUUAUGCUAAACAGAGCAAUUUC (MI0005320, SEQ ID NO:365); mmu-mir-216a, UUGGUUUAAUCUCAGCUGGCAACUGUGAGAUGUCCCUAUCAUUCCUCACA GUGGUCUCUGGGAUUAUGCUAA (MI0000699, SEQ ID NO:366); mmu-mir-216b, UUGGCAGACUGGGAAAUCUCUGCAGGCAAAUGUGAUGUCACUGAAGAAAC CACACACUUACCUGUAGAGAUUCUUCAGUCUGACAA (MI0004126, SEQ ID NO:367); ppa-mir-216, GAUGGCUGUGAGUUGGCUUAAUCUCAGCUGGCAACU GUGAGAUGUUCAUACAAUCCCUCACAGUGGUCUCUGGGAUUAUGCUAAAC AGAGCAAUUUCCUAGCCCUCACGA (MI0002865, SEQ ID NO:368); ppy-mir-216, GAUGGCUGUGAGUUGGCUUAAUCUCAGCUGGCAACUGUGAGAUGUUCAUA CAAUCCCUCACAGUGGUCUCUGGGAUUAUGCUAAACAGAGCAAUUUCCUU GCCCUCACGA (MI0002864, SEQ ID NO:369); ptr-mir-216, GAUGGCUGUGAGUUGGCUUAUCUCAGCUGGCAACUGUGAGAUGUUCAUAC AAUCCCUCACAGUGGUCUCUGGGAUUAAACUAAACAGAGCAAUUUCCUAG CCCUCACGA (MI0002862, SEQ ID NO:370); rno-mir-216, GUUAGC UAUGAGUUAGUUUAAUCUCAGCUGGCAACUGUGAGAUGUCCCUAUCAUUC CUCACAGUGGUCUCUGGGAUUAUGCUAAACAGAGCAAUUUCCUUGACCUC (MI0000955, SEQ ID NO:371); ssc-mir-216, GAUGGCUGUGAGUUG GCUUAAUCUCAGCUGGCAACUGUGAGAUGUUCAUACAAUCCCCCACAGUG GUCUCUGGGAUUAUGCUAAACAGAGCAAUUUCCUUGCCCU (MI0002424, SEQ ID NO:372); tni-mir-216a, UUGGUGAAAUCUCAGCUGGCAACUGUGAGUCG UUCACUAGCUGCUCUCACAAUGGCCUCUGGGAUUAUGCUAA (MI0003292, SEQ ID NO:373); tni-mir-216b, UGACUGUUUAAUCUCUGCAGGCAAC UGUGAUGGUGAUUUUUAUUCUCACAAUCACCUGGAGAGAUUCUGCAGUUU AU (MI0003294, SEQ ID NO:374); xtr-mir-216, UGGCUGUGAAUUGGCUUAAU CUCAGCUGGCAACUGUGAGCAGUUAAUAAAUUAUCUCACAGUGGUCUCUG GGAUUAUACUAAACACAGCAA (MI0004869, SEQ ID NO:375) or complement thereof.

Stem-loop sequences of miR-331, family members include hsa-mir-331, GAGUUUGGUUUUGUUUGGGUUUGUUCUAGGUAUGGUCCCAGGGAUCCCAG AUCAAACCAGGCCCCUGGGCCUAUCCUAGAACCAACCUAAGCUC (MI0000812, SEQ ID NO:376); bta-mir-331, GAGUUUGGUUUUGUU UGGGUUUGUUCUAGGUAUGGUCCCAGGGAUCCCAGAUCAAACCAGGCCCC UGGGCCUAUCCUAGAACCAACCUAA (MI0005463, SEQ ID NO:377); mmu-mir-331, GAGUCUGGUUUUGUUUGGGUUUGUUCUAGGUAUGGUCCCAGGGAU CCCAGAUCAAACCAGGCCCCUGGGCCUAUCCUAGAACCAACCUAAACCCGU (MI0000609, SEQ ID NO:378); mo-mir-331, GAGUCUGGUCUUG UUUGGGUUUGUUCUAGGUAUGGUCCCAGGGAUCCCAGAUCAAACCAGGCC CCUGGGCCUAUCCUAGAACCAACCUAAACCCAU (MI0000608, SEQ ID NO:379) or complement thereof.

Stem-loop sequences of miR-292-3p family members include mmu-mir-292, CAGCCUGUGAUACUCAAACUGGGGGCUCUUUUGGAUUUUCAUCGGAAGAA AAGUGCCGCCAGGUUUUGAGUGUCACCGGUUG (MI0000390, SEQ ID NO:380); hsa-mir-371, GUGGCACUCAAACUGUGGGGGCACUUUCUGCUCUCUGG UGAAAGUGCCGCCAUCUUUUGAGUGUUAC (MI0000779, SEQ ID NO:381); hsa-mir-372, GUGGGCCUCAAAUGUGGAGCACUAUUCUGAUGUCCAAGUGG AAAGUGCUGCGACAUUUGAGCGUCAC (MI0000780, SEQ ID NO:382); mmu-mir-290, CUCAUCUUGCGGUACUCAAACUAUGGGGGCACUUUUUUUUUUCUU UAAAAAGUGCCGCCUAGUUUUAAGCCCCGCCGGUUGAG (MI0000388, SEQ ID NO:383); mmu-mir-291a, CCUAUGUAGCGGCCAUCAAAGUGGAGGCCCUCUCU UGAGCCUGAAUGAGAAAGUGCUUCCACUUUGUGUGCCACUGCAUGGG (MI0000389, SEQ ID NO:384); mmu-mir-291b, ACAUACAGUGUCGAUCAAAGUGGAGGCCCUCUCCGCGGCUUGGCGGGAAA GUGCAUCCAUUUUGUUUGUCUCUGUGUGU (MI0003539, SEQ ID NO:385); mmu-mir-293, UUCAAUCUGUGGUACUCAAACUGUGUGACAUUUUG UUCUUUGUAAGAAGUGCCGCAGAGUUUGUAGUGUUGCCGAUUGAG (MI0000391, SEQ ID NO:386); mmu-mir-294, UUCCAUAUAGCCA UACUCAAAAUGGAGGCCCUAUCUAAGCUUUUAAGUGGAAAGUGCUUCCCU UUUGUGUGUUGCCAUGUGGAG (MI0000392, SEQ ID NO:387); mmu-mir-295, GGUGAGACUCAAAUGUGGGGCACACUUCUGGACUGUACAUAGAAAGUGCU ACUACUUUUGAGUCUCUCC (MI0000393, SEQ ID NO:388); mo-mir-290, UCAUCUUGCGGUUCUCAAACUAUGGGGGCACUUUUUUUUUCUUUAAAAAG UGCCGCCAGGUUUUAGGGCCUGCCGGUUGAG (MI0000964, SEQ ID NO:389); mo-mir-291, CCGGUGUAGUAGCCAUCAAAGUGGAGGCCCUCUCUUG GGCCCGAGCUAGAAAGUGCUUCCACUUUGUGUGCCACUGCAUGGG (MI0000965, SEQ ID NO:390); rno-mir-292, CAACCUGUGAUACUCAAACUGGGGGCUCUUUUGGGUUUUCUUUGGAAGAA AAGUGCCGCCAGGUUUUGAGUGUUACCGAUUG, M10000966, SEQ ID NO:391) or a complement thereof.

In a further aspect, “a miR-15, miR-26, miR-31, miR-145, miR-147, miR-188, miR-215, miR-216, miR-331, or mmu-miR-292-3p nucleic acid sequence” generally includes all or a segment of the full length precursor of miR-15, miR-26, miR-31, miR-145, miR-147, miR-188, miR-215, miR-216, miR-331, or mmu-miR-292-3p family members.

In certain aspects, a nucleic acid miR-15, miR-26, miR-31, miR-145, miR-147, miR-188, miR-215, miR-216, miR-331, or mmu-miR-292-3p nucleic acid, or a segment or a mimetic thereof, will comprise 5, 6, 7, 8, 9, 10, 11, 12, 13, 14, 15, 16, 17, 18, 19, 20, 21, 22, 23, 24, 25, 26, 27, 28, 29 or more nucleotides of the precursor miRNA or its processed sequence, including all ranges and integers there between. In certain embodiments, the miR-15, miR-26, miR-31, miR-145, miR-147, miR-188, miR-215, miR-216, miR-331, or mmu-miR-292-3p nucleic acid sequence contains the full-length processed miRNA sequence and is referred to as the “miR-15, miR-26, miR-31, miR-145, miR-147, miR-188, miR-215, miR-216, miR-331, or mmu-miR-292-3p full-length processed nucleic acid sequence.” In still further aspects, a miR-15, miR-26, miR-31, miR-145, miR-147, miR-188, miR-215, miR-216, miR-331, or mmu-miR-292-3p comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment of miR-15, miR-26, miR-31, miR-145, miR-147, miR-188, miR-215, miR-216, miR-331, or mmu-miR-292-3p that is at least 75, 80, 85, 90, 95, 98, 99 or 100% identical to SEQ ID NOs provided herein.

In specific embodiments, a miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor containing nucleic acid is miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor, or a variation thereof. miR-15, miR-26, miR-31, miR-145, miR-147, miR-188, miR-215, miR-216, miR-331, or mmu-miR-292-3p can be hsa-miR-15, hsa-miR-26, hsa-miR-31, hsa-miR-145, hsa-miR-147, hsa-miR-188, hsa-miR-215, hsa-miR-216, hsa-miR-331, or mmu-miR-292-3p, respectively.

In a further aspect, a miR-15, miR-26, miR-31, miR-145, miR-147, miR-188, miR-215, miR-216, miR-331, or mmu-miR-292-3p nucleic acid or miR-15, miR-26, miR-31, miR-145, miR-147, miR-188, miR-215, miR-216, miR-331, or mmu-miR-292-3p inhibitor can be administered with 1, 2, 3, 4, 5, 6, 7, 8, 9, 10 or more miRNAs or miRNA inhibitors. miRNAs or their complements can be administer concurrently, in sequence or in an ordered progression. In certain aspects, a miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor can be administered in combination with one or more of let-7, miR-15, miR-16, miR-20, miR-21, miR-26a, miR-34a, miR-126, miR-143, miR-147, miR-188, miR-200, miR-215, miR-216, miR-292-3p, and/or miR-331 nucleic acids or inhibitors thereof. All or combinations of miRNAs or inhibitors thereof may be administered in a single formulation. Administration may be before, during or after a second therapy.

miR-15, miR-26, miR-31, miR-145, miR-147, miR-188, miR-215, miR-216, miR-331, or mmu-miR-292-3p nucleic acids or complement thereof may also include various heterologous nucleic acid sequence, i.e., those sequences not typically found operatively coupled with miR-15, miR-26, miR-31, miR-145, miR-147, miR-188, miR-215, miR-216, miR-331, or mmu-miR-292-3p in nature, such as promoters, enhancers, and the like. The miR-15, miR-26, miR-31, miR-145, miR-147, miR-188, miR-215, miR-216, miR-331, or mmu-miR-292-3p nucleic acid is a recombinant nucleic acid, and can be a ribonucleic acid or a deoxyribonucleic acid. The recombinant nucleic acid may comprise a miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor expression cassette, i.e., a nucleic acid segment that expresses a nucleic acid when introduce into an environment containing components for nucleic acid synthesis. In a further aspect, the expression cassette is comprised in a viral vector, or plasmid DNA vector or other therapeutic nucleic acid vector or delivery vehicle, including liposomes and the like. In a particular aspect, the miR-15, miR-26, miR-31, miR-145, miR-147, miR-188, miR-215, miR-216, miR-331, or mmu-miR-292-3p nucleic acid is a synthetic nucleic acid. Moreover, nucleic acids of the invention may be fully or partially synthetic. In certain aspects, viral vectors can be administered at 1×10², 1×10³, 1×10⁴ 1×10⁵, 1×10⁶, 1×10⁷, 1×10⁸, 1×10⁹, 1×10¹⁰, 1×10¹¹, 1×10¹², 1×10¹³, 1×10¹⁴ pfu or viral particle (vp).

In a particular aspect, the miR-15, miR-26, miR-31, miR-145, miR-147, miR-188, miR-215, miR-216, miR-331, or mmu-miR-292-3p nucleic acid or miR-15, miR-26, miR-31, miR-145, miR-147, miR-188, miR-215, miR-216, miR-331, or mmu-miR-292-3p inhibitor is a synthetic nucleic acid. Moreover, nucleic acids of the invention may be fully or partially synthetic. In still further aspects, a nucleic acid of the invention or a DNA encoding a nucleic acid of the invention can be administered at 0.001, 0.01, 0.1, 1, 10, 20, 30, 40, 50, 100, 200, 400, 600, 800, 1000, 2000, to 4000 μg or mg, including all values and ranges there between. In yet a further aspect, nucleic acids of the invention, including synthetic nucleic acid, can be administered at 0.001, 0.01, 0.1, 1, 10, 20, 30, 40, 50, 100, to 200 μg or mg per kilogram (kg) of body weight. Each of the amounts described herein may be administered over a period of time, including 0.5, 1, 2, 3, 4, 5, 6, 7, 8, 9, 10, minutes, hours, days, weeks, months or years, including all values and ranges there between.

In certain embodiments, administration of the composition(s) can be enteral or parenteral. In certain aspects, enteral administration is oral. In further aspects, parenteral administration is intralesional, intravascular, intracranial, intrapleural, intratumoral, intraperitoneal, intramuscular, intralymphatic, intraglandular, subcutaneous, topical, intrabronchial, intratracheal, intranasal, inhaled, or instilled. Compositions of the invention may be administered regionally or locally and not necessarily directly into a lesion.

In certain aspects, the gene or genes modulated comprises 1, 2, 3, 4, 5, 6, 7, 8, 9, 10, 11, 12, 13, 14, 15, 20, 25, 30, 35, 40, 45, 50, 100, 150, 200 or more genes or combinations of genes identified in Tables 1, 3, and/or 4. In still further aspects, the gene or genes modulated may exclude 1, 2, 3, 4, 5, 6, 7, 8, 9, 10, 11, 12, 13, 14, 15, 20, 25, 30, 35, 40, 45, 50, 100, 150, 175 or more genes or combinations of genes identified in Tables 1, 3, and/or 4. Modulation includes modulating transcription, mRNA levels, mRNA translation, and/or protein levels in a cell, tissue, or organ. In certain aspects the expression of a gene or level of a gene product, such as mRNA or encoded protein, is down-regulated or up-regulated. In a particular aspect the gene modulated comprises or is selected from (and may even exclude) 1, 2, 3, 4, 5, 6, 7, 8, 9, 10, 11, 12, 13, 14, 15, 16, 17, 18, 19, 20, 21, 22, 23, 24, 25, 26. 27, 28, or all of the genes identified in Tables 1, 3, and/or 4, or any combinations thereof. In certain embodiments a gene modulated or selected to be modulated is from Table 1. In further embodiments a gene modulated or selected to be modulated is from Table 3. In still further embodiments a gene modulated or selected to be modulated is from Table 4. In certain aspects of the invention one or more genes may be excluded from the claimed invention.

Embodiments of the invention may also include obtaining or assessing a gene expression profile or miRNA profile of a target cell prior to selecting the mode of treatment, e.g., administration of a miR-15, miR-26, miR-31, miR-145, miR-147, miR-188, miR-215, miR-216, miR-331, or mmu-miR-292-3p nucleic acid, inhibitor of miR-15, miR-26, miR-31, miR-145, miR-147, miR-188, miR-215, miR-216, miR-331, or mmu-miR-292-3p, or mimetics thereof. The database content related to all nucleic acids and genes designated by an accession number or a database submission are incorporated herein by reference as of the filing date of this application. In certain aspects of the invention one or more miRNA or miRNA inhibitor may modulate a single gene. In a further aspect, one or more genes in one or more genetic, cellular, or physiologic pathways can be modulated by one or more miRNAs or complements thereof, including miR-15, miR-26, miR-31, miR-145, miR-147, miR-188, miR-215, miR-216, miR-331, or mmu-miR-292-3p nucleic acids in combination with other miRNAs.

A further embodiment of the invention is directed to methods of modulating a cellular pathway comprising administering to the cell an amount of an isolated nucleic acid comprising a miR-15, miR-26, miR-31, miR-145, miR-147, miR-188, miR-215, miR-216, miR-331, or mmu-miR-292-3p nucleic acids and miR-15, miR-26, miR-31, miR-145, miR-147, miR-188, miR-215, miR-216, miR-331, or mmu-miR-292-3p inhibitors in combination with other miRNAs or miRNA inhibitors.

miR-15, miR-26, miR-31, miR-145, miR-147, miR-188, miR-215, miR-216, miR-331, or mmu-miR-292-3p nucleic acids may also include various heterologous nucleic acid sequence, i.e., those sequences not typically found operatively coupled with miR-15, miR-26, miR-31, miR-145, miR-147, miR-188, miR-215, miR-216, miR-331, or mmu-miR-292-3p in nature, such as promoters, enhancers, and the like. The miR-15, miR-26, miR-31, miR-145, miR-147, miR-188, miR-215, miR-216, miR-331, or mmu-miR-292-3p nucleic acid is a recombinant nucleic acid, and can be a ribonucleic acid or a deoxyribonucleic acid. The recombinant nucleic acid may comprise a miR-15, miR-26, miR-31, miR-145, miR-147, miR-188, miR-215, miR-216, miR-331, or mmu-miR-292-3p expression cassette. In a further aspect, the expression cassette is comprised in a viral, or plasmid DNA vector or other therapeutic nucleic acid vector or delivery vehicle, including liposomes and the like. In a particular aspect, the miR-15, miR-26, miR-31, miR-145, miR-147, miR-188, miR-215, miR-216, miR-331, or mmu-miR-292-3p nucleic acid is a synthetic nucleic acid. Moreover, nucleic acids of the invention may be fully or partially synthetic.

A further embodiment of the invention is directed to methods of modulating a cellular pathway comprising administering to the cell an amount of an isolated nucleic acid comprising a miR-15, miR-26, miR-31, miR-145, miR-147, miR-188, miR-215, miR-216, miR-331, or mmu-miR-292-3p nucleic acid sequence in an amount sufficient to modulate the expression, function, status, or state of a cellular pathway, in particular those pathways described in Table 2 or the pathways known to include one or more genes from Table 1, 3, and/or 4. Modulation of a cellular pathway includes, but is not limited to modulating the expression of one or more gene. Modulation of a gene can include inhibiting the function of an endogenous miRNA or providing a functional miRNA to a cell, tissue, or subject. Modulation refers to the expression levels or activities of a gene or its related gene product or protein, e.g., the mRNA levels may be modulated or the translation of an mRNA may be modulated, etc. Modulation may increase or up regulate a gene or gene product or it may decrease or down regulate a gene or gene product.

Still a further embodiment includes methods of treating a patient with a pathological condition comprising one or more of step (a) administering to the patient an amount of an isolated nucleic acid comprising a miR-15, miR-26, miR-31, miR-145, miR-147, miR-188, miR-215, miR-216, miR-331, or mmu-miR-292-3p nucleic acid sequence in an amount sufficient to modulate the expression of a cellular pathway; and (b) administering a second therapy, wherein the modulation of the cellular pathway sensitizes the patient to the second therapy. A cellular pathway may include, but is not limited to one or more pathway described in Table 2 below or a pathway that is know to include one or more genes of Tables 1, 3, and/or 4. A second therapy can include administration of a second miRNA or therapeutic nucleic acid, or may include various standard therapies, such as chemotherapy, radiation therapy, drug therapy, immunotherapy, and the like. Embodiments of the invention may also include the determination or assessment of a gene expression profile for the selection of an appropriate therapy.

Embodiments of the invention include methods of treating a subject with a pathological condition comprising one or more of the steps of (a) determining an expression profile of one or more genes selected from Table 1, 3, and/or 4; (b) assessing the sensitivity of the subject to therapy based on the expression profile; (c) selecting a therapy based on the assessed sensitivity; and (d) treating the subject using selected therapy. Typically, the pathological condition will have as a component, indicator, or result the mis-regulation of one or more gene of Table 1, 3, and/or 4.

Further embodiments include the identification and assessment of an expression profile indicative of miR-15, miR-26, miR-31, miR-145, miR-147, miR-188, miR-215, miR-216, miR-331, or mmu-miR-292-3p status in a cell or tissue comprising expression assessment of one or more gene from Table 1, 3, and/or 4, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers from Tables 1, 3, and/or 4); it is contemplated that the nucleic acid profile can be obtained using a set of RNAs, using for example nucleic acid amplification or hybridization techniques well know to one of ordinary skill in the art. The difference in the expression profile in the sample from the patient and a reference expression profile, such as an expression profile from a normal or non-pathologic sample, is indicative of a pathologic, disease, or cancerous condition. A nucleic acid or probe set comprising or identifying a segment of a corresponding mRNA can include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more nucleotides, including any integer or range derivable there between, of a gene, or genetic marker, or a nucleic acid, mRNA or a probe representative thereof that is listed in Tables 1, 3, and/or 4, or identified by the methods described herein.

Certain embodiments of the invention are directed to compositions and methods for assessing, prognosing, or treating a pathological condition in a patient comprising measuring or determining an expression profile of one or more marker(s) in a sample from the patient, wherein a difference in the expression profile in the sample from the patient and an expression profile of a normal sample or reference expression profile is indicative of pathological condition and particularly cancer. In certain aspects of the invention, the cellular pathway, gene, or genetic marker is or is representative of one or more pathway or marker described in Table 1, 3, and/or 4, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of Table 1, 3, and/or 4, including any combination thereof.

TABLE 1A
Genes with increased (positive values) or decreased (negative values)
expression following transfection of human cancer cells with pre-miR hsa-miR-15a

| | RefSeq Transcript ID | |
|---|---|---|
| Gene Symbol | (Pruitt et al., 2005) | Δ log₂ |
| ABCA1 | NM_005502 | 0.706584 |
| ABCB6 /// ATG9A | NM_005689 /// NM_024085 | −0.893191 |
| ABLIM3 | NM_014945 | 0.807167 |
| ACOX2 | NM_003500 | −0.884661 |
| ADARB1 | NM_001033049 /// NM_001112 /// | 1.67209 |
| | NM_015833 /// NM_015834 | |
| ADM | NM_001124 | 0.982052 |
| ADRB2 | NM_000024 | 1.04898 |
| AKAP12 | NM_005100 /// NM_144497 | 0.807181 |
| AKAP2 /// PALM2- | NM_001004065 /// NM_007203 /// NM_147150 | 1.07515 |
| AKAP2 | | |
| ANKRD46 | NM_198401 | 0.725941 |
| ANTXR1 | NM_018153 /// NM_032208 /// NM_053034 | 0.951172 |
| AOX1 | NM_001159 | 1.27456 |
| AP1S2 | NM_003916 | 0.722522 |
| APOH | NM_000042 | −0.778363 |
| APP | NM_000484 /// NM_201413 /// NM_201414 | 0.710494 |
| AQP3 | NM_004925 | −1.0108 |
| ARHGDIA | NM_004309 | −1.43641 |
| ARHGDIB | NM_001175 | 0.829838 |
| ARL2 | NM_001667 | −1.94907 |
| ARL2BP | NM_012106 | 1.20234 |
| ATP6V0E | NM_003945 | 1.30096 |
| AXL | NM_001699 /// NM_021913 | 1.26935 |
| BAG5 | NM_001015048 /// NM_001015049 /// NM_004873 | −0.731695 |
| BAMBI | NM_012342 | −0.882718 |
| BCL2A1 | NM_004049 | 0.801198 |
| BEAN | XM_375359 | 1.14936 |
| BIRC3 | NM_001165 /// NM_182962 | 0.984482 |
| BTN3A2 | NM_007047 | 0.819101 |
| C4BPB | NM_000716 /// NM_001017364 /// NM_001017365 | 2.02325 |
| | ///NM_001017366 /// NM_001017367 | |
| C6orf216 | NM_206908 /// NM_206910 /// NM_206911 /// | 1.05448 |
| | NM_206912 /// XR_000259 | |
| C8orf1 | NM_004337 | −0.702374 |
| CA12 | NM_001218 /// NM_206925 | −1.26277 |
| CCL20 | NM_004591 | 0.853408 |
| CCND1 | NM_053056 | −0.889303 |
| CCND3 | NM_001760 | −1.05519 |
| CCNG2 | NM_004354 | 1.00993 |
| CDC37L1 | NM_017913 | −0.876288 |
| CDCA4 | NM_017955 /// NM_145701 | −0.773713 |
| CDH17 | NM_004063 | −1.09072 |
| CDH4 | NM_001794 | 0.830142 |
| CDKN2C | NM_001262 /// NM_078626 | −1.00104 |
| CDS2 | NM_003818 | −1.19113 |
| CFH /// CFHL1 | NM_000186 /// NM_001014975 /// NM_002113 | −0.888088 |
| CGI-38 | NM_015964 /// NM_016140 | −0.758479 |
| CGI-48 | NM_016001 | 1.58316 |
| CHAF1A | NM_005483 | −0.714709 |
| CHUK | NM_001278 | −1.04118 |
| CLCN4 | NM_001830 | −0.915403 |
| CLIC4 | NM_013943 | 0.899491 |
| COL11A1 | NM_001854 /// NM_080629 /// NM_080630 | 1.21281 |
| COL4A1 | NM_001845 | 0.721033 |
| COL4A2 | NM_001846 | 0.752816 |
| COL5A1 | NM_000093 | 0.781154 |
| COL6A1 | NM_001848 | 0.708164 |
| CPM | NM_001005502 /// NM_001874 /// NM_198320 | 1.03293 |
| CTGF | NM_001901 | 1.44017 |
| CTSS | NM_004079 | 0.753473 |
| CXCL1 | NM_001511 | 1.13774 |
| CXCL2 | NM_002089 | 0.914747 |
| CXCL5 | NM_002994 | 0.832592 |
| CXCR4 | NM_001008540 /// NM_003467 | 0.946256 |
| CYP4F11 | NM_021187 | −1.17394 |
| CYP4F3 | NM_000896 | −1.39695 |
| CYR61 | NM_001554 | 0.801016 |
| DAAM1 | NM_014992 | 1.11752 |
| DAF | NM_000574 | 0.749996 |
| DDAH1 | NM_012137 | 1.11882 |
| DHPS | NM_001930 /// NM_013406 /// NM_013407 | −0.749475 |
| DIO2 | NM_000793 /// NM_001007023 /// NM_013989 | 1.05322 |
| DOCK4 | NM_014705 | 0.715045 |
| DSU | NM_018000 | 0.832877 |
| DUSP1 | NM_004417 | 0.901714 |
| DUSP10 | NM_007207 /// NM_144728 /// NM_144729 | 0.802771 |
| DUSP5 | NM_004419 | 1.06893 |
| DUSP6 | NM_001946 /// NM_022652 | 0.762807 |
| E2F8 | NM_024680 | −1.09486 |
| EEF1D | NM_001960 /// NM_032378 | 1.09981 |
| EFEMP1 | NM_004105 /// NM_018894 | 1.53793 |
| EIF4E | NM_001968 | −0.706986 |
| ENO1 | NM_001428 | 1.06282 |
| EPAS1 | NM_001430 | 1.14112 |
| FAM18B | NM_016078 | −0.710266 |
| FBN1 | NM_000138 | 0.864655 |
| FBXO11 | NM_012167 /// NM_018693 /// NM_025133 | 1.10195 |
| FGF2 | NM_002006 | −1.38337 |
| FGFR4 | NM_002011 /// NM_022963 /// NM_213647 | −0.706112 |
| FKBP1B | NM_004116 /// NM_054033 | −0.953076 |
| FLJ13910 | NM_022780 | 0.733455 |
| FNBP1 | NM_015033 | 0.943991 |
| FSTL1 | NM_007085 | 0.814388 |
| GALNT7 | NM_017423 | −1.08105 |
| GBP1 | NM_002053 | 0.94431 |
| GCLC | NM_001498 | −0.735984 |
| GFPT1 | NM_002056 | −0.88304 |
| GLIPR1 | NM_006851 | 0.739398 |
| GTSE1 | NM_016426 | −0.789888 |
| HAS2 | NM_005328 | −0.875224 |
| HEG | XM_087386 | 0.947872 |
| HMGA2 | NM_001015886 /// NM_003483 /// NM_003484 | 1.10974 |
| HMGCS1 | NM_002130 | 1.13726 |
| HSPA1B | NM_005346 | −1.2135 |
| IER3IP1 | NM_016097 | 1.02762 |
| IFI16 | NM_005531 | 1.10866 |
| IGFBP3 | NM_000598 /// NM_001013398 | 0.767581 |
| IL6 | NM_000600 | 1.18471 |
| IL6ST | NM_002184 /// NM_175767 | 0.726757 |
| IL8 | NM_000584 | 1.10422 |
| INHBB | NM_002193 | −0.950023 |
| INHBC | NM_005538 | 0.898337 |
| INSIG1 | NM_005542 /// NM_198336 /// NM_198337 | 0.74226 |
| INSL4 | NM_002195 | −1.11623 |
| IQGAP2 | NM_006633 | −0.783372 |
| IRF1 | NM_002198 | 0.72684 |
| ITPR2 | NM_002223 | 0.740631 |
| KCNJ2 | NM_000891 | 1.35987 |
| KIAA0485 | — | 1.10255 |
| KIAA0754 | — | 0.899045 |
| KLF4 | NM_004235 | −0.749759 |
| KRT7 | NM_005556 | 1.21091 |
| LAMC2 | NM_005562 /// NM_018891 | 0.733084 |
| LCN2 | NM_005564 | −0.794915 |
| LOC153561 | NM_207331 | 0.794392 |
| LOC348162 | XM_496132 | 0.774096 |
| LOXL2 | NM_002318 | 0.740607 |
| LRP12 | NM_013437 | −0.784206 |
| LYPD1 | NM_144586 | 1.24908 |
| MAP3K2 | NM_006609 | 0.733667 |
| MAP7 | NM_003980 | −1.16472 |
| MAZ | NM_002383 | −0.725569 |
| MCL1 | NM_021960 /// NM_182763 | 1.65586 |
| MEG3 | XR_000167 /// XR_000277 | 0.800336 |
| MGC5618 | — | 0.912493 |
| MPPE1 | NM_023075 /// NM_138608 | −0.72104 |
| MYL9 | NM_006097 /// NM_181526 | 0.795096 |
| NALP1 | NM_001033053 /// NM_014922 /// NM_033004 /// | 1.06065 |
| | NM_033006 /// NM_033007 | |
| NAV3 | NM_014903 | 0.773472 |
| NF1 | NM_000267 | −1.44283 |
| NFE2L3 | NM_004289 | 0.884419 |
| NFKB2 | NM_002502 | 0.773655 |
| NID1 | NM_002508 | 0.892766 |
| NMT2 | NM_004808 | 0.828083 |
| NNMT | NM_006169 | 1.1372 |
| NPC1 | NM_000271 | 1.36826 |
| NTE | NM_006702 | −0.726337 |
| NUCKS | NM_022731 | 2.22615 |
| NUPL1 | NM_001008564 /// NM_001008565 /// NM_014089 | −0.806715 |
| PDZK1IP1 | NM_005764 | 1.08475 |
| PFAAP5 | NM_014887 | 0.792392 |
| PGK1 | NM_000291 | 1.87681 |
| PHACTR2 | NM_014721 | −0.81188 |
| PLA2G4A | NM_024420 | −0.87476 |
| PLSCR4 | NM_020353 | −1.89975 |
| PMCH | NM_002674 | 1.04416 |
| PNMA2 | NM_007257 | 0.704085 |
| PODXL | NM_001018111 /// NM_005397 | 1.257 |
| PPP1R11 | NM_021959 /// NM_170781 | −0.806236 |
| PRO1843 | — | 1.19666 |
| PTENP1 | — | 1.07135 |
| PTGS2 | NM_000963 | −1.0791 |
| PTK9 | NM_002822 /// NM_198974 | 1.20386 |
| PTPRE | NM_006504 /// NM_130435 | 0.703589 |
| QKI | NM_006775 /// NM_206853 /// NM_206854 /// | 0.73124 |
| | NM_206855 | |
| RAB2 | NM_002865 | 1.39501 |
| RAFTLIN | NM_015150 | 1.67418 |
| RARRES3 | NM_004585 | 0.757518 |
| RASGRP1 | NM_005739 | 1.08021 |
| RBL1 | NM_002895 /// NM_183404 | −0.842142 |
| RDX | NM_002906 | 0.700954 |
| RGS2 | NM_002923 | 0.823743 |
| RHEB | NM_005614 | 1.07333 |
| RIP | NM_001033002 /// NM_032308 | 1.51241 |
| ROR1 | NM_005012 | 0.824907 |
| RPL14 | NM_001034996 /// NM_003973 | 0.969345 |
| RPL38 | NM_000999 | 1.50078 |
| RPS11 | NM_001015 | 1.37758 |
| RPS6KA3 | NM_004586 | −1.21197 |
| RPS6KA5 | NM_004755 /// NM_182398 | 0.938506 |
| S100P | NM_005980 | −1.06668 |
| SEMA3C | NM_006379 | 0.845374 |
| SEPT6 /// N-PAC | NM_015129 /// NM_032569 /// NM_145799 | 1.04331 |
| | /// NM_145800 /// NM_145802 | |
| SKP2 | NM_005983 /// NM_032637 | 0.74694 |
| SLC11A2 | NM_000617 | −1.0072 |
| SLC26A2 | NM_000112 | 0.711837 |
| SMA4 | NM_021652 | 0.789119 |
| SMARCA2 | NM_003070 /// NM_139045 | 1.09406 |
| SNAI2 | NM_003068 | 0.817633 |
| SNAP23 | NM_003825 /// NM_130798 | 0.815178 |
| SOCS2 | NM_003877 | 0.886257 |
| SPARC | NM_003118 | 1.44472 |
| SPFH2 | NM_001003790 /// NM_001003791 /// NM_007175 | −0.730905 |
| SPOCK | NM_004598 | 0.834427 |
| STC1 | NM_003155 | 1.05196 |
| STX3A | NM_004177 | 0.910285 |
| SULT1C1 | NM_001056 /// NM_176825 | 0.793242 |
| SUMO2 | NM_001005849 /// NM_006937 | 0.867526 |
| SYNE1 | NM_015293 /// NM_033071 /// | 1.33924 |
| | NM_133650 /// NM_182961 | |
| TACC1 | NM_006283 | −1.05059 |
| TAF15 | NM_003487 /// NM_139215 | 0.941963 |
| TAGLN | NM_001001522 /// NM_003186 | 1.54875 |
| TFG | NM_001007565 /// NM_006070 | 0.894314 |
| THBD | NM_000361 | 1.18344 |
| THBS1 | NM_003246 | −0.871039 |
| THUMPD1 | NM_017736 | −0.772288 |
| TM7SF1 | NM_003272 | 0.879449 |
| TMEM45A | NM_018004 | −0.851551 |
| TNFAIP6 | NM_007115 | 0.758707 |
| TNFSF9 | NM_003811 | −1.51814 |
| TOP1 | NM_003286 | 0.717449 |
| TOX | NM_014729 | 1.57101 |
| TPM1 | NM_000366 /// NM_001018004 /// NM_001018005 | 1.07102 |
| | /// NM_001018006 /// NM_001018007 // | |
| TRA1 | NM_003299 | 2.20518 |
| TRIM22 | NM_006074 | 1.39642 |
| TRIO | NM_007118 | 0.767064 |
| TTC3 | NM_001001894 /// NM_003316 | 0.713917 |
| TTMP | NM_024616 | 1.06102 |
| TUBB4 | NM_006087 | −0.757438 |
| TXN | NM_003329 | 1.62493 |
| UBE2I | NM_003345 /// NM_194259 ///NM_194260 /// | 0.882595 |
| | NM_194261 | |
| UBE2L6 | NM_004223 /// NM_198183 | 0.84659 |
| UGCG | NM_003358 | 0.848697 |
| USP34 | NM_014709 | 1.0433 |
| VAV3 | NM_006113 | −0.868484 |
| VDAC3 | NM_005662 | 1.05842 |
| VIL2 | NM_003379 | 1.03829 |
| VPS4A | NM_013245 | −0.876444 |
| VTI1B | NM_006370 | −1.07453 |
| WISP2 | NM_003881 | 0.998185 |
| WNT7B | NM_058238 | −0.81257 |
| WSB2 | NM_018639 | 0.835972 |
| XTP2 | NM_015172 | 1.07659 |
| YRDC | NM_024640 | −0.747991 |
| ZBED2 | NM_024508 | 1.17703 |

TABLE 1B
Genes with increased (positive values) or decreased (negative values)
expression following transfection of human cancer cells with pre-miR hsa-miR-26.

| | RefSeq Transcript ID | |
|---|---|---|
| Gene Symbol | (Pruitt et al., 2005) | Δ log₂ |
| ABR | NM_001092 /// NM_021962 | −0.833053 |
| ACTR2 | NM_001005386 /// NM_005722 | 0.784523 |
| AER61 | NM_173654 | 1.17093 |
| AHNAK | NM_001620 /// NM_024060 | −1.19295 |
| AKAP12 | NM_005100 /// NM_144497 | 0.869987 |
| AKAP2 /// PALM2- | NM_001004065 /// NM_007203 /// NM_147150 | 0.815452 |
| AKAP2 | | |
| ALDH5A1 | NM_001080 /// NM_170740 | −1.37495 |
| ANKRD12 | NM_015208 | 1.0142 |
| ANTXR1 | NM_018153 /// NM_032208 /// NM_053034 | 1.41894 |
| ARFRP1 | NM_003224 | −0.72603 |
| ARG2 | NM_001172 | 0.886422 |
| ARHGDIA | NM_004309 | −1.08013 |
| ARHGDIB | NM_001175 | 1.17986 |
| ARL2BP | NM_012106 | 0.975481 |
| ARTS-1 | NM_016442 | 0.747895 |
| ATP6V0E | NM_003945 | 1.10054 |
| ATP9A | NM_006045 | −0.960651 |
| AXL | NM_001699 /// NM_021913 | 1.36117 |
| B4GALT4 | NM_003778 /// NM_212543 | −1.0873 |
| BCAT1 | NM_005504 | 1.00482 |
| BCL2L1 | NM_001191 /// NM_138578 | −1.45177 |
| BID | NM_001196 /// NM_197966 /// NM_197967 | −1.04896 |
| BNC2 | NM_017637 | 1.2229 |
| C14orf10 | NM_017917 | −1.11148 |
| C1orf116 | NM_023938 | −0.834587 |
| C1orf24 | NM_022083 /// NM_052966 | 1.15962 |
| C1R | NM_001733 | 0.83181 |
| C2orf23 | NM_022912 | 1.15358 |
| C3 | NM_000064 | 0.78698 |
| C4BPB | NM_000716 /// NM_001017364 /// | 0.992525 |
| | NM_001017365 /// NM_001017366 /// NM_001017367 | |
| C5orf13 | NM_004772 | 0.966799 |
| C6orf210 | NM_020381 | −0.820329 |
| C6orf216 | NM_206908 /// NM_206910 /// NM_206911 | 1.04882 |
| | /// NM_206912 /// XR_000259 | |
| C8orf1 | NM_004337 | −1.30736 |
| CA12 | NM_001218 /// NM_206925 | −0.904882 |
| CCDC28A | NM_015439 | −1.62476 |
| CCL2 | NM_002982 | 0.911105 |
| CDH1 | NM_004360 | −1.13232 |
| CDH4 | NM_001794 | −0.745807 |
| CDK8 | NM_001260 | −1.16149 |
| CFH | NM_000186 /// NM_001014975 | 0.968934 |
| CGI-38 | NM_015964 /// NM_016140 | −0.742848 |
| CGI-48 | NM_016001 | 1.0641 |
| CHAF1A | NM_005483 | −0.939655 |
| CHGB | NM_001819 | 0.920022 |
| CHORDC1 | NM_012124 | −1.22107 |
| CLDN3 | NM_001306 | −0.982855 |
| CLGN | NM_004362 | 1.28034 |
| CLIC4 | NM_013943 | 1.37928 |
| CLU | NM_001831 /// NM_203339 | 1.18464 |
| CMKOR1 | NM_020311 | 0.74412 |
| COL11A1 | NM_001854 /// NM_080629 /// NM_080630 | 0.813938 |
| COL13A1 | NM_005203 /// NM_080798 /// NM_080799 /// | 1.16345 |
| | NM_080800 /// NM_080801 /// NM_080802 | |
| COL1A1 | NM_000088 | 0.821137 |
| COL3A1 | NM_000090 | 1.09758 |
| COL6A1 | NM_001848 | 0.968416 |
| COMMD8 | NM_017845 | −1.05693 |
| CPE | NM_001873 | 1.07766 |
| CREBL2 | NM_001310 | −1.79105 |
| CRIP2 | NM_001312 | −1.11007 |
| CSPG2 | NM_004385 | −0.911751 |
| CTGF | NM_001901 | 1.25393 |
| CTNND1 | NM_001331 | −0.715801 |
| CXCL1 | NM_001511 | 0.845021 |
| CXCL2 | NM_002089 | 1.01158 |
| CXCL5 | NM_002994 | 0.704588 |
| CYP1B1 | NM_000104 | 0.828644 |
| CYP3A5 | NM_000777 | 0.703318 |
| CYR61 | NM_001554 | 0.764686 |
| DAAM1 | NM_014992 | 0.976142 |
| DAF | NM_000574 | 0.76146 |
| DAPK3 | NM_001348 | −0.779372 |
| DHPS | NM_001930 /// NM_013406 /// NM_013407 | −1.00747 |
| DHRS2 | NM_005794 /// NM_182908 | 1.43654 |
| DIO2 | NM_000793 /// NM_001007023 /// NM_013989 | 0.791523 |
| DKFZP564F0522 | NM_015475 | −1.0877 |
| DPYD | NM_000110 | 1.41139 |
| DST | NM_001723 /// NM_015548 /// | −0.836643 |
| | NM_020388 /// NM_183380 | |
| DZIP1 | NM_014934 /// NM_198968 | 1.03592 |
| E2F5 | NM_001951 | −0.796317 |
| E2F8 | NM_024680 | 1.00205 |
| EEF1D | NM_001960 /// NM_032378 | 0.703203 |
| EFEMP1 | NM_004105 /// NM_018894 | 1.4837 |
| EHD1 | NM_006795 | −0.910559 |
| EIF2C2 | NM_012154 | 1.09581 |
| EIF2S1 | NM_004094 | −1.88674 |
| EIF4E | NM_001968 | −1.2231 |
| ELF3 | NM_004433 | −0.780173 |
| ENPP4 | NM_014936 | 1.19671 |
| EPB41L1 | NM_012156 /// NM_177996 | −1.12118 |
| EPHA2 | NM_004431 | −1.07269 |
| F3 | NM_001993 | 1.31706 |
| FA2H | NM_024306 | −1.34489 |
| FAS | NM_000043 /// NM_152871 /// NM_152872 /// | 0.748072 |
| | NM_152873 /// NM_152874 /// NM_152875 | |
| FBN1 | NM_000138 | 0.87804 |
| FBXO11 | NM_012167 /// NM_018693 /// NM_025133 | 1.06424 |
| FBXW2 | NM_012164 | −1.05455 |
| FDXR | NM_004110 /// NM_024417 | −0.723062 |
| FGB | NM_005141 | 1.38093 |
| FLJ13910 | NM_022780 | 1.05579 |
| FLJ20035 | NM_017631 | 0.859671 |
| FLJ21159 | NM_024826 | −0.829431 |
| FLOT2 | NM_004475 | −0.708745 |
| FOXD1 | NM_004472 | 1.05024 |
| FSTL1 | NM_007085 | 0.989345 |
| FXYD2 | NM_001680 /// NM_021603 | −1.16617 |
| FZD7 | NM_003507 | 1.06154 |
| G0S2 | NM_015714 | 0.906439 |
| GABRA5 | NM_000810 | 0.750404 |
| GALC | NM_000153 | 0.936774 |
| GATA6 | NM_005257 | 1.09725 |
| GCH1 | NM_000161 /// NM_001024024 /// | 0.891087 |
| | NM_001024070 /// NM_001024071 | |
| GFPT2 | NM_005110 | 0.913412 |
| GGT1 | NM_001032364 /// NM_001032365 /// | −0.712035 |
| | NM_005265 /// NM_013430 | |
| GLIPR1 | NM_006851 | 2.13759 |
| GLUL | NM_001033044 /// NM_001033056 /// NM_002065 | −0.849756 |
| GMDS | NM_001500 | −2.14521 |
| GOLPH4 | NM_014498 | 0.95472 |
| GPR64 | NM_005756 | 0.771741 |
| GRB10 | NM_001001549 /// NM_001001550 /// | −1.03799 |
| | NM_001001555 /// NM_005311 | |
| HAS2 | NM_005328 | 0.731898 |
| HECTD3 | NM_024602 | −1.23335 |
| HES1 | NM_005524 | 0.825981 |
| HIC2 | NM_015094 | 0.785963 |
| HIST1H3H | NM_003536 | −0.823929 |
| HKDC1 | NM_025130 | −1.33618 |
| HMGA1 | NM_002131 /// NM_145899 /// NM_145901 /// | −1.408 |
| | NM_145902 /// NM_145903 /// NM_145904 | |
| HMGA2 | NM_001015886 /// NM_003483 /// NM_003484 | −0.91126 |
| HNMT | NM_001024074 /// NM_001024075 /// NM_006895 | 0.734274 |
| HOXA10 | NM_018951 /// NM_153715 | 0.834274 |
| HSPG2 | NM_005529 | −0.747033 |
| HUMPPA | NM_014603 | −1.38414 |
| IDS | NM_000202 /// NM_006123 | −0.798159 |
| IER3IP1 | NM_016097 | 0.804619 |
| IFI16 | NM_005531 | 0.942019 |
| IFIT1 | NM_001001887 /// NM_001548 | −0.752143 |
| IGFBP1 | NM_000596 /// NM_001013029 | −0.79273 |
| IGFBP3 | NM_000598 /// NM_001013398 | 0.842426 |
| IL15 | NM_000585 /// NM_172174 /// NM_172175 | 1.07245 |
| IL27RA | NM_004843 | 1.30764 |
| IL6R | NM_000565 /// NM_181359 | 0.896767 |
| IL6ST | NM_002184 /// NM_175767 | 0.939897 |
| IL8 | NM_000584 | 1.09477 |
| INHBB | NM_002193 | −1.52081 |
| ITGB4 | NM_000213 /// NM_001005619 /// NM_001005731 | −1.21785 |
| ITPR2 | NM_002223 | 0.746339 |
| KCNK3 | NM_002246 | 1.55402 |
| KDELC1 | NM_024089 | 1.18441 |
| KIAA0152 | NM_014730 | −0.941345 |
| KIAA0485 | — | 1.07753 |
| KIAA0527 | XM_171054 | 1.96041 |
| KIAA0830 | XM_290546 | 1.06806 |
| LEPR | NM_001003679 /// NM_001003680 /// NM_002303 | −0.770574 |
| LHX2 | NM_004789 | 1.22767 |
| LMNB1 | NM_005573 | 1.19247 |
| LOC153561 | NM_207331 | 0.764558 |
| LOC389435 | XM_371853 | 0.810852 |
| LOC93349 | NM_138402 | 0.812908 |
| LOXL2 | NM_002318 | −1.38541 |
| LUM | NM_002345 | 1.1044 |
| LYPD1 | NM_144586 | 0.815066 |
| MAPK6 | NM_002748 | −1.20395 |
| MATN3 | NM_002381 | −1.34865 |
| MAZ | NM_002383 | −1.00548 |
| MCAM | NM_006500 | 0.723075 |
| MCL1 | NM_021960 /// NM_182763 | 1.13287 |
| METAP2 | NM_006838 | −1.14678 |
| MGC35048 | NM_153208 | −0.946659 |
| MGC4707 | NM_001003676 /// NM_001003677 | −1.05407 |
| | /// NM_001003678 /// NM_024113 | |
| MRS2L | NM_020662 | −0.910868 |
| MTX2 | NM_001006635 /// NM_006554 | −1.18578 |
| MVP | NM_005115 /// NM_017458 | −1.2441 |
| MYBL1 | NM_034274 | 0.740775 |
| MYCBP | NM_012333 | −1.57357 |
| MYL9 | NM_006097 /// NM_181526 | 1.76885 |
| NAB1 | NM_005966 | −0.838872 |
| NID1 | NM_002508 | 0.705762 |
| NID2 | NM_007361 | 1.93735 |
| NR2F1 | NM_005654 | 1.07657 |
| NR4A2 | NM_006186 /// NM_173171 /// | 0.839422 |
| | NM_173172 /// NM_173173 | |
| NR5A2 | NM_003822 /// NM_205860 | −0.738757 |
| NRG1 | NM_004495 /// NM_013956 /// NM_013957 /// | −1.15784 |
| | NM_013958 /// NM_013959 /// NM_013960 | |
| NRIP1 | NM_003489 | 1.05135 |
| NT5E | NM_002526 | 1.0583 |
| NTE | NM_006702 | −1.02896 |
| NUCKS | NM_022731 | 1.85433 |
| OLFM1 | NM_006334 /// NM_014279 /// NM_058199 | 1.11853 |
| PAPPA | NM_002581 | 1.06925 |
| PBX1 | NM_002585 | 0.715565 |
| PDCD4 | NM_014456 /// NM_145341 | 0.832384 |
| PDE4D | NM_006203 | 0.756904 |
| PDGFRL | NM_006207 | 1.1499 |
| PDK4 | NM_002612 | 0.705278 |
| PDXK | NM_003681 | −1.40137 |
| PDZK1 | NM_002614 | −1.0713 |
| PEG10 | XM_496907 /// XM_499343 | 1.31009 |
| PEX10 | NM_002617 /// NM_153818 | −0.808955 |
| PGK1 | NM_000291 | 1.36181 |
| PHACTR2 | NM_014721 | 0.768814 |
| PLAU | NM_002658 | 0.790224 |
| PLEKHA1 | NM_001001974 /// NM_021622 | 0.925551 |
| PLOD2 | NM_000935 /// NM_182943 | −0.824097 |
| PLSCR4 | NM_020353 | 1.14232 |
| PMCH | NM_002674 | 1.18614 |
| POLR3G | NM_006467 | −1.6809 |
| PPAP2B | NM_003713 /// NM_177414 | 1.04907 |
| PSMB9 | NM_002800 /// NM_148954 | 0.73459 |
| PTGER4 | NM_000958 | 0.799802 |
| PTK9 | NM_002822 /// NM_198974 | 0.841813 |
| PTPN12 | NM_002835 | 1.13139 |
| PTX3 | NM_002852 | 0.958806 |
| PXN | NM_002859 | −0.779877 |
| QKI | NM_006775 /// NM_206853 /// | 0.913473 |
| | NM_206854 /// NM_206855 | |
| RAB11FIP1 | NM_001002233 /// NM_001002814 /// NM_025151 | −1.11162 |
| RAB2 | NM_002865 | 1.08268 |
| RAB21 | NM_014999 | −0.782285 |
| RARRES1 | NM_002888 /// NM_206963 | 0.703277 |
| RCBTB2 | NM_001268 | 1.24665 |
| RDX | NM_002906 | 1.00725 |
| RECK | NM_021111 | 1.34241 |
| RGS2 | NM_002923 | 1.12076 |
| RHEB | NM_005614 | 1.01911 |
| RHOQ | NM_012249 | −1.43035 |
| RHOQ /// LOC284988 | NM_012249 /// NM_209429 | −1.20819 |
| RIP | NM_001033002 /// NM_032308 | 1.25909 |
| ROR1 | NM_005012 | 0.797888 |
| RPL38 | NM_000999 | 0.986019 |
| RPS11 | NM_001015 | 0.786637 |
| RPS6KA5 | NM_004755 /// NM_182398 | 0.783023 |
| S100A2 | NM_005978 | 1.10878 |
| SC4MOL | NM_001017369 /// NM_006745 | −2.06161 |
| SCARB2 | NM_005506 | 0.713034 |
| SCG2 | NM_003469 | 2.1007 |
| SE57-1 | NM_025214 | −1.06691 |
| SEMA3C | NM_006379 | 1.02281 |
| SEPT6 /// N-PAC | NM_015129 /// NM_032569 /// NM_145799 | 0.938411 |
| | /// NM_145800 /// NM_145802 | |
| SEPT9 | NM_006640 | −0.701167 |
| SERPINB9 | NM_004155 | 1.0629 |
| SERPINE2 | NM_006216 | 0.728703 |
| SH3GLB2 | NM_020145 | −0.822875 |
| SHOX2 | NM_003030 /// NM_006884 | 1.22331 |
| SLC26A2 | NM_000112 | 0.70957 |
| SLC2A3 | NM_006931 | −1.3362 |
| SLC2A3 /// SLC2A14 | NM_006931 /// NM_153449 | −0.931892 |
| SLC33A1 | NM_004733 | −1.06356 |
| SMA4 | NM_021652 | 1.11134 |
| SMARCA2 | NM_003070 /// NM_139045 | 0.761273 |
| SNAI2 | NM_003068 | 1.08823 |
| SNAP25 | NM_003081 /// NM_130811 | 1.51132 |
| SORBS3 | NM_001018003 /// NM_005775 | −0.796389 |
| SPANXA1 /// | NM_013453 /// NM_022661 /// NM_032461 /// | 1.53664 |
| SPANXB1 /// | NM_145662 /// NM_145664 | |
| SPANXA2 /// SPANXC | | |
| /// SPANXB2 | | |
| SPARC | NM_003118 | 1.19943 |
| SPOCK | NM_004598 | 1.09606 |
| SRD5A1 | NM_001047 | −1.13979 |
| SRPX | NM_006307 | 1.1299 |
| SSH1 | NM_018984 | 1.02542 |
| STC1 | NM_003155 | 1.13679 |
| STK39 | NM_013233 | −1.35492 |
| SUMO2 | NM_001005849 /// NM_006937 | 0.890434 |
| SYNCRIP | NM_006372 | 1.25513 |
| TAF15 | NM_003487 /// NM_139215 | 0.956591 |
| TAGLN | NM_001001522 /// NM_003186 | 1.32797 |
| TCF4 | NM_003199 | 1.09944 |
| TCF8 | NM_030751 | 0.704819 |
| TGFBR3 | NM_003243 | 1.50748 |
| THBD | NM_000361 | 0.825199 |
| TIMM17A | NM_006335 | −1.14153 |
| TNC | NM_002160 | 2.27045 |
| TNFRSF9 | NM_001561 | 1.08911 |
| TPR | NM_003292 | 0.726403 |
| TRA1 | NM_003299 | 1.64234 |
| TRAPPC4 | NM_016146 | −1.07164 |
| TUBB4 | NM_006087 | −1.39921 |
| TXN | NM_003329 | 1.07471 |
| UGT1A8 /// UGT1A9 | NM_019076 /// NM_021027 | −1.1245 |
| ULK1 | NM_003565 | −1.31566 |
| UQCRB | NM_006294 | −1.12095 |
| VAV3 | NM_006113 | −0.951341 |
| VDAC1 | NM_003374 | −0.976178 |
| VDR | NM_000376 /// NM_001017535 | 1.09287 |
| VEGFC | NM_005429 | 1.05478 |
| WDR76 | NM_024908 | 0.710363 |
| XTP2 | NM_015172 | 0.775788 |
| YDD19 | — | −1.14172 |
| YDD19 /// C6orf68 /// | NM_138459 /// XM_372205 /// XR_000254 | −1.23685 |
| LOC389850 /// | | |
| LOC440128 | | |
| ZNF259 | NM_003904 | −1.00795 |
| ZNF551 | NM_138347 | 0.884017 |
| ZNF573 | NM_152360 | 1.31557 |

TABLE 1C
Genes with increased (positive values) or decreased (negative values)
expression following transfection of human cancer cells with
anti-hsa-miR-31.

| | RefSeq | |
|---|---|---|
| Gene Symbol | Transcript ID (Pruitt et al., 2005) | Δ log₂ |
| AKAP2 /// PALM2- | NM_001004065 /// NM_007203 /// | 0.881687 |
| AKAP2 | NM_147150 | |
| ANPEP | NM_001150 | 0.773871 |
| AXL | NM_001699 /// NM_021913 | 0.867317 |
| BIRC3 | NM_001165 /// NM_182962 | 0.736116 |
| CXCL1 | NM_001511 | 1.18869 |
| CXCL2 | NM_002089 | 1.1814 |
| CXCL3 | NM_002090 | 0.800224 |
| CXCL5 | NM_002994 | 0.844167 |
| HIPK3 | NM_005734 | 0.761797 |
| IL6ST | NM_002184 /// NM_175767 | 0.85816 |
| IL8 | NM_000584 | 1.54253 |
| LRP12 | NM_013437 | 0.745576 |
| MAFF | NM_012323 /// NM_152878 | 0.873461 |
| NID1 | NM_002508 | 0.818989 |
| OPLAH | NM_017570 | 0.721461 |
| PTGS2 | NM_000963 | 0.832017 |
| PTPN12 | NM_002835 | 0.727176 |
| QKI | NM_006775 /// NM_206853 /// | 0.773843 |
| | NM_206854 /// NM_206855 | |
| RDX | NM_002906 | 0.936655 |
| SLC26A2 | NM_000112 | 0.784073 |
| SOD2 | NM_000636 /// NM_001024465 /// | 1.12431 |
| | NM_001024466 | |
| SPTBN1 | NM_003128 /// NM_178313 | 0.723649 |
| STC1 | NM_003155 | 0.904092 |
| TNC | NM_002160 | 0.715844 |
| TNFAIP3 | NM_006290 | 0.788213 |

TABLE 1D
Genes with increased (positive values) or decreased (negative values)
expression following transfection of human cancer cells with pre-miR
hsa-miR-145.

| Gene | RefSeq Transcript | |
|---|---|---|
| Symbol | ID (Pruitt et al., 2005) | Δ log₂ |
| AXL | NM_001699 /// NM_021913 | 0.775236939 |
| CGI-48 | NM_016001 | 0.771224792 |
| CXCL3 | NM_002090 | 0.742720639 |
| IL8 | NM_000584 | 0.769997216 |
| LMO4 | NM_006769 | −0.715738257 |
| NUCKS | NM_022731 | 0.763122861 |
| PGK1 | NM_000291 | 0.847051401 |
| PMCH | NM_002674 | 0.865940473 |
| RAB2 | NM_002865 | 0.807863694 |
| RDX | NM_002906 | 0.743529157 |
| RPL38 | NM_000999 | 0.739789501 |
| TRA1 | NM_003299 | 1.107966463 |
| TXN | NM_003329 | 0.843252007 |

TABLE 1E
Genes with increased (positive values) or decreased (negative values)
expression following transfection of human cancer cells with pre-miR hsa-miR-147.

| Gene Symbol | RefSeq Transcript ID (Pruitt et al., 2005) | Δ log₂ |
|---|---|---|
| ABCA1 | NM_005502 | −1.0705079 |
| ALDH6A1 | NM_005589 | 0.921996293 |
| ANK3 | NM_001149 /// NM_020987 | 1.175319831 |
| ANKRD46 | NM_198401 | 0.798089258 |
| ANTXR1 | NM_018153 /// NM_032208 /// NM_053034 | −1.290010791 |
| ANXA10 | NM_007193 | −0.76954436 |
| APOH | NM_000042 | 1.116058445 |
| AQP3 | NM_004925 | 1.293583496 |
| ARG2 | NM_001172 | 2.214496965 |
| ARHGDIA | NM_004309 | −0.71895894 |
| ARID5B | NM_032199 | 1.249175823 |
| ARL2BP | NM_012106 | 0.852981303 |
| ARL7 | NM_005737 | −1.097275914 |
| ARTS-1 | NM_016442 | −0.754098539 |
| ATF5 | NM_012068 | −0.716057584 |
| ATP6V0E | NM_003945 | −0.84096275 |
| ATP9A | NM_006045 | 0.752911182 |
| AXL | NM_001699 /// NM_021913 | 0.793637153 |
| B4GALT1 | NM_001497 | −0.776574082 |
| BCL2A1 | NM_004049 | −2.000359314 |
| BCL6 | NM_001706 /// NM_138931 | 0.751950658 |
| BICD2 | NM_001003800 /// NM_015250 | −0.818215213 |
| BTG3 | NM_006806 | −1.374399564 |
| BTN3A2 | NM_007047 | −1.06699734 |
| C19orf2 | NM_003796 /// NM_134447 | −0.876512872 |
| C1orf24 | NM_022083 /// NM_052966 | −0.78341048 |
| C21orf25 | NM_199050 | −1.053798237 |
| C2orf17 | NM_024293 | −1.039115573 |
| C2orf31 | — | 0.791392536 |
| C6orf120 | NM_001029863 | −0.832480385 |
| CA12 | NM_001218 /// NM_206925 | −0.989153023 |
| CA2 | NM_000067 | 0.733866747 |
| CASP7 | NM_001227 /// NM_033338 /// NM_033339 /// | −0.780385444 |
| | NM_033340 | |
| CCL2 | NM_002982 | −1.182060911 |
| CCND1 | NM_053056 | −1.435105691 |
| CCNG1 | NM_004060 /// NM_199246 | 0.928408016 |
| CDC37L1 | NM_017913 | −1.026820179 |
| CDH4 | NM_001794 | −1.027487702 |
| COBLL1 | NM_014900 | 0.931189433 |
| COL3A1 | NM_000090 | 0.969777477 |
| COL4A1 | NM_001845 | −1.178971961 |
| COL4A2 | NM_001846 | −1.459851683 |
| COQ2 | NM_015697 | −0.83915296 |
| CRIPT | NM_014171 | −1.110146535 |
| CSNK1A1 | NM_001025105 /// NM_001892 | −0.717262814 |
| CSPG2 | NM_004385 | −1.037433363 |
| CTDSP2 | NM_005730 | 1.103871011 |
| CTH | NM_001902 /// NM_153742 | 1.482227168 |
| CTSS | NM_004079 | −0.704674455 |
| CXCL5 | NM_002994 | 0.758779818 |
| DAZAP2 | NM_014764 | −1.232967024 |
| DAZAP2 /// | NM_014764 /// XM_376165 | −0.876163094 |
| LOC401029 | | |
| DCBLD2 | NM_080927 | −0.813731475 |
| DCP2 | NM_152624 | 1.187108067 |
| DDAH1 | NM_012137 | 1.133236922 |
| DHCR24 | NM_014762 | 0.962804049 |
| DIO2 | NM_000793 /// NM_001007023 /// NM_013989 | −0.809284862 |
| DKFZP586A0522 | NM_014033 | 0.957989488 |
| DNAJB6 | NM_005494 /// NM_058246 | −1.120505456 |
| DNAJC15 | NM_013238 | 1.186534996 |
| DOCK4 | NM_014705 | −0.824536256 |
| DPYSL4 | NM_006426 | 0.800773508 |
| DSC2 | NM_004949 /// NM_024422 | 1.11600402 |
| DST | NM_001723 /// NM_015548 /// | 1.317689575 |
| | NM_020388 /// NM_183380 | |
| DUSP1 | NM_004417 | −1.036787804 |
| EIF2C1 | NM_012199 | −0.849818302 |
| EIF2S1 | NM_004094 | −1.211812274 |
| EIF5A2 | NM_020390 | −0.703223281 |
| EPHB2 | NM_004442 /// NM_017449 | −1.171343772 |
| EREG | NM_001432 | −1.346940189 |
| ETS2 | NM_005239 | −0.783135629 |
| F2RL1 | NM_005242 | −0.861042737 |
| FAM18B | NM_016078 | −0.768704947 |
| FAM45B /// | NM_018472 /// NM_207009 | −0.905122961 |
| FAM45A | | |
| FAM46A | NM_017633 | 1.189436349 |
| FGB | NM_005141 | 1.133519364 |
| FGFR3 | NM_000142 /// NM_022965 | 1.175488465 |
| FGFR4 | NM_002011 /// NM_022963 /// NM_213647 | 0.778320037 |
| FGG | NM_000509 /// NM_021870 | 1.161946748 |
| FGL1 | NM_004467 /// NM_147203 /// | 0.920382947 |
| | NM_201552 /// NM_201553 | |
| FJX1 | NM_014344 | −1.631423993 |
| FLJ13910 | NM_022780 | 0.874893502 |
| FLJ21159 | NM_024826 | −0.836849616 |
| FLJ31568 | NM_152509 | 1.050523485 |
| FLRT3 | NM_013281 /// NM_198391 | 1.084587332 |
| FOSL1 | NM_005438 | −1.004370563 |
| FTS | NM_001012398 /// NM_022476 | −1.105648276 |
| FYCO1 | NM_024513 | −1.849492859 |
| FZD7 | NM_003507 | 0.730854769 |
| G1P2 | NM_005101 | −1.070255287 |
| GABRA5 | NM_000810 | −1.370874696 |
| GATA6 | NM_005257 | 1.250224603 |
| GK | NM_000167 /// NM_203391 | 0.823046538 |
| GLI2 | NM_005270 /// NM_030379 /// | −0.770685407 |
| | NM_030380 /// NM_030381 | |
| GLIPR1 | NM_006851 | −1.047885319 |
| GLUL | NM_001033044 /// NM_001033056 /// | 0.889617404 |
| | NM_002065 | |
| GNS | NM_002076 | −1.07857689 |
| GOLPH2 | NM_016548 /// NM_177937 | −0.926612282 |
| GYG2 | NM_003918 | 0.975758283 |
| HAS2 | NM_005328 | −1.136601383 |
| HCCS | NM_005333 | −1.169843196 |
| HIC2 | NM_015094 | 1.040798749 |
| HKDC1 | NM_025130 | −0.742677043 |
| HMGCS1 | NM_002130 | 0.710761737 |
| HN1 | NM_001002032 /// NM_001002033 /// | −1.288713253 |
| | NM_016185 | |
| ID4 | NM_001546 | 1.050108032 |
| IDS | NM_000202 /// NM_006123 | −0.765358291 |
| IGFBP1 | NM_000596 /// NM_001013029 | −1.279099713 |
| IGFBP4 | NM_001552 | −0.739326913 |
| IL11 | NM_000641 | −2.089747129 |
| IL15 | NM_000585 /// NM_172174 /// NM_172175 | −0.854711689 |
| IL8 | NM_000584 | −1.711808874 |
| IQGAP2 | NM_006633 | 0.913042194 |
| ITGB4 | NM_000213 /// NM_001005619 /// | −1.186739806 |
| | NM_001005731 | |
| JAK1 | NM_002227 | −1.059987123 |
| JUN | NM_002228 | −0.846308702 |
| KCNMA1 | NM_001014797 /// NM_002247 | −1.281096095 |
| KCNS3 | NM_002252 | 0.763898782 |
| KIAA0494 | NM_014774 | −1.372898343 |
| KIAA0882 | NM_015130 | −0.980703295 |
| KLF10 | NM_001032282 /// NM_005655 | −1.116428 |
| KRT4 | NM_002272 | 1.064537576 |
| LEPROT | NM_017526 | −1.018363603 |
| LHFP | NM_005780 | −1.0271939 |
| LIMK1 | NM_002314 /// NM_016735 | −1.803777658 |
| LRP12 | NM_013437 | −0.743603255 |
| LRRC54 | NM_015516 | −0.77656268 |
| M6PR | NM_002355 | −1.386148277 |
| MAP3K1 | XM_042066 | 0.759959443 |
| MAP3K2 | NM_006609 | −1.363559174 |
| MARCH6 | NM_005885 | −1.202139411 |
| MATN3 | NM_002381 | 0.903494673 |
| MGAM | NM_004668 | 1.167350858 |
| MGC11332 | NM_032718 | −1.007976707 |
| MICA | NM_000247 | −1.41026822 |
| MICAL2 | NM_014632 | −0.823900817 |
| MOBK1B | NM_018221 | −1.127633961 |
| NAGK | NM_017567 | −1.06761962 |
| NAV3 | NM_014903 | −0.701500848 |
| NES | NM_006617 | 0.824166211 |
| NID1 | NM_002508 | 0.712358426 |
| NPAS2 | NM_002518 | −1.314671396 |
| NPTX1 | NM_002522 | −1.366083158 |
| NUPL1 | NM_001008564 /// NM_001008565 /// | −0.927879559 |
| | NM_014089 | |
| OBSL1 | XM_051017 | 1.078419022 |
| OLFML3 | NM_020190 | −0.772616072 |
| OLR1 | NM_002543 | 0.783582212 |
| OSTM1 | NM_014028 | −1.349848003 |
| OXTR | NM_000916 | −1.248290182 |
| P8 | NM_012385 | 1.102960353 |
| PDCD4 | NM_014456 /// NM_145341 | 0.732196292 |
| PDZK1 | NM_002614 | 1.13249347 |
| PDZK1IP1 | NM_005764 | −0.764992528 |
| PELI2 | NM_021255 | 1.052234224 |
| PFKP | NM_002627 | −1.304130926 |
| PKP2 | NM_001005242 /// NM_004572 | 0.957319593 |
| PLAU | NM_002658 | −1.546762739 |
| POLR3G | NM_006467 | −1.758348197 |
| PON2 | NM_000305 /// NM_001018161 | −0.891886921 |
| PSMB9 | NM_002800 /// NM_148954 | −0.764503658 |
| PTHLH | NM_002820 /// NM_198964 /// | −0.85479181 |
| | NM_198965 /// NM_198966 | |
| RAB11FIP1 | NM_001002233 /// NM_001002814 /// | −0.710783895 |
| | NM_025151 | |
| RAB22A | NM_020673 | −1.287081241 |
| RARRES1 | NM_002888 /// NM_206963 | 0.766334915 |
| RBKS | NM_022128 | −1.116205272 |
| RGC32 | NM_014059 | 0.956745628 |
| RHOC | NM_175744 | −1.073877719 |
| RNH1 | NM_002939 /// NM_203383 /// NM_203384 | −1.119287238 |
| | /// | |
| | NM_203385 /// NM_203386 /// NM_203387 | |
| RRM2 | NM_001034 | −1.047471119 |
| S100P | NM_005980 | 1.564388795 |
| SERF1A /// | NM_021967 /// NM_022978 | −1.00166157 |
| SERF1B | | |
| SERPINE1 | NM_000602 | −2.401636366 |
| SGPL1 | NM_003901 | −0.977828602 |
| SKP2 | NM_005983 /// NM_032637 | 0.7230064 |
| SLC26A2 | NM_000112 | −0.804718831 |
| SPANXA1 /// | NM_013453 /// NM_022661 /// NM_032461 | 0.723441371 |
| SPANXB1 /// | /// | |
| SPANXA2 /// | NM_145662 /// NM_145664 | |
| SPANXC /// | | |
| SPANXB2 | | |
| SPARC | NM_003118 | 1.275598165 |
| SPOCK | NM_004598 | −1.416025909 |
| STC1 | NM_003155 | −1.031822774 |
| STX3A | NM_004177 | 0.738540782 |
| SYNE1 | NM_015293 /// NM_033071 /// | −0.986137779 |
| | NM_133650 /// NM_182961 | |
| TBC1D2 | NM_018421 | −1.036883659 |
| TGFBR2 | NM_0010248471 /// NM_003242 | −1.121957889 |
| TJP2 | NM_004817 /// NM_201629 | 1.028659136 |
| TM4SF20 | NM_024795 | 0.857516073 |
| TM4SF4 | NM_004617 | −0.844385261 |
| TM7SF1 | NM_003272 | −1.650275939 |
| TMC5 | NM_024780 | −0.810437274 |
| TMEPAI | NM_020182 /// NM_199169 /// | −1.096653239 |
| | NM_199170 /// NM_199171 | |
| TNFAIP6 | NM_007115 | −1.865722451 |
| TNFRSF12A | NM_016639 | −0.842444428 |
| TNRC9 | XM_049037 | 0.870669505 |
| TSPAN8 | NM_004616 | 0.735887176 |
| TXLNA | NM_175852 | −0.882047143 |
| UEV3 | NM_018314 | −1.113012978 |
| ULK1 | NM_003565 | −0.728593583 |
| USP46 | NM_022832 | −1.598797937 |
| VANGL1 | NM_138959 | −1.036428715 |
| VDR | NM_000376 /// NM_001017535 | −0.744474059 |
| VLDLR | NM_001018056 /// NM_003383 | −1.105779636 |
| VTN | NM_000638 | 0.969767951 |
| WBSCR22 | NM_017528 | −0.703785254 |
| ZBTB10 | NM_023929 | 0.853410353 |
| ZNF467 | NM_207336 | 1.07813993 |

TABLE 1F
Genes with increased (positive values) or decreased (negative values)
expression following transfection of human cancer cells with pre-miR hsa-miR-188.

| Gene Symbol | RefSeq Transcript ID (Pruitt et al., 2005) | □ log₂ |
|---|---|---|
| — | XM_371853 | 0.79767725 |
| 15E1.2 | NM_176818 | −1.141638876 |
| ADARB1 | NM_001033049 /// NM_001112 /// | 0.744410733 |
| | NM_015833 /// NM_015834 | |
| AER61 | NM_173654 | −0.899131245 |
| AKAP2 /// PALM2-AKAP2 | NM_001004065 /// NM_007203 /// | −0.941957418 |
| | NM_147150 | |
| ANKRD46 | NM_198401 | 0.834094665 |
| ANTXR1 | NM_018153 /// NM_032208 /// | 0.757775366 |
| | NM_053034 | |
| AR | NM_000044 /// NM_001011645 | −0.805079746 |
| ARL2BP | NM_012106 | 0.797577768 |
| ATP2B4 | NM_001001396 /// NM_001684 | −1.153875577 |
| ATP6V0E | NM_003945 | 1.113609299 |
| ATXN1 | NM_000332 | −1.225362507 |
| AXL | NM_001699 /// NM_021913 | 0.741305367 |
| B4GALT1 | NM_001497 | −0.787396891 |
| B4GALT4 | NM_003778 /// NM_212543 | −0.797950275 |
| BAMBI | NM_012342 | −0.832397669 |
| BCL6 | NM_001706 /// NM_138931 | −0.807800523 |
| BPGM | NM_001724 /// NM_199186 | −1.729772661 |
| C3 | NM_000064 | 0.776240618 |
| C6orf120 | NM_001029863 | −1.427214532 |
| C8orf1 | NM_004337 | −0.783453122 |
| CACNA1G | NM_018896 /// NM_198376 /// | −0.707185799 |
| | NM_198377 /// | |
| | NM_198378 /// NM_198379 /// | |
| | NM_198380 | |
| CAP1 | NM_006367 | −1.13643337 |
| CBFB | NM_001755 /// NM_022845 | −1.261357593 |
| CCDC6 | NM_005436 | −1.009649239 |
| CCNA2 | NM_001237 | −0.791748727 |
| CD2AP | NM_012120 | −1.121212839 |
| CDH1 | NM_004360 | −0.977612615 |
| CDK2AP1 | NM_004642 | −1.537435476 |
| CGI-48 | NM_016001 | 1.035693465 |
| CLU | NM_001831 /// NM_203339 | −1.205042129 |
| COL1A1 | NM_000088 | −1.058828289 |
| COL6A1 | NM_001848 | 0.735178781 |
| CREB3L2 | NM_194071 | −1.092835167 |
| CSNK1A1 | NM_001025105 /// NM_001892 | −1.183929257 |
| CSPG2 | NM_004385 | −0.850672076 |
| CXCL1 | NM_001511 | 0.876432556 |
| CXCL2 | NM_002089 | 0.797235609 |
| DAAM1 | NM_014992 | −0.859090846 |
| DCP2 | NM_152624 | 0.972517476 |
| DDAH1 | NM_012137 | 0.885174702 |
| DHRS2 | NM_005794 /// NM_182908 | 1.085977439 |
| DIO2 | NM_000793 /// NM_001007023 /// | 0.979459766 |
| | NM_013989 | |
| DKFZp564K142 | NM_032121 | −1.413051709 |
| DLG5 | NM_004747 | −1.157557972 |
| EDEM1 | NM_014674 | −1.180379773 |
| EIF2S1 | NM_004094 | −1.263958652 |
| ELF3 | NM_004433 | −1.133314137 |
| ELOVL6 | NM_024090 | −0.722875346 |
| EMP1 | NM_001423 | −0.83814704 |
| ENPP4 | NM_014936 | 0.744738095 |
| ETS2 | NM_005239 | −1.020837722 |
| FAM18B | NM_016078 | −0.717468957 |
| FEM1B | NM_015322 | −1.158919916 |
| FGF2 | NM_002006 | −0.843439627 |
| FGG | NM_000509 /// NM_021870 | −0.763121708 |
| FLJ13910 | NM_022780 | 0.818728904 |
| FN5 | NM_020179 | −1.270232536 |
| GABRA5 | NM_000810 | 0.772270023 |
| GATAD1 | NM_021167 | −1.295620295 |
| GPR125 | NM_145290 | −1.243715655 |
| GREM1 | NM_013372 | −1.068628761 |
| H2AFY | NM_004893 /// NM_138609 /// | −0.93507394 |
| | NM_138610 | |
| HDAC3 | NM_003883 | −0.73639501 |
| HIPK3 | NM_005734 | 0.892438313 |
| HNRPA0 | NM_006805 | −1.164494165 |
| IDS | NM_000202 /// NM_006123 | −1.270124871 |
| IER3IP1 | NM_016097 | 0.707420006 |
| IGFBP3 | NM_000598 /// NM_001013398 | 0.707305602 |
| IL11 | NM_000641 | −1.199790518 |
| IL13RA1 | NM_001560 | −1.079298214 |
| IL6ST | NM_002184 /// NM_175767 | −1.000365688 |
| IL8 | NM_000584 | 1.192438588 |
| INHBC | NM_005538 | 0.947119793 |
| ITGAV | NM_002210 | −0.830296216 |
| KCNJ2 | NM_000891 | 0.756259837 |
| KLF4 | NM_004235 | −1.094778613 |
| LGALS8 | NM_006499 /// NM_201543 /// | −1.161162739 |
| | NM_201544 /// NM_201545 | |
| LOC348162 | XM_496132 | −0.754126245 |
| LOC440118 | XM_498554 | 1.068888477 |
| LOC492304 | NM_001007139 | −0.993171411 |
| LZTFL1 | NM_020347 | 1.067917522 |
| M6PR | NM_002355 | −0.702214209 |
| MAP4K5 | NM_006575 /// NM_198794 | −1.315004609 |
| MARCKS | NM_002356 | −1.719459875 |
| MCL1 | NM_021960 /// NM_182763 | 0.851818869 |
| NEFL | NM_006158 | 0.894724681 |
| NUCKS | NM_022731 | 0.809644166 |
| PALM2-AKAP2 | NM_007203 /// NM_147150 | −0.952675045 |
| PCAF | NM_003884 | −0.884319067 |
| PCTP | NM_021213 | −1.860357999 |
| PDZK1IP1 | NM_005764 | 0.814065246 |
| PER2 | NM_003894 /// NM_022817 | −0.820618961 |
| PGK1 | NM_000291 | 1.458841167 |
| PHACTR2 | NM_014721 | −0.994794647 |
| PLEKHA1 | NM_001001974 /// NM_021622 | −1.087541297 |
| PMCH | NM_002674 | 0.891819035 |
| PPAP2B | NM_003713 /// NM_177414 | 1.09654097 |
| PRKCA | NM_002737 | −0.74986976 |
| PTEN | NM_000314 | −1.18340148 |
| RAB22A | NM_020673 | −0.857364776 |
| RASSF3 | NM_178169 | −1.056858481 |
| RBL1 | NM_002895 /// NM_183404 | −1.832181472 |
| RGS20 | NM_003702 /// NM_170587 | −1.031805989 |
| RHEB | NM_005614 | 1.046807861 |
| RIP | NM_001033002 /// NM_032308 | 1.002233258 |
| RNASE4 | NM_002937 /// NM_194430 /// | −1.041252911 |
| | NM_194431 | |
| RPL38 | NM_000999 | 1.018133464 |
| RPS11 | NM_001015 | 0.711318114 |
| RRAGD | NM_021244 | 1.032780698 |
| RSAD1 | NM_018346 | −1.158852158 |
| SDC4 | NM_002999 | −0.827651439 |
| SEMA3C | NM_006379 | 0.728585504 |
| SFRS7 | NM_001031684 /// NM_006276 | −1.839856588 |
| SLC39A9 | NM_018375 | −1.641258804 |
| SLC4A4 | NM_003759 | −0.735121994 |
| SNAP25 | NM_003081 /// NM_130811 | 0.867961925 |
| SOCS2 | NM_003877 | 0.794942635 |
| SOX18 | NM_018419 | 2.106732425 |
| ST13 | NM_003932 | −1.524583796 |
| STC1 | NM_003155 | 0.734717673 |
| SYNJ2BP | NM_018373 | −1.080440275 |
| TAPBP | NM_003190 /// NM_172208 /// | −1.960164768 |
| | NM_172209 | |
| TBL1X | NM_005647 | −0.868396691 |
| TM4SF4 | NM_004617 | 1.144720409 |
| TMBIM1 | NM_022152 | −1.287361343 |
| TNRC9 | XM_049037 | −0.771759846 |
| TOX | NM_014729 | 0.758056848 |
| TP73L | NM_003722 | −1.07919526 |
| TRA1 | NM_003299 | 1.168505036 |
| TRPC1 | NM_003304 | −1.27624829 |
| TXN | NM_003329 | 1.396905762 |
| VAPB | NM_004738 | −1.101210395 |
| VAV3 | NM_006113 | −1.259645983 |
| WDR39 | NM_004804 | −1.124206635 |
| WDR41 | NM_018268 | −0.858885381 |
| WISP2 | NM_003881 | 1.240802507 |
| WSB2 | NM_018639 | 0.725624688 |
| ZNF281 | NM_012482 | −1.086219759 |

TABLE 1G
Genes with increased (positive values) or decreased (negative values)
expression following transfection of human cancer cells with pre-miR hsa-miR-215.

| Gene Symbol | RefSeq Transcript ID (Pruitt et al., 2005) | Δ log₂ |
|---|---|---|
| AASDHPPT | NM_015423 | −1.494197703 |
| ABHD3 | NM_138340 | 0.854113684 |
| ABLIM3 | NM_014945 | 0.952575867 |
| ACADSB | NM_001609 | −1.055415881 |
| ADCY7 | NM_001114 | −1.016445175 |
| ADRB2 | NM_000024 | 1.151729447 |
| AER61 | NM_173654 | −0.750205603 |
| AKAP2 /// PALM2-AKAP2 | NM_001004065 /// NM_007203 /// | 0.998820355 |
| | NM_147150 | |
| ANG /// RNASE4 | NM_001145 /// NM_002937 /// | −0.789162296 |
| | NM_194430 /// NM_194431 | |
| ANKRD12 | NM_015208 | 0.83611804 |
| ANTXR1 | NM_018153 /// NM_032208 /// | −0.989899193 |
| | NM_053034 | |
| AOX1 | NM_001159 | 1.057940273 |
| APP | NM_000484 /// NM_201413 /// | 1.032937045 |
| | NM_201414 | |
| AQP3 | NM_004925 | −1.164146946 |
| ARF7 | NM_025047 | 1.114359532 |
| ARHGAP11A | NM_014783 /// NM_199357 | −1.073287033 |
| ARHGAP29 | NM_004815 | −1.569413849 |
| ARL2BP | NM_012106 | 0.786926841 |
| ARTS-1 | NM_016442 | 0.852001464 |
| ATP2B4 | NM_001001396 /// NM_001684 | 0.723181241 |
| ATP6V0E | NM_003945 | 1.51677341 |
| B4GALT6 | NM_004775 | −0.766238067 |
| BCL2L13 | NM_015367 | −0.983341665 |
| BDKRB2 | NM_000623 | −0.828248001 |
| BUB1 | NM_004336 | −0.827828304 |
| C1D | NM_006333 /// NM_173177 | −1.20890231 |
| C21orf25 | NM_199050 | 0.786708643 |
| C3 | NM_000064 | 0.827896244 |
| C6orf210 | NM_020381 | −0.782879379 |
| C6orf216 | NM_206908 /// NM_206910 /// | 1.416623897 |
| | NM_206911 /// | |
| | NM_206912 /// XR_000259 | |
| C9orf95 | NM_017881 | 1.031138782 |
| CALB2 | NM_001740 /// NM_007087 /// | 1.14387436 |
| | NM_007088 | |
| CBFB | NM_001755 /// NM_022845 | −1.091964495 |
| CCNG1 | NM_004060 /// NM_199246 | 1.083676653 |
| CD38 | NM_001775 | −0.830682734 |
| CD44 | NM_000610 /// NM_001001389 /// | 0.790659843 |
| | NM_001001390 /// | |
| | NM_001001391 /// NM_001001392 | |
| CDCA4 | NM_017955 /// NM_145701 | −1.041629919 |
| CDH1 | NM_004360 | −0.718140698 |
| CGI-48 | NM_016001 | 1.375743217 |
| CHAF1A | NM_005483 | −0.810171421 |
| CKLFSF6 | NM_017801 | −1.05964196 |
| CLCN4 | NM_001830 | −0.769302492 |
| CLN8 | NM_018941 | 0.858122772 |
| COL6A1 | NM_001848 | 0.849959567 |
| COPS7A | NM_016319 | −1.253849195 |
| CPNE1 | NM_003915 /// NM_152925 /// | −1.009304194 |
| | NM_152926 /// | |
| | NM_152927 /// NM_152928 /// | |
| | NM_152929 | |
| CPS1 | NM_001875 | −1.3665196 |
| CRISPLD2 | NM_031476 | 0.892157417 |
| CRSP2 | NM_004229 | −1.210756034 |
| CTAGE5 | NM_005930 /// NM_203354 /// | 0.841770238 |
| | NM_203355 | |
| | /// NM_203356 /// NM_203357 | |
| CTH | NM_001902 /// NM_153742 | −0.80511771 |
| CTSS | NM_004079 | 0.943772117 |
| CYP3A5 | NM_000777 | 1.043569459 |
| DAAM1 | NM_014992 | 0.727241047 |
| DDAH1 | NM_012137 | 0.808782614 |
| DDEF1 | NM_018482 | 0.792377983 |
| DEAF1 | NM_021008 | −1.007418894 |
| DIAPH2 | NM_006729 /// NM_007309 | −1.008176565 |
| DICER1 | NM_030621 /// NM_177438 | −1.012881586 |
| DIO2 | NM_000793 /// NM_001007023 /// | −0.739784298 |
| | NM_013989 | |
| DLG5 | NM_004747 | −0.912864833 |
| DMN | NM_015286 /// NM_145728 | −0.821232265 |
| DST | NM_001723 /// NM_015548 /// | −1.187600467 |
| | NM_020388 /// NM_183380 | |
| DTL | NM_016448 | −0.782239408 |
| E2F8 | NM_024680 | −1.548471897 |
| EEF1D | NM_001960 /// NM_032378 | 1.078924091 |
| EFEMP1 | NM_004105 /// NM_018894 | −1.878885511 |
| EHF | NM_012153 | 0.790943966 |
| ELOVL5 | NM_021814 | −1.417385236 |
| ENO1 | NM_001428 | 0.904531556 |
| EREG | NM_001432 | −1.0039753 |
| ETS2 | NM_005239 | −0.782193852 |
| F3 | NM_001993 | 0.890038387 |
| FAS | NM_000043 /// NM_152871 /// | 1.109878838 |
| | NM_152872 /// | |
| | NM_152873 /// NM_152874 /// | |
| | NM_152875 | |
| FBLN1 | NM_001996 /// NM_006485 /// | −1.198559916 |
| | NM_006486 /// NM_006487 | |
| FGB | NM_005141 | −0.988027206 |
| FGF2 | NM_002006 | −1.547807242 |
| FGFR1 | NM_000604 /// NM_015850 /// | −1.080430655 |
| | NM_023105 /// | |
| | NM_023106 /// NM_023107 /// | |
| | NM_023108 | |
| FGFR4 | NM_002011 /// NM_022963 /// | −0.817299388 |
| | NM_213647 | |
| FGG | NM_000509 /// NM_021870 | −1.492473759 |
| FGL1 | NM_004467 /// NM_147203 /// | −0.713631566 |
| | NM_201552 /// NM_201553 | |
| FLJ10719 | NM_018193 | −1.059202598 |
| FLJ13910 | NM_022780 | 0.926035164 |
| FLRT3 | NM_013281 /// NM_198391 | −0.81081052 |
| FOSL1 | NM_005438 | 0.703562091 |
| FOXD1 | NM_004472 | −1.464576387 |
| GART | NM_000819 /// NM_175085 | −1.020828467 |
| GATM | NM_001482 | −0.747694817 |
| GFPT2 | NM_005110 | 0.747425943 |
| GLIPR1 | NM_006851 | 0.715270052 |
| GOLGA4 | NM_002078 | 1.126845538 |
| GREB1 | NM_014668 /// NM_033090 /// | 1.160784669 |
| | NM_148903 | |
| GREM1 | NM_013372 | −0.844806788 |
| HAS2 | NM_005328 | −0.755637003 |
| HBXIP | NM_006402 | −1.154923271 |
| HNMT | NM_001024074 /// NM_001024075 /// | 0.873425234 |
| | NM_006895 | |
| HOXA10 | NM_018951 /// NM_153715 | −1.218730945 |
| HSA9761 | NM_014473 | −1.431312039 |
| IGFBP3 | NM_000598 /// NM_001013398 | −0.704019291 |
| IGFBP4 | NM_001552 | −0.960491248 |
| IL11 | NM_000641 | −2.157215444 |
| IL1R1 | NM_000877 | −1.407994856 |
| IL32 | NM_001012631 /// NM_001012632 /// | 0.860970201 |
| | NM_001012633 /// NM_001012634 /// | |
| | NM_001012635 | |
| IL8 | NM_000584 | 0.968483336 |
| INSIG1 | NM_005542 /// NM_198336 /// | −0.984471288 |
| | NM_198337 | |
| INSL4 | NM_002195 | −1.023618945 |
| IQGAP2 | NM_006633 | −1.034719984 |
| KIAA0485 | — | 1.003889745 |
| KIAA0754 | — | 0.761240845 |
| KIAA1641 | NM_020970 | 1.551418203 |
| KIAA1659 | — | 0.952705814 |
| KRT7 | NM_005556 | 0.783287062 |
| LAMB3 | NM_000228 /// NM_001017402 | 0.872667082 |
| LAMP1 | NM_005561 | −0.860008347 |
| LEPREL1 | NM_018192 | −1.226360629 |
| LMAN1 | NM_005570 | −1.531831162 |
| LOC137886 | XM_059929 | −1.199916073 |
| LOC153561 | NM_207331 | 1.182493824 |
| LOC348162 | XM_496132 | 0.803798804 |
| LOC440118 | XM_498554 | 1.75097398 |
| LOC93349 | NM_138402 | 0.878494103 |
| LXN | NM_020169 | −1.043500775 |
| MAP3K2 | NM_006609 | 0.771218938 |
| MAPKAPK2 | NM_004759 /// NM_032960 | −1.273812576 |
| MAZ | NM_002383 | −1.129157916 |
| MCM10 | NM_018518 /// NM_182751 | −0.744055676 |
| MCM3 | NM_002388 | −0.834267511 |
| MCM5 | NM_006739 | −0.77427783 |
| MGC3196 | XM_495878 | −0.799900884 |
| MGC4172 | NM_024308 | −1.029995038 |
| MLF1 | NM_022443 | −1.114462589 |
| MMP7 | NM_002423 | 0.712659835 |
| MNS1 | NM_018365 | −1.105575972 |
| MRPL13 | NM_014078 | −1.117162909 |
| MTUS1 | NM_001001924 /// NM_001001925 /// | −1.185855107 |
| | NM_001001927 /// NM_001001931 /// | |
| | NM_020749 | |
| NBN | NM_001024688 /// NM_002485 | −1.29949281 |
| NEFL | NM_006158 | −1.114077323 |
| NID1 | NM_002508 | 0.714548541 |
| NMU | NM_006681 | −1.182060395 |
| NNMT | NM_006169 | −1.49611684 |
| NR4A2 | NM_006186 /// NM_173171 /// | −0.793716522 |
| | NM_173172 /// NM_173173 | |
| NRG1 | NM_004495 /// NM_013956 /// | 1.150084193 |
| | NM_013957 /// | |
| | NM_013958 /// NM_013959 /// | |
| | NM_013960 | |
| NSF | NM_006178 | −1.042729954 |
| NUCKS | NM_022731 | 2.389945045 |
| NUDT15 | NM_018283 | −1.259671613 |
| OSBPL8 | NM_001003712 /// NM_020841 | −1.501841923 |
| PABPC4 | NM_003819 | −1.625270339 |
| PALM2-AKAP2 | NM_007203 /// NM_147150 | 0.75334143 |
| PCAF | NM_003884 | −1.01303745 |
| PDCD2 | NM_002598 /// NM_144781 | −0.821025736 |
| PDCD4 | NM_014456 /// NM_145341 | 1.207560012 |
| PDGFRL | NM_006207 | −0.728417971 |
| PEG10 | XM_496907 /// XM_499343 | −0.850603677 |
| PFAAP5 | NM_014887 | 1.00995749 |
| PGK1 | NM_000291 | 1.653917029 |
| PHTF2 | NM_020432 | −1.435962859 |
| PIP5K2B | NM_003559 /// NM_138687 | −1.176282316 |
| PLAU | NM_002658 | −0.824554099 |
| PMCH | NM_002674 | 0.871730513 |
| PPM1H | XM_350880 | −1.013741351 |
| PPP1CA | NM_001008709 /// NM_002708 /// | −1.894131186 |
| | NM_206873 | |
| PPP1CB | NM_002709 /// NM_206876 /// | −1.783955222 |
| | NM_206877 | |
| PPP1R12A | NM_002480 | −1.084874225 |
| PRNP | NM_000311 /// NM_183079 | −0.958358216 |
| PRO1843 | — | 1.041783261 |
| PSMD6 | NM_014814 | −1.13875629 |
| PTENP1 | — | 0.854304606 |
| PTGS2 | NM_000963 | −1.166655131 |
| PTPN12 | NM_002835 | 0.98401718 |
| PTS | NM_000317 | −1.077350104 |
| RAB2 | NM_002865 | −1.472842476 |
| RAB40B | NM_006822 | −0.724439401 |
| RARRES1 | NM_002888 /// NM_206963 | −0.872731167 |
| RARRES3 | NM_004585 | 0.937698042 |
| RB1 | NM_000321 | −1.019393484 |
| RBP4 | NM_006744 | −1.206604909 |
| RHEB | NM_005614 | 1.24347853 |
| RHOB | NM_004040 | 0.867434204 |
| RIP | NM_001033002 /// NM_032308 | 1.275556601 |
| RNF141 | NM_016422 | −0.805841944 |
| RP2 | NM_006915 | 0.833754103 |
| RPE | NM_006916 /// NM_199229 | −0.862237229 |
| RPE /// LOC440001 | NM_006916 /// NM_199229 /// | −0.882376602 |
| | XM_495848 | |
| RPL14 | NM_001034996 /// NM_003973 | 0.951492657 |
| RPL38 | NM_000999 | 1.594089757 |
| RPL4 | NM_000968 | −1.286483789 |
| RPS11 | NM_001015 | 1.344642602 |
| RRAGC | NM_022157 | 0.841252149 |
| SERPINE1 | NM_000602 | −0.906971559 |
| SESN1 | NM_014454 | 0.969021079 |
| SFRP4 | NM_003014 | −0.839989487 |
| SIRT1 | NM_012238 | −0.95785137 |
| SLC19A2 | NM_006996 | −1.425040844 |
| SLC1A4 | NM_003038 | −1.046830827 |
| SLC26A2 | NM_000112 | −0.789593004 |
| SLC2A3 | NM_006931 | 0.741688417 |
| SLC2A3 /// SLC2A14 | NM_006931 /// NM_153449 | 0.777277784 |
| SLC39A6 | NM_012319 | −0.991063322 |
| SLC39A9 | NM_018375 | −0.845810525 |
| SLC3A2 | NM_001012661 /// NM_001012662 /// | −0.760455682 |
| | NM_001012663 | |
| | /// NM_001012664 /// NM_001013251 | |
| SLC7A5 | NM_003486 | −0.805655634 |
| SMA4 | NM_021652 | 1.751441623 |
| SNAP25 | NM_003081 /// NM_130811 | −1.144869946 |
| SNRPD1 | NM_006938 | −1.238252269 |
| SNX13 | NM_015132 | −1.077547837 |
| SOAT1 | NM_003101 | −1.4130946 |
| SOX18 | NM_018419 | 2.548865238 |
| SPARC | NM_003118 | 0.701774899 |
| SRD5A1 | NM_001047 | −0.797620547 |
| SS18 | NM_001007559 /// NM_005637 | −0.748405362 |
| STX3A | NM_004177 | 0.847465024 |
| SUMO2 | NM_001005849 /// NM_006937 | 0.824463508 |
| TAF15 | NM_003487 /// NM_139215 | 1.023517036 |
| TARDBP | NM_007375 | −0.757464386 |
| TBC1D16 | NM_019020 | −1.153829054 |
| TBL1X | NM_005647 | −1.08552769 |
| TDG | NM_001008411 /// NM_003211 | 1.007246808 |
| TDO2 | NM_005651 | 1.231162585 |
| TFG | NM_001007565 /// NM_006070 | 0.864211334 |
| TGFBR2 | NM_001024847 /// NM_003242 | 0.718443392 |
| TGFBR3 | NM_003243 | 1.353282976 |
| THBD | NM_000361 | 1.050136118 |
| TM4SF20 | NM_024795 | −1.548256638 |
| TMEM45A | NM_018004 | −1.349843947 |
| TncRNA | — | 1.647849806 |
| TNFSF9 | NM_003811 | 1.103380988 |
| TOR1AIP1 | NM_015602 | −2.805037892 |
| TOX | NM_014729 | 0.928096328 |
| TPD52 | NM_001025252 /// NM_001025253 /// | −0.860388426 |
| | NM_005079 | |
| TRA1 | NM_003299 | 1.978956869 |
| TRIM22 | NM_006074 | 0.78338348 |
| TRIM23 | NM_001656 /// NM_033227 /// | −0.762495255 |
| | NM_033228 | |
| TRIP13 | NM_004237 | −1.331218004 |
| TSC | NM_017899 | −0.770711093 |
| TTMP | NM_024616 | −0.733612685 |
| TUBB-PARALOG | NM_178012 | −0.940699781 |
| TXN | NM_003329 | 1.502649699 |
| UBTF | NM_014233 | −0.732165826 |
| USP3 | NM_006537 | 0.785643243 |
| USP46 | NM_022832 | −1.013275727 |
| VDAC3 | NM_005662 | 1.1884143 |
| VEZATIN | NM_017599 | 1.049647153 |
| WIG1 | NM_022470 /// NM_152240 | −1.303047287 |
| WSB2 | NM_018639 | 0.898521363 |
| XTP2 | NM_015172 | 1.647838848 |
| ZBED2 | NM_024508 | 1.160901101 |
| ZBTB10 | NM_023929 | −0.946044115 |
| ZFHX1B | NM_014795 | −0.71121339 |
| ZNF609 | NM_015042 | 1.118504396 |

TABLE 1H
Genes with increased (positive values) or decreased (negative values)
expression following transfection of human cancer cells with pre-miR hsa-miR-216.

| Gene Symbol | RefSeq Transcript ID (Pruitt et al., 2005) | Δ log₂ |
|---|---|---|
| ANKRD46 | NM_198401 | 1.205064294 |
| ANPEP | NM_001150 | 1.05249117 |
| ANTXR1 | NM_018153 /// NM_032208 /// NM_053034 | 1.46843778 |
| ARID5B | NM_032199 | 0.844356546 |
| ATP2B4 | NM_001001396 /// NM_001684 | −0.840229649 |
| ATP6V0E | NM_003945 | −0.767172561 |
| AXL | NM_001699 /// NM_021913 | 0.716372713 |
| B4GALT1 | NM_001497 | 0.748412221 |
| B4GALT6 | NM_004775 | −0.751906998 |
| BCL10 | NM_003921 | −1.045655594 |
| BNIP3L | NM_004331 | −1.532819556 |
| BRCA1 | NM_007294 /// NM_007295 /// NM_007296 /// | −1.140217631 |
| | NM_007297 /// NM_007298 /// NM_007299 | |
| C6orf120 | NM_001029863 | 0.876394834 |
| C6orf155 | NM_024882 | 2.201467936 |
| C6orf210 | NM_020381 | −1.311623155 |
| CAV2 | NM_001233 /// NM_198212 | −1.248062997 |
| CCDC28A | NM_015439 | −1.961620584 |
| CCL2 | NM_002982 | 0.948633123 |
| CCNG1 | NM_004060 /// NM_199246 | 0.727459368 |
| CD38 | NM_001775 | 1.149396658 |
| CDK4 | NM_000075 | −0.963112257 |
| CDK8 | NM_001260 | −0.707005685 |
| CFH /// CFHL1 | NM_000186 /// NM_001014975 /// NM_002113 | 0.705005921 |
| CHMP5 | NM_016410 | −1.113320389 |
| COL11A1 | NM_001854 /// NM_080629 /// NM_080630 | 1.06415718 |
| CPM | NM_001005502 /// NM_001874 /// NM_198320 | −0.727000106 |
| CPS1 | NM_001875 | 0.890327068 |
| CREB3L2 | NM_194071 | −1.147859524 |
| CTH | NM_001902 /// NM_153742 | −0.724838822 |
| CXCL3 | NM_002090 | 0.905175084 |
| CXCL5 | NM_002994 | 1.237295089 |
| DIO2 | NM_000793 /// NM_001007023 /// NM_013989 | −0.731070381 |
| DKFZp434H1419 | — | −1.213095446 |
| EGFR | NM_005228 /// NM_201282 /// | 0.873087099 |
| | NM_201283 /// NM_201284 | |
| EI24 | NM_001007277 /// NM_004879 | −1.056093529 |
| EIF2S1 | NM_004094 | −0.894987495 |
| F5 | NM_000130 | 0.983748404 |
| FAM45B /// | NM_018472 /// NM_207009 | −1.216895124 |
| FAM45A | | |
| FAS | NM_000043 /// NM_152871 /// NM_152872 /// | 0.720304251 |
| | NM_152873 /// NM_152874 /// NM_152875 | |
| FCHO1 | NM_015122 | −1.035564154 |
| FEZ2 | NM_005102 | −1.540032542 |
| FLJ13912 | NM_022770 | −1.058436981 |
| GALNT1 | NM_020474 | −1.03022635 |
| GLIPR1 | NM_006851 | 0.771047501 |
| GMDS | NM_001500 | −0.706432221 |
| GPR107 | NM_020960 | 1.329247979 |
| GPR64 | NM_005756 | 1.226872143 |
| GREM1 | NM_013372 | −2.141146329 |
| HDAC3 | NM_003883 | −1.188428452 |
| HIC2 | NM_015094 | 0.848647375 |
| HIST1H2BC | NM_003526 | 1.138396492 |
| IDI1 | NM_004508 | −0.952048161 |
| IL6ST | NM_002184 /// NM_175767 | 0.825888288 |
| IQGAP2 | NM_006633 | 0.922666241 |
| ITGB6 | NM_000888 | 0.972580772 |
| JUN | NM_002228 | −0.989407999 |
| KCNJ16 | NM_018658 /// NM_170741 /// NM_170742 | 0.70784406 |
| LOC440118 | XM_498554 | 1.029719744 |
| MAP7 | NM_003980 | 0.710328186 |
| METAP2 | NM_006838 | −0.781506981 |
| MGC4172 | NM_024308 | −0.801783402 |
| MPHOSPH6 | NM_005792 | −1.053817598 |
| NCF2 | NM_000433 | −0.762923633 |
| NF1 | NM_000267 | −1.659565398 |
| NFYC | NM_014223 | −0.96189603 |
| NR2F1 | NM_005654 | 0.769244922 |
| NTS | NM_006183 | 1.139774547 |
| NUDT15 | NM_018283 | −1.037811863 |
| PAPPA | NM_002581 | 0.762370796 |
| PCTK1 | NM_006201 /// NM_033018 | −1.324652844 |
| PDCD2 | NM_002598 /// NM_144781 | −1.515603224 |
| PHF10 | NM_018288 /// NM_133325 | −1.030400448 |
| PIR | NM_001018109 /// NM_003662 | −2.705431095 |
| PLA2G4A | NM_024420 | 0.8022221 |
| PLEKHA1 | NM_001001974 /// NM_021622 | −0.700145946 |
| PPP1CB | NM_002709 /// NM_206876 /// NM_206877 | −0.864483881 |
| PSF1 | NM_021067 | −1.366589197 |
| PTGS2 | NM_000963 | 0.764713826 |
| RARRES1 | NM_002888 /// NM_206963 | 0.703593775 |
| RGC32 | NM_014059 | 0.744611688 |
| RP2 | NM_006915 | −0.882482368 |
| RPS6KA5 | NM_004755 /// NM_182398 | −0.712952845 |
| RRAGC | NM_022157 | 0.713512091 |
| RRM2 | NM_001034 | −0.876164389 |
| SCD | NM_005063 | 0.888437407 |
| SDC4 | NM_002999 | −1.014133325 |
| SEMA3C | NM_006379 | 0.768322613 |
| SESN1 | NM_014454 | 0.717889134 |
| SGPP1 | NM_030791 | −1.162308463 |
| SLC1A1 | NM_004170 | −0.788724519 |
| SLC2A3 | NM_006931 | −0.708665576 |
| SNAP25 | NM_003081 /// NM_130811 | 1.297734799 |
| SNRPD1 | NM_006938 | −1.550409311 |
| SOX18 | NM_018419 | 1.809239926 |
| SPRY4 | NM_030964 | 1.038107336 |
| SSB | NM_003142 | −1.245450605 |
| ST7 | NM_018412 /// NM_021908 | −1.117947704 |
| SWAP70 | NM_015055 | −0.918387597 |
| SYT1 | NM_005639 | 0.719749608 |
| TEAD1 | NM_021961 | 1.268097038 |
| TGFBR3 | NM_003243 | 0.773893351 |
| TIPRL | NM_001031800 /// NM_152902 | −1.922938983 |
| TMC5 | NM_024780 | −0.874298517 |
| TNC | NM_002160 | 0.923411097 |
| TOP1 | NM_003286 | 0.738270072 |
| TTC10 | NM_006531 /// NM_175605 | −0.799418273 |
| TTMP | NM_024616 | 0.867103058 |
| TTRAP | NM_016614 | −1.148845268 |
| UBE2V2 | NM_003350 | −0.750839256 |
| UBN1 | NM_016936 | −1.060787199 |
| VAV3 | NM_006113 | 0.753855057 |
| WIG1 | NM_022470 /// NM_152240 | 0.737324985 |
| WISP2 | NM_003881 | −0.724955794 |

TABLE 1I
Genes with increased (positive values) or decreased (negative values)
expression following transfection of human cancer cells with pre-miR hsa-miR-331.

| | RefSeq Transcript ID | |
|---|---|---|
| Gene Symbol | (Pruitt et al., 2005) | Δ log₂ |
| ADAM9 | NM_001005845 /// NM_003816 | −1.018202582 |
| AMBP | NM_001633 | 0.713506969 |
| ANKRD46 | NM_198401 | 0.758769458 |
| AQP3 | NM_004925 | −1.251852727 |
| AR | NM_000044 /// NM_001011645 | −0.778339604 |
| AREG | NM_001657 | −0.753449628 |
| ARHGDIA | NM_004309 | −0.951679694 |
| ARL2BP | NM_012106 | 0.996494605 |
| ATP6V0E | NM_003945 | 1.367616054 |
| AVPI1 | NM_021732 | −0.751596798 |
| B4GALT4 | NM_003778 /// NM_212543 | −0.753713587 |
| BAMBI | NM_012342 | −1.255265115 |
| BCL2L1 | NM_001191 /// NM_138578 | −0.886454677 |
| BICD2 | NM_001003800 /// NM_015250 | −1.182358353 |
| C19orf10 | NM_019107 | −1.53899451 |
| C1orf24 | NM_022083 /// NM_052966 | −0.704802929 |
| C2orf25 | NM_015702 | −1.081072862 |
| CASP7 | NM_001227 /// NM_033338 /// | −1.026901276 |
| | NM_033339 /// NM_033340 | |
| CCNG1 | NM_004060 /// NM_199246 | 0.897682498 |
| CDS1 | NM_001263 | −0.795343714 |
| CDS2 | NM_003818 | −0.781611289 |
| CFH | NM_000186 /// NM_001014975 | −0.703427241 |
| CGI-48 | NM_016001 | 1.289624084 |
| CLN5 | NM_006493 | −1.466578653 |
| COL4A2 | NM_001846 | −0.805438025 |
| COMMD9 | NM_014186 | −1.028582082 |
| COQ2 | NM_015697 | −1.037753576 |
| CSF2RA | NM_006140 /// NM_172245 /// NM_172246 /// | −0.820735805 |
| | NM_172247 /// NM_172248 /// NM_172249 | |
| CXCL1 | NM_001511 | 0.989718005 |
| D15Wsu75e | NM_015704 | −1.230678591 |
| DAF | NM_000574 | −1.116320814 |
| DDAH1 | NM_012137 | 0.702333256 |
| DIO2 | NM_000793 /// NM_001007023 /// NM_013989 | −0.818111915 |
| DSU | NM_018000 | 0.921680342 |
| EEF1D | NM_001960 /// NM_032378 | 0.754057576 |
| EFNA1 | NM_004428 /// NM_182685 | 0.811485975 |
| EHD1 | NM_006795 | −1.128885271 |
| EIF5A2 | NM_020390 | −1.220164668 |
| EMP1 | NM_001423 | −1.148241753 |
| ENO1 | NM_001428 | 0.78630193 |
| EREG | NM_001432 | −0.762145502 |
| FAM63B | NM_019092 | −1.181178296 |
| FBXO11 | NM_012167 /// NM_018693 /// NM_025133 | 0.812682335 |
| FGFR1 | NM_000604 /// NM_015850 /// NM_023105 | −1.002378067 |
| | /// NM_023106 /// NM_023107 /// NM_023108 | |
| FOSL1 | NM_005438 | −0.913695565 |
| GALNT7 | NM_017423 | −0.745195648 |
| GATA6 | NM_005257 | −1.045711005 |
| GGT1 | NM_001032364 /// NM_001032365 /// | −1.113140527 |
| | NM_005265 /// NM_013430 | |
| GLRB | NM_000824 | −1.060497998 |
| GPR64 | NM_005756 | −0.758625112 |
| GUK1 | NM_000858 | −1.13218881 |
| HAS2 | NM_005328 | −0.762816377 |
| HKDC1 | NM_025130 | −0.949792861 |
| HLRC1 | NM_031304 | −1.097296685 |
| HMGA1 | NM_002131 /// NM_145899 /// NM_145901 /// | −0.880292199 |
| | NM_145902 /// NM_145903 /// NM_145904 | |
| HSPA4 | NM_002154 /// NM_198431 | 0.728696496 |
| HSPB8 | NM_014365 | −0.759977773 |
| HSPC009 | — | −1.03607819 |
| IGFBP3 | NM_000598 /// NM_001013398 | −0.845378586 |
| IL13RA1 | NM_001560 | −2.196282315 |
| IL32 | NM_001012631 /// NM_001012632 /// | 0.833485752 |
| | NM_001012633 /// NM_001012634 /// NM_001012635 | |
| IL6R | NM_000565 /// NM_181359 | −0.914757761 |
| IL8 | NM_000584 | 0.913397477 |
| INHBC | NM_005538 | 0.858995384 |
| ITGB4 | NM_000213 /// NM_001005619 /// NM_001005731 | −0.85799549 |
| KIAA0090 | NM_015047 | −1.164407472 |
| KIAA1164 | NM_019092 | −1.23704637 |
| KIAA1641 | NM_020970 | −0.836514008 |
| KLF4 | NM_004235 | −1.055039556 |
| LMO4 | NM_006769 | −1.107321559 |
| LOC137886 | XM_059929 | −1.123182493 |
| LOXL2 | NM_002318 | −1.209767441 |
| LRP3 | NM_002333 | −0.715117868 |
| MARCKS | NM_002356 | −1.469677149 |
| MAZ | NM_002383 | −1.126821745 |
| MCL1 | NM_021960 /// NM_182763 | 0.942257941 |
| MGAM | NM_004668 | −0.814502675 |
| MGC3196 | XM_495878 | −1.126417939 |
| MGC3260 | — | −1.025699392 |
| MGC4172 | NM_024308 | −0.913455714 |
| MICAL2 | NM_014632 | −1.082050523 |
| MTMR1 | NM_003828 /// NM_176789 | −0.735120951 |
| NEFL | NM_006158 | −0.717701382 |
| NPTX1 | NM_002522 | 0.75531673 |
| NR5A2 | NM_003822 /// NM_205860 | −0.986400711 |
| NUCKS | NM_022731 | 1.878690008 |
| NUDT15 | NM_018283 | −0.73413178 |
| OXTR | NM_000916 | −0.706995427 |
| P4HB | NM_000918 | −1.115420821 |
| PDCD4 | NM_014456 /// NM_145341 | −0.703141449 |
| PDPK1 | NM_002613 /// NM_031268 | −0.997800492 |
| PDZK1IP1 | NM_005764 | 0.899109852 |
| PGK1 | NM_000291 | 1.458474231 |
| PHLPP | NM_194449 | −1.08805252 |
| PIG8 | NM_014679 | −1.143792856 |
| PLD3 | NM_001031696 /// NM_012268 | −1.061520584 |
| PLEC1 | NM_000445 /// NM_201378 /// NM_201379 | −0.861657517 |
| | /// NM_201380 /// NM_201381 /// NM_201382 | |
| PLEKHA1 | NM_001001974 /// NM_021622 | −0.814352719 |
| PMCH | NM_002674 | 1.23471474 |
| PODXL | NM_001018111 /// NM_005397 | −0.759679646 |
| PPL | NM_002705 | −0.863943433 |
| PRCC | NM_005973 /// NM_199416 | −1.560043378 |
| PRO1843 | — | 1.024656281 |
| PTENP1 | — | 0.843987346 |
| PTPN12 | NM_002835 | 0.720770416 |
| PXN | NM_002859 | −0.906771926 |
| RAB2 | NM_002865 | 1.21822883 |
| RGS2 | NM_002923 | −0.751864654 |
| RHEB | NM_005614 | 1.032801782 |
| RHOBTB1 | NM_001032380 /// NM_014836 /// NM_198225 | −1.461092343 |
| RIP | NM_001033002 /// NM_032308 | 1.32081268 |
| RPA2 | NM_002946 | −1.930005451 |
| RPE | NM_006916 /// NM_199229 | −1.035661937 |
| RPE /// | NM_006916 /// NM_199229 /// XM_495848 | −1.348584718 |
| LOC440001 | | |
| RPL14 | NM_001034996 /// NM_003973 | 0.889103758 |
| RPL38 | NM_000999 | 1.195046989 |
| RPS11 | NM_001015 | 0.966761487 |
| RRBP1 | NM_004587 | −1.58296738 |
| SAV1 | NM_021818 | −1.200930354 |
| SDC4 | NM_002999 | −0.943854956 |
| SDHB | NM_003000 | −0.795591847 |
| SH3YL1 | NM_015677 | 0.797572491 |
| SLC7A1 | NM_003045 | −1.030604814 |
| SMA4 | NM_021652 | −0.777526871 |
| SS18 | NM_001007559 /// NM_005637 | −1.164712195 |
| STX6 | NM_005819 | −0.793475858 |
| SUMO2 | NM_001005849 /// NM_006937 | 0.809404068 |
| SYNJ2BP | NM_018373 | −1.058973759 |
| TBC1D16 | NM_019020 | −0.823007164 |
| TBC1D2 | NM_018421 | −0.805664472 |
| TFG | NM_001007565 /// NM_006070 | 0.963221751 |
| TFPI | NM_001032281 /// NM_006287 | −0.848767621 |
| TGFB2 | NM_003238 | −1.04497232 |
| THBS1 | NM_003246 | −1.083274383 |
| TMC5 | NM_024780 | −1.012924338 |
| TMEM2 | NM_013390 | −1.011217086 |
| TMEM45A | NM_018004 | −0.789448041 |
| TMF1 | NM_007114 | −1.180142228 |
| TNC | NM_002160 | −0.703964402 |
| TNFAIP6 | NM_007115 | −1.1186537 |
| TNFSF9 | NM_003811 | −0.982271707 |
| TOR1AIP1 | NM_015602 | −0.919343306 |
| TOX | NM_014729 | −0.723074509 |
| TRA1 | NM_003299 | 1.696864298 |
| TRFP | NM_004275 | −1.030283612 |
| TRIP13 | NM_004237 | −0.809487394 |
| TRPC1 | NM_003304 | −0.751661455 |
| TTC3 | NM_001001894 /// NM_003316 | −0.703114676 |
| TXLNA | NM_175852 | −1.477978781 |
| TXN | NM_003329 | 1.338245007 |
| UGT1A8 /// | NM_019076 /// NM_021027 | −0.881758515 |
| UGT1A9 | | |
| USP46 | NM_022832 | −1.106506898 |
| VANGL1 | NM_138959 | −0.946441805 |
| VDAC3 | NM_005662 | 0.840449353 |
| VIL2 | NM_003379 | 0.706193269 |
| WDR1 | NM_005112 /// NM_017491 | −0.739441224 |
| WNT7B | NM_058238 | −0.891232207 |
| WSB2 | NM_018639 | 0.720487526 |
| XTP2 | NM_015172 | 0.708257434 |
| YRDC | NM_024640 | −1.09546979 |
| ZMYM6 | NM_007167 | −1.435718926 |
| ZNF259 | NM_003904 | −1.233812004 |
| ZNF395 | NM_018660 | −1.233741599 |
| | NM_006640 | −1.476797247 |

TABLE 1J
Genes with increased (positive values) or decreased (negative values)
expression following transfection of human cancer cells with pre-miR mmu-miR-292-3p.

| Gene Symbol | RefSeq Transcript ID (Pruitt et al., 2005) | Δ log₂ |
|---|---|---|
| ABCA12 | NM_015657 /// NM_173076 | 1.274537758 |
| ACAA1 | NM_001607 | −1.341988411 |
| ADRB2 | NM_000024 | 0.734681598 |
| AHNAK | NM_001620 /// NM_024060 | −1.068047951 |
| AKR7A2 | NM_003689 | −1.260890028 |
| ALDH3A2 | NM_000382 /// NM_001031806 | −1.149835407 |
| ALDH6A1 | NM_005589 | 0.707556281 |
| AP1G1 | NM_001030007 /// NM_001128 | −1.091995963 |
| AP1S2 | NM_003916 | −1.261719242 |
| AR | NM_000044 /// NM_001011645 | −1.016538203 |
| ARCN1 | NM_001655 | −1.394989314 |
| ARHGDIA | NM_004309 | −1.088113999 |
| ARL2BP | NM_012106 | 0.850663075 |
| ASNS | NM_001673 /// NM_133436 /// NM_183356 | −1.143388594 |
| ATF5 | NM_012068 | −1.313158757 |
| ATP6V0E | NM_003945 | 1.7283045 |
| B3GNT3 | NM_014256 | −0.749527176 |
| B4GALT6 | NM_004775 | −0.977953158 |
| BCL2A1 | NM_004049 | 1.206247671 |
| BDKRB2 | NM_000623 | 1.061713745 |
| BICD2 | NM_001003800 /// NM_015250 | −1.258118547 |
| BIRC3 | NM_001165 /// NM_182962 | 1.060985056 |
| BPGM | NM_001724 /// NM_199186 | −1.860577967 |
| BRP44 | NM_015415 | −1.286540106 |
| BTG2 | NM_006763 | 1.379663209 |
| C14orf2 | NM_004894 | −1.247503837 |
| C19orf2 | NM_003796 /// NM_134447 | −1.41536794 |
| C1GALT1C1 | NM_001011551 /// NM_152692 | −1.194583625 |
| C1orf121 | NM_016076 | −0.734943568 |
| C1R | NM_001733 | 1.15987472 |
| C20orf27 | NM_017874 | −0.745064444 |
| C21orf25 | NM_199050 | 0.743360022 |
| C2orf17 | NM_024293 | −1.510848665 |
| C2orf26 | NM_023016 | −1.019347994 |
| C3 | NM_000064 | 2.06034744 |
| C6orf210 | NM_020381 | −1.32460427 |
| C8orf1 | NM_004337 | 0.722461307 |
| CA11 | NM_001217 | −0.871451676 |
| CALM1 | NM_006888 | −1.352507852 |
| CASP7 | NM_001227 /// NM_033338 /// | −0.810273138 |
| | NM_033339 /// NM_033340 | |
| CCL20 | NM_004591 | 1.15656517 |
| CCND3 | NM_001760 | −0.782111615 |
| CCNG1 | NM_004060 /// NM_199246 | 1.387659998 |
| CD44 | NM_000610 /// NM_001001389 /// | 0.719455355 |
| | NM_001001390 | |
| | /// NM_001001391 /// NM_001001392 | |
| CDH4 | NM_001794 | −1.430091267 |
| CEBPD | NM_005195 | 1.006214661 |
| CFH /// CFHL1 | NM_000186 /// NM_001014975 /// NM_002113 | −1.50657812 |
| CGI-48 | NM_016001 | 1.518000296 |
| CLIC4 | NM_013943 | 1.141308993 |
| CLU | NM_001831 /// NM_203339 | −0.808510733 |
| COL5A1 | NM_000093 | 0.838721257 |
| COPS6 | NM_006833 | −2.469125346 |
| COQ2 | NM_015697 | −1.820118826 |
| CPM | NM_001005502 /// NM_001874 /// NM_198320 | 1.811763795 |
| CSF1 | NM_000757 /// NM_172210 /// NM_172211 /// | 1.093739444 |
| | NM_172212 | |
| CTDSP2 | NM_005730 | 1.1038569 |
| CXCL1 | NM_001511 | 1.373132066 |
| CXCL2 | NM_002089 | 1.348536544 |
| CXCL3 | NM_002090 | 1.015075683 |
| CXCL5 | NM_002994 | 0.943452807 |
| CYP4F3 | NM_000896 | −0.944098228 |
| CYP51A1 | NM_000786 | 1.017134253 |
| DAAM1 | NM_014992 | 1.296531572 |
| DAZAP2 | NM_014764 | −1.658661628 |
| DAZAP2 /// | NM_014764 /// XM_376165 | −1.087782444 |
| LOC401029 | | |
| DCP2 | NM_152624 | 1.77586343 |
| DIPA | NM_006848 | −0.93403737 |
| DKFZP564J0123 | NM_199069 /// NM_199070 /// NM_199073 | −1.383450396 |
| | /// NM_199074 /// NM_199417 | |
| DKK3 | NM_001018057 /// NM_013253 /// NM_015881 | 0.878239299 |
| DMN | NM_015286 /// NM_145728 | −1.141858838 |
| DNAJB4 | NM_007034 | −1.296695319 |
| DPYSL4 | NM_006426 | 1.395487959 |
| DST | NM_001723 /// NM_015548 /// | 0.826671369 |
| | NM_020388 /// NM_183380 | |
| DSU | NM_018000 | 0.850899944 |
| DTYMK | NM_012145 | −1.318162355 |
| DUSP3 | NM_004090 | −1.089273702 |
| E2F8 | NM_024680 | −1.013925338 |
| EEF1D | NM_001960 /// NM_032378 | 0.921658799 |
| EFEMP1 | NM_004105 /// NM_018894 | 0.72566566 |
| EFNA1 | NM_004428 /// NM_182685 | 2.046925472 |
| EGFL4 | NM_001410 | −1.078181988 |
| EHF | NM_012153 | −0.797518709 |
| EIF2C1 | NM_012199 | −1.057953517 |
| ELOVL6 | NM_024090 | 0.700401502 |
| ENO1 | NM_001428 | 0.815326156 |
| ENTPD7 | NM_020354 | 1.034032191 |
| FAM46A | NM_017633 | 0.898362379 |
| FAM63B | NM_019092 | 0.727540952 |
| FAS | NM_000043 /// NM_152871 /// NM_152872 /// | 1.579115853 |
| | NM_152873 /// NM_152874 /// NM_152875 | |
| FBLN1 | NM_001996 /// NM_006485 /// | −1.342132018 |
| | NM_006486 /// NM_006487 | |
| FBXO11 | NM_012167 /// NM_018693 /// NM_025133 | 0.981097713 |
| FDXR | NM_004110 /// NM_024417 | 1.164440342 |
| FEZ2 | NM_005102 | −0.975086128 |
| FGFBP1 | NM_005130 | 0.74848828 |
| FLJ11259 | NM_018370 | 0.775722888 |
| FLJ13236 | NM_024902 | −1.279533014 |
| FLJ13910 | NM_022780 | 0.737477028 |
| FLJ22662 | NM_024829 | −1.298342375 |
| FNBP1 | NM_015033 | 0.792859874 |
| FOSL1 | NM_005438 | 0.70494518 |
| GALE | NM_000403 /// NM_001008216 | −1.680052376 |
| GAS2L1 | NM_006478 /// NM_152236 /// NM_152237 | −1.089734346 |
| GCLC | NM_001498 | −1.212645403 |
| GFPT2 | NM_005110 | 0.739403227 |
| GLT25D1 | NM_024656 | −1.128968664 |
| GLUL | NM_001033044 /// NM_001033056 /// | 0.707890594 |
| | NM_002065 | |
| GMDS | NM_001500 | −1.062449288 |
| GMPR2 | NM_001002000 /// NM_001002001 /// | −1.139237339 |
| | NM_001002002 /// NM_016576 | |
| GNA13 | NM_006572 | 1.236589519 |
| GOLPH2 | NM_016548 /// NM_177937 | −1.086755929 |
| GPI | NM_000175 | −1.259439873 |
| GPNMB | NM_001005340 /// NM_002510 | −1.007595602 |
| GREB1 | NM_014668 /// NM_033090 /// NM_148903 | 1.352108534 |
| GSPT1 | NM_002094 | −1.044364422 |
| HAS2 | NM_005328 | 0.947721212 |
| HBXIP | NM_006402 | −1.031037958 |
| HIC2 | NM_015094 | 1.023623547 |
| HIST1H2AC | NM_003512 | −1.008238017 |
| HLA-DMB | NM_002118 | −0.775827225 |
| HMGA2 | NM_001015886 /// NM_003483 /// NM_003484 | 1.304771857 |
| HMGCR | NM_000859 | 1.27304615 |
| HMGCS1 | NM_002130 | 1.012886882 |
| HMMR | NM_012484 /// NM_012485 | −0.70033762 |
| HMOX1 | NM_002133 | −1.35301396 |
| HNMT | NM_001024074 /// NM_001024075 /// | 1.041235328 |
| | NM_006895 | |
| HSPCA | NM_001017963 /// NM_005348 | −1.074857802 |
| ID1 | NM_002165 /// NM_181353 | −1.025496584 |
| ID2 | NM_002166 | −0.705177884 |
| IDI1 | NM_004508 | 1.219263646 |
| IDS | NM_000202 /// NM_006123 | −1.077198338 |
| IER3IP1 | NM_016097 | 0.940286614 |
| IGFBP3 | NM_000598 /// NM_001013398 | −1.610733561 |
| IL1RAP | NM_002182 /// NM_134470 | 1.347581197 |
| IL32 | NM_001012631 /// NM_001012632 /// | 2.250504431 |
| | NM_001012633 /// NM_001012634 /// | |
| | NM_001012635 | |
| IL6R | NM_000565 /// NM_181359 | 1.202516814 |
| IL8 | NM_000584 | 1.738888969 |
| INHBB | NM_002193 | −0.789026545 |
| INHBC | NM_005538 | 1.054375714 |
| INSIG1 | NM_005542 /// NM_198336 /// NM_198337 | 1.312569861 |
| INSL4 | NM_002195 | −0.968255432 |
| IPO7 | NM_006391 | −1.137292191 |
| ITGB4 | NM_000213 /// NM_001005619 /// | −1.241875014 |
| | NM_001005731 | |
| KCNJ16 | NM_018658 /// NM_170741 /// NM_170742 | −0.994177169 |
| KIAA0317 | NM_014821 | −1.954785599 |
| KIAA0485 | — | 0.803437158 |
| KIAA0882 | NM_015130 | 0.886522516 |
| KIAA1164 | NM_019092 | 1.106110788 |
| KLC2 | NM_022822 | −0.929423697 |
| KRT7 | NM_005556 | 0.876412052 |
| LAMP1 | NM_005561 | −1.347563751 |
| LEPR | NM_001003679 /// NM_001003680 /// | −0.883786823 |
| | NM_002303 | |
| LMO4 | NM_006769 | −0.899001385 |
| LOC440118 | XM_498554 | 2.659402205 |
| LRP8 | NM_001018054 /// NM_004631 /// | −0.913541429 |
| | NM_017522 /// NM_033300 | |
| MAFF | NM_012323 /// NM_152878 | 1.037660909 |
| MAP3K6 | NM_004672 | −1.020561565 |
| MAPKAPK2 | NM_004759 /// NM_032960 | −0.851240177 |
| MARCH2 | NM_001005415 /// NM_001005416 /// | −1.340797948 |
| | NM_016496 | |
| MAT2B | NM_013283 /// NM_182796 | −1.010823059 |
| MCAM | NM_006500 | 0.761721492 |
| MCL1 | NM_021960 /// NM_182763 | 1.676669192 |
| MDM2 | NM_002392 /// NM_006878 /// NM_006879 /// | 1.177412993 |
| | NM_006880 /// NM_006881 /// NM_006882 | |
| MERTK | NM_006343 | 0.794000917 |
| MGC2574 | NM_024098 | −1.346847468 |
| MGC5508 | NM_024092 | −1.272547011 |
| MGC5618 | — | 1.428865355 |
| MICAL-L1 | NM_033386 | 1.230207682 |
| MPV17 | NM_002437 | −1.076584476 |
| MR1 | NM_001531 | 1.030488179 |
| MTDH | NM_178812 | −1.117806598 |
| MVP | NM_005115 /// NM_017458 | −0.709666753 |
| NALP1 | NM_001033053 /// NM_014922 /// NM_033004 | 0.805360321 |
| | /// NM_033006 /// NM_033007 | |
| NEFL | NM_006158 | 0.936792696 |
| NID1 | NM_002508 | 1.050433438 |
| NMU | NM_006681 | −0.895973974 |
| NPR3 | NM_000908 | 0.847545931 |
| NR2F2 | NM_021005 | −1.05195379 |
| NR4A2 | NM_006186 /// NM_173171 /// NM_173172 /// | −0.784394334 |
| | NM_173173 | |
| NUCKS | NM_022731 | 2.054851809 |
| NUMA1 | NM_006185 | −0.935775914 |
| NUPL1 | NM_001008564 /// NM_001008565 /// | 0.995356442 |
| | NM_014089 | |
| OPTN | NM_001008211 /// NM_001008212 /// | 1.062219148 |
| | NM_001008213 /// NM_021980 | |
| ORMDL2 | NM_014182 | −1.234447987 |
| P4HA2 | NM_001017973 /// NM_001017974 /// | 0.911666974 |
| | NM_004199 | |
| PAFAH1B2 | NM_002572 | −1.046822403 |
| PAPPA | NM_002581 | 0.729791369 |
| PAQR3 | NM_177453 | −1.033326915 |
| PDCD2 | NM_002598 /// NM_144781 | −0.961233896 |
| PDCD4 | NM_014456 /// NM_145341 | 0.7201252 |
| PDCD6IP | NM_013374 | −1.196552647 |
| PDGFRL | NM_006207 | 0.893046656 |
| PEX10 | NM_002617 /// NM_153818 | −1.116287896 |
| PGK1 | NM_000291 | 1.670142045 |
| PHTF2 | NM_020432 | 0.925243951 |
| PIGK | NM_005482 | −1.409798998 |
| PLAT | NM_000930 /// NM_000931 /// NM_033011 | 0.929497265 |
| PLAU | NM_002658 | 1.066687801 |
| PLEKHA1 | NM_001001974 /// NM_021622 | 0.910943491 |
| PLSCR4 | NM_020353 | 0.724455918 |
| PMCH | NM_002674 | 1.270137987 |
| PODXL | NM_001018111 /// NM_005397 | 1.036062602 |
| POLR3D | NM_001722 | −1.115693639 |
| POLR3G | NM_006467 | −0.761975143 |
| PON2 | NM_000305 /// NM_001018161 | −1.276679882 |
| PON3 | NM_000940 | −0.74811781 |
| PPAP2C | NM_003712 /// NM_177526 /// NM_177543 | −1.291995651 |
| PPM1D | NM_003620 | 1.299946946 |
| PRDX6 | NM_004905 | −1.304368229 |
| PREI3 | NM_015387 /// NM_199482 | −1.905696629 |
| PRNP | NM_0003111 /// NM_183079 | −1.121128917 |
| PRO1843 | — | 1.272144805 |
| PSIP1 | NM_021144 /// NM_033222 | −1.013912911 |
| PTEN | NM_000314 | −1.24087728 |
| PTER | NM_001001484 /// NM_030664 | −1.11747507 |
| PTK9 | NM_002822 /// NM_198974 | 1.126567447 |
| PTMS | NM_002824 | −0.888918542 |
| PTP4A1 | NM_003463 | 1.05405477 |
| PTPN12 | NM_002835 | 0.974469072 |
| PTX3 | NM_002852 | 1.329740901 |
| PXDN | XM_056455 | 1.024115421 |
| QKI | NM_006775 /// NM_206853 /// | 0.851419246 |
| | NM_206854 /// NM_206855 | |
| RAB13 | NM_002870 | −1.03691008 |
| RAB2 | NM_002865 | 1.28227173 |
| RAB32 | NM_006834 | −1.021658289 |
| RAB4A | NM_004578 | −1.275775048 |
| RAP140 | NM_015224 | −1.085805474 |
| RASGRP1 | NM_005739 | 1.023197964 |
| RBP4 | NM_006744 | 1.066069203 |
| RDX | NM_002906 | 1.366314325 |
| RHEB | NM_005614 | 1.061183478 |
| RIG | — | 1.098716654 |
| RIP | NM_001033002 /// NM_032308 | 1.131269937 |
| RNF141 | NM_016422 | −1.263130303 |
| RPL14 | NM_001034996 /// NM_003973 | 0.872264327 |
| RPL38 | NM_000999 | 1.275185495 |
| RPS11 | NM_001015 | 0.988294482 |
| RRAD | NM_004165 | 0.714605352 |
| RRAGC | NM_022157 | 1.010062922 |
| RRAGD | NM_021244 | 1.271449795 |
| RRM2 | NM_001034 | −1.903220473 |
| SAMD4 | NM_015589 | 1.225116813 |
| SC4MOL | NM_001017369 /// NM_006745 | 1.373112547 |
| SCARB2 | NM_005506 | 1.116638678 |
| SCD | NM_005063 | 1.110346934 |
| SCML1 | NM_006746 | 1.225870611 |
| SDHA | NM_004168 | −1.052892397 |
| SEC23A | NM_006364 | −0.818184343 |
| SESN1 | NM_014454 | 1.543653494 |
| SH3GLB2 | NM_020145 | −0.903986408 |
| SKP2 | NM_005983 /// NM_032637 | 1.381913073 |
| SLC11A2 | NM_000617 | 0.946254297 |
| SLC2A3 | NM_006931 | 1.313395241 |
| SLC2A3 /// | NM_006931 /// NM_153449 | 1.052490023 |
| SLC2A14 | | |
| SLC30A9 | NM_006345 | −1.322099941 |
| SLC35A3 | NM_012243 | −1.013644493 |
| SMARCA2 | NM_003070 /// NM_139045 | 0.801377135 |
| SNRPD1 | NM_006938 | −0.865130985 |
| SOD2 | NM_000636 /// NM_001024465 /// | 1.214392447 |
| | NM_001024466 | |
| SORBS3 | NM_001018003 /// NM_005775 | −1.090614527 |
| SOX18 | NM_018419 | 4.148048165 |
| SPARC | NM_003118 | 1.52156486 |
| SPHAR | NM_006542 | −0.926094726 |
| SQLE | NM_003129 | 1.043028372 |
| SRPX | NM_006307 | 0.79067552 |
| STC1 | NM_003155 | 1.02010396 |
| STK24 | NM_001032296 /// NM_003576 | −0.828653609 |
| STS | NM_000351 | −1.150824058 |
| STX3A | NM_004177 | 0.959801577 |
| SUCLG2 | NM_003848 | −1.642142769 |
| SUMO2 | NM_001005849 /// NM_006937 | 0.867682532 |
| SVIL | NM_003174 /// NM_021738 | 0.760443698 |
| SYT1 | NM_005639 | −1.220961769 |
| TAF15 | NM_003487 /// NM_139215 | 0.839954321 |
| TBC1D2 | NM_018421 | −0.925351913 |
| TDG | NM_001008411 /// NM_003211 | 0.810140453 |
| TFG | NM_001007565 /// NM_006070 | 1.057373538 |
| TFPI | NM_001032281 /// NM_006287 | 0.999943519 |
| TFRC | NM_003234 | −1.062533788 |
| TGFBR3 | NM_003243 | 1.021115746 |
| THBS1 | NM_003246 | −1.182821435 |
| TJP2 | NM_004817 /// NM_201629 | 0.832785426 |
| TK2 | NM_004614 | −1.219573893 |
| TM4SF20 | NM_024795 | −1.052929883 |
| TM4SF4 | NM_004617 | −1.214905307 |
| TM7SF1 | NM_003272 | −0.921538795 |
| TncRNA | — | 1.510437605 |
| TNFAIP3 | NM_006290 | 1.049000444 |
| TNFAIP6 | NM_007115 | −1.137303144 |
| TNFRSF10B | NM_003842 /// NM_147187 | 1.00601181 |
| TNFRSF9 | NM_001561 | 0.879508972 |
| TNS1 | NM_022648 | 1.429582253 |
| TPD52L1 | NM_001003395 /// NM_001003396 /// | −1.052818746 |
| | NM_001003397 /// NM_003287 | |
| TPI1 | NM_000365 | −1.042595069 |
| TPM4 | NM_003290 | −1.1018669 |
| TRA1 | NM_003299 | 2.06266927 |
| TRIM14 | NM_014788 /// NM_033219 /// | −1.348327164 |
| | NM_033220 /// NM_033221 | |
| TTMP | NM_024616 | −0.79505753 |
| TXLNA | NM_175852 | −0.989673731 |
| TXN | NM_003329 | 1.418205452 |
| UBE2V2 | NM_003350 | −1.116103021 |
| USP46 | NM_022832 | −1.625223999 |
| VDAC1 | NM_003374 | −1.70629034 |
| VDAC3 | NM_005662 | 0.95727826 |
| VIL2 | NM_003379 | −1.38536373 |
| VPS4A | NM_013245 | −0.759414556 |
| WBSCR22 | NM_017528 | −1.011859709 |
| WDR7 | NM_015285 /// NM_052834 | −1.206634395 |
| WEE1 | NM_003390 | 1.163396761 |
| WIG1 | NM_022470 /// NM_152240 | 0.700863484 |
| WIZ | XM_372716 | −1.129981905 |
| WNT7B | NM_058238 | −1.794403919 |
| WSB2 | NM_018639 | 1.487026325 |
| XTP2 | NM_015172 | 0.895652638 |
| YIPF3 | NM_015388 | −1.060355879 |
| YOD1 | NM_018566 | 1.018605664 |
| ZNF259 | NM_003904 | −0.79681991 |
| ZNF652 | NM_014897 | 0.854709863 |

A further embodiment of the invention is directed to methods of modulating a cellular pathway comprising administering to the cell an amount of an isolated nucleic acid comprising a miR-15, miR-26, miR-31, miR-145, miR-147, miR-188, miR-215, miR-216, miR-331, or mmu-miR-292-3p nucleic acid sequence or a miR-15, miR-26, miR-31, miR-145, miR-147, miR-188, miR-215, miR-216, miR-331, or mmu-miR-292-3p inhibitor. A cell, tissue, or subject may be a cancer cell, a cancerous tissue or harbor cancerous tissue, or a cancer patient. The database content related to all nucleic acids and genes designated by an accession number or a database submission are incorporated herein by reference as of the filing date of this application.

A further embodiment of the invention is directed to methods of modulating a cellular pathway comprising administering to the cell an amount of an isolated nucleic acid comprising a miR-15, miR-26, miR-31, miR-145, miR-147, miR-188, miR-215, miR-216, miR-331, or mmu-miR-292-3p nucleic acid sequence in an amount sufficient to modulate the expression, function, status, or state of a cellular pathway, in particular those pathways described in Table 2 or the pathways known to include one or more genes from Table 1, 3, and/or 4. Modulation of a cellular pathway includes, but is not limited to modulating the expression of one or more gene(s). Modulation of a gene can include inhibiting the function of an endogenous miRNA or providing a functional miRNA to a cell, tissue, or subject. Modulation refers to the expression levels or activities of a gene or its related gene product (e.g., mRNA) or protein, e.g., the mRNA levels may be modulated or the translation of an mRNA may be modulated. Modulation may increase or up regulate a gene or gene product or it may decrease or down regulate a gene or gene product (e.g., protein levels or activity).

Still a further embodiment includes methods of administering an miRNA or mimic thereof, and/or treating a subject or patient having, suspected of having, or at risk of developing a pathological condition comprising one or more of step (a) administering to a patient or subject an amount of an isolated nucleic acid comprising a miR-15, miR-26, miR-31, miR-145, miR-147, miR-188, miR-215, miR-216, miR-331, or mmu-miR-292-3p nucleic acid sequence or a miR-15, miR-26, miR-31, miR-145, miR-147, miR-188, miR-215, miR-216, miR-331, or mmu-miR-292-3p inhibitor in an amount sufficient to modulate expression of a cellular pathway; and (b) administering a second therapy, wherein the modulation of the cellular pathway sensitizes the patient or subject, or increases the efficacy of a second therapy. An increase in efficacy can include a reduction in toxicity, a reduced dosage or duration of the second therapy, or an additive or synergistic effect. A cellular pathway may include, but is not limited to one or more pathway described in Table 2 below or a pathway that is know to include one or more genes of Tables 1, 3, and/or 4. The second therapy may be administered before, during, and/or after the isolated nucleic acid or miRNA or inhibitor is administered.

A second therapy can include administration of a second miRNA or therapeutic nucleic acid such as a siRNA or antisense oligonucleotide, or may include various standard therapies, such as pharmaceuticals, chemotherapy, radiation therapy, drug therapy, immunotherapy, and the like. Embodiments of the invention may also include the determination or assessment of gene expression or gene expression profile for the selection of an appropriate therapy. In a particular aspect, a second therapy is chemotherapy. A chemotherapy can include, but is not limited to paclitaxel, cisplatin, carboplatin, doxorubicin, oxaliplatin, larotaxel, taxol, lapatinib, docetaxel, methotrexate, capecitabine, vinorelbine, cyclophosphamide, gemcitabine, amrubicin, cytarabine, etoposide, camptothecin, dexamethasone, dasatinib, tipifarnib, bevacizumab, sirolimus, temsirolimus, everolimus, lonafamib, cetuximab, erlotinib, gefitinib, imatinib mesylate, rituximab, trastuzumab, nocodazole, sorafenib, sunitinib, bortezomib, alemtuzumab, gemtuzumab, tositumomab or ibritumomab.

Embodiments of the invention include methods of treating a subject with a disease or condition comprising one or more of the steps of (a) determining an expression profile of one or more genes selected from Table 1, 3, and/or 4; (b) assessing the sensitivity of the subject to therapy based on the expression profile; (c) selecting a therapy based on the assessed sensitivity; and (d) treating the subject using a selected therapy. Typically, the disease or condition will have as a component, indicator, or resulting mis-regulation of one or more gene of Table 1, 3, and/or 4.

In certain aspects, 2, 3, 4, 5, 6, 7, 8, 9, 10, or more miRNA may be used in sequence or in combination; for instance, any combination of miR-15, miR-26, miR-31, miR-145, miR-147, miR-188, miR-215, miR-216, miR-331, or mmu-miR-292-3p or a miR-15, miR-26, miR-31, miR-145, miR-147, miR-188, miR-215, miR-216, miR-331, or mmu-miR-292-3p inhibitor with another miRNA or miRNA inhibitor. Further embodiments include the identification and assessment of an expression profile indicative of miR-15, miR-26, miR-31, miR-145, miR-147, miR-188, miR-215, miR-216, miR-331, or mmu-miR-292-3p status in a cell or tissue comprising expression assessment of one or more gene from Table 1, 3, and/or 4, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker or miRNA in the sample (e.g., a plurality of nucleic acid probes that identify one or more markers from Tables 1, 3, and/or 4); it is contemplated that the nucleic acid profile can be obtained using a set of RNAs, using for example nucleic acid amplification or hybridization techniques well know to one of ordinary skill in the art. The difference in the expression profile in the sample from the patient and a reference expression profile, such as an expression profile of one or more genes or miRNAs, are indicative of which miRNAs to be administered.

In certain aspects, miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor and let-7 or let-7 inhibitor can be administered to patients with acute lymphoblastic leukemia, acute myeloid leukemia, angiosarcoma, breast carcinoma, bladder carcinoma, cervical carcinoma, carcinoma of the head and neck, chronic lymphoblastic leukemia, chronic myeloid leukemia, colorectal carcinoma, endometrial carcinoma, glioma, glioblastoma, gastric carcinoma, hepatocellular carcinoma, Hodgkin lymphoma, Kaposi's sarcoma, leukemia, lung carcinoma, leiomyosarcoma, melanoma, medulloblastoma, myxofibrosarcoma, multiple myeloma, neuroblastoma, non-Hodgkin lymphoma, non-small cell lung carcinoma, ovarian carcinoma, oesophageal carcinoma, pancreatic carcinoma, prostate carcinoma, squamous cell carcinoma of the head and neck, salivary gland tumor, thyroid carcinoma, and/or urothelial carcinoma.

Further aspects include administering miR-26, miR-31, miR-145, miR-147, miR-188, miR-215, miR-216, miR-331, or mmu-miR-292-3p or miR-26, miR-31, miR-145, miR-147, miR-188, miR-215, miR-216, miR-331, or mmu-miR-292-3p inhibitor and miR-15 or miR-15 inhibitor to patients with astrocytoma, acute myeloid leukemia, breast carcinoma, B-cell lymphoma, bladder carcinoma, cervical carcinoma, carcinoma of the head and neck, chronic myeloid leukemia, colorectal carcinoma, endometrial carcinoma, glioma, glioblastoma, gastric carcinoma, hepatoblastoma, hepatocellular carcinoma, Hodgkin lymphoma, lung carcinoma, laryngeal squamous cell carcinoma, larynx carcinoma, melanoma, mantle cell lymphoma, myxofibrosarcoma, myeloid leukemia, multiple myeloma, neuroblastoma, neurofibroma, non-Hodgkin lymphoma, non-small cell lung carcinoma, ovarian carcinoma, oesophageal carcinoma, pancreatic carcinoma, prostate carcinoma, pheochromocytoma, renal cell carcinoma, rhabdomyosarcoma, squamous cell carcinoma of the head and neck, and/or thyroid carcinoma.

In still further aspects, miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor and miR-16 or miR-16 inhibitor are administered to patients with astrocytoma, breast carcinoma, B-cell lymphoma, bladder carcinoma, colorectal carcinoma, endometrial carcinoma, glioblastoma, gastric carcinoma, hepatoblastoma, hepatocellular carcinoma, Hodgkin lymphoma, laryngeal squamous cell carcinoma, melanoma, medulloblastoma, mantle cell lymphoma, myxofibrosarcoma, myeloid leukemia, multiple myeloma, neurofibroma, non-small cell lung carcinoma, ovarian carcinoma, oesophageal carcinoma, pancreatic carcinoma, prostate carcinoma, pheochromocytoma, renal cell carcinoma, rhabdomyosarcoma, squamous cell carcinoma of the head and neck, and/or thyroid carcinoma.

In certain aspects, miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor and miR-20 or miR-20 inhibitor are administered to patients with astrocytoma, acute myeloid leukemia, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, neuroblastoma, non-Hodgkin lymphoma, non-small cell lung carcinoma, ovarian carcinoma, oesophageal carcinoma, osteosarcoma, pancreatic carcinoma, prostate carcinoma, squamous cell carcinoma of the head and neck, thyroid carcinoma, and/or urothelial carcinoma.

Aspects of the invention include methods where miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor and miR-21 or miR-21 inhibitor are administered to patients with astrocytoma, acute lymphoblastic leukemia, acute myeloid leukemia, breast carcinoma, Burkitt's lymphoma, bladder carcinoma, colorectal carcinoma, endometrial carcinoma, glioma, glioblastoma, gastric carcinoma, hepatocellular carcinoma, melanoma, mantle cell lymphoma, myeloid leukemia, neuroblastoma, neurofibroma, non-small cell lung carcinoma, ovarian carcinoma, oesophageal carcinoma, pancreatic carcinoma, prostate carcinoma, pheochromocytoma, renal cell carcinoma, rhabdomyosarcoma, and/or squamous cell carcinoma of the head and neck.

In still further aspects, miR-15, miR-31, miR-145, miR-147, miR-188, miR-215, miR-216, miR-331, or mmu-miR-292-3p or miR-15, miR-31, miR-145, miR-147, miR-188, miR-215, miR-216, miR-331, or mmu-miR-292-3p inhibitor and miR-26a or miR-26a inhibitor are administered to patients with anaplastic large cell lymphoma, acute lymphoblastic leukemia, acute myeloid leukemia, angiosarcoma, breast carcinoma, B-cell lymphoma, Burkitt's lymphoma, bladder carcinoma, cervical carcinoma, carcinoma of the head and neck, chronic lymphoblastic leukemia, chronic myeloid leukemia, colorectal carcinoma, glioma, glioblastoma, gastric carcinoma, hepatocellular carcinoma, Kaposi's sarcoma, leukemia, lung carcinoma, leiomyosarcoma, larynx carcinoma, melanoma, multiple myeloma, neuroblastoma, non-Hodgkin lymphoma, non-small cell lung carcinoma, ovarian carcinoma, oesophageal carcinoma, osteosarcoma, pancreatic carcinoma, prostate carcinoma, renal cell carcinoma, rhabdomyosarcoma, small cell lung cancer, and/or testicular tumor.

In yet a further aspect, miR-15, miR-26, miR-31, miR-145, miR-147, miR-188, miR-215, miR-216, miR-331, or mmu-miR-292-3p or miR-15, miR-26, miR-31, miR-145, miR-147, miR-188, miR-215, miR-216, miR-331, or mmu-miR-292-3p inhibitor and miR-34a or miR-34a inhibitor are administered to patients with astrocytoma, anaplastic large cell lymphoma, acute lymphoblastic leukemia, acute myeloid leukemia, angiosarcoma, breast carcinoma, B-cell lymphoma, bladder carcinoma, cervical carcinoma, carcinoma of the head and neck, chronic lymphoblastic leukemia, chronic myeloid leukemia, colorectal carcinoma, endometrial carcinoma, glioma, glioblastoma, gastric carcinoma, gastrinoma, hepatoblastoma, hepatocellular carcinoma, Hodgkin lymphoma, Kaposi's sarcoma, leukemia, lung carcinoma, leiomyosarcoma, laryngeal squamous cell carcinoma, melanoma, mucosa-associated lymphoid tissue B-cell lymphoma, medulloblastoma, mantle cell lymphoma, myeloid leukemia, multiple myeloma, high-risk myelodysplastic syndrome, mesothelioma, neurofibroma, non-Hodgkin lymphoma, non-small cell lung carcinoma, ovarian carcinoma, oesophageal carcinoma, osteosarcoma, pancreatic carcinoma, prostate carcinoma, pheochromocytoma, rhabdomyosarcoma, squamous cell carcinoma of the head and neck, Schwanomma, small cell lung cancer, salivary gland tumor, sporadic papillary renal carcinoma, thyroid carcinoma, testicular tumor, and/or urothelial carcinoma.

In yet further aspects, miR-15, miR-26, miR-31, miR-145, miR-147, miR-188, miR-215, miR-216, miR-331, or mmu-miR-292-3p, or miR-15, miR-26, miR-31, miR-145, miR-147, miR-188, miR-215, miR-216, miR-331, or mmu-miR-292-3p inhibitor and miR-126 or miR-126 inhibitor are administered to patients with astrocytoma, acute myeloid leukemia, breast carcinoma, Burkitt's lymphoma, bladder carcinoma, cervical carcinoma, colorectal carcinoma, endometrial carcinoma, Ewing's sarcoma, glioma, glioblastoma, gastric carcinoma, gastrinoma, hepatoblastoma, hepatocellular carcinoma, Hodgkin lymphoma, leukemia, lung carcinoma, melanoma, mantle cell lymphoma, myeloid leukemia, mesothelioma, neurofibroma, non-Hodgkin lymphoma, non-small cell lung carcinoma, ovarian carcinoma, oesophageal carcinoma, osteosarcoma, pancreatic carcinoma, prostate carcinoma, pheochromocytoma, renal cell carcinoma, rhabdomyosarcoma, squamous cell carcinoma of the head and neck, Schwanomma, small cell lung cancer, sporadic papillary renal carcinoma, and/or thyroid carcinoma.

In a further aspect, miR-15, miR-26, miR-31, miR-145, miR-147, miR-188, miR-215, miR-216, miR-331, or mmu-miR-292-3p, or miR-15, miR-26, miR-31, miR-145, miR-147, miR-188, miR-215, miR-216, miR-331, or mmu-miR-292-3p inhibitor and miR-143 or miR-143 inhibitor are administered to patients with astrocytoma, anaplastic large cell lymphoma, acute lymphoblastic leukemia, acute myeloid leukemia, breast carcinoma, B-cell lymphoma, bladder carcinoma, cervical carcinoma, chronic lymphoblastic leukemia, chronic myeloid leukemia, colorectal carcinoma, endometrial carcinoma, glioma, glioblastoma, gastric carcinoma, hepatocellular carcinoma, Hodgkin lymphoma, leukemia, lung carcinoma, melanoma, medulloblastoma, mantle cell lymphoma, multiple myeloma, non-Hodgkin lymphoma, non-small cell lung carcinoma, ovarian carcinoma, oesophageal carcinoma, osteosarcoma, pancreatic carcinoma, prostate carcinoma, renal cell carcinoma, squamous cell carcinoma of the head and neck, small cell lung cancer, thyroid carcinoma, and/or testicular tumor.

In still a further aspect, miR-15, miR-26, miR-31, miR-145, miR-188, miR-215, miR-216, miR-331, or mmu-miR-292-3p, or miR-15, miR-26, miR-31, miR-145, miR-188, miR-215, miR-216, miR-331, or mmu-miR-292-3p inhibitor and miR-147 or miR-147 inhibitor are administered to patients with astrocytoma, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, non-Hodgkin lymphoma, non-small cell lung carcinoma, ovarian carcinoma, oesophageal carcinoma, osteosarcoma, pancreatic carcinoma, prostate carcinoma, renal cell carcinoma, squamous cell carcinoma of the head and neck, and/or thyroid carcinoma.

In yet another aspect, miR-15, miR-26, miR-31, miR-145, miR-147, miR-215, miR-216, miR-331, or mmu-miR-292-3p, or miR-15, miR-26, miR-31, miR-145, miR-147, miR-215, miR-216, miR-331, or mmu-miR-292-3p inhibitor and miR-188 or miR-188 inhibitor are administered to patients with astrocytoma, anaplastic large cell lymphoma, acute myeloid leukemia, breast carcinoma, B-cell lymphoma, Burkitt's lymphoma, bladder carcinoma, cervical carcinoma, chronic lymphoblastic leukemia, colorectal carcinoma, endometrial carcinoma, esophageal squamous cell carcinoma, glioma, glioblastoma, gastric carcinoma, hepatocellular carcinoma, leukemia, lung carcinoma, melanoma, multiple myeloma, non-Hodgkin lymphoma, non-small cell lung carcinoma, ovarian carcinoma, oesophageal carcinoma, pancreatic carcinoma, prostate carcinoma, renal cell carcinoma, squamous cell carcinoma of the head and neck, thyroid carcinoma, and/or testicular tumor.

In other aspects, miR-15, miR-26, miR-31, miR-145, miR-147, miR-188, miR-215, miR-216, miR-331, or mmu-miR-292-3p, or miR-15, miR-26, miR-31, miR-145, miR-147, miR-188, miR-215, miR-216, miR-331, or mmu-miR-292-3p inhibitor and miR-200 or miR-200 inhibitor are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, lipoma, multiple myeloma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, and/or testicular tumor

In other aspects, miR-15, miR-26, miR-31, miR-145, miR-147, miR-188, miR-216, miR-331, or mmu-miR-292-3p, or miR-15, miR-26, miR-31, miR-145, miR-147, miR-188, miR-216, miR-331, or mmu-miR-292-3p inhibitor and miR-215 or miR-215 inhibitor are administered to patients with astrocytoma, anaplastic large cell lymphoma, acute lymphoblastic leukemia, acute myeloid leukemia, angiosarcoma, breast carcinoma, B-cell lymphoma, bladder carcinoma, cervical carcinoma, chronic lymphoblastic leukemia, chronic myeloid leukemia, colorectal carcinoma, endometrial carcinoma, esophageal squamous cell carcinoma, Ewing's sarcoma, glioma, glioblastoma, gastric carcinoma, gastrinoma, hepatoblastoma, hepatocellular carcinoma, Hodgkin lymphoma, Kaposi's sarcoma, leukemia, lung carcinoma, lipoma, leiomyosarcoma, liposarcoma, melanoma, mucosa-associated lymphoid tissue B-cell lymphoma, mantle cell lymphoma, myxofibrosarcoma, myeloid leukemia, multiple myeloma, neuroblastoma, neurofibroma, non-Hodgkin lymphoma, nasopharyngeal carcinoma, non-small cell lung carcinoma, ovarian carcinoma, oesophageal carcinoma, osteosarcoma, pancreatic carcinoma, prostate carcinoma, pheochromocytoma, renal cell carcinoma, rhabdomyosarcoma, squamous cell carcinoma of the head and neck, Schwanomma, small cell lung cancer, thyroid carcinoma, testicular tumor, urothelial carcinoma, and/or Wilm's tumor.

In certain aspects, miR-15, miR-26, miR-31, miR-145, miR-147, miR-188, miR-215, miR-331, or mmu-miR-292-3p or miR-15, miR-26, miR-31, miR-145, miR-147, miR-188, miR-215, miR-331, or mmu-miR-292-3p inhibitor and miR-216 or miR-216 inhibitor are administered to patients with astrocytoma, breast carcinoma, cervical carcinoma, carcinoma of the head and neck, colorectal carcinoma, endometrial carcinoma, glioma, glioblastoma, gastric carcinoma, hepatocellular carcinoma, Hodgkin lymphoma, leukemia, lung carcinoma, mucosa-associated lymphoid tissue B-cell lymphoma, myeloid leukemia, neurofibroma, non-Hodgkin lymphoma, non-small cell lung carcinoma, ovarian carcinoma, oesophageal carcinoma, osteosarcoma, prostate carcinoma, pheochromocytoma, squamous cell carcinoma of the head and neck, and/or testicular tumor.

In a further aspect, miR-15, miR-26, miR-31, miR-145, miR-147, miR-188, miR-215, miR-216, or miR-331, or miR-15, miR-26, miR-31, miR-145, miR-147, miR-188, miR-215, miR-216, or miR-331 inhibitor and miR-292-3p or miR-292-3p inhibitor are administered to patients with astrocytoma, anaplastic large cell lymphoma, acute lymphoblastic leukemia, acute myeloid leukemia, angiosarcoma, breast carcinoma, B-cell lymphoma, bladder carcinoma, cervical carcinoma, chronic myeloid leukemia, colorectal carcinoma, endometrial carcinoma, Ewing's sarcoma, glioma, glioblastoma, gastric carcinoma, hepatoblastoma, hepatocellular carcinoma, Kaposi's sarcoma, leukemia, lung carcinoma, lipoma, leiomyosarcoma, liposarcoma, laryngeal squamous cell carcinoma, melanoma, myxofibrosarcoma, multiple myeloma, neuroblastoma, non-Hodgkin lymphoma, nasopharyngeal carcinoma, non-small cell lung carcinoma, ovarian carcinoma, oesophageal carcinoma, osteosarcoma, pancreatic carcinoma, prostate carcinoma, renal cell carcinoma, rhabdomyosarcoma, squamous cell carcinoma of the head and neck, Schwanomma, small cell lung cancer, thyroid carcinoma, testicular tumor, urothelial carcinoma, and/or Wilm's tumor.

In still a further aspect, miR-15, miR-26, miR-31, miR-145, miR-147, miR-188, miR-215, miR-216, or mmu-miR-292-3p or miR-15, miR-26, miR-31, miR-145, miR-147, miR-188, miR-215, miR-216, or mmu-miR-292-3p inhibitor and miR-331 or miR-331 inhibitor are administered to patients with astrocytoma, anaplastic large cell lymphoma, acute lymphoblastic leukemia, acute myeloid leukemia, angiosarcoma, breast carcinoma, B-cell lymphoma, bladder carcinoma, cervical carcinoma, carcinoma of the head and neck, chronic lymphoblastic leukemia, colorectal carcinoma, endometrial carcinoma, glioma, glioblastoma, gastric carcinoma, gastrinoma, hepatocellular carcinoma, Kaposi's sarcoma, leukemia, lung carcinoma, leiomyosarcoma, laryngeal squamous cell carcinoma, larynx carcinoma, melanoma, myxofibrosarcoma, myeloid leukemia, multiple myeloma, neuroblastoma, neurofibroma, non-Hodgkin lymphoma, ovarian carcinoma, oesophageal carcinoma, osteosarcoma, pancreatic carcinoma, prostate carcinoma, pheochromocytoma, renal cell carcinoma, rhabdomyosarcoma, squamous cell carcinoma of the head and neck, small cell lung cancer, thyroid carcinoma, and/or testicular tumor.

It is contemplated that when miR-15, miR-26, miR-31, miR-145, miR-147, miR-188, miR-215, miR-216, miR-331, or mmu-miR-292-3p or a miR-15, miR-26, miR-31, miR-145, miR-147, miR-188, miR-215, miR-216, miR-331, or mmu-miR-292-3p inhibitor is given in combination with one or more other miRNA molecules, the multiple different miRNAs or inhibitors may be given at the same time or sequentially. In some embodiments, therapy proceeds with one miRNA or inhibitor and that therapy is followed up with therapy with the other miRNA or inhibitor 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, or 1, 2, 3, 4, 5, 6, 7, 8, 9, 10, 11, or 12 months or any such combination later.

Further embodiments include the identification and assessment of an expression profile indicative of miR-15, miR-26, miR-31, miR-145, miR-147, miR-188, miR-215, miR-216, miR-331, or mmu-miR-292-3p status in a cell or tissue comprising expression assessment of one or more gene from Table 1, 3, and/or 4, or any combination thereof.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers or genes from Tables 1, 3, and/or 4); it is contemplated that the nucleic acid profile can be obtained using a set of RNAs, using for example nucleic acid amplification or hybridization techniques well know to one of ordinary skill in the art. The difference in the expression profile in the sample from a patient and a reference expression profile, such as an expression profile from a normal or non-pathologic sample, or a digitized reference, is indicative of a pathologic, disease, or cancerous condition. In certain aspects the expression profile is an indicator of a propensity to or probability of (i.e., risk factor for a disease or condition) developing such a condition(s). Such a risk or propensity may indicate a treatment, increased monitoring, prophylactic measures, and the like. A nucleic acid or probe set may comprise or identify a segment of a corresponding mRNA and may include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more segments, including any integer or range derivable there between, of a gene or genetic marker, or a nucleic acid, mRNA or a probe representative thereof that is listed in Tables 1, 3, and/or 4 or identified by the methods described herein.

Certain embodiments of the invention are directed to compositions and methods for assessing, prognosing, or treating a pathological condition in a patient comprising measuring or determining an expression profile of one or more miRNA or marker(s) in a sample from the patient, wherein a difference in the expression profile in the sample from the patient and an expression profile of a normal sample or reference expression profile is indicative of pathological condition and particularly cancer (e.g., In certain aspects of the invention, the miRNAs, cellular pathway, gene, or genetic marker is or is representative of one or more pathway or marker described in Table 1, 2, 3, and/or 4, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of markers in Table 1, 3, and/or 4, including any combination thereof.

Certain embodiments of the invention include determining expression of one or more marker, gene, or nucleic acid segment representative of one or more genes, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Proteins are typically assayed by immunoblotting, chromatography, or mass spectrometry or other methods known to those of ordinary skill in the art.

The present invention also concerns kits containing compositions of the invention or compositions to implement methods of the invention. In some embodiments, kits can be used to evaluate one or more marker molecules, and/or express one or more miRNA or miRNA inhibitor. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA or miRNA inhibitor to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on a miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

TABLE 2A
Significantly affected functional cellular pathways following hsa-miR-15
over-expression in human cancer cells.

| Number | |
|---|---|
| of Genes | Pathway Functions |
| 18 | Cancer, Tumor Morphology, Cellular Growth and Proliferation |
| 16 | Cell Cycle, Cancer, Skeletal and Muscular Disorders |
| 15 | Cellular Movement, Cellular Assembly and Organization, Cellular Compromise |
| 15 | Inflammatory Disease, Cell Morphology, Dermatological Diseases and Conditions |
| 15 | Cellular Movement, Cell-To-Cell Signaling and Interaction, Tissue Development |
| 5 | Cardiovascular System Development and Function, Gene Expression, Cancer |
| 1 | Cancer, Cell Morphology, Cell-To-Cell Signaling and Interaction |
| 1 | Cancer, Cardiovascular System Development and Function, Cell-To-Cell Signaling |
| | and Interaction |
| 1 | Cancer, Cell Cycle, Cellular Movement |
| 1 | Cellular Assembly and Organization, Neurological Disease, Psychological Disorders |
| 1 | Cell Death, Cell-To-Cell Signaling and Interaction, Cellular Growth and Proliferation |
| 1 | Cell-To-Cell Signaling and Interaction, Cellular Development, Connective Tissue |
| | Development and Function |
| 1 | Cellular Assembly and Organization, Cell Morphology, Molecular Transport |

TABLE 2B
Significantly affected functional cellular pathways following
hsa-miR-26 over-expression in human cancer cells.

| Number | |
|---|---|
| of Genes | Pathway Functions |
| 18 | Cellular Movement, Cancer, Cell Death |
| 16 | Cellular Development, Cellular Growth and Proliferation, |
| | Connective Tissue Development and Function |
| 16 | Cellular Movement, Cellular Growth and Proliferation, |
| | Cardiovascular System Development and Function |
| 15 | Cell Signaling, Cancer, Molecular Transport |
| 14 | Cell Morphology, Digestive System Development and |
| | Function, Renal and Urological System Development |
| | and Function |
| 14 | Carbohydrate Metabolism, Cell Signaling, Energy Production |
| 14 | Cell Signaling, Gene Expression, Cellular Growth and |
| | Proliferation |
| 13 | Cancer, Cell-To-Cell Signaling and Interaction, Cellular |
| | Assembly and Organization |
| 12 | Cell Death, Cancer, Cellular Movement |
| 1 | Cancer, Drug Metabolism, Genetic Disorder |
| 1 | Cellular Assembly and Organization, RNA |
| | Post-Transcriptional Modification |
| 1 | Molecular Transport, Protein Trafficking, Cell-To-Cell |
| | Signaling and Interaction |

TABLE 2C
Significantly affected functional cellular pathways following
inhibition of hsa-miR-31 expression in human cancer cells.

| Number | |
|---|---|
| of Genes | Pathway Functions |
| 5 | Hematological System Development and Function, Immune |
| | Response, Immune and Lymphatic System Development |
| | and Function |

TABLE 2D
Significantly affected functional cellular pathways following
hsa-miR-145 over-expression in human cancer cells.

| Number | |
|---|---|
| of Genes | Pathway Functions |
| 1 | Cancer, Cell Morphology, Dermatological Diseases and |
| | Conditions |
| 1 | Tissue Morphology, Hematological System Development |
| | and Function, Immune and Lymphatic System Development |
| | and Function |

TABLE 2E
Significantly affected functional cellular pathways following
hsa-miR-147 over-expression in human cancer cells.

| Number | |
|---|---|
| of Genes | Pathway Functions |
| 16 | Cardiovascular System Development and Function, Cellular |
| | Movement, Cellular Growth and Proliferation |
| 15 | Cancer, Cell Morphology, Dermatological Diseases and |
| | Conditions |
| 15 | Cellular Assembly and Organization, Cardiovascular Disease, |
| | Cell Death |
| 14 | Cellular Movement, Renal and Urological System |
| | Development and Function, Cancer |
| 14 | Hematological Disease, Cellular Growth and Proliferation, |
| | Lipid Metabolism |
| 12 | Cellular Compromise, Immune Response, Cancer |
| 7 | Cell Morphology, Cellular Development, Cell-To-Cell |
| | Signaling and Interaction |
| 1 | Cell-To-Cell Signaling and Interaction, Cellular Assembly and |
| | Organization, Nervous System Development and Function |
| 1 | Cell-To-Cell Signaling and Interaction, Cellular Function and |
| | Maintenance, Connective Tissue Development and Function |
| 1 | Cellular Assembly and Organization, Cellular Function and |
| | Maintenance, Cell-To-Cell Signaling and Interaction |

TABLE 2F
Significantly affected functional cellular pathways following
hsa-miR-188 over-expression in human cancer cells.

| Number | |
|---|---|
| of Genes | Pathway Functions |
| 15 | Cardiovascular System Development and Function, |
| | Cell-To-Cell Signaling and Interaction, Tissue Development |
| 14 | Tissue Development, Cell Death, Renal and Urological |
| | Disease |
| 13 | Cell Cycle, Cellular Growth and Proliferation, Endocrine |
| | System Development and Function |
| 8 | Cell Death, DNA Replication, Recombination, and Repair, |
| | Cellular Growth and Proliferation |
| 1 | Cell Morphology, Cellular Assembly and Organization, |
| | Psychological Disorders |
| 1 | Cell Cycle, Dermatological Diseases and Conditions, |
| | Genetic Disorder |
| 1 | Amino Acid Metabolism, Post-Translational Modification, |
| | Small Molecule Biochemistry |
| 1 | Molecular Transport, Protein Trafficking, Cell-To-Cell |
| | Signaling and Interaction |

TABLE 2G
Significantly affected functional cellular pathways following
hsa-miR-215 over-expression in human cancer cells.

| Number | |
|---|---|
| of Genes | Pathway Functions |
| 21 | Cellular Growth and Proliferation, Cell Death, Lipid |
| | Metabolism |
| 16 | Cellular Function and Maintenance, Hematological System |
| | Development and Function, Immune and Lymphatic System |
| | Development and Function |
| 15 | Cell Death, Cancer, Connective Tissue Disorders |
| 14 | Cellular Growth and Proliferation, Connective Tissue |
| | Development and Function, Cellular Assembly |
| | and Organization |
| 13 | Cancer, Cell Cycle, Reproductive System Disease |
| 13 | Cellular Growth and Proliferation, Cell Death, Hematological |
| | System Development and Function |
| 11 | Cancer, Gene Expression, Cardiovascular Disease |
| 1 | Neurological Disease, Skeletal and Muscular Disorders, |
| | Cellular Function and Maintenance |
| 1 | Cardiovascular System Development and Function, Cell |
| | Morphology, Cellular Development |
| 1 | Cell Death, Cell-To-Cell Signaling and Interaction, |
| | Cellular Growth and Proliferation |
| 1 | Hematological Disease, Genetic Disorder, Hematological |
| | System Development and Function |

TABLE 2H
Significantly affected functional cellular pathways following
hsa-miR-216 over-expression in human cancer cells.

| Number | |
|---|---|
| of Genes | Pathway Functions |
| 14 | Molecular Transport, Small Molecule Biochemistry, |
| | Cellular Development |
| 13 | Gene Expression, Cellular Growth and Proliferation, |
| | Connective Tissue Development and Function |
| 5 | Cell Death, DNA Replication, Recombination, and Repair, |
| | Cancer |
| 1 | Cell-To-Cell Signaling and Interaction, Cellular Function and |
| | Maintenance, Connective Tissue Development and Function |

TABLE 2I
Significantly affected functional cellular pathways following
hsa-miR-331 over-expression in human cancer cells.

| Number | |
|---|---|
| of Genes | Pathway Functions |
| 13 | Cell Death, Dermatological Diseases and Conditions, |
| | Cancer |
| 12 | Developmental Disorder, Cancer, Cell Death |
| 11 | Cancer, Cardiovascular Disease, Cell Morphology |
| 8 | Cell Signaling, Gene Expression, Cancer |
| 1 | Behavior, Connective Tissue Development and Function, |
| | Developmental Disorder |
| 1 | Cancer, Hair and Skin Development and Function, |
| | Nervous System Development and Function |
| 1 | Cellular Function and Maintenance |
| 1 | Lipid Metabolism, Small Molecule Biochemistry, Cancer |
| 1 | Molecular Transport, Protein Trafficking, Cell-To-Cell |
| | Signaling and Interaction |
| 1 | Cellular Assembly and Organization, Cell Morphology, |
| | Molecular Transport |
| 1 | Cell Cycle, Cellular Movement, Cell Morphology |
| 1 | Cell Signaling, Neurological Disease, Cell Morphology |

TABLE 2J
Significantly affected functional cellular pathways following
mmu-miR-292-3p over-expression in human cancer cells.

| Number | |
|---|---|
| of Genes | Pathway Functions |
| 35 | Cellular Growth and Proliferation, Cancer, Cell Death |
| 21 | DNA Replication, Recombination, and Repair, Cellular |
| | Growth and Proliferation, Lipid Metabolism |
| 18 | Cancer, Cell Death, Connective Tissue Disorders |
| 17 | DNA Replication, Recombination, and Repair, Cellular |
| | Function and Maintenance, Cell-To-Cell Signaling |
| | and Interaction |
| 17 | Gene Expression, Cancer, Connective Tissue Disorders |
| 15 | Cellular Assembly and Organization, Nervous System |
| | Development and Function, Cellular Movement |
| 14 | Cell Morphology, Cancer, Cell Death |
| 14 | Cell Morphology, Renal and Urological System Development |
| | and Function, Cancer |
| 13 | Cellular Assembly and Organization, Cellular Compromise, |
| | Gene Expression |
| 5 | Gene Expression, Lipid Metabolism, Small Molecule |
| | Biochemistry |
| 1 | Gene Expression |
| 1 | Reproductive System Development and Function, |
| | Cell-To-Cell Signaling and Interaction |
| 1 | |
| 1 | Cancer, Cardiovascular System Development and |
| | Function, Cell-To-Cell Signaling and Interaction |
| 1 | Cellular Function and Maintenance |
| 1 | Post-Translational Modification, Gene Expression, |
| | Protein Synthesis |
| 1 | Nervous System Development and Function, Nucleic Acid |
| | Metabolism, Cellular Movement |
| 1 | Genetic Disorder, Metabolic Disease, Cellular Assembly |
| | and Organization |
| 1 | Lipid Metabolism, Small Molecule Biochemistry, |
| | Cellular Development |

TABLE 3A
Predicted hsa-miR-15 targets that exhibited altered mRNA expression levels
in human cancer cells after transfection with pre-miR hsa-miR-15.

| | RefSeq | |
|---|---|---|
| | Transcript ID | |
| Gene Symbol | (Pruitt et al, 2005) | Description |
| ABCA1 | NM_005502 | ATP-binding cassette, sub-family A member 1 |
| ADARB1 | NM_001033049 | RNA-specific adenosine deaminase B1 isoform 4 |
| ADRB2 | NM_000024 | adrenergic, beta-2-, receptor, surface |
| AKAP12 | NM_005100 | A-kinase anchor protein 12 isoform 1 |
| ANKRD46 | NM_198401 | ankyrin repeat domain 46 |
| AP1S2 | NM_003916 | adaptor-related protein complex 1 sigma 2 |
| ARHGDIA | NM_004309 | Rho GDP dissociation inhibitor (GDI) alpha |
| ARL2 | NM_001667 | ADP-ribosylation factor-like 2 |
| BAG5 | NM_001015048 | BCL2-associated athanogene 5 isoform b |
| CA12 | NM_001218 | carbonic anhydrase XII isoform 1 precursor |
| CCND1 | NM_053056 | cyclin D1 |
| CCND3 | NM_001760 | cyclin D3 |
| CDC37L1 | NM_017913 | cell division cycle 37 homolog (S. |
| CDCA4 | NM_017955 | cell division cycle associated 4 |
| CDS2 | NM_003818 | phosphatidate cytidylyltransferase 2 |
| CGI-38 | NM_015964 | hypothetical protein LOC51673 |
| CHUK | NM_001278 | conserved helix-loop-helix ubiquitous kinase |
| COL6A1 | NM_001848 | collagen, type VI, alpha 1 precursor |
| CYP4F3 | NM_000896 | cytochrome P450, family 4, subfamily F, |
| DDAH1 | NM_012137 | dimethylarginine dimethylaminohydrolase 1 |
| DUSP6 | NM_001946 | dual specificity phosphatase 6 isoform a |
| EIF4E | NM_001968 | eukaryotic translation initiation factor 4E |
| FAM18B | NM_016078 | hypothetical protein LOC51030 |
| FGF2 | NM_002006 | fibroblast growth factor 2 |
| FGFR4 | NM_002011 | fibroblast growth factor receptor 4 isoform 1 |
| FKBP1B | NM_004116 | FK506-binding protein 1B isoform a |
| FSTL1 | NM_007085 | follistatin-like 1 precursor |
| GCLC | NM_001498 | glutamate-cysteine ligase, catalytic subunit |
| GFPT1 | NM_002056 | glucosamine-fructose-6-phosphate |
| GTSE1 | NM_016426 | G-2 and S-phase expressed 1 |
| HAS2 | NM_005328 | hyaluronan synthase 2 |
| HMGA2 | NM_001015886 | high mobility group AT-hook 2 isoform c |
| HSPA1B | NM_005346 | heat shock 70 kDa protein 1B |
| IGFBP3 | NM_000598 | insulin-like growth factor binding protein 3 |
| KCNJ2 | NM_000891 | potassium inwardly-rectifying channel J2 |
| LCN2 | NM_005564 | lipocalin 2 (oncogene 24p3) |
| LOXL2 | NM_002318 | lysyl oxidase-like 2 precursor |
| LRP12 | NM_013437 | suppression of tumorigenicity |
| MAP7 | NM_003980 | microtubule-associated protein 7 |
| NTE | NM_006702 | neuropathy target esterase |
| PLSCR4 | NM_020353 | phospholipid scramblase 4 |
| PODXL | NM_001018111 | podocalyxin-like precursor isoform 1 |
| PPP1R11 | NM_021959 | protein phosphatase 1, regulatory (inhibitor) |
| QKI | NM_206853 | quaking homolog, KH domain RNA binding isoform |
| RAFTLIN | NM_015150 | raft-linking protein |
| RPS6KA3 | NM_004586 | ribosomal protein S6 kinase, 90 kDa, polypeptide |
| RPS6KA5 | NM_004755 | ribosomal protein S6 kinase, 90 kDa, polypeptide |
| SLC11A2 | NM_000617 | solute carrier family 11 (proton-coupled |
| SLC26A2 | NM_000112 | solute carrier family 26 member 2 |
| SNAP23 | NM_003825 | synaptosomal-associated protein 23 isoform |
| SPARC | NM_003118 | secreted protein, acidic, cysteine-rich |
| SPFH2 | NM_007175 | SPFH domain family, member 2 isoform 1 |
| STC1 | NM_003155 | stanniocalcin 1 precursor |
| SYNE1 | NM_015293 | nesprin 1 isoform beta |
| TACC1 | NM_006283 | transforming, acidic coiled-coil containing |
| TAF15 | NM_003487 | TBP-associated factor 15 isoform 2 |
| TFG | NM_001007565 | TRK-fused gene |
| THUMPD1 | NM_017736 | THUMP domain containing 1 |
| TNFSF9 | NM_003811 | tumor necrosis factor (ligand) superfamily, |
| TPM1 | NM_001018004 | tropomyosin 1 alpha chain isoform 3 |
| UBE2I | NM_003345 | ubiquitin-conjugating enzyme E2I |
| VIL2 | NM_003379 | villin 2 |
| VTI1B | NM_006370 | vesicle transport through interaction with |
| YRDC | NM_024640 | ischemia/reperfusion inducible protein |

TABLE 3B
Predicted hsa-miR-26 targets that exhibited altered mRNA expression
levels in human cancer cells after transfection with pre-miR hsa-miR-26.

| | RefSeq | |
|---|---|---|
| | Transcript ID | |
| Gene Symbol | (Pruitt et al., 2005) | Description |
| ABR | NM_001092 | active breakpoint cluster region-related |
| ALDH5A1 | NM_001080 | aldehyde dehydrogenase 5A1 precursor, isoform 2 |
| ATP9A | NM_006045 | ATPase, Class II, type 9A |
| B4GALT4 | NM_003778 | UDP-Gal:betaGlcNAc beta 1,4- |
| BCAT1 | NM_005504 | branched chain aminotransferase 1, cytosolic |
| C14orf10 | NM_017917 | chromosome 14 open reading frame 10 |
| C1orf116 | NM_023938 | specifically androgen-regulated protein |
| C8orf1 | NM_004337 | hypothetical protein LOC734 |
| CCDC28A | NM_015439 | hypothetical protein LOC25901 |
| CDH4 | NM_001794 | cadherin 4, type 1 preproprotein |
| CDK8 | NM_001260 | cyclin-dependent kinase 8 |
| CHAF1A | NM_005483 | chromatin assembly factor 1, subunit A (p150) |
| CHORDC1 | NM_012124 | cysteine and histidine-rich domain |
| CLDN3 | NM_001306 | claudin 3 |
| CREBL2 | NM_001310 | cAMP responsive element binding protein-like 2 |
| CTGF | NM_001901 | connective tissue growth factor |
| EFEMP1 | NM_004105 | EGF-containing fibulin-like extracellular matrix |
| EHD1 | NM_006795 | EH-domain containing 1 |
| EIF2S1 | NM_004094 | eukaryotic translation initiation factor 2, |
| EPHA2 | NM_004431 | ephrin receptor EphA2 |
| FBXO11 | NM_025133 | F-box only protein 11 isoform 1 |
| GALC | NM_000153 | galactosylceramidase isoform a precursor |
| GMDS | NM_001500 | GDP-mannose 4,6-dehydratase |
| GRB10 | NM_001001549 | growth factor receptor-bound protein 10 isoform |
| HAS2 | NM_005328 | hyaluronan synthase 2 |
| HECTD3 | NM_024602 | HECT domain containing 3 |
| HES1 | NM_005524 | hairy and enhancer of split 1 |
| HMGA1 | NM_002131 | high mobility group AT-hook 1 isoform b |
| HMGA2 | NM_001015886 | high mobility group AT-hook 2 isoform c |
| HNMT | NM_001024074 | histamine N-methyltransferase isoform 2 |
| KIAA0152 | NM_014730 | hypothetical protein LOC9761 |
| LOC153561 | NM_207331 | hypothetical protein LOC153561 |
| MAPK6 | NM_002748 | mitogen-activated protein kinase 6 |
| MCL1 | NM_021960 | myeloid cell leukemia sequence 1 isoform 1 |
| METAP2 | NM_006838 | methionyl aminopeptidase 2 |
| MYCBP | NM_012333 | c-myc binding protein |
| NAB1 | NM_005966 | NGFI-A binding protein 1 |
| NR5A2 | NM_003822 | nuclear receptor subfamily 5, group A, member 2 |
| NRG1 | NM_013958 | neuregulin 1 isoform HRG-beta3 |
| NRIP1 | NM_003489 | receptor interacting protein 140 |
| PAPPA | NM_002581 | pregnancy-associated plasma protein A |
| PDCD4 | NM_014456 | programmed cell death 4 isoform 1 |
| PHACTR2 | NM_014721 | phosphatase and actin regulator 2 |
| PTK9 | NM_002822 | twinfilin isoform 1 |
| RAB11FIP1 | NM_001002233 | Rab coupling protein isoform 2 |
| RAB21 | NM_014999 | RAB21, member RAS oncogene family |
| RECK | NM_021111 | RECK protein precursor |
| RHOQ | NM_012249 | ras-like protein TC10 |
| SC4MOL | NM_001017369 | sterol-C4-methyl oxidase-like isoform 2 |
| SLC26A2 | NM_000112 | solute carrier family 26 member 2 |
| SLC2A3 | NM_006931 | solute carrier family 2 (facilitated glucose |
| SRD5A1 | NM_001047 | steroid-5-alpha-reductase 1 |
| STK39 | NM_013233 | serine threonine kinase 39 (STE20/SPS1 homolog, |
| TIMM17A | NM_006335 | translocase of inner mitochondrial membrane 17 |
| TRAPPC4 | NM_016146 | trafficking protein particle complex 4 |
| ULK1 | NM_003565 | unc-51-like kinase 1 |
| UQCRB | NM_006294 | ubiquinol-cytochrome c reductase binding |
| ZNF259 | NM_003904 | zinc finger protein 259 |

TABLE 3C
Predicted hsa-miR-31 targets that exhibited altered mRNA
expression levels in human cancer cells after transfection
with pre-miR hsa-miR-31.

| Gene Symbol | RefSeq Transcript ID (Pruitt et al., 2005) | Δ log₂ |
|---|---|---|
| AKAP2 /// | NM_001004065 /// NM_007203 /// | 0.881687 |
| PALM2- | NM_147150 | |
| AKAP2 | | |
| CXCL3 | NM_002090 | 0.800224 |
| IL8 | NM_000584 | 1.54253 |
| MAFF | NM_012323 /// NM_152878 | 0.873461 |
| QKI | NM_006775 /// NM_206853 /// | 0.773843 |
| | NM_206854 /// NM_206855 | |
| SLC26A2 | NM_000112 | 0.784073 |
| STC1 | NM_003155 | 0.904092 |

TABLE 3D
Predicted hsa-miR-145 targets that exhibited altered mRNA
expression levels in human cancer cells after transfection
with pre-miR hsa-miR-145.

| Gene | RefSeq Transcript ID | |
|---|---|---|
| Symbol | (Pruitt et al., 2005) | Description |
| CXCL3 | NM_002090 | chemokine (C—X—C motif) ligand 3 |

TABLE 3E
Predicted hsa-miR-147 targets that exhibited altered
mRNA expression levels in human cancer cells after
transfection with pre-miR hsa-miR-147.

| | RefSeq | |
|---|---|---|
| | Transcript ID | |
| Gene Symbol | (Pruitt et al., 2005) | Description |
| ANK3 | NM_001149 | ankyrin 3 isoform 2 |
| ANTXR1 | NM_032208 | tumor endothelial marker 8 isoform 1 precursor |
| ARID5B | NM_032199 | AT rich interactive domain 5B (MRF1-like) |
| ATP9A | NM_006045 | ATPase, Class II, type 9A |
| B4GALT1 | NM_001497 | UDP-Gal:betaGlcNAc beta 1,4- |
| C1orf24 | NM_052966 | niban protein isoform 2 |
| C21orf25 | NM_199050 | hypothetical protein LOC25966 |
| C6orf120 | NM_001029863 | hypothetical protein LOC387263 |
| CCND1 | NM_053056 | cyclin D1 |
| COL4A2 | NM_001846 | alpha 2 type IV collagen preproprotein |
| DCP2 | NM_152624 | DCP2 decapping enzyme |
| DPYSL4 | NM_006426 | dihydropyrimidinase-like 4 |
| EIF2C1 | NM_012199 | eukaryotic translation initiation factor 2C, 1 |
| ETS2 | NM_005239 | v-ets erythroblastosis virus E26 oncogene |
| F2RL1 | NM_005242 | coagulation factor II (thrombin) receptor-like 1 |
| FYCO1 | NM_024513 | FYVE and coiled-coil domain containing 1 |
| FZD7 | NM_003507 | frizzled 7 |
| GLUL | NM_001033044 | glutamine synthetase |
| GNS | NM_002076 | glucosamine (N-acetyl)-6-sulfatase precursor |
| GOLPH2 | NM_016548 | golgi phosphoprotein 2 |
| GYG2 | NM_003918 | glycogenin 2 |
| HAS2 | NM_005328 | hyaluronan synthase 2 |
| HIC2 | NM_015094 | hypermethylated in cancer 2 |
| KCNMA1 | NM_001014797 | large conductance calcium-activated potassium |
| LHFP | NM_005780 | lipoma HMGIC fusion partner |
| LIMK1 | NM_002314 | LIM domain kinase 1 |
| MAP3K2 | NM_006609 | mitogen-activated protein kinase kinase kinase |
| MICAL2 | NM_014632 | microtubule associated monoxygenase, calponin |
| NAV3 | NM_014903 | neuron navigator 3 |
| NPTX1 | NM_002522 | neuronal pentraxin I precursor |
| NUPL1 | NM_001008564 | nucleoporin like 1 isoform b |
| OLR1 | NM_002543 | oxidised low density lipoprotein (lectin-like) |
| OXTR | NM_000916 | oxytocin receptor |
| PDCD4 | NM_014456 | programmed cell death 4 isoform 1 |
| PLAU | NM_002658 | urokinase plasminogen activator preproprotein |
| PTHLH | NM_002820 | parathyroid hormone-like hormone isoform 2 |
| RAB22A | NM_020673 | RAS-related protein RAB-22A |
| RHTOC | NM_175744 | ras homolog gene family, member C |
| SPARC | NM_003118 | secreted protein, acidic, cysteine-rich |
| STC1 | NM_003155 | stanniocalcin 1 precursor |
| TGFBR2 | NM_001024847 | TGF-beta type II receptor isoform A precursor |
| TM4SF20 | NM_024795 | transmembrane 4 L six family member 20 |
| TNFRSF12A | NM_016639 | type I transmembrane protein Fn14 |
| ULK1 | NM_003565 | unc-51-like kinase 1 |

TABLE 3F
Predicted hsa-miR-188 targets that exhibited altered mRNA
expression levels in human cancer cells after transfection
with pre-miR hsa-miR-188.

| | RefSeq | |
|---|---|---|
| | Transcript ID | |
| Gene Symbol | (Pruitt et al., 2005) | Description |
| ANKRD46 | NM_198401 | ankyrin repeat domain 46 |
| ANTXR1 | NM_018153 | tumor endothelial marker 8 isoform 3 precursor |
| ATXN1 | NM_000332 | ataxin 1 |
| AXL | NM_001699 | AXL receptor tyrosine kinase isoform 2 |
| BPGM | NM_001724 | 2,3-bisphosphoglycerate mutase |
| C6orf120 | NM_001029863 | hypothetical protein LOC387263 |
| C8orf1 | NM_004337 | hypothetical protein LOC734 |
| CBFB | NM_001755 | core-binding factor, beta subunit isoform 2 |
| CCDC6 | NM_005436 | coiled-coil domain containing 6 |
| CD2AP | NM_012120 | CD2-associated protein |
| CDK2AP1 | NM_004642 | CDK2-associated protein 1 |
| CLU | NM_001831 | clusterin isoform 1 |
| CREB3L2 | NM_194071 | cAMP responsive element binding protein 3-like |
| DAAM1 | NM_014992 | dishevelled-associated activator of |
| DCP2 | NM_152624 | DCP2 decapping enzyme |
| DKFZp564K142 | NM_032121 | implantation-associated protein |
| DLG5 | NM_004747 | discs large homolog 5 |
| EDEM1 | NM_014674 | ER degradation enhancer, mannosidase alpha-like |
| ELOVL6 | NM_024090 | ELOVL family member 6, elongation of long chain |
| EMP1 | NM_001423 | epithelial membrane protein 1 |
| ETS2 | NM_005239 | v-ets erythroblastosis virus E26 oncogene |
| GATAD1 | NM_021167 | GATA zinc finger domain containing 1 |
| GPR125 | NM_145290 | G protein-coupled receptor 125 |
| GREM1 | NM_013372 | gremlin-1 precursor |
| HDAC3 | NM_003883 | histone deacetylase 3 |
| HNRPA0 | NM_006805 | heterogeneous nuclear ribonucleoprotein A0 |
| IER3IP1 | NM_016097 | immediate early response 3 interacting protein |
| IL13RA1 | NM_001560 | interleukin 13 receptor, alpha 1 precursor |
| ITGAV | NM_002210 | integrin alpha-V precursor |
| M6PR | NM_002355 | cation-dependent mannose-6-phosphate receptor |
| MAP4K5 | NM_006575 | mitogen-activated protein kinase kinase kinase |
| MARCKS | NM_002356 | myristoylated alanine-rich protein kinase C |
| PALM2-AKAP2 | NM_007203 | PALM2-AKAP2 protein isoform 1 |
| PCAF | NM_003884 | p300/CBP-associated factor |
| PCTP | NM_021213 | phosphatidylcholine transfer protein |
| PER2 | NM_022817 | period 2 isoform 1 |
| PHACTR2 | NM_014721 | phosphatase and actin regulator 2 |
| PLEKHA1 | NM_001001974 | pleckstrin homology domain containing, family A |
| PRKCA | NM_002737 | protein kinase C, alpha |
| PTEN | NM_000314 | phosphatase and tensin homolog |
| RGS20 | NM_003702 | regulator of G-protein signalling 20 isoform b |
| RNASE4 | NM_002937 | ribonuclease, RNase A family, 4 precursor |
| RSAD1 | NM_018346 | radical S-adenosyl methionine domain containing |
| SFRS7 | NM_001031684 | splicing factor, arginine/serine-rich 7, 35 kDa |
| SLC39A9 | NM_018375 | solute carrier family 39 (zinc transporter), |
| SLC4A4 | NM_003759 | solute carrier family 4, sodium bicarbonate |
| ST13 | NM_003932 | heat shock 70 kD protein binding protein |
| STC1 | NM_003155 | stanniocalcin 1 precursor |
| SYNJ2BP | NM_018373 | synaptojanin 2 binding protein |
| TAPBP | NM_003190 | tapasin isoform 1 precursor |
| TBL1X | NM_005647 | transducin beta-like 1X |
| TMBIM1 | NM_022152 | transmembrane BAX inhibitor motif containing 1 |
| TP73L | NM_003722 | tumor protein p73-like |
| TRPC1 | NM_003304 | transient receptor potential cation channel, |
| VAV3 | NM_006113 | vav 3 oncogene |
| WDR39 | NM_004804 | WD repeat domain 39 |
| ZNF281 | NM_012482 | zinc finger protein 281 |

TABLE 3G
Predicted hsa-miR-215 targets that exhibited altered mRNA expression levels
in human cancer cells after transfection with pre-miR hsa-miR-215.

| | RefSeq | |
|---|---|---|
| | Transcript ID (Pruitt | |
| Gene Symbol | et al., 2005) | Description |
| ACADSB | NM_001609 | acyl-Coenzyme A dehydrogenase, short/branched |
| ADCY7 | NM_001114 | adenylate cyclase 7 |
| ARL2BP | NM_012106 | binder of Arl Two |
| ATP2B4 | NM_001001396 | plasma membrane calcium ATPase 4 isoform 4a |
| C1D | NM_006333 | nuclear DNA-binding protein |
| C6orf120 | NM_001029863 | hypothetical protein LOC387263 |
| CDCA4 | NM_017955 | cell division cycle associated 4 |
| COL6A1 | NM_001848 | collagen, type VI, alpha 1 precursor |
| COPS7A | NM_016319 | COP9 complex subunit 7a |
| CRSP2 | NM_004229 | cofactor required for Sp1 transcriptional |
| CTAGE5 | NM_005930 | CTAGE family, member 5 isoform 1 |
| CTH | NM_001902 | cystathionase isoform 1 |
| DICER1 | NM_030621 | dicer 1 |
| DMN | NM_015286 | desmuslin isoform B |
| EFEMP1 | NM_004105 | EGF-containing fibulin-like extracellular matrix |
| EREG | NM_001432 | epiregulin precursor |
| FBLN1 | NM_006487 | fibulin 1 isoform A precursor |
| FGF2 | NM_002006 | fibroblast growth factor 2 |
| FGFR1 | NM_023107 | fibroblast growth factor receptor 1 isoform 5 |
| GREB1 | NM_148903 | GREB1 protein isoform c |
| HOXA10 | NM_018951 | homeobox A10 isoform a |
| HSA9761 | NM_014473 | dimethyladenosine transferase |
| IL11 | NM_000641 | interleukin 11 precursor |
| IL1R1 | NM_000877 | interleukin 1 receptor, type I precursor |
| LMAN1 | NM_005570 | lectin, mannose-binding, 1 precursor |
| LOC153561 | NM_207331 | hypothetical protein LOC153561 |
| MAPKAPK2 | NM_004759 | mitogen-activated protein kinase-activated |
| MCM10 | NM_018518 | minichromosome maintenance protein 10 isoform 2 |
| MCM3 | NM_002388 | minichromosome maintenance protein 3 |
| NID1 | NM_002508 | nidogen (enactin) |
| NSF | NM_006178 | N-ethylmaleimide-sensitive factor |
| NUDT15 | NM_018283 | nudix-type motif 15 |
| PABPC4 | NM_003819 | poly A binding protein, cytoplasmic 4 |
| PIP5K2B | NM_003559 | phosphatidylinositol-4-phosphate 5-kinase type |
| PLAU | NM_002658 | urokinase plasminogen activator preproprotein |
| PPP1CA | NM_001008709 | protein phosphatase 1, catalytic subunit, alpha |
| PPP1CB | NM_002709 | protein phosphatase 1, catalytic subunit, beta |
| PRNP | NM_000311 | prion protein preproprotein |
| PTS | NM_000317 | 6-pyruvoyltetrahydropterin synthase |
| RAB2 | NM_002865 | RAB2, member RAS oncogene family |
| RAB40B | NM_006822 | RAB40B, member RAS oncogene family |
| RB1 | NM_000321 | retinoblastoma 1 |
| RNF141 | NM_016422 | ring finger protein 141 |
| RPL4 | NM_000968 | ribosomal protein L4 |
| SLC19A2 | NM_006996 | solute carrier family 19, member 2 |
| SLC1A4 | NM_003038 | solute carrier family 1, member 4 |
| SLC26A2 | NM_000112 | solute carrier family 26 member 2 |
| SLC39A6 | NM_012319 | solute carrier family 39 (zinc transporter), |
| SMA4 | NM_021652 | SMA4 |
| SOAT1 | NM_003101 | sterol O-acyltransferase (acyl-Coenzyme A: |
| SPARC | NM_003118 | secreted protein, acidic, cysteine-rich |
| SRD5A1 | NM_001047 | steroid-5-alpha-reductase 1 |
| SS18 | NM_001007559 | synovial sarcoma translocation, chromosome 18 |
| TBC1D16 | NM_019020 | TBC1 domain family, member 16 |
| TDG | NM_001008411 | thymine-DNA glycosylase isoform 2 |
| TM4SF20 | NM_024795 | transmembrane 4 L six family member 20 |
| TOR1AIP1 | NM_015602 | lamina-associated polypeptide 1B |
| TRIM22 | NM_006074 | tripartite motif-containing 22 |
| TRIP13 | NM_004237 | thyroid hormone receptor interactor 13 |
| WIG1 | NM_022470 | p53 target zinc finger protein isoform 1 |
| ZFHX1B | NM_014795 | zinc finger homeobox 1b |
| ZNF609 | NM_015042 | zinc finger protein 609 |

TABLE 3H
Predicted hsa-miR-216 targets that exhibited altered mRNA expression levels
in human cancer cells after transfection with pre-miR hsa-miR-216.

| | RefSeq | |
|---|---|---|
| | Transcript ID | |
| Gene Symbol | (Pruitt et al, 2005) | Description |
| AXL | NM_001699 | AXL receptor tyrosine kinase isoform 2 |
| BCL10 | NM_003921 | B-cell CLL/lymphoma 10 |
| BNIP3L | NM_004331 | BCL2/adenovirus E1B 19 kD-interacting protein |
| CREB3L2 | NM_194071 | cAMP responsive element binding protein 3-like |
| CTH | NM_001902 | cystathionase isoform 1 |
| DIO2 | NM_000793 | deiodinase, iodothyronine, type II isoform a |
| EIF2S1 | NM_004094 | eukaryotic translation initiation factor 2, |
| FCHO1 | NM_015122 | FCH domain only 1 |
| FEZ2 | NM_005102 | zygin 2 |
| GREM1 | NM_013372 | gremlin-1 precursor |
| HDAC3 | NM_003883 | histone deacetylase 3 |
| IDI1 | NM_004508 | isopentenyl-diphosphate delta isomerase |
| MGC4172 | NM_024308 | short-chain dehydrogenase/reductase |
| NFYC | NM_014223 | nuclear transcription factor Y, gamma |
| PAPPA | NM_002581 | pregnancy-associated plasma protein A |
| PIR | NM_001018109 | pirin |
| PLEKHA1 | NM_001001974 | pleckstrin homology domain containing, family A |
| RP2 | NM_006915 | XRP2 protein |
| SCD | NM_005063 | stearoyl-CoA desaturase |
| SLC2A3 | NM_006931 | solute carrier family 2 (facilitated glucose |
| SNRPD1 | NM_006938 | small nuclear ribonucleoprotein D1 polypeptide |
| SSB | NM_003142 | autoantigen La |
| TEAD1 | NM_021961 | TEA domain family member 1 |
| TGFBR3 | NM_003243 | transforming growth factor, beta receptor III |
| TIPRL | NM_152902 | TIP41, TOR signalling pathway regulator-like |
| TMC5 | NM_024780 | transmembrane channel-like 5 |
| UBE2V2 | NM_003350 | ubiquitin-conjugating enzyme E2 variant 2 |
| VAV3 | NM_006113 | vav 3 oncogene |
| WIG1 | NM_022470 | p53 target zinc finger protein isoform 1 |

TABLE 3I
Predicted hsa-miR-331 targets that exhibited altered mRNA expression levels
in human cancer cells after transfection with pre-miR hsa-miR-331.

| | RefSeq | |
|---|---|---|
| | Transcript ID | |
| Gene Symbol | (Pruitt et al., 2005) | Description |
| AQP3 | NM_004925 | aquaporin 3 |
| B4GALT4 | NM_003778 | UDP-Gal:betaGlcNAc beta 1,4- |
| BCL2L1 | NM_001191 | BCL2-like 1 isoform 2 |
| BICD2 | NM_001003800 | bicaudal D homolog 2 isoform 1 |
| C19orf10 | NM_019107 | chromosome 19 open reading frame 10 |
| CASP7 | NM_033340 | caspase 7 isoform beta |
| CDS2 | NM_003818 | phosphatidate cytidylyltransferase 2 |
| COL4A2 | NM_001846 | alpha 2 type IV collagen preproprotein |
| COMMD9 | NM_014186 | COMM domain containing 9 |
| CXCL1 | NM_001511 | chemokine (C—X—C motif) ligand 1 |
| D15Wsu75e | NM_015704 | hypothetical protein LOC27351 |
| DDAH1 | NM_012137 | dimethylarginine dimethylaminohydrolase 1 |
| EFNA1 | NM_004428 | ephrin A1 isoform a precursor |
| EHD1 | NM_006795 | EH-domain containing 1 |
| EIF5A2 | NM_020390 | eIF-5A2 protein |
| ENO1 | NM_001428 | enolase 1 |
| EREG | NM_001432 | epiregulin precursor |
| FAM63B | NM_019092 | hypothetical protein LOC54629 |
| FGFR1 | NM_000604 | fibroblast growth factor receptor 1 isoform 1 |
| GALNT7 | NM_017423 | polypeptide N-acetylgalactosaminyltransferase 7 |
| HLRC1 | NM_031304 | HEAT-like (PBS lyase) repeat containing 1 |
| IL13RA1 | NM_001560 | interleukin 13 receptor, alpha 1 precursor |
| IL32 | NM_001012631 | interleukin 32 isoform B |
| IL6R | NM_000565 | interleukin 6 receptor isoform 1 precursor |
| ITGB4 | NM_000213 | integrin beta 4 isoform 1 precursor |
| KIAA0090 | NM_015047 | hypothetical protein LOC23065 |
| KIAA1641 | NM_020970 | hypothetical protein LOC57730 |
| MGC4172 | NM_024308 | short-chain dehydrogenase/reductase |
| NPTX1 | NM_002522 | neuronal pentraxin I precursor |
| NR5A2 | NM_003822 | nuclear receptor subfamily 5, group A, member 2 |
| PDPK1 | NM_002613 | 3-phosphoinositide dependent protein kinase-1 |
| PHLPP | NM_194449 | PH domain and leucine rich repeat protein |
| PLEC1 | NM_000445 | plectin 1 isoform 1 |
| PODXL | NM_001018111 | podocalyxin-like precursor isoform 1 |
| PXN | NM_002859 | Paxillin |
| RHOBTB1 | NM_001032380 | Rho-related BTB domain containing 1 |
| RPA2 | NM_002946 | replication protein A2, 32 kDa |
| RPE | NM_006916 | ribulose-5-phosphate-3-epimerase isoform 2 |
| SDC4 | NM_002999 | syndecan 4 precursor |
| SLC7A1 | NM_003045 | solute carrier family 7 (cationic amino acid |
| STX6 | NM_005819 | syntaxin 6 |
| TBC1D16 | NM_019020 | TBC1 domain family, member 16 |
| THBS1 | NM_003246 | thrombospondin 1 precursor |
| TMEM2 | NM_013390 | transmembrane protein 2 |
| TMEM45A | NM_018004 | transmembrane protein 45A |
| TNC | NM_002160 | tenascin C (hexabrachion) |
| TNFSF9 | NM_003811 | tumor necrosis factor (ligand) superfamily, |
| TRFP | NM_004275 | Trf (TATA binding protein-related |
| TXLNA | NM_175852 | Taxilin |
| USP46 | NM_022832 | ubiquitin specific protease 46 |
| VANGL1 | NM_138959 | vang-like 1 |
| WDR1 | NM_005112 | WD repeat-containing protein 1 isoform 2 |
| WNT7B | NM_058238 | wingless-type MMTV integration site family, |
| WSB2 | NM_018639 | WD SOCS-box protein 2 |
| YRDC | NM_024640 | ischemia/reperfusion inducible protein |
| ZNF259 | NM_003904 | zinc finger protein 259 |
| ZNF395 | NM_018660 | zinc finger protein 395 |

TABLE 3J
Predicted mmu-miR-292-3p targets that exhibited altered mRNA expression
levels in human cancer cells after transfection with pre-miR mmu-miR-292-3p.

| | RefSeq | |
|---|---|---|
| | Transcript ID | |
| Gene Symbol | (Pruitt et al., 2005) | Description |
| AP1G1 | NM_001030007 | adaptor-related protein complex 1, gamma 1 |
| AKR7A2 | NM_003689 | aldo-keto reductase family 7, member A2 |
| ALDH3A2 | NM_000382 | aldehyde dehydrogenase 3A2 isoform 2 |
| ARCN1 | NM_001655 | Archain |
| ARL2BP | NM_012106 | binder of Arl Two |
| BDKRB2 | NM_000623 | bradykinin receptor B2 |
| BICD2 | NM_001003800 | bicaudal D homolog 2 isoform 1 |
| BPGM | NM_001724 | 2,3-bisphosphoglycerate mutase |
| BRP44 | NM_015415 | brain protein 44 |
| BTG2 | NM_006763 | B-cell translocation gene 2 |
| C14orf2 | NM_004894 | hypothetical protein LOC9556 |
| C1GALT1C1 | NM_001011551 | C1GALT1-specific chaperone 1 |
| C2orf17 | NM_024293 | hypothetical protein LOC79137 |
| CASP7 | NM_033340 | caspase 7 isoform beta |
| CDH4 | NM_001794 | cadherin 4, type 1 preproprotein |
| COPS6 | NM_006833 | COP9 signalosome subunit 6 |
| COQ2 | NM_015697 | para-hydroxybenzoate-polyprenyltransferase, |
| CYP4F3 | NM_000896 | cytochrome P450, family 4, subfamily F, |
| DAZAP2 | NM_014764 | DAZ associated protein 2 |
| DMN | NM_015286 | desmuslin isoform B |
| DNAJB4 | NM_007034 | DnaJ (Hsp40) homolog, subfamily B, member 4 |
| DPYSL4 | NM_006426 | dihydropyrimidinase-like 4 |
| DTYMK | NM_012145 | deoxythymidylate kinase (thymidylate kinase) |
| DUSP3 | NM_004090 | dual specificity phosphatase 3 |
| EFNA1 | NM_004428 | ephrin A1 isoform a precursor |
| EIF2C1 | NM_012199 | eukaryotic translation initiation factor 2C, 1 |
| FBLN1 | NM_006486 | fibulin 1 isoform D |
| FEZ2 | NM_005102 | zygin 2 |
| FLJ13236 | NM_024902 | hypothetical protein FLJ13236 |
| FLJ22662 | NM_024829 | hypothetical protein LOC79887 |
| GALE | NM_000403 | UDP-galactose-4-epimerase |
| GAS2L1 | NM_152237 | growth arrest-specific 2 like 1 isoform b |
| GCLC | NM_001498 | glutamate-cysteine ligase, catalytic subunit |
| GLT25D1 | NM_024656 | glycosyltransferase 25 domain containing 1 |
| GLUL | NM_001033044 | glutamine synthetase |
| GMPR2 | NM_001002000 | guanosine monophosphate reductase 2 isoform 2 |
| GNA13 | NM_006572 | guanine nucleotide binding protein (G protein), |
| GPI | NM_000175 | glucose phosphate isomerase |
| GREB1 | NM_033090 | GREB1 protein isoform b |
| HBXIP | NM_006402 | hepatitis B virus x-interacting protein |
| HIC2 | NM_015094 | hypermethylated in cancer 2 |
| HMOX1 | NM_002133 | heme oxygenase (decyclizing) 1 |
| ID1 | NM_002165 | inhibitor of DNA binding 1 isoform a |
| IGFBP3 | NM_000598 | insulin-like growth factor binding protein 3 |
| INSIG1 | NM_005542 | insulin induced gene 1 isoform 1 |
| IPO7 | NM_006391 | importin 7 |
| KCNJ16 | NM_018658 | potassium inwardly-rectifying channel J16 |
| LAMP1 | NM_005561 | lysosomal-associated membrane protein 1 |
| LMO4 | NM_006769 | LIM domain only 4 |
| LRP8 | NM_001018054 | low density lipoprotein receptor-related protein |
| MAPKAPK2 | NM_004759 | mitogen-activated protein kinase-activated |
| MCL1 | NM_021960 | myeloid cell leukemia sequence 1 isoform 1 |
| NID1 | NM_002508 | nidogen (enactin) |
| NR2F2 | NM_021005 | nuclear receptor subfamily 2, group F, member 2 |
| ORMDL2 | NM_014182 | ORMDL2 |
| PAFAH1B2 | NM_002572 | platelet-activating factor acetylhydrolase, |
| PIGK | NM_005482 | phosphatidylinositol glycan, class K precursor |
| PODXL | NM_001018111 | podocalyxin-like precursor isoform 1 |
| POLR3D | NM_001722 | RNA polymerase III 53 kDa subunit RPC4 |
| PON2 | NM_000305 | paraoxonase 2 isoform 1 |
| PPAP2C | NM_003712 | phosphatidic acid phosphatase type 2C isoform 1 |
| PRDX6 | NM_004905 | peroxiredoxin 6 |
| PREI3 | NM_015387 | preimplantation protein 3 isoform 1 |
| PRNP | NM_000311 | prion protein preproprotein |
| PSIP1 | NM_033222 | PC4 and SFRS1 interacting protein 1 isoform 2 |
| PTER | NM_001001484 | phosphotriesterase related |
| QKI | NM_006775 | quaking homolog, KH domain RNA binding isoform |
| RAB13 | NM_002870 | RAB13, member RAS oncogene family |
| RAB32 | NM_006834 | RAB32, member RAS oncogene family |
| RAB4A | NM_004578 | RAB4A, member RAS oncogene family |
| RNF141 | NM_016422 | ring finger protein 141 |
| RRM2 | NM_001034 | ribonucleotide reductase M2 polypeptide |
| SDHA | NM_004168 | succinate dehydrogenase complex, subunit A, |
| SEC23A | NM_006364 | SEC23-related protein A |
| SLC11A2 | NM_000617 | solute carrier family 11 (proton-coupled |
| SLC30A9 | NM_006345 | solute carrier family 30 (zinc transporter), |
| SLC35A3 | NM_012243 | solute carrier family 35 |
| SORBS3 | NM_001018003 | vinexin beta (SH3-containing adaptor molecule-1) |
| STS | NM_000351 | steryl-sulfatase precursor |
| SYT1 | NM_005639 | synaptotagmin I |
| TBC1D2 | NM_018421 | TBC1 domain family, member 2 |
| TFRC | NM_003234 | transferrin receptor |
| TGFBR3 | NM_003243 | Transforming growth factor, beta receptor III |
| TPI1 | NM_000365 | triosephosphate isomerase 1 |
| TXLNA | NM_175852 | Taxilin |
| UBE2V2 | NM_003350 | ubiquitin-conjugating enzyme E2 variant 2 |
| USP46 | NM_022832 | ubiquitin specific protease 46 |
| VDAC1 | NM_003374 | voltage-dependent anion channel 1 |
| VIL2 | NM_003379 | villin 2 |
| WBSCR22 | NM_017528 | Williams Beuren syndrome chromosome region 22 |
| WDR7 | NM_015285 | Rabconnectin-3 beta isoform 1 |
| WNT7B | NM_058238 | wingless-type MMTV integration site family, |
| YIPF3 | NM_015388 | natural killer cell-specific antigen KLIP1 |

TABLE 4A
Tumor associated mRNAs altered by hsa-miR-15 having prognostic or therapeutic value for the treatment of various
malignancies.

| Gene | | Cellular | | |
|---|---|---|---|---|
| Symbol | Gene Title | Process | Cancer Type | Reference |
| AKAP12 | Akap12/SSeCKS/ | Signal | CRC, PC, LC, GC, | (Xia et al., 2001b; Wikman et al., 2002; Boultwood et al., 2004; Choi et |
| | Gravin | transduction | AML, CML | al., 2004; Mori et al., 2006) |
| CCND3 | cyclin D3 | cell cycle | EC, TC, BldC, CRC, | (Florenes et al., 2000; Ito et al., 2001; Filipits et al., 2002; Bai et al., |
| | | | LSCC, BCL, PaC, M | 2003; Pruneri et al., 2005; Tanami et al., 2005; Lopez-Beltran et al., 2006; |
| | | | | Troncone et al., 2006; Wu et al., 2006b) |
| CCNG2 | cyclin G2 | cell cycle | TC, SCCHN | (Alevizos et al., 2001; Ito et al., 2003b) |
| CDKN2C | CDK inhibitor 2C | cell cycle | HB, MB, HCC, HL, | (Iolascon et al., 1998; Kulkarni et al., 2002; Morishita et al., 2004; |
| | | | MM | Sanchez-Aguilera et al., 2004) |
| CHUK | IKK alpha | Signal | LSCC, BC | (Cao et al., 2001; Nakayama et al., 2001; Romieu-Mourez et al., 2001) |
| | | transduction | | |
| CTGF | CTGF/IGFBP-8 | cell adhesion, | BC, GB, OepC, RMS, | (Hishikawa et al., 1999; Shimo et al., 2001; Koliopanos et al., 2002; Pan |
| | | migration | CRC, PC | et al., 2002; Croci et al., 2004; Lin et al., 2005; Yang et al., 2005) |
| EPAS1 | EPAS-1 | transcription | RCC, BldC, HCC | (Xia et al., 2001a; Xia et al., 2002; Bangoura et al., 2004) |
| FGF2 | FGF-2 | Signal | BC, RCC, OC, M, | (Chandler et al., 1999) |
| | | transduction | NSCLC | |
| HSPA1B | HSP-70-1 | protein | HCC, CRC, BC | (Ciocca et al., 1993; Lazaris et al., 1995; Lazaris et al., 1997; Takashima |
| | | chaperone | | et al., 2003) |
| IGFBP3 | IGFBP-3 | Signal | BC, PC, LC, CRC | (Firth and Baxter, 2002) |
| | | transduction | | |
| IL8 | IL-8 | Signal | BC, CRC, PaC, | (Akiba et al., 2001; Sparmann and Bar-Sagi, 2004) |
| | | transduction | NSCLC, PC, HCC | |
| LCN2 | lipocalin 2/NGAL | cell adhesion | PaC, CRC, HCC, BC, | (Bartsch and Tschesche, 1995; Furutani et al., 1998; Fernandez et al., |
| | | | OC | 2005; Lee et al., 2006) |
| MCL1 | Mcl-1 | apoptosis | HCC, MM, TT, CLL, | (Krajewska et al., 1996; Kitada et al., 1998; Cho-Vega et al., 2004; Rust |
| | | | ALCL, BCL, PC | et al., 2005; Sano et al., 2005; Wuilleme-Toumi et al., 2005; Sieghart et |
| | | | | al., 2006) |
| NF1 | NF-1 | Signal | G, AC, NF, PCC, ML | (Rubin and Gutmann, 2005) |
| | | transduction | | |
| RBL1 | p107 | cell cycle | BCL, PC, CRC, TC | (Takimoto et al., 1998; Claudio et al., 2002; Wu et al., 2002; Ito et al., |
| | | | | 2003a) |
| TACC1 | TACC1 | cell cycle | BC, OC | (Cully et al., 2005; Lauffart et al., 2005) |
| TXN | thioredoxin (trx) | thioredoxin | LC, PaC, CeC, HCC | (Marks, 2006) |
| | | redox system | | |
| VAV3 | Vav3 | Signal | PC | (Dong et al., 2006) |
| | | transduction | | |
| WISP2 | WISP-2 | Signal | CRC, BC | (Pennica et al., 1998; Saxena et al., 2001) |
| | | transduction | | |
| CCND1 | cyclin D1 | cell cycle | MCL, BC, SCCHN, | (Donnellan and Chetty, 1998) |
| | | | OepC, HCC, CRC, | |
| | | | BldC, EC, OC, M, | |
| | | | AC, GB, GC, PaC | |
| EIF4E | eIF-4e | Translation | BC, CRC, NHL, NB, | (Graff and Zimmer, 2003) |
| | | | CHN, LXC, BldC, PC, | |
| | | | GC | |
| FGFR4 | FGF-R4 | Signal | TC, BC, OC, PaC | (Jaakkola et al., 1993; Shah et al., 2002; Ezzat et al., 2005) |
| | | transduction | | |
| SKP2 | SKP-2 | proteasomal | PaC, OC, BC, MFS, | (Kamata et al., 2005; Saigusa et al., 2005; Shibahara et al., 2005; |
| | | degradation | GB, EC, NSCLC, PC | Takanami, 2005; Einama et al., 2006; Huang et al., 2006; Sui et al., 2006; |
| | | | | Traub et al., 2006) |
| WNT7B | Wnt-7b | Signal | BC, BldC | (Huguet et al., 1994; Bui et al., 1998) |
| | | transduction | | |

Abbreviations:
AC, astrocytoma;
ALCL, anaplastic large cell lymphoma;
AML, acute myeloid leukemia;
BC, breast carcinoma;
BCL, B-cell lymphoma;
BldC, bladder carcinoma;
CeC, cervical carcinoma;
CHN, carcinoma of the head and neck;
CLL, chronic lymphoblastic leukemia;
CML, chronic myeloid leukemia;
CRC, colorectal carcinoma;
EC, endometrial carcinoma;
G, glioma;
GB, glioblastoma;
GC, gastric carcinoma;
HB, hepatoblastoma;
HCC, hepatocellular carcinoma;
HL, Hodgkin lymphoma;
LC, lung carcinoma;
LSCC, laryngeal squamous cell carcinoma;
LXC, larynx carcinoma;
M, melanoma;
MB, medulloblastoma;
MCL, mantle cell lymphoma;
MFS, myxofibrosarcoma;
ML, myeloid leukemia;
MM, multiple myeloma;
NB, neuroblastoma;
NF, neurofibroma;
NHL, non-Hodgkin lymphoma;
NSCLC, non-small cell lung carcinoma;
OC, ovarian carcinoma;
OepC, oesophageal carcinoma;
PaC, pancreatic carcinoma;
PC, prostate carcinoma;
PCC, pheochromocytoma;
RCC, renal cell carcinoma;
RMS, rhabdomyosarcoma;
SCCHN, squamous cell carcinoma of the head and neck;
TC, thyroid carcinoma;
TT, testicular tumor.

TABLE 4B
Tumor associated mRNAs altered by hsa-miR-26 having prognostic or therapeutic value for the treatment of various
malignancies.

| Gene | | | | |
|---|---|---|---|---|
| Symbol | Gene Title | Cellular Process | Cancer Type | Reference |
| AKAP12 | Akap-12/ | signal | CRC, PC, LC, GC, | (Xia et al., 2001; Wikman et al., 2002; Boultwood et al., 2004; Choi et al., |
| | SSeCKS/Gravin | transduction | AML, CML | 2004; Mori et al., 2006) |
| BCL2L1 | BCL-XL | apoptosis | NSCLC, SCLC, CRC, | (Manion and Hockenbery, 2003) |
| | | | BC, BldC, RCC, HL, | |
| | | | NHL, AML, ALL, | |
| | | | HCC, OC, MB, G, | |
| | | | ODG, My, OepC | |
| CTGF | CTGF/IGFBP-8 | cell adhesion, | BC, GB, OepC, RMS, | (Hishikawa et al., 1999; Shimo et al., 2001; Koliopanos et al., 2002; Pan |
| | | migration | CRC, PC | et al., 2002; Croci et a., 2004; Lin et al., 2005; Yang et al., 2005) |
| EIF4E | eIF-4e | Translation | BC, CRC, NHL, NB, | (Graff and Zimmer, 2003) |
| | | | CHN, LXC, BldC, PC, | |
| | | | GC | |
| EPHA2 | EPH receptor A2 | cell adhesion | M, NSCLC, BC, PC, | (Walker-Daniels et al., 2003; Ireton and Chen, 2005; Landen et al., 2005) |
| | | | CRC, OC | |
| FAS | Fas | Apoptosis | NSCLC, G, L, CRC, | (Moller et al., 1994; Gratas et al., 1998; Martinez-Lorenzo et al., 1998; |
| | | | OepC | Shinoura et al., 2000; Viard-Leveugle et al., 2003) |
| FZD7 | Frizzled-7 | signal | OepC, GC, HCC | (Tanaka et al., 1998; Kirikoshi et al., 2001; Merle et al., 2004) |
| | | transduction | | |
| GRB10 | GRB10 | signal | CeC | (Okino et al., 2005) |
| | | transduction | | |
| IGFBP1 | IGFBP-1 | signal | BC, CRC | (Firth and Baxter, 2002) |
| | | transduction | | |
| IGFBP3 | IGFBP-3 | signal | BC, PC, LC, CRC | (Firth and Baxter, 2002) |
| | | transduction | | |
| IL8 | IL-8 | signal | BC, CRC, PaC, | (Akiba et al., 2001; Sparmann and Bar-Sagi, 2004) |
| | | transduction | NSCLC, PC, HCC | |
| MCAM | MCAM | cell adhesion | M, AS, KS, LMS | (McGary et al., 2002) |
| MCL1 | Mcl-1 | Apoptosis | HCC, MM, TT, CLL, | (Krajewska et al., 1996; Kitada et al., 1998; Cho-Vega et al., 2004; Rust |
| | | | ALCL, BCL, PC | et al., 2005; Sano et al., 2005; Wuilleme-Toumi et al., 2005; Fleischer et |
| | | | | al., 2006; Sieghart et al., 2006) |
| MVP | major vault | multi drug | AML, CML, ALL, OC, | (Mossink et al., 2003) |
| | protein | resistance | BC, M, OS, NB, | |
| | | | NSCLC | |
| MYBL1 | A-Myb | Transcription | BL | (Golay et al., 1996) |
| NRG1 | Neuregulin 1 | signal | BC, PaC, G | (Adelaide et al., 2003; Ritch et al., 2003; Prentice et al., 2005) |
| | | transduction | | |
| PBX1 | PBX-1 | Transcription | ALL | (Aspland et al., 2001) |
| PDCD4 | Pdcd-4 | Apoptosis | G, HCC, L, RCC | (Chen et al., 2003; Jansen et al., 2004; Zhang et al., 2006; Gao et al., |
| | | | | 2007) |
| PDGFRL | PDGFR-like | signal | CRC, NSCLC, HCC, | (Fujiwara et al., 1995; Komiya et al., 1997) |
| | | transduction | PC | |
| PXN | Paxillin | cell adhesion, | SCLC, M | (Salgia et al., 1999; Hamamura et al., 2005) |
| | | motility | | |
| RARRES1 | RAR responder 1 | migration, | CRC, PC | (Zhang et al., 2004; Wu et al., 2006a) |
| | | invasion | | |
| TGFBR3 | TGF beta receptor | signal | CeC, high grade NHL, | (Venkatasubbarao et al., 2000; Bandyopadhyay et al., 2002; Woszczyk et |
| | III | transduction | CRC, BC | al., 2004; Zhang et al., 2004; Soufla et al., 2005; Wu et al., 2006a) |
| TXN | thioredoxin (trx) | thioredoxin | LC, PaC, CeC, HCC | (Marks, 2006) |
| | | redox system | | |
| VAV3 | Vav3 | signal | PC | (Dong et al., 2006) |
| | | transduction | | |

Abbreviations:
ALCL, anaplastic large cell lymphoma;
ALL, acute lymphoblastic leukemia;
AML, acute myeloid leukemia;
AS, angiosarcoma;
BC, breast carcinoma;
BCL, B-cell lymphoma;
BL, Burkitt's lymphoma;
BldC, bladder carcinoma;
CeC, cervical carcinoma;
CHN, carcinoma of the head and neck;
CLL, chronic lymphoblastic leukemia;
CML, chronic myeloid leukemia;
CRC, colorectal carcinoma;
G, glioma;
GB, glioblastoma;
GC, gastric carcinoma;
HCC, hepatocellular carcinoma;
HL, Hodgkin lymphoma;
KS, Kaposi's sarcoma;
L, leukemia;
LC, lung carcinoma;
LMS, leiomyosarcoma;
LXC, larynx carcinoma;
M, melanoma;
MB, medulloblastoma;
MM, multiple myeloma;
My, myeloma;
NB, neuroblastoma;
NHL, non-Hodgkin lymphoma;
NSCLC, non-small cell lung carcinoma;
OC, ovarian carcinoma;
ODG, oligodendrogliomas;
OepC, oesophageal carcinoma;
OS, osteosarcoma;
PaC, pancreatic carcinoma;
PC, prostate carcinoma;
RCC, renal cell carcinoma;
RMS, rhabdomyosarcoma;
SCLC, small cell lung cancer;
TT, testicular tumor.

TABLE 4C
Tumor associated mRNAs altered by hsa-miR-147 having prognostic or therapeutic value for the treatment of various
malignancies.

| Gene | | Cellular | | |
|---|---|---|---|---|
| Symbol | Gene Title | Process | Cancer Type | Reference |
| BCL6 | BCL-6 | Apoptosis | NHL | (Carbone et al., 1998; Butler et al., 2002) |
| BTG3 | B-cell | cell cycle | ALL | (Gottardo et al., 2007) |
| | translocation | | | |
| | gene 3 | | | |
| CCND1 | cyclin D1 | cell cycle | MCL, BC, SCCHN, OepC, | (Donnellan and Chetty, 1998) |
| | | | HCC, CRC, BldC, EC, OC, | |
| | | | M, | |
| | | | AC, GB, GC, PaC | |
| CCNG1 | cyclin G1 | cell cycle | OS, BC, PC | (Skotzko et al., 1995; Reimer et al., 1999) |
| EPHB2 | EPH receptor B2 | signal | PC, GC, CRC, OC, G, BC | (Huusko et al., 2004; Nakada et al., 2004; Wu et al., 2004; Jubb et al., |
| | | transduction | | 2005; Guo et al., 2006; Kokko et al., 2006; Wu et al., 2006c; Davalos et |
| | | | | al., 2007) |
| EREG | epiregulin | signal | BldC, CRC, PaC, PC | (Baba et al., 2000; Torring et al., 2000; Zhu et al., 2000; Thogersen et al., |
| | | transduction | | 2001) |
| ETS2 | ETS-2 | Transcription | CeC, PC, TC, CRC, ESCC | (Simpson et al., 1997; Sementchenko et al., 1998; de Nigris et al., 2001; |
| | | | | Ito et al., 2002; Li et al., 2003) |
| FGFR3 | FGF-R3 | signal | BldC, CRC, CeC, MM | (L'Hote and Knowles, 2005) |
| | | transduction | | |
| FGFR4 | FGF receptor-4 | signal | TC, BC, OC, PaC | (Jaakkola et al., 1993; Shah et al., 2002; Ezzat et al., 2005) |
| | | transduction | | |
| FZD7 | Frizzled-7 | signal | OepC, GC, HCC | (Tanaka et al., 1998; Kirikoshi et al., 2001; Merle et al., 2004) |
| | | transduction | | |
| ID4 | inhibitor of DNA | Transcription | BC, GC, L | (Chan et al., 2003; Yu et al., 2005; de Candia et al., 2006) |
| | binding 4 | | | |
| IGFBP1 | IGFBP-1 | signal | BC, CRC | (Firth and Baxter, 2002) |
| | | transduction | | |
| IL8 | IL-8 | signal | BC, CRC, PaC, NSCLC, | (Akiba et al., 2001; Sparmann and Bar-Sagi, 2004) |
| | | transduction | PC, HCC | |
| JAK1 | Janus kinase 1 | signal | PC | (Rossi et al., 2005) |
| | | transduction | | |
| JUN | c-Jun | Transcription | HL, HCC | (Eferl et al., 2003; Weiss and Bohmann, 2004) |
| LHFP | lipoma HMGIC | Transcription | Li | (Petit et al., 1999) |
| | fusion partner | | | |
| LIMK1 | LIM kinase 1 | cell motility, | BC, PC | (Yoshioka et al., 2003) |
| | | invasion | | |
| P8 | P8 | Transcription | BC, TC, PaC | (Ree et al., 1999; Su et al., 2001; Ito et al., 2005) |
| PDCD4 | Pdcd-4 | Apoptosis | G, HCC, L, RCC | (Chen et al., 2003; Jansen et al., 2004; Zhang et al., 2006; Gao et al., |
| | | | | 2007) |
| RARRES1 | RAR responder 1 | migration, | CRC, PC | (Zhang et al., 2004; Wu et al., 2006a) |
| | | invasion | | |
| RHOC | RhoC | cell motility, | SCCHN, OepC, CRC, M, | (Bellovin et al., 2006; Faried et al., 2006; Kleer et al., 2006; Ruth et al., |
| | | invasion | PC | 2006; Yao et al., 2006) |
| SKP2 | SKP-2 | proteasomal | PaC, OC, BC, MFS, GB, | (Kamata et al., 2005; Saigusa et al., 2005; Shibahara et al., 2005; |
| | | degradation | EC, NSCLC, PC | Takanami, 2005; Einama et al., 2006; Huang et al., 2006; Sui et al., 2006; |
| | | | | Traub et al., 2006) |
| TGFBR2 | TGF beta | signal | BC, CRC | (Markowitz, 2000; Lucke et al., 2001; Biswas et al., 2004) |
| | receptor type II | transduction | | |
| VTN | vitronectin | cell adhesion | CRC, G, OC, M, BC | (Tomasini-Johansson et al., 1994; Carreiras et al., 1996; Lee et al., 1998; |
| | | | | Carreiras et al., 1999; Uhm et al., 1999; Aaboe et al., 2003) |

Abbreviations:
AC, astrocytoma;
ALL, acute lymphoblastic leukemia;
BC, breast carcinoma;
BldC, bladder carcinoma;
CeC, cervical carcinoma;
CRC, colorectal carcinoma;
EC, endometrial carcinoma;
ESCC, esophageal squamous cell carcinoma;
G, glioma;
GB, glioblastoma;
GC, gastric carcinoma;
HCC, hepatocellular carcinoma;
HL, Hodgkin lymphoma;
L, leukemia;
Li, lipoma;
M, melanoma;
MCL, mantle cell lymphoma;
MFS, myxofibrosarcoma;
MM, multiple myeloma;
NHL, non-Hodgkin lymphoma;
NSCLC, non-small cell lung carcinoma;
OC, ovarian carcinoma;
OepC, oesophageal carcinoma;
Os, osteosarcoma;
PaC, pancreatic carcinoma;
PC, prostate carcinoma;
RCC, renal cell carcinoma;
SCCHN, squamous cell carcinoma of the head and neck;
TC, thyroid carcinoma

TABLE 4D
Tumor associated mRNAs altered by hsa-miR-188 having prognostic or therapeutic value for the treatment of various
malignancies.

| | | Cellular | | |
|---|---|---|---|---|
| Gene Symbol | Gene Title | Process | Cancer Type | Reference |
| AR | Androgen | Transcription | PC | (Feldman and Feldman, 2001) |
| | receptor | | | |
| BCL6 | BCL-6 | Apoptosis | NHL | (Carbone et al., 1998; Butler et al., 2002) |
| | | | | (Simpson et al., 1997; Sementchenko et al., 1998; de Nigris et al., |
| ETS2 | ETS-2 | Transcription | CeC, PC, TC, CRC, ESCC | 2001; Ito et al., 2002; Li et al., 2003) |
| FGF2 | FGF-2 | signal | BC, RCC, OC, M, NSCLC | (Chandler et al., 1999) |
| | | transduction | | |
| PTEN | PTEN | signal | GB, OC, BC, EC, HCC, M, LC, | (Guanti et al., 2000; Shin et al., 2001; Simpson and Parsons, 2001; |
| | | transduction | TC, NHL, PC, BldC, CRC | Vivanco and Sawyers, 2002) |
| ST13 | suppression of | signal | CRC | (Wang et al., 2005) |
| | tumorigenicity 13 | transduction | CeC, PC, SCCHN, LC, BldC, | |
| TP73L | p63 | Transcription | BC, GC | (Moll and Slade, 2004) |
| | | thioredoxin | | |
| TXN | thioredoxin (trx) | redox system | LC, PaC, CeC, HCC | (Marks, 2006) |
| VAV3 | Vav3 | signal | PC | (Dong et al., 2006) |
| | | transduction | | |
| WISP2 | WISP-2 | signal | CRC, BC | (Pennica et al., 1998; Saxena et al., 2001) |
| | | transduction | | |
| CCNA2 | cyclin A2 | cell cycle | AML | (Qian et al., 2002) |
| HDAC3 | HDAC-3 | Transcription | CRC, AC | (Liby et al., 2006; Wilson et al., 2006) |
| IGFBP3 | IGFBP-3 | signal | BC, PC, LC, CRC | (Firth and Baxter, 2002) |
| | | transduction | | |
| IL8 | IL-8 | signal | BC, CRC, PaC, NSCLC, PC, | (Akiba et al., 2001; Sparmann and Bar-Sagi, 2004) |
| | | transduction | HCC | |
| MCL1 | Mcl-1 | Apoptosis | HCC, MM, TT, CLL, ALCL, | (Krajewska et al., 1996; Kitada et al., 1998; Cho-Vega et al., 2004; |
| | | | BCL, PC | Rust et al., 2005; Sano et al., 2005; Wuilleme-Toumi et al., 2005; |
| | | | | Fleischer et al., 2006; Sieghart et al., 2006) |
| PRKCA | PKC alpha | signal | BldC, PC, EC, BC, CRC, HCC, | (Weichert et al., 2003; Jiang et al., 2004; Lahn and Sundell, 2004; |
| | | transduction | M, GC, OC | Koivunen et al., 2006) |
| RBL1 | p107 | cell cycle | BCL, PC, CRC, TC | (Takimoto et al., 1998; Claudio et al., 2002; Wu et al., 2002; Ito et |
| | | | | al, 2003a) |

Abbreviations:
AC, astrocytoma;
ALCL, anaplastic large cell lymphoma;
AML, acute myeloid leukemia;
BC, breast carcinoma;
BCL, B-cell lymphoma;
BldC, bladder carcinoma;
CeC, cervical carcinoma;
CLL, chronic lymphoblastic leukemia;
CRC, colorectal carcinoma;
BC, endometrial carcinoma;
ESCC, esophageal squamous cell carcinoma;
GB, glioblastoma;
GC, gastric carcinoma;
HCC, hepatocellular carcinoma;
LC, lung carcinoma;
M, melanoma;
MM, multiple myeloma;
NHL, non-Hodgkin lymphoma;
NSCLC, non-small cell lung carcinoma;
OC, ovarian carcinoma;
PaC, pancreatic carcinoma;
PC, prostate carcinoma;
RCC, renal cell carcinoma;
SCCHN, squamous cell carcinoma of the head and neck;
TC, thyroid carcinoma;
TT, testicular tumor

TABLE 4E
Tumor associated mRNAs altered by hsa-miR-215 having prognostic or therapeutic value for the treatment of various
malignancies.

| Gene | | Cellular | | |
|---|---|---|---|---|
| Symbol | Gene Title | Process | Cancer Type | Reference |
| ANG | angiogenin | angiogenesis | BC, OC, M, PaC, UC, | (Barton et al., 1997; Montero et al., 1998; Hartmann et al., 1999; |
| | | | CeC | Miyake et al., 1999; Shimoyama et al., 1999; Bodner-Adler et al., |
| | | | | 2001) |
| BUB1 | BUB1 | chromosomal | AML, SGT, ALL, HL, | (Cahill et al., 1998; Qian et al., 2002; Ru et al., 2002; Grabsch et al., |
| | | stability | L, CRC, GC | 2003; Shigeishi et al., 2006) |
| CCNG1 | cyclin G1 | cell cycle | OS, BC, PC | (Skotzko et al., 1995; Reimer et al., 1999) |
| EREG | epiregulin | signal | BldC, CRC, PaC, PC | (Baba et al., 2000; Torring et al., 2000; Zhu et al., 2000; Thogersen et |
| | | transduction | | al., 2001) |
| ETS2 | ETS-2 | transcription | CeC, PC, TC, CRC, | (Simpson et al., 1997; Sementchenko et al., 1998; de Nigris et al., |
| | | | ESCC | 2001; Ito et al., 2002; Li et al., 2003) |
| FAS | Fas | apoptosis | NSCLC, G, L, CRC, | (Moller et al., 1994; Gratas et al., 1998; Martinez-Lorenzo et al., 1998; |
| | | | OepC | Shinoura et al., 2000; Viard-Leveugle et al., 2003) |
| FGF2 | FGF-2 | signal | BC, RCC, OC, M, | (Chandler et al., 1999) |
| | | transduction | NSCLC | |
| FGFR1 | FGF receptor-1 | signal | L, CRC, BC, RCC, OC, | (Chandler et al., 1999) |
| | | transduction | M, NSCLC | |
| FGFR4 | FGF receptor-4 | signal | TC, BC, OC, PaC | (Jaakkola et al., 1993; Shah et al., 2002; Ezzat et al., 2005) |
| | | transduction | | |
| IGFBP3 | IGFBP-3 | signal | BC, PC, LC, CRC | (Firth and Baxter, 2002) |
| | | transduction | | |
| IL8 | IL-8 | signal | BC, CRC, PaC, | (Akiba et al., 2001; Sparmann and Bar-Sagi, 2004) |
| | | transduction | NSCLC, PC, HCC | |
| MLF1 | myeloid leukemia | cell cycle | AML | (Matsumoto et al., 2000) |
| | factor 1 | | | |
| NRG1 | neuregulin 1 | signal | BC, PaC, G | (Adelaide et al., 2003; Ritch et al., 2003; Prentice et al., 2005) |
| | | transduction | | |
| PDCD4 | Pdcd-4 | apoptosis | G, HCC, L, RCC | (Chen et al., 2003; Jansen et al., 2004; Zhang et al., 2006; Gao et al., |
| | | | | 2007) |
| PDGFRL | PDGFR-like | signal | CRC, NSCLC, HCC, | (Fujiwara et al., 1995; Komiya et al., 1997) |
| | | transduction | PC | |
| RARRES1 | RAR responder 1 | migration, | CRC, PC | (Zhang et al, 2004; Wu et al., 2006a) |
| | | invasion | | |
| RB1 | Rb | cell cycle | RB, SCLC, NSCLC | (Sherr and McCormick, 2002; Dyer and Bremner, 2005) |
| SFRP4 | secreted frizzled- | signal | MT, CLL, SCCHN | (Lee et al., 2004; Liu et al., 2006; Marsit et al., 2006) |
| | related protein 4 | transduction | | |
| TGFBR2 | TGF beta receptor | signal | BC, CRC | (Markowitz, 2000; Lucke et al., 2001; Biswas et al., 2004) |
| | type II | transduction | | |
| TGFBR3 | TGF beta receptor | signal | CeC, high grade NHL, | (Venkatasubbarao et al., 2000; Bandyopadhyay et al., 2002; Woszczyk |
| | III | transduction | CRC, BC | et al., 2004; Soufla et al., 2005) |
| TPD52 | tumor protein D52 | signal | BC, LC, PC, OC, EC, | (Boutros et al., 2004) |
| | | transduction | HCC | |
| TXN | thioredoxin (trx) | thioredoxin | LC, PaC, CeC, HCC | (Marks, 2006) |
| | | redox system | | |

Abbreviations:
ALL, acute lymphoblastic leukemia;
AML, acute myeloid leukemia;
BC, breast carcinoma;
BldC, bladder carcinoma;
CeC, cervical carcinoma;
CLL, chronic lymphoblastic leukemia;
CRC, colorectal carcinoma;
EC, endometrial carcinoma;
ESCC, esophageal squamous cell carcinoma;
G, glioma;
GC, gastric carcinoma;
HCC, hepatocellular carcinoma;
HL, Hodgkin lymphoma;
L, leukemia;
LC, lung carcinoma;
M, melanoma;
MT, mesothelioma;
NHL, non-Hodgkin lymphoma;
NSCLC, non-small cell lung carcinoma;
OC, ovarian carcinoma;
OepC, oesophageal carcinoma;
OS, osteosarcoma;
PaC, pancreatic carcinoma;
PC, prostate carcinoma;
RB, retinoblastoma;
RCC, renal cell carcinoma;
SCCHN, squamous cell carcinoma of the head and neck;
SCLC, small cell lung cancer;
SGT, salivary gland tumor;
TC, thyroid carcinoma;
UC, urothelial carcinoma;

TABLE 4F
Tumor associated mRNAs altered by hsa-miR-216 having prognostic or therapeutic value for the treatment of various malignancies.

| Gene | | Cellular | | |
|---|---|---|---|---|
| Symbol | Gene Title | Process | Cancer Type | Reference |
| BCL10 | BCL-10 | signal | MALT BCL | (Thome, 2004) |
| | | transduction | | |
| BRCA1 | BRCA-1 | chromosomal | BC, OC | (Wooster and Weber, 2003) |
| | | stability | | |
| CCNG1 | cyclin G1 | cell cycle | OS, BC, PC | (Skotzko et al., 1995; Reimer et al., 1999) |
| CDK4 | CDK-4 | cell cycle | G, GB, BC, LC, GC, EC, L, | (Malumbres and Barbacid, 2001) |
| | | | OS, OC, TT, HCC, CHN | |
| EGFR | EGFR | signal | SCCHN, G, BC, LC, OC, | (Hynes and Lane, 2005) |
| | | transduction | NSCLC | |
| FAS | Fas | Apoptosis | NSCLC, G, L, CRC, OepC | (Moller et al., 1994; Gratas et al., 1998; Martinez-Lorenzo et al., 1998; |
| | | | | Shinoura et al., 2000; Viard-Leveugle et al., 2003) |
| HDAC3 | HDAC-3 | Transcription | CRC, AC | (Liby et al., 2006; Wilson et al., 2006) |
| JUN | c-Jun | Transcription | HL, HCC | (Eferl et al., 2003; Weiss and Bohmann, 2004) |
| NF1 | NF-1 | signal | G, AC, NF, PCC, ML | (Rubin and Gutmann, 2005) |
| | | transduction | | |
| RARRES1 | RAR responder 1 | migration, | CRC, PC | (Zhang et al., 2004; Wu et al., 2006a) |
| | | invasion | | |
| ST7 | suppressor of | Unknown | PC, BC | (Hooi et al., 2006) |
| | tumorigenicity 7 | | | |
| TGFBR3 | TGF beta receptor | signal | CeC, high grade NHL, CRC, | (Venkatasubbarao et al., 2000; Bandyopadhyay et al., 2002; Woszczyk |
| | III | transduction | BC | et al., 2004; Soufla et al., 2005) |
| VAV3 | Vav3 | signal | PC | (Dong et al., 2006) |
| | | transduction | | |
| WISP2 | WISP-2 | signal | CRC, BC | (Pennica et al., 1998; Saxena et al., 2001) |
| | | transduction | | |

Abbreviations:
AC, astrocytoma;
BC, breast carcinoma;
CeC, cervical carcinoma;
CHN, carcinoma of the head and neck;
CRC, colorectal carcinoma;
EC, endometrial carcinoma;
G, glioma;
GB, glioblastoma;
GC, gastric carcinoma;
HCC, hepatocellular carcinoma;
HL, Hodgkin lymphoma;
L, leukemia;
LC, lung carcinoma;
MALT BCL, mucosa-associated lymphoid tissue B-cell lymphoma;
ML, myeloid leukemia;
NF, neurofibroma;
NHL, non-Hodgkin lymphoma;
NSCLC, non-small cell lung carcinoma;
OC, ovarian carcinoma;
OepC, oesophageal carcinoma;
OS, osteosarcoma;
PC, prostate carcinoma;
PCC, pheochromocytoma;
SCCHN, squamous cell carcinoma of the head and neck;
TT, testicular tumor

TABLE 4G
Tumor associated mRNAs altered by hsa-miR-331 having prognostic or therapeutic value for the treatment of various
malignancies.

| | | Cellular | | |
|---|---|---|---|---|
| Gene Symbol | Gene Title | Process | Cancer Type | Reference |
| AR | Androgen | transcription | PC | (Feldman and Feldman, 2001) |
| AREG | receptor | signal | HCC, NSCLC, MM, | (Kitadai et al., 1993; Ebert et al., 1994; Solic and Davies, 1997; |
| | amphiregulin | transduction | PC, OC, CRC, PaC, GC | D'Antonio et al., 2002; Bostwick et al., 2004; Ishikawa et al., 2005; |
| | | | | Mahtouk et al., 2005; Castillo et al., 2006) |
| CCNG1 | cyclin G1 | cell cycle | OS, BC, PC | (Skotzko et al., 1995; Reimer et al., 1999) |
| EREG | epiregulin | signal | BldC, CRC, PaC, PC | (Baba et al., 2000; Torring et al., 2000; Zhu et al., 2000; Thogersen et |
| | | transduction | | al., 2001) |
| FGFR1 | FGF receptor-1 | signal | L, CRC, BC, RCC, OC, | (Chandler et al., 1999) |
| | | transduction | M, NSCLC | |
| IGFBP3 | IGFBP-3 | signal | BC, PC, LC, CRC | (Firth and Baxter, 2002) |
| | | transduction | | |
| IL8 | IL-8 | signal | BC, CRC, PaC, | (Akiba et al., 2001; Sparmann and Bar-Sagi, 2004) |
| | | transduction | NSCLC, PC, HCC | |
| PDCD4 | Pdcd-4 | Apoptosis | G, HCC, L, RCC | (Chen et al., 2003; Jansen et al., 2004; Zhang et al., 2006; Gao et al., |
| | | | | 2007) |
| PDPK1 | PDK-1 | signal | BC | (Zeng et al., 2002; Tseng et al., 2006; Xie et al., 2006) |
| | | transduction | | |
| PHLPP | PHLPP | signal | CRC, GB | (Matsumoto et al., 2000) |
| | | transduction | | |
| PXN | paxillin | cell adhesion, | SCLC, M | (Salgia et al., 1999; Hamamura et al., 2005) |
| | | motility | | |
| SKP2 | SKP-2 | proteasomal | PaC, OC, EC, MFS, | (Kamata et al., 2005; Saigusa et al., 2005; Shibahara et al., 2005; |
| | | degradation | GB, EC, NSCLC, PC | Takanami, 2005; Einama et al., 2006; Huang et al., 2006; Sui et al., |
| | | | | 2006; Traub et al., 2006) |
| TGFB2 | TGF beta-2 | signal | PaC, CRC, BC, M | (Krasagakis et al., 1998; Jonson et al., 2001; Nakagawa et al., 2004; |
| | | transduction | | Beisner et al., 2006) |
| TXN | thioredoxin (trx) | thioredoxin | LC, PaC, CeC, HCC | (Marks, 2006) |
| | | redox system | | |
| WNT7B | Wnt-7b | signal | BC, BldC | (Huguet et al., 1994; Bui et al., 1998) |
| | | transduction | | |
| BCL2L1 | BCL-XL | apoptosis | NSCLC, SCLC, CRC, | (Manion and Hockenbery, 2003) |
| | | | BC, BldC, RCC, HL, | |
| | | | NHL, AML, ALL, | |
| | | | HCC, OC, MB, G, | |
| | | | ODG, My, OepC | |
| LMO4 | Lmo-4 | transcription | BC, SCCHN, SCLC | (Visvader et al., 2001; Mizunuma et al., 2003; Taniwaki et al., 2006) |

Abbreviations:
ALL, acute lymphoblastic leukemia;
AML, acute myeloid leukemia;
BC, breast carcinoma;
BldC, bladder carcinoma;
CeC, cervical carcinoma;
CRC, colorectal carcinoma;
EC, endometrial carcinoma;
G, glioma;
GB, glioblastoma;
GC, gastric carcinoma;
HCC, hepatocellular carcinoma;
HL, Hodgkin lymphoma;
L, leukemia;
LC, lung carcinoma;
LSCC, laryngeal squamous cell carcinoma;
M, melanoma;
MB, medulloblastoma;
MFS, myxofibrosarcoma;
MM, multiple myeloma;
My, myeloma;
NHL, non-Hodgkin lymphoma;
NSCLC, non-small cell lung carcinoma;
OC, ovarian carcinoma;
ODG, oligodendrogliomas;
OepC, oesophageal carcinoma;
OS, osteosarcoma;
PaC, pancreatic carcinoma;
PC, prostate carcinoma;
RCC, renal cell carcinoma;
SCCHN, squamous cell carcinoma of the head and neck;
SCLC, small cell lung cancer

TABLE 4H
Tumor associated mRNAs altered by mmu-miR-292-3p having prognostic or therapeutic value for the treatment of various
malignancies.

| | | Cellular | | |
|---|---|---|---|---|
| Gene Symbol | Gene Title | Process | Cancer Type | Reference |
| AR | Androgen | Transcription | PC | (Feldman and Feldman, 2001) |
| | receptor | | | |
| CCND3 | cyclin D3 | cell cycle | EC, TC, BldC, CRC, LSCC, | (Florenes et al., 2000; Ito et al., 2001; Filipits et al., 2002; Bai et al., |
| | | | BCL, PaC, M | 2003; Pruneri et al., 2005; Tanami et al., 2005; Lopez-Beltran et al., |
| | | | | 2006; Troncone et al., 2006; Wu et al., 2006b) |
| CCNG1 | cyclin G1 | cell cycle | OS, BC, PC | (Skotzko et al., 1995; Reimer et al., 1999) |
| CEBPD | C/EBP delta | Transcription | PC | (Yang et al., 2001) |
| CSF1 | CSF-1 | signal | HCC, LC | (Budhu et al., 2006; Uemura et al., 2006) |
| | | transduction | | |
| FAS | Fas | Apoptosis | NSCLC, G, L, CRC, OepC | (Moller et al., 1994; Gratas et al., 1998; Martinez-Lorenzo et al., 1998; |
| | | | | Shinoura et al., 2000; Viard-Leveugle et al., 2003) |
| FGFBP1 | FGF-BP | signal | SCCHN, BC, CRC, PC, PaC | (Abuharbeid et al., 2006; Tassi et al., 2006) |
| | | transduction | | |
| HSPCA | Hsp90 1alpha | Invasion | FS | (Eustace et al., 2004) |
| IGFBP3 | IGFBP-3 | signal | BC, PC, LC, CRC | (Firth and Baxter, 2002) |
| | | transduction | | |
| IL8 | IL-8 | signal | BC, CRC, PaC, NSCLC, PC, | (Akiba et al., 2001; Sparmann and Bar-Sagi, 2004) |
| | | transduction | HCC | |
| LMO4 | Lmo-4 | Transcription | BC, SCCHN, SCLC | (Visvader et al., 2001; Mizunuma et al., 2003; Taniwaki et al., 2006) |
| MCAM | MCAM | cell adhesion | M, AS, KS, LMS | (McGary et al., 2002) |
| MCL1 | Mcl-1 | Apoptosis | HCC, MM, TT, CLL, ALCL, | (Krajewska et al., 1996; Kitada et al., 1998; Cho-Vega et al., 2004; Rust |
| | | | BCL, PC | et al., 2005; Sano et al., 2005; Wuilleme-Toumi et al., 2005; Fleischer |
| | | | | et al., 2006; Sieghart et al., 2006) |
| MDM2 | Mdm2 | proteasomal | AC, GB, BC, CeC, OepC, L, | (Momand et al., 1998) |
| | | degradation | HB, NSCLC, NPC, NB, OS, | |
| | | | OC, EWS, Li, LS, Schw, TT, | |
| | | | UC, WT, RMS | |
| MVP | major vault | multi drug | AML, CML, ALL, OC, BC, | (Mossink et al., 2003) |
| | protein | resistance | M, OS, NB, NSCLC | |
| PDCD4 | Pdcd-4 | Apoptosis | G, HCC, L, RCC | (Chen et al., 2003; Jansen et al., 2004; Zhang et al., 2006; Gao et al., |
| | | | | 2007) |
| PDGFRL | PDGFR-like | signal | CRC, NSCLC, HCC, PC | (Fujiwara et al., 1995; Komiya et al., 1997) |
| | | transduction | | |
| PTEN | PTEN | signal | GB, OC, BC, EC, HCC, M, | (Guanti et al., 2000; Shin et al., 2001; Simpson and Parsons, 2001; |
| | | transduction | LC, TC, NHL, PC, BldC, | Vivanco and Sawyers, 2002) |
| | | | CRC | |
| SKP2 | SKP-2 | proteasomal | PaC, OC, BC, MFS, GB, EC, | (Kamata et al., 2005; Saigusa et al., 2005; Shibahara et al., 2005; |
| | | degradation | NSCLC, PC | Takanami, 2005; Einama et al., 2006; Huang et al., 2006; Sui et al., |
| | | | | 2006; Traub et al., 2006) |
| TGFBR3 | TGF beta | signal | CeC, high grade NHL, CRC, | (Venkatasubbarao et al., 2000; Bandyopadhyay et al., 2002; Woszczyk |
| | receptor III | transduction | BC | et al., 2004; Soufla et al., 2005) |
| TNFRSF10B | TRAIL-R2 | Apoptosis | NSCLC, SCCHN, GC, BC, | (Adams et al., 2005) |
| | | | NHL | |
| TPD52L1 | Tumor | cell cycle | BC | (Boutros and Byrne, 2005) |
| | protein D52- | | | |
| | like 1 | | | |
| TXN | thioredoxin | thioredoxin | LC, PaC, CeC, HCC | (Marks, 2006) |
| | (trx) | redox system | | |
| WEE1 | Wee-1 kinase | cell cycle | NSCLC | (Yoshida et al., 2004) |
| WNT7B | Wnt-7b | signal | BC, BldC | (Huguet et al., 1994; Bui et al., 1998) |
| | | transduction | | |

Abbreviations:
AC, astrocytoma;
ALCL, anaplastic large cell lymphoma;
ALL, acute lymphoblastic leukemia;
AML, acute myeloid leukemia;
AS, angiosarcoma;
BC, breast carcinoma;
BCL, B-cell lymphoma;
BldC, bladder carcinoma;
CeC, cervical carcinoma;
CLL, chronic lymphoblastic leukemia;
CML, chronic myeloid leukemia;
CRC, colorectal carcinoma;
EC, endometrial carcinoma;
EWS, Ewing's sarcoma;
FS, fibrosarcoma;
G, glioma;
GB, glioblastoma;
GC, gastric carcinoma;
HB, hepatoblastoma;
HCC, hepatocellular carcinoma;
KS, Kaposi's sarcoma;
L, leukemia;
LC, lung carcinoma;
Li, lipoma;
LMS, leiomyosarcoma;
LS, liposarcoma;
LSCC, laryngeal squamous cell carcinoma;
M, melanoma;
MFS, myxofibrosarcoma;
MM, multiple myeloma;
NB, neuroblastoma;
NHL, non-Hodgkin lymphoma;
NPC, nasopharyngeal carcinoma;
NSCLC, non-small cell lung carcinoma;
OC, ovarian carcinoma;
OepC, oesophageal carcinoma;
OS, osteosarcoma;
PaC, pancreatic carcinoma;
PC, prostate carcinoma;
RCC, renal cell carcinoma;
RMS, rhabdomyosarcoma;
SCCHN, squamous cell carcinoma of the head and neck;
Schw, schwannoma;
SCLC, small cell lung cancer;
TC, thyroid carcinoma;
TT, testicular tumor;
UC, urothelial carcinoma;
WT, Wilm's tumor

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in Table 1, 3, and/or 4.

It is contemplated that any method or composition described herein can be implemented with respect to any other method or composition described herein and that different embodiments may be combined. Certain embodiments of the invention include determining expression of one or more marker, gene, or nucleic acid representative thereof, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Protein is typically assayed by immunoblotting, chromatography, or mass spectrometry or other methods known to those of ordinary skill in the art.

The present invention also concerns kits containing compositions of the invention or compositions to implement methods of the invention. In some embodiments, kits can be used to evaluate one or more marker molecules, and/or express one or more miRNA. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on a miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in Table 1, 3, and/or 4.

It is contemplated that any method or composition described herein can be implemented with respect to any other method or composition described herein and that different embodiments may be combined. It is specifically contemplated that any methods and compositions discussed herein with respect to miRNA molecules, miRNA, genes and nucleic acids representative of genes may be implemented with respect to synthetic nucleic acids. In some embodiments the synthetic nucleic acid is exposed to the proper conditions to allow it to become a processed or mature nucleic acid, such as a miRNA under physiological circumstances. The claims originally filed are contemplated to cover claims that are multiply dependent on any filed claim or combination of filed claims.

Also, any embodiment of the invention involving specific genes (including representative fragments there of), mRNA, or miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA.

It will be further understood that shorthand notations are employed such that a generic description of a gene or marker, or of a miRNA refers to any of its gene family members or representative fragments, unless otherwise indicated. It is understood by those of skill in the art that a “gene family” refers to a group of genes having similar coding sequence or miRNA coding sequence. Typically, miRNA members of a gene family are identified by a number following the initial designation. For example, miR-16-1 and miR-16-2 are members of the miR-16 gene family and “mir-7” refers to miR-7-1, miR-7-2 and miR-7-3. Moreover, unless otherwise indicated, a shorthand notation refers to related miRNAs (distinguished by a letter). Exceptions to these shorthand notations will be otherwise identified.

Other embodiments of the invention are discussed throughout this application. Any embodiment discussed with respect to one aspect of the invention applies to other aspects of the invention as well and vice versa. The embodiments in the Example and Detailed Description section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Percent (%) proliferation of hsa-miR-147 treated human lung cancer cells relative to cells treated with negative control miRNA (100%). Abbreviations: miR-147, hsa-miR-147; siEg5, siRNA against the motor protein kinesin 11 (Eg5); Etopo, etoposide; NC, negative control miRNA. Standard deviations are indicated in the graph.

FIG. 2 Percent (%) proliferation of hsa-miR-147 treated luciferase-expressing human lung cancer cells relative to cells treated with negative control miRNA (100%). Abbreviations: miR-147, hsa-miR-147; siEg5, siRNA against the motor protein kinesin 11 (Eg5); Etopo, etoposide; NC, negative control miRNA. Standard deviations are indicated in the graph.

FIG. 3 Dose dependent inhibition of A549 and H1299 human lung cancer cell lines by hsa-miR-147 using Alamar Blue proliferation assays. Cell proliferation is reported as % proliferation relative to % proliferation of mock-transfected cells (0 μM=100% proliferation). Standard deviations are indicated in the graph. Abbreviations: miR-147, hsa-miR-147; NC, negative control miRNA

FIG. 4 Percent (%) proliferation of H460 lung cancer cells following administration of various combinations of microRNAs. A positive sign under each bar in the graph indicates that the miRNA was present in the administered combination. Standard deviations are shown in the graph. Abbreviations: miR-124a, hsa-miR-124a; miR-126, hsa-miR-126; miR-147, hsa-miR-147; let-7b, hsa-let-7b; let-7c, hsa-let-7c; let-7g, hsa-let-7g; Etopo, etoposide; NC, negative control miRNA.

FIG. 5 Average tumor volumes in groups of five (n=5) mice carrying human A549 lung cancer xenografts treated with hsa-miR-147 (black diamonds) or with a negative control miRNA (NC, white squares). Standard deviations are shown in the graph. The p value, indicating statistical significance, is shown for values obtained on day 20 (p=0.01357). Abbreviation: miR-147, hsa-miR-147; NC, negative control miRNA.

FIG. 6 Long-term effects of hsa-miR-147 on cultured human H226 lung cancer cells. Equal numbers of cells were electroporated with 1.6 μM hsa-miR-147 (white squares) or negative control miRNA (NC, black diamonds), seeded and propagated in regular growth medium. When the control cells reached confluence (days 6, 17 and 25), cells were harvested, counted and electroporated again with the respective miRNAs. The population doubling and cumulative cell counts was calculated and plotted on a linear scale. Arrows represent electroporation days. Experiments were carried out in triplicates. Standard deviations are shown in the graph. Abbreviation: miR-147, hsa-miR-147; NC, negative control miRNA.

FIG. 7 Average tumor volumes in groups of six (n=6) mice carrying human H460 lung cancer xenografts. Palpable tumors were treated with hsa-miR-147 (white squares) or with a negative control miRNA (NC, black diamonds) on days 11, 14, and 17 (arrows). Standard deviations are shown in the graph. Data points with p values<0.01 and <0.05 are indicated by an asterisk or circles, respectively. Abbreviation: miR-147, hsa-miR-147; NC, negative control miRNA.

FIG. 8 Percent (%) proliferation of hsa-miR-147 treated human prostate cancer cells relative to cells treated with negative control miRNA (100%). Abbreviations: miR-147, hsa-miR-147; siEg5, siRNA against the motor protein kinesin 11 (Eg5); Etopo, etoposide; NC, negative control miRNA. Standard deviations are indicated in the graph.

FIG. 9 Long-term effects of hsa-miR-147 on cultured human PC3 and Du145 prostate cancer cells. Equal numbers of cells were electroporated with 1.6 μM hsa-miR-147 (white squares) or negative control miRNA (NC, black diamonds), seeded and propagated in regular growth medium. When the control cells reached confluence (days 7 and 14), cells were harvested, counted and electroporated again with the respective miRNAs. The population doubling and cumulative cell counts was calculated and plotted on a linear scale. Arrows represent electroporation days. Experiments with PC3 and Du145 cells were carried out in triplicates. Standard deviations are shown in the graphs. Abbreviation: miR-147, hsa-miR-147; NC, negative control miRNA.

FIG. 10 Proliferation effects of hsa-miR-15a on cultured human prostate cancer cells. Equal numbers of cells were electroporated with 1.6 μM hsa-miR-15a (white squares) or negative control miRNA (NC, black diamonds), seeded and propagated in regular growth medium. When the control cells reached confluence (days 7 and 14), cells were harvested, counted and electroporated again with the respective miRNAs. The population doubling and cumulative cell counts was calculated and plotted on a linear scale. Arrows represent electroporation days. Experiments were carried out in triplicates. Standard deviations are shown in the graphs. Abbreviation: miR-15a, hsa-miR-15a; NC, negative control miRNA

### DETAILED DESCRIPTION OF THE INVENTION

The present invention is directed to compositions and methods relating to the identification and characterization of genes and biological pathways related to these genes as represented by the expression of the identified genes, as well as use of miRNAs related to such, for therapeutic, prognostic, and diagnostic applications, particularly those methods and compositions related to assessing and/or identifying pathological conditions directly or indirectly related to miR-15, miR-26, miR-31, miR-145, miR-147, miR-188, miR-215, miR-216, miR-331, or mmu-miR-292-3p expression or the aberrant expression thereof.

In certain aspects, the invention is directed to methods for the assessment, analysis, and/or therapy of a cell or subject where certain genes have a reduced or increased expression (relative to normal) as a result of an increased or decreased expression of any one or a combination of miR-15, miR-26, miR-31, miR-145, miR-147, miR-188, miR-215, miR-216, miR-331, or mmu-miR-292-3p family members (including, but not limited to SEQ ID NO: 1 to SEQ ID NO:391) and/or genes with an increased expression (relative to normal) as a result of decreased expression thereof. The expression profile and/or response to miR-15, miR-26, miR-31, miR-145, miR-147, miR-188, miR-215, miR-216, miR-331, or mmu-miR-292-3p expression or inhibition may be indicative of a disease or pathological condition, e.g., cancer.

Prognostic assays featuring any one or combination of the miRNAs listed or the markers listed (including nucleic acids representative thereof) could be used in assessment of a patient to determine what if any treatment regimen is justified. As with the diagnostic assays mentioned above, the absolute values that define low expression will depend on the platform used to measure the miRNA(s). The same methods described for the diagnostic assays could be used for prognostic assays.

### I. THERAPEUTIC METHODS

Embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors of miRNA in a cell. The term “short” refers to a length of a single polynucleotide that is 15, 16, 17, 18, 19, 20, 21, 22, 23, 24, 25, 50, 100, or 150 nucleotides or fewer, including all integers or ranges derivable there between. The nucleic acid molecules are typically synthetic. The term “synthetic” refers to a nucleic acid molecule that is not produced naturally in a cell. In certain aspects the chemical structure deviates from a naturally-occurring nucleic acid molecule, such as an endogenous precursor miRNA or miRNA molecule or complement thereof. While in some embodiments, nucleic acids of the invention do not have an entire sequence that is identical or complementary to a sequence of a naturally-occurring nucleic acid, such molecules may encompass all or part of a naturally-occurring sequence or a complement thereof. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA or an inhibitor thereof. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together. In certain aspects, synthetic miRNA of the invention are RNA or RNA analogs. miRNA inhibitors may be DNA or RNA, or analogs thereof. miRNA and miRNA inhibitors of the invention are collectively referred to as “synthetic nucleic acids.”

In some embodiments, there is a miRNA or a synthetic miRNA having a length of between 17 and 130 residues. The present invention concerns miRNA or synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range there between.

In certain embodiments, synthetic miRNA have (a) a “miRNA region” whose sequence or binding region from 5′ to 3′ is identical or complementary to all or a segment of a mature miRNA sequence, and (b) a “complementary region” whose sequence from 5′ to 3′ is between 60% and 100% complementary to the miRNA sequence in (a). In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence or a complement thereof. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA or complement thereof.

The term “complementary region” or “complement” refers to a region of a nucleic acid or mimetic that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the miRNA sequence that is complementary to the sequence of a mature miRNA as the sequence for a miRNA inhibitor. Moreover, that portion of the nucleic acid sequence can be altered so that it is still comprises the appropriate percentage of complementarity to the sequence of a mature miRNA.

In some embodiments, of the invention, a synthetic miRNA or inhibitor contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an aminohexyl phosphate group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification, a 2′F modification, a 2′H modification, a 2′amino modification, a 4′thioribose modification, or a phosphorothioate modification on the carboxy group linked to the carbon at position 6′. In further embodiments, there are one or more sugar modifications in the first or last 2 to 4 residues of the complementary region or the first or last 4 to 6 residues of the complementary region. This design element can also be used with a miRNA inhibitor. Thus, an miRNA inhibitor can have this design element and/or a replacement group on the nucleotide at the 5′ terminus, as discussed above.

In other embodiments of the invention, there is a synthetic miRNA or inhibitor in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a miRNA inhibitor (which may be described generally herein as an miRNA, so that a description of miRNA, where appropriate, also will refer to a miRNA inhibitor); or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or the miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA or induced or inhibited miRNA function. It is contemplated, however, that the miRNA molecule introduced into a cell is not a mature miRNA but is capable of becoming or functioning as a mature miRNA under the appropriate physiological conditions. In cases in which a particular corresponding miRNA is being inhibited by a miRNA inhibitor, the particular miRNA will be referred to as the “targeted miRNA.” It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell. The inventors contemplate that a combination of miRNA may act at one or more points in cellular pathways of cells with aberrant phenotypes and that such combination may have increased efficacy on the target cell while not adversely effecting normal cells. Thus, a combination of miRNA may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a synthetic nucleic acid that is provided to a cell or organism is an “effective amount,” which refers to an amount needed (or a sufficient amount) to achieve a desired goal, such as inducing a particular cellular characteristic(s). Certain embodiments of the methods include providing or introducing to a cell a nucleic acid molecule corresponding to a mature miRNA in the cell in an amount effective to achieve a desired physiological result.

Moreover, methods can involve providing synthetic or nonsynthetic miRNA molecules. It is contemplated that in these embodiments, that the methods may or may not be limited to providing only one or more synthetic miRNA molecules or only one or more nonsynthetic miRNA molecules. Thus, in certain embodiments, methods may involve providing both synthetic and nonsynthetic miRNA molecules. In this situation, a cell or cells are most likely provided a synthetic miRNA molecule corresponding to a particular miRNA and a nonsynthetic miRNA molecule corresponding to a different miRNA. Furthermore, any method articulated using a list of miRNAs using Markush group language may be articulated without the Markush group language and a disjunctive article (i.e., or) instead, and vice versa.

In some embodiments, there is a method for reducing or inhibiting cell proliferation in a cell comprising introducing into or providing to the cell an effective amount of (i) an miRNA inhibitor molecule or (ii) a synthetic or nonsynthetic miRNA molecule that corresponds to a miRNA sequence. In certain embodiments the methods involves introducing into the cell an effective amount of (i) a miRNA inhibitor molecule having a 5′ to 3′ sequence that is at least 90% complementary to the 5′ to 3′ sequence of one or more mature miRNA.

Certain embodiments of the invention include methods of treating a pathologic condition, in particular cancer, e.g., lung or liver cancer. In one aspect, the method comprises contacting a target cell with one or more nucleic acid, synthetic miRNA, or miRNA comprising at least one nucleic acid segment having all or a portion of a miRNA sequence. The segment may be 5, 6, 7, 8, 9, 10, 11, 12, 13, 14, 15, 16, 17, 18, 19, 20, 21, 22, 23, 24, 25, 30 or more nucleotides or nucleotide analog, including all integers there between. An aspect of the invention includes the modulation of gene expression, miRNA expression or function or mRNA expression or function within a target cell, such as a cancer cell.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA or gene sequence. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may affect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

It will be understood in methods of the invention that a cell or other biological matter such as an organism (including patients) can be provided a miRNA or miRNA molecule corresponding to a particular miRNA by administering to the cell or organism a nucleic acid molecule that functions as the corresponding miRNA once inside the cell. The form of the molecule provided to the cell may not be the form that acts a miRNA once inside the cell. Thus, it is contemplated that in some embodiments, a synthetic miRNA or a nonsynthetic miRNA is provided such that it becomes processed into a mature and active miRNA once it has access to the cell's miRNA processing machinery. In certain embodiments, it is specifically contemplated that the miRNA molecule provided is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understand that the term “providing” an agent is used to include “administering” the agent to a patient.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In some embodiments, the miRNA targeted to be modulated is a miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA or its targets.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability). Consequently, in some methods of the invention there is a step of identifying a patient in need of treatment that can be provided by the miRNA modulator(s). It is contemplated that an effective amount of a miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the miRNA compositions may be provided as part of a therapy to a patient, in conjunction with traditional therapies or preventative agents. Moreover, it is contemplated that any method discussed in the context of therapy may be applied preventatively, particularly in a patient identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, 5-fluorouracil, alemtuzumab, amrubicin, bevacizumab, bleomycin, bortezomib, busulfan, camptothecin, capecitabine, carboplatin, cetuximab, chlorambucil, cisplatin (CDDP), COX-2 inhibitors (e.g., celecoxib), cyclophosphamide, cytarabine, dactinomycin, dasatinib, daunorubicin, dexamethasone, docetaxel, doxorubicin (adriamycin), EGFR inhibitors (gefitinib and cetuximab), erlotinib, estrogen receptor binding agents, etoposide (VP16), everolimus, farnesyl-protein transferase inhibitors, gefitinib, gemcitabine, gemtuzumab, ibritumomab, ifosfamide, imatinib mesylate, larotaxel, lapatinib, lonafarnib, mechlorethamine, melphalan, methotrexate, mitomycin, navelbine, nitrosurea, nocodazole, oxaliplatin, paclitaxel, plicomycin, procarbazine, raloxifene, rituximab, sirolimus, sorafenib, sunitinib, tamoxifen, taxol, taxotere, temsirolimus, tipifamib, tositumomab, transplatinum, trastuzumab, vinblastin, vincristin, or vinorelbine or any analog or derivative variant of the foregoing.

Generally, inhibitors of miRNAs can be given to decrease the activity of an endogenous miRNA. For example, inhibitors of miRNA molecules that increase cell proliferation can be provided to cells to decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, activating or inhibiting a kinase (e.g., Erk), activating/inducing or inhibiting hTert, inhibit stimulation of growth promoting pathway (e.g., Stat 3 signaling), reduce or increase viable cell number, and increase or decrease number of cells at a particular phase of the cell cycle. Methods of the invention are generally contemplated to include providing or introducing one or more different nucleic acid molecules corresponding to one or more different miRNA molecules. It is contemplated that the following, at least the following, or at most the following number of different nucleic acid or miRNA molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. This also applies to the number of different miRNA molecules that can be provided or introduced into a cell.

### II. PHARMACEUTICAL FORMULATIONS AND DELIVERY

Methods of the present invention include the delivery of an effective amount of a miRNA or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct injection, intratumoral injection, or injection into tumor vasculature is specifically contemplated for discrete, solid, accessible tumors, or other accessible target areas. Local, regional, or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as a single dose comprise about 0.1 to about 0.5 ml volumes. Compositions of the invention may be administered in multiple injections to a tumor or a targeted site. In certain aspects, injections may be spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a miRNA or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catherization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well and often depend on tumor type, tumor location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the tumor or affected area being treated may not, at least initially, be respectable. Treatments with compositions of the invention may increase the respectability of the tumor due to shrinkage at the margins or by elimination of certain particularly invasive portions. Following treatments, resection may be possible. Additional treatments subsequent to resection may serve to eliminate microscopic residual disease at the tumor or targeted site.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of μg or mg of miRNA or miRNA mimetic. Alternatively, the amount specified may be the amount administered as the average daily, average weekly, or average monthly dose.

miRNA can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery of a miRNA or an expression construct encoding such or combinations thereof is via systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158, 5,641,515, and 5,399,363 (each specifically incorporated herein by reference in its entirety).

Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a tumor suppressor protein or a nucleic acid encoding the same is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

C. Combination Treatments

In certain embodiments, the compositions and methods of the present invention involve a miRNA, or expression construct encoding such. These miRNA compositions can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the miRNA or second therapy at the same or different time. This may be achieved by contacting the cell with one or more compositions or pharmacological formulation that includes or more of the agents, or by contacting the cell with two or more distinct compositions or formulations, wherein one composition provides (1) miRNA; and/or (2) a second therapy. A second composition or method may be administered that includes a chemotherapy, radiotherapy, surgical therapy, immunotherapy or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

b. Antimetabolites

Antimetabolites disrupt DNA and RNA synthesis. Unlike alkylating agents, they specifically influence the cell cycle during S phase. They have been used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

d. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

e. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, normal cells are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Table 5 is a non-limiting list of several known anti-cancer immunotherapeutic agents and their targets. It is contemplated that one or more of these therapies may be employed with the miRNA therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

TABLE 5
Examples of known anti-cancer immunotherapeutic agents
and their targets

| Generic Name | Target |
|---|---|
| Cetuximab | EGFR |
| Panitumumab | EGFR |
| Trastuzumab | erbB2 receptor |
| Bevacizumab | VEGF |
| Alemtuzumab | CD52 |
| Gemtuzumab ozogamicin | CD33 |
| Rituximab | CD20 |
| Tositumomab | CD20 |
| Matuzumab | EGFR |
| Ibritumomab tiuxetan | CD20 |
| Tositumomab | CD20 |
| HuPAM4 | MUC1 |
| MORAb-009 | Mesothelin |
| G250 | carbonic anhydrase IX |
| mAb 8H9 | 8H9 antigen |
| M195 | CD33 |
| Ipilimumab | CTLA4 |
| HuLuc63 | CS1 |
| Alemtuzumab | CD53 |
| Epratuzumab | CD22 |
| BC8 | CD45 |
| HuJ591 | Prostate specific membrane antigen |
| hA20 | CD20 |
| Lexatumumab | TRAIL receptor-2 |
| Pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| Volociximab | anti-α5β1 integrin |
| GC1008 | TGFβ |
| HCD122 | CD40 |
| Siplizumab | CD2 |
| MORAb-003 | Folate receptor alpha |
| CNTO 328 | IL-6 |
| MDX-060 | CD30 |
| Ofatumumab | CD20 |
| SGN-33 | CD33 |

4. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### III. miRNA MOLECULES

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene or its gene product. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

A. Array Preparation

Certain embodiments of the present invention concerns the preparation and use of mRNA or nucleic acid arrays, miRNA or nucleic acid arrays, and/or miRNA or nucleic acid probe arrays, which are macroarrays or microarrays of nucleic acid molecules (probes) that are fully or nearly complementary (over the length of the prove) or identical (over the length of the prove) to a plurality of nucleic acid, mRNA or miRNA molecules, precursor miRNA molecules, or nucleic acids derived from the various genes and gene pathways modulated by miR-15, miR-26, miR-31, miR-145, miR-147, miR-188, miR-215, miR-216, miR-331, or mmu-miR-292-3p miRNAs and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of marker RNA and/or miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect miRNA, or genes or nucleic acid representative of genes; consequently, methods and compositions may be used with a variety of different types of nucleic acid arrays.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to mRNA and/or miRNA targets in one or more different organisms or cell types. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

B. Sample Preparation

It is contemplated that the RNA and/or miRNA of a wide variety of samples can be analyzed using the arrays, index of probes, or array technology of the invention. While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells, particularly cancer or hyperproliferative cells. In certain embodiments, samples may be, but are not limited to, biopsy, or cells purified or enriched to some extent from a biopsy or other bodily fluids or tissues. Alternatively, the sample may not be a biological sample, but be a chemical mixture, such as a cell-free reaction mixture (which may contain one or more biological enzymes).

C. Hybridization

After an array or a set of probes is prepared and/or the nucleic acid in the sample or probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

D. Differential Expression Analyses

Arrays of the invention can be used to detect differences between two samples. Specifically contemplated applications include identifying and/or quantifying differences between miRNA or gene expression from a sample that is normal and from a sample that is not normal, between a disease or condition and a cell not exhibiting such a disease or condition, or between two differently treated samples. Also, miRNA or gene expression may be compared between a sample believed to be susceptible to a particular disease or condition and one believed to be not susceptible or resistant to that disease or condition. A sample that is not normal is one exhibiting phenotypic or genotypic trait(s) of a disease or condition, or one believed to be not normal with respect to that disease or condition. It may be compared to a cell that is normal with respect to that disease or condition. Phenotypic traits include symptoms of, or susceptibility to, a disease or condition of which a component is or may or may not be genetic, or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

Particularly, arrays can be used to evaluate samples with respect to pathological condition such as cancer and related conditions. It is specifically contemplated that the invention can be used to evaluate differences between stages or sub-classifications of disease, such as between benign, cancerous, and metastatic tissues or tumors.

Phenotypic traits to be assessed include characteristics such as longevity, morbidity, expected survival, susceptibility or receptivity to particular drugs or therapeutic treatments (drug efficacy), and risk of drug toxicity. Samples that differ in these phenotypic traits may also be evaluated using the compositions and methods described.

In certain embodiments, miRNA and/or expression profiles may be generated to evaluate and correlate those profiles with pharmacokinetics or therapies. For example, these profiles may be created and evaluated for patient tumor and blood samples prior to the patient's being treated or during treatment to determine if there are miRNA or genes whose expression correlates with the outcome of the patient's treatment. Identification of differential miRNAs or genes can lead to a diagnostic assay for evaluation of tumor and/or blood samples to determine what drug regimen the patient should be provided. In addition, it can be used to identify or select patients suitable for a particular clinical trial. If an expression profile is determined to be correlated with drug efficacy or drug toxicity, that profile is relevant to whether that patient is an appropriate patient for receiving a drug, for receiving a combination of drugs, or for a particular dosage of the drug.

In addition to the above prognostic assay, samples from patients with a variety of diseases can be evaluated to determine if different diseases can be identified based on miRNA and/or related gene expression levels. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005 in the names of David Brown, Lance Ford, Angie Cheng and Rich Jarvis, which is hereby incorporated by reference in its entirety.

E. Other Assays

In addition to the use of arrays and microarrays, it is contemplated that a number of different assays could be employed to analyze miRNAs or related genes, their activities, and their effects. Such assays include, but are not limited to, nucleic acid amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### IV. NUCLEIC ACIDS

The present invention concerns nucleic acids, modified nucleic acids, nucleic acid mimetics, miRNAs, mRNAs, genes, and representative fragments thereof that can be labeled, used in array analysis, or employed in diagnostic, therapeutic, or prognostic applications, particularly those related to pathological conditions such as cancer. The molecules may have been endogenously produced by a cell, or been synthesized or produced chemically or recombinantly. They may be isolated and/or purified. Each of the miRNAs described herein include the corresponding SEQ ID NO and accession numbers for these miRNA sequences. The name of a miRNA is often abbreviated and referred to without a “hsa-” prefix and will be understood as such, depending on the context. Unless otherwise indicated, miRNAs referred to in the application are human sequences identified as miR-X or let-X, where X is a number and/or letter.

In certain aspects, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the world wide web at sanger.ac.uk. Moreover, in some embodiments, a miRNA probe is used that does not correspond to a known human miRNA. It is contemplated that these non-human miRNA probes may be used in embodiments of the invention or that there may exist a human miRNA that is homologous to the non-human miRNA. In other embodiments, any mammalian cell, biological sample, or preparation thereof may be employed.

In some embodiments of the invention, methods and compositions involving miRNA may concern miRNA, markers (mRNAs), and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, mRNA, mRNA probes, control nucleic acids, and other probes and primers.

In many embodiments, miRNA are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or other nucleic acid or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of SEQ ID NOs described herein, accession number, or any other sequence disclosed herein. Typically, the commonly used name of the miRNA is given (with its identifying source in the prefix, for example, “hsa” for human sequences) and the processed miRNA sequence. Unless otherwise indicated, a miRNA without a prefix will be understood to refer to a human miRNA. Moreover, a lowercase letter in a miRNA name may or may not be lowercase; for example, hsa-mir-130b can also be referred to as miR-130B. The term “miRNA probe” refers to a nucleic acid probe that can identify a particular miRNA or structurally related miRNAs.

It is understood that some nucleic acids are derived from genomic sequences or a gene. In this respect, the term “gene” is used for simplicity to refer to the genomic sequence encoding the precursor nucleic acid or miRNA for a given miRNA or gene. However, embodiments of the invention may involve genomic sequences of a miRNA that are involved in its expression, such as a promoter or other regulatory sequences.

The term “recombinant” may be used and this generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, miRNA nucleic acids may encompass a molecule that comprises one or more complementary or self-complementary strand(s) or “complement(s)” of a particular sequence. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

It is understood that a “synthetic nucleic acid” of the invention means that the nucleic acid does not have all or part of a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

While embodiments of the invention may involve synthetic miRNAs or synthetic nucleic acids, in some embodiments of the invention, the nucleic acid molecule(s) need not be “synthetic.” In certain embodiments, a non-synthetic nucleic acid or miRNA employed in methods and compositions of the invention may have the entire sequence and structure of a naturally occurring mRNA or miRNA precursor or the mature mRNA or miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA and not a non-synthetic miRNA (that is, not a miRNA that qualifies as “synthetic”); though in other embodiments, the invention specifically involves a non-synthetic miRNA and not a synthetic miRNA. Any embodiments discussed with respect to the use of synthetic miRNAs can be applied with respect to non-synthetic miRNAs, and vice versa.

It will be understood that the term “naturally occurring” refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA.” Corresponding miRNA sequences that can be used in the context of the invention include, but are not limited to, all or a portion of those sequences in the SEQ IDs provided herein, as well as any other miRNA sequence, miRNA precursor sequence, or any sequence complementary thereof. In some embodiments, the sequence is or is derived from or contains all or part of a sequence identified herein to target a particular miRNA (or set of miRNAs) that can be used with that sequence. Any 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130 140, 150, 160, 170, 180, 190, 200, 210, 220, 230, 240, 250, 260 or any number or range of sequences there between may be selected to the exclusion of all non-selected sequences.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

A. Nucleobase, Nucleoside, Nucleotide, and Modified Nucleotides

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring. Different types of covalent attachment(s) of a nucleobase to a nucleobase linker moiety are known in the art (Kornberg and Baker, 1992).

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids include those in: U.S. Pat. Nos. 5,681,947, 5,652,099 and 5,763,167, 5,614,617, 5,670,663, 5,872,232, 5,859,221, 5,446,137, 5,886,165, 5,714,606, 5,672,697, 5,466,786, 5,792,847, 5,223,618, 5,470,967, 5,378,825, 5,777,092, 5,623,070, 5,610,289, 5,602,240, 5,858,988, 5,214,136, 5,700,922, 5,708,154, 5,728,525, 5,637,683, 6,251,666, 5,480,980, and 5,728,525, each of which is incorporated herein by reference in its entirety.

Labeling methods and kits of the invention specifically contemplate the use of nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, Biosearch Technologies and NEN. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

B. Preparation of Nucleic Acids

A nucleic acid may be made by any technique known to one of ordinary skill in the art, such as for example, chemical synthesis, enzymatic production, or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

Alternatively, nucleic acid synthesis is performed according to standard methods. See, for example, Itakura and Riggs (1980) and U.S. Pat. Nos. 4,704,362, 5,221,619, and 5,583,013, each of which is incorporated herein by reference. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. See also Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

C. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried and resuspended in a liquid and volume appropriate for subsequent manipulation.

### V. LABELS AND LABELING TECHNIQUES

In some embodiments, the present invention concerns miRNA that are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

A. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed. Terminal transferase catalyzes the addition of nucleotides to the 3′ terminus of a nucleic acid. Polynucleotide phosphorylase can polymerize nucleotide diphosphates without the need for a primer.

B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

C. Visualization Techniques

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### VI. KITS

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from blood samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (acute myeloid leukemia), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ IDs described herein. In certain embodiments, a kit or array of the invention can contain one or more probes for the miRNAs identified by the SEQ IDs described herein. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Genes, Gene Pathways, and Cancer-Related Genes with Altered Expression Following Transfection with hsa-miR-15a

miRNAs are believed to regulate gene expression by binding to target mRNA transcripts and (1) initiating transcript degradation or (2) altering protein translation from the transcript. Translational regulation leading to an up or down change in protein expression may lead to changes in activity and expression of downstream gene products and genes that are in turn regulated by those proteins. These numerous regulatory effects may be revealed as changes in the global mRNA expression profile. Microarray gene expression analyses were performed to identify genes that are mis-regulated by hsa-miR-15a expression.

Synthetic pre-miR-15a (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1A.

Manipulation of the expression levels of the genes listed in Table 1A represents a potentially useful therapy for cancer and other diseases in which increased or reduced expression of hsa-miR-15a has a role in the disease.

The mis-regulation of gene expression by hsa-miR-15a (Table 1A) affects many cellular pathways that represent potential therapeutic targets for the control of cancer and other diseases and disorders. The inventors determined the identity and nature of the cellular genetic pathways affected by the regulatory cascade induced by hsa-miR-15a expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-15a in A549 cells are shown in Table 2A.

These data demonstrate that hsa-miR-15a directly or indirectly affects the expression of several, cellular proliferation-, development-, and cell growth-related genes and thus primarily effects functional pathways related to cellular growth and cellular development. Those cellular processes have integral roles in the development and progression of various cancers. Manipulation of the expression levels of genes in the cellular pathways shown in Table 2A represents a potentially useful therapy for cancer and other diseases in which increased or reduced expression of hsa-miR-15a has a role in the disease.

Gene targets for binding of and regulation by hsa-miR-15a were predicted using the proprietary algorithm miRNATarget™ (Asuragen), which is an implementation of the method proposed by Krek et al. (2005). The predicted gene targets that exhibited altered mRNA expression levels in human cancer cells, following transfection with pre-miR hsa-miR-15a, are shown in Table 3A.

The verified gene targets of hsa-miR-15a in Table 3A represent particularly useful candidates for cancer therapy and therapy of other diseases through manipulation of their expression levels.

Cell proliferation and growth pathways are commonly altered in tumors (Hanahan and Weinberg, 2000). The inventors have shown that hsa-miR-15a directly or indirectly regulates the transcripts of proteins that are critical in the regulation of these pathways. Many of these targets have inherent oncogenic or tumor suppressor activity and are frequently deregulated in human cancer. Hsa-miR-15a targets that have prognostic and/or therapeutic value for the treatment of various malignancies are shown in Table 4A. Based on this review of the genes and related pathways that are regulated by miR-15a, introduction of hsa-miR-15a or an anti-hsa-miR-15a into a variety of cancer cell types would likely result in a therapeutic response.

### Example 2

### Delivery of Synthetic hsa-miR-15a Inhibits Proliferation of Human Prostate Cancer Cells

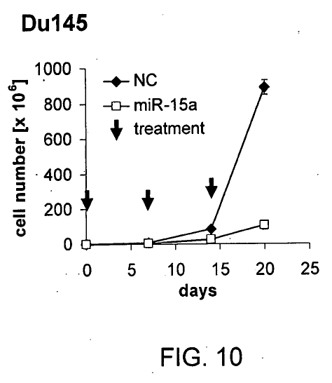

The inventors assessed the therapeutic effect of hsa-miR-15a for prostate cancer by using the Du145 human prostate cancer cell line, derived from a brain metastasis (Stone et al., 1978). The inventors conducted growth curve experiments in the presence of miRNA for up to 20 days. Since in vitro transfections of naked interfering RNAs, such as synthetic miRNA, are transient by nature and compromised by the dilution of the miRNA during ongoing cell divisions, miRNA was administered at multiple time points (Bartlett et al., 2006; Bartlett et al., 2007). To accommodate miRNA delivery into a large quantity of cells, the inventors employed the electroporation method for delivery of hsa-miR-15a or negative control miRNA into Du145 human prostate cancer cells. Briefly, 0.5×10⁶ Du145 cells were electroporated with 1.6 μM hsa-miR-15a or negative control using the BioRad Gene Pulser Xcell™ instrument (BioRad Laboratories Inc., Hercules, Calif., USA), seeded and propagated in regular growth medium. Experiments were carried out in triplicates. When the control cells reached confluence (days 7 and 14), cells were harvested, counted and electroporated again with the respective miRNAs. To ensure similar treatment of both conditions as well as to accommodate exponential growth, the cell numbers used for the second and third electroporation were titrated down to the lowest count. The population doubling was calculated from these electroporation events using the formula PD=ln(Nf/N0)/ln2 and adjusting for the fact that approximately 72% of newly seeded cells adhere to the plate. Cell counts were extrapolated and plotted on a linear scale (FIG. 10). Arrows represent electroporation days. Standard deviations are included in the graphs.

Repeated administration of hsa-miR-15a robustly inhibited proliferation of human prostate cancer cells (FIG. 10, white squares). In contrast, cells treated with negative control miRNA showed normal exponential growth (FIG. 10, black diamonds). hsa-miR-15a treatment resulted in 88.2% inhibition of Du145 cell growth on day 20 (11.8% cells relative to cells electroporated with negative control miRNA) relative to the proliferation of control cells (100%).

The data suggest that hsa-miR-15a provides a useful therapeutic tool in the treatment of human patients with prostate cancer and potentially other diseases.

### Example 3

### Genes, Gene Pathways, and Cancer-Related Genes with Altered Expression Following Transfection with hsa-miR-26a

As mentioned above in Example 1, the regulatory effects of miRNAs are revealed through changes in global gene expression profiles following miRNA expression or inhibition of miRNA expression. Microarray gene expression analyses were performed to identify genes that are mis-regulated by hsa-miR-26a expression. Synthetic pre-miR-26a (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1B.

Manipulation of the expression levels of the genes listed in Table 1B represents a potentially useful therapy for cancer and other diseases in which increased or reduced expression of hsa-miR-26a has a role in the disease.

The mis-regulation of gene expression by hsa-miR-26a (Table 1B) affects many cellular pathways that represent potential therapeutic targets for the control of cancer and other diseases and disorders. The inventors determined the identity and nature of the cellular genetic pathways affected by the regulatory cascade induced by hsa-miR-26a expression. Cellular pathway analyses were performed using Ingenuity Pathways Analysis (Version 4.0, Ingenuity Systems, Redwood City, Calif.). Alteration of a given pathway was determined by Fisher's Exact test (Fisher, 1922). The most significantly affected pathways following over-expression of hsa-miR-26a in A549 cells are shown in Table 2B.

These data demonstrate that hsa-miR-26a directly or indirectly affects the expression of numerous cellular proliferation-, development-, cell growth, and cancer-related genes and thus primarily affects functional pathways related to cancer, cell signaling, cellular growth, and cellular development. Those cellular processes have integral roles in the development and progression of various cancers. Manipulation of the expression levels of genes in the cellular pathways shown in Table 2B represents a potentially useful therapy for cancer and other diseases in which increased or reduced expression of hsa-miR-26a has a role in the disease.

Gene targets for binding of and regulation by hsa-miR-26a were predicted using the proprietary algorithm miRNATarget™ (Asuragen), which is an implementation of the method proposed by Krek et al. (2005). The predicted gene targets that exhibited altered mRNA expression levels in human cancer cells, following transfection with pre-miR hsa-miR-26a, are shown in Table 3B.

The verified gene targets of hsa-miR-26a in Table 3B represent particularly useful candidates for cancer therapy and therapy of other diseases through manipulation of their expression levels.

Cell proliferation and survival pathways are commonly altered in tumors (Hanahan and Weinberg, 2000). The inventors have shown that hsa-miR-26a directly or indirectly regulates the transcripts of proteins that are critical in the regulation of these pathways. Many of these targets have inherent oncogenic or tumor suppressor activity and are frequently deregulated in human cancer. Hsa-miR-26a targets that have prognostic and/or therapeutic value for the treatment of various malignancies are shown in Table 4B. Based on this review of the genes and related pathways that are regulated by miR-26a, introduction of hsa-miR-26a or an anti-hsa-miR-26a into a variety of cancer cell types would likely result in a therapeutic response.

### Example 4

### Genes, Gene Pathways, and Cancer-Related Genes with Altered Expression Following Transfection with Anti-hsa-miR-31

Microarray gene expression analyses were performed to identify genes that are mis-regulated by inhibition of hsa-miR-31 expression. Synthetic anti-miR-31 (Ambion) or a negative control anti-miRNA (anti-miR-NC1, Ambion cat. no. AM17010) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on an Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1C.

Manipulation of the expression levels of the genes listed in Table 1C represents a potentially useful therapy for cancer and other diseases in which increased or reduced expression of hsa-miR-31 has a role in the disease.

The mis-regulation of gene expression by anti-hsa-miR-31 (Table 1C) affects many cellular pathways that represent potential therapeutic targets for the control of cancer and other diseases and disorders. The inventors determined the identity and nature of the cellular genetic pathways affected by the regulatory cascade induced by the inhibition of hsa-miR-31 expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following inhibition of hsa-miR-31 in A549 cells are shown in Table 2C.

These data demonstrate that hsa-miR-31 directly or indirectly affects primarily cellular development-related genes and thus primarily affects functional pathways related to cellular development. Cellular development has an integral role in the progression of various cancers. Manipulation of the expression levels of genes in the cellular pathways shown in Table 2C represents a potentially useful therapy for cancer and other diseases in which increased or reduced expression of hsa-miR-31 has a role in the disease.

Gene targets for binding of and regulation by hsa-miR-31 were predicted using the proprietary algorithm miRNATarget™ (Asuragen), which is an implementation of the method proposed by Krek et al. (2005). The predicted gene targets that exhibited altered mRNA expression levels in human cancer cells, following transfection with anti-hsa-miR-31, are shown in Table 3C.

miRNAs are believed to regulate gene expression by binding to target mRNA transcripts and (1) initiating transcript degradation or (2) altering protein translation from the transcript. Inhibition of hsa-miR-31 would likely inhibit degradation of target transcripts. As expected, the inventors observed that the predicted targets of hsa-miR-31 exhibiting altered mRNA expression upon transfection with anti-hsa-miR-31 all showed an increase in transcript levels. The verified gene targets of hsa-miR-31 in Table 3C represent particularly useful candidates for cancer therapy and therapy of other diseases through manipulation of their expression levels.

### Example 5

### Genes, Gene Pathways, and Cancer-Related Genes with Altered Expression Following Transfection with hsa-miR-145

As mentioned above in Example 1, the regulatory effects of miRNAs are revealed through changes in global gene expression profiles following miRNA expression or inhibition of miRNA expression. Microarray gene expression analyses were performed to identify genes that are mis-regulated by hsa-miR-145 expression. Synthetic pre-miR-145 (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1D.

Manipulation of the expression levels of the genes listed in Table 1D represents a potentially useful therapy for cancer and other diseases in which increased or reduced expression of hsa-miR-145 has a role in the disease.

The mis-regulation of gene expression by hsa-miR-145 (Table 1D) affects many cellular pathways that represent potential therapeutic targets for the control of cancer and other diseases and disorders. The inventors determined the identity and nature of the cellular genetic pathways affected by the regulatory cascade induced by hsa-145 expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-145 in A549 cells are shown in Table 2D.

These data demonstrate that hsa-miR-145 directly or indirectly affects the expression of development- and cancer-related genes. Those cellular processes have integral roles in the development and progression of various cancers. Manipulation of the expression levels of genes in the cellular pathways shown in Table 2D represents a potentially useful therapy for cancer and other diseases in which increased or reduced expression of hsa-miR-145 has a role in the disease.

Gene targets for binding of and regulation by hsa-miR-145 were predicted using the proprietary algorithm miRNATarget™ (Asuragen), which is an implementation of the method proposed by Krek et al. (2005). The predicted gene targets that exhibited altered mRNA expression levels in human cancer cells, following transfection with pre-miR hsa-miR-145, are shown in Table 3D.

The verified gene target of hsa-miR-145 in Table 3D represents a particularly useful candidate for cancer therapy and therapy of other diseases through manipulation of its expression levels.

### Example 6

### Genes, Gene Pathways, and Cancer-Related Genes with Altered Expression Following Transfection with hsa-miR-147

As mentioned above in Example 1, the regulatory effects of miRNAs are revealed through changes in global gene expression profiles following miRNA expression or inhibition of miRNA expression. Microarray gene expression analyses were performed to identify genes that are mis-regulated by hsa-miR-147 expression. Synthetic pre-miR-147 (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageArp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1E.

Manipulation of the expression levels of the genes listed in Table 1E represents a potentially useful therapy for cancer and other diseases in which increased or reduced expression of hsa-miR-147 has a role in the disease.

The mis-regulation of gene expression by hsa-miR-147 (Table 1E) affects many cellular pathways that represent potential therapeutic targets for the control of cancer and other diseases and disorders. The inventors determined the identity and nature of the cellular genetic pathways affected by the regulatory cascade induced by hsa-miR-147 expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-147 in A549 cells are shown in Table 2E.

These data demonstrate that hsa-miR-147 directly or indirectly affects the expression of numerous cellular development-, cell growth-, and cancer-related genes and thus primarily affects functional pathways related to cellular growth and cellular development. Those cellular processes have integral roles in the development and progression of various cancers. Manipulation of the expression levels of genes in the cellular pathways shown in Table 2E represents a potentially useful therapy for cancer and other diseases in which increased or reduced expression of hsa-miR-147 has a role in the disease.

Gene targets for binding of and regulation by hsa-miR-147 were predicted using the proprietary algorithm miRNATarget™ (Asuragen), which is an implementation of the method proposed by Krek et al. (2005). The predicted gene targets that exhibited altered mRNA expression levels in human cancer cells, following transfection with pre-miR hsa-miR-147, are shown in Table 3E.

The verified gene targets of hsa-miR-147 in Table 3E represent particularly useful candidates for cancer therapy and therapy of other diseases through manipulation of their expression levels.

Cell proliferation and survival pathways are commonly altered in tumors (Hanahan and Weinberg, 2000). The inventors have shown that hsa-miR-147 directly or indirectly regulates the transcripts of proteins that are critical in the regulation of these pathways. Many of these targets have inherent oncogenic or tumor suppressor activity and are frequently deregulated in human cancer. Hsa-miR-147 targets that have prognostic and/or therapeutic value for the treatment of various malignancies are shown in Table 4C. Based on this review of the genes and related pathways that are regulated by miR-147, introduction of hsa-miR-147 or an anti-hsa-miR-147 into a variety of cancer cell types would likely result in a therapeutic response.

### Example 7

### Delivery of Synthetic hsa-miR-147 Inhibits Proliferation of Parental and Metastatic Human Lung Cancer Cell Lines

The inventors have previously demonstrated that miRNAs described in this application are involved with the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005, each incorporated herein by reference in its entirety). For example, overexpression of hsa-miR-147 decreases the proliferation and/or viability of certain normal or cancerous cell lines.

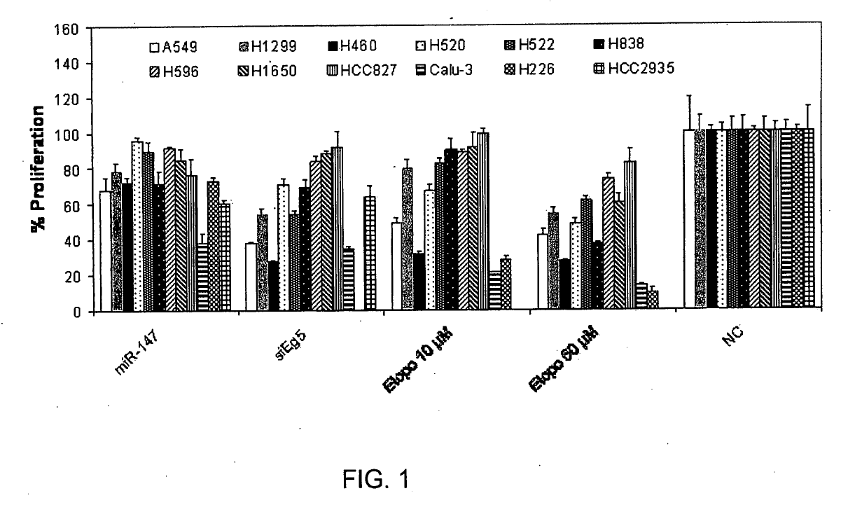

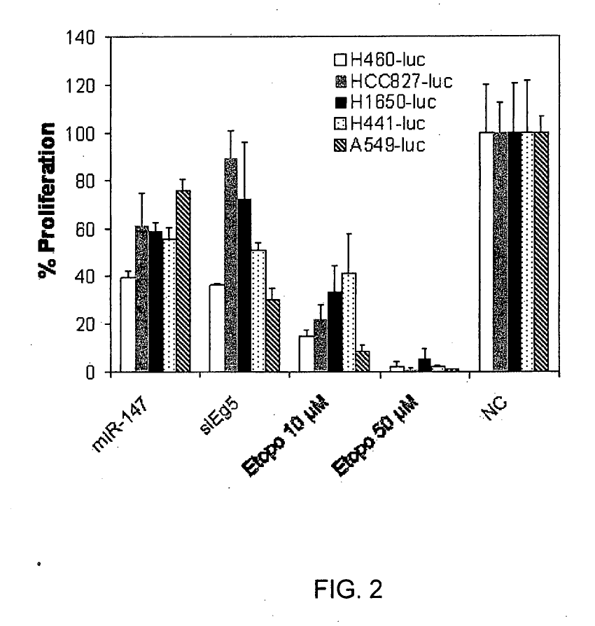

The development of effective therapeutic regimes typically involves demonstrating efficacy and utility of the therapeutic in various cancer models and multiple cancer cell lines that represent the same disease. The inventors assessed the therapeutic effect of hsa-miR-147 for lung cancer by using 12 individual lung cancer cell lines. To measure cellular proliferation of lung cancer cells, the following parental non-small cell lung cancer (NSCLC) cells were used: cells derived from lung adenocarcinoma (A549, H1299, H522, H838, Calu-3, HCC827, HCC2935), cells derived from lung squamous cell carcinoma (H520, H226), cells derived from lung adenosquamous cell carcinoma (H596), cells derived from lung bronchioalveolar carcinoma (H1650), and cells derived from lung large cell carcinoma (H460). In addition to these parental cell lines, highly metastatic NSCLC cells were used that stably express the firefly luciferase gene: A549-luc, H460-luc, HCC827-luc, H1650-luc, H441-luc. Unlike the parental cell lines, these metastatic cells readily migrate to distant sites of the test animal and form metastases upon subcutaneous, orthotopic, or intravenous injection. Synthetic hsa-miR-147 or negative control miRNA was delivered via lipid-based transfection into A549, H1299, H522, H838, Calu-3, HCC827, HCC2935, H520, H596, H1650, H460, A549-luc, H460-luc, HCC827-luc, H1650-luc, H441-luc cells and via electroporation into H226 cells. Lipid-based reverse transfection was carried out in triplicates according to a published protocol and the following parameters: 5000-12000 cells per 96 well, 0.1-0.2 μl lipofectamine-2000 (Invitrogen, Carlsbad, Calif.) in 20 μl OptiMEM (Invitrogen), 30 n™ final concentration of miRNA in 100 μl (Ovcharenko et al., 2005). Electroporation of H226 cells was carried out using the BioRad GenePulserXcell™ instrument with the following settings: 5×10⁶ cells with 5 μg miRNA in 200 μl OptiMEM, square wave pulse at 250 V for 5 ms. Electroporated H226 cells were seeded at 7000 cells per 96-well in a total volume of 100 μl. All cells except for Calu-3 cells were harvested 72 hours post transfection or electroporation for assessment of cellular proliferation. Calu-3 cells were harvested 10 days post transfection. Proliferation assays were performed using Alamar Blue (Invitrogen) following the manufacturer's instructions. As a control for inhibition of cellular proliferation, siRNA against the motor protein kinesin 11, also known as Eg5, was used. Eg5 is essential for cellular survival of most eukaryotic cells and a lack thereof leads to reduced cell proliferation and cell death (Weil et al., 2002). siEg5 was used in lipid-based transfection following the same experimental parameters that apply to miRNA. The inventors also used the topoisomerase II inhibitor etoposide at a final concentration of 10 μM and 50 μM as an internal standard for the potency of miRNAs. Etoposide is an FDA-approved topoisomerase II inhibitor in the treatment of lung cancer. IC₅₀ values for various lung cancer cells have been reported to range between <1-25 μM for SCLC and NSCLC cells (Tsai et al., 1993; Ohsaki et al., 1992). Values obtained from the Alamar Blue assay were normalized to values from cells treated with negative control miRNA. FIG. 1, FIG. 2, Table 6, and Table 7 show % proliferation of hsa-miR-147 treated cells relative to cells treated with negative control miRNA (=100%). Standard deviations are indicated in the graphs and tables.

TABLE 6
Percent (%) proliferation of parental human lung cancer cell lines treated with hsa-
miR-147, Eg5-specific siRNA (siEg5), etoposide, or negative control miRNA (NC).

| | hsa-miR-147 | | | | etoposide | | etoposide | | | |
|---|---|---|---|---|---|---|---|---|---|---|
| | (30 nM) | | siEg5 (30 nM) | | (10 μM) | | (50 μM) | | NC (30 nM) | |
| | % | % | % | % | % | | % | % | % | % |
| Cells | proliferation | SD | proliferation | SD | proliferation | % SD | proliferation | SD | proliferation | SD |
| A549 | 67.78 | 6.75 | 37.84 | 1.06 | 49.13 | 2.55 | 42.18 | 3.57 | 100.00 | 19.53 |
| H1299 | 78.22 | 4.64 | 54.32 | 2.83 | 79.65 | 5.02 | 54.38 | 2.73 | 100.00 | 8.89 |
| H460 | 72.11 | 2.29 | 27.97 | 0.33 | 32.13 | 1.14 | 27.82 | 0.58 | 100.00 | 2.52 |
| H520 | 95.64 | 1.96 | 70.40 | 3.49 | 66.80 | 3.93 | 48.53 | 2.54 | 100.00 | 4.15 |
| H522 | 89.21 | 5.44 | 53.45 | 2.35 | 82.13 | 3.14 | 61.08 | 2.65 | 100.00 | 7.48 |
| H838 | 71.44 | 7.12 | 69.14 | 4.15 | 89.71 | 6.17 | 36.97 | 0.62 | 100.00 | 7.74 |
| H596 | 91.60 | 0.62 | 83.48 | 2.82 | 88.75 | 1.11 | 73.39 | 2.67 | 100.00 | 1.89 |
| H1650 | 84.61 | 5.91 | 87.96 | 1.73 | 90.98 | 8.44 | 60.31 | 4.59 | 100.00 | 7.21 |
| HCC827 | 76.18 | 9.05 | 91.68 | 8.89 | 98.95 | 3.00 | 82.53 | 7.75 | 100.00 | 4.32 |
| Calu-3 | 37.62 | 6.21 | 34.59 | 1.33 | 20.81 | 0.19 | 13.53 | 0.64 | 100.00 | 5.54 |
| H226 | 72.82 | 1.76 | n.d. | n.d. | 28.17 | 2.32 | 9.33 | 2.70 | 100.00 | 2.43 |
| HCC2935 | 60.35 | 1.80 | 63.61 | 6.12 | n.d. | n.d. | n.d. | n.d. | 100.00 | 13.92 |

Values are normalized to values obtained from cells transfected with negative control miRNA (100% proliferation).
NC, negative control miRNA;
siEg5, Eg5-specific siRNA;
SD, standard deviation;
n.d., not determined.

Delivery of hsa-miR-147 inhibits cellular proliferation of the parental lung cancer cells A549, H1299, H522, H838, Calu-3, HCC827, HCC2935, H520, H596, H1650, H460, H226, as well as the metastatic lung cancer cells A549-luc, H460-luc, HCC827-luc, H1650-luc and H441-luc (FIG. 1 and FIG. 2). On average, hsa-miR-147 inhibits cellular proliferation of parental lung cancer cells by 25% (FIG. 1), and inhibits cell growth of metastatic lung cancer cells by 42% (FIG. 2). Hsa-miR-147 has maximal inhibitory activity in Calu-3 and H460-luc cells. The growth-inhibitory activity of hsa-miR-147 is comparable to the one of etoposide at concentrations >10 μM. Since hsa-miR-147 induces a therapeutic response in all lung cancer cell tested, hsa-miR-147 may provide a therapeutic benefit to patients with lung cancer and other malignancies.

TABLE 7
Percent (%) proliferation of metastatic human lung cancer cell lines treated with hsa-
miR-147, Eg5-specific siRNA (siEg5), etoposide, or negative control miRNA (NC).

| | hsa-miR-147 | | | | etoposide | | etoposide | | | |
|---|---|---|---|---|---|---|---|---|---|---|
| | (30 nM) | | siEg5 (30 nM) | | (10 μM) | | (50 μM) | | NC (30 nM) | |
| | % | % | % | % | % | | % | % | % | % |
| Cells | proliferation | SD | proliferation | SD | proliferation | % SD | proliferation | SD | proliferation | SD |
| H460-luc | 39.54 | 2.72 | 36.46 | 0.39 | 15.04 | 2.53 | 2.34 | 1.95 | 100.00 | 20.04 |
| HCC827-luc | 61.15 | 13.50 | 89.34 | 11.08 | 21.92 | 6.24 | 0.75 | 0.68 | 100.00 | 12.41 |
| H1650-luc | 59.27 | 3.36 | 72.38 | 23.57 | 33.78 | 10.90 | 5.59 | 4.14 | 100.00 | 20.50 |
| H441-luc | 55.53 | 4.94 | 50.98 | 3.04 | 41.22 | 16.27 | 1.99 | 0.75 | 100.00 | 21.36 |
| A549-luc | 75.69 | 4.93 | 30.14 | 4.53 | 8.56 | 2.41 | 0.72 | 0.20 | 100.00 | 6.56 |

Values are normalized to values obtained from cells transfected with negative control miRNA (100% proliferation).
NC, negative control miRNA;
siEg5, Eg5-specific siRNA;
SD, standard deviation;
n.d., not determined.

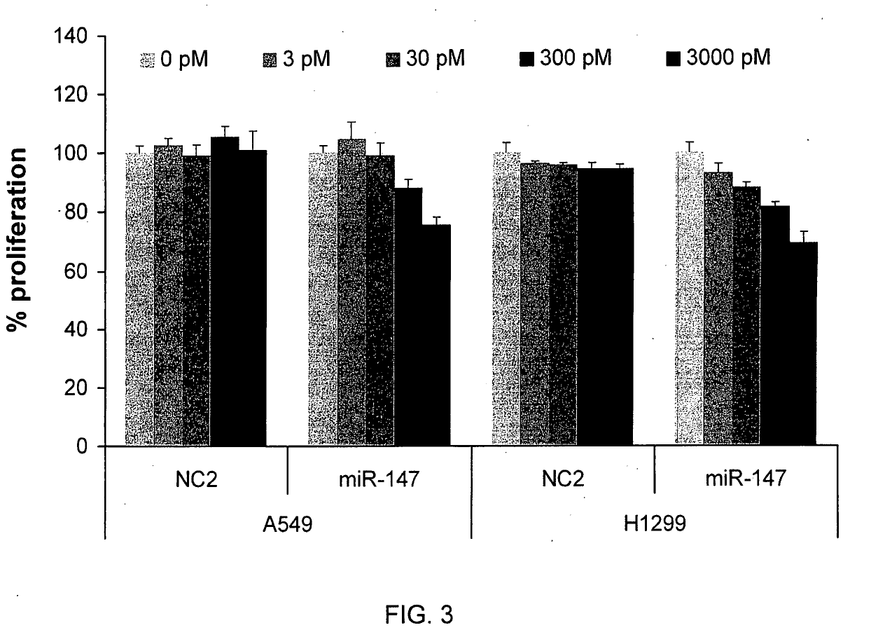

The inventors determined sensitivity and specificity of hsa-miR-147 by administering hsa-miR-147 or negative control miRNA at increasing concentrations, ranging from 0 pM to 3 nM. Delivery of miRNA and cellular proliferation of A549 and H1299 cells was assessed as described above. Alamar Blue values were normalized to values obtained from mock-transfected cells (0 pM=100% proliferation). As shown in FIG. 3 and Table 8, increasing amounts of negative control miRNA had no effect on cellular proliferation of A549 or H1299 cells. In contrast, the growth-inhibitory phenotype of hsa-miR-147 is dose-dependent and correlates with increasing amounts of hsa-miR-147. Hsa-miR-147 induces a therapeutic response at concentrations as low as 300 pM.

TABLE 8
Dose-dependent inhibition of cellular proliferation of lung cancer cell lines by hsa-
miR-147.

| | A549 Cells | | | | H1299 Cells | | | |
|---|---|---|---|---|---|---|---|---|
| | hsa-miR-147 | | NC | | hsa-miR-147 | | NC | |
| Concentration | % | % | % | % | % | % | % | % |
| [pM] | proliferation | SD | proliferation | SD | proliferation | SD | proliferation | SD |
| 0 | 100.00 | 2.61 | 100.00 | 2.61 | 100.00 | 3.28 | 100.00 | 3.28 |
| 3 | 104.77 | 5.79 | 102.82 | 2.23 | 93.08 | 3.13 | 96.51 | 0.51 |
| 30 | 99.22 | 4.23 | 99.36 | 3.51 | 88.20 | 1.59 | 95.89 | 0.61 |
| 300 | 88.24 | 2.63 | 105.53 | 3.72 | 81.82 | 1.46 | 94.45 | 1.99 |
| 3,000 | 75.78 | 2.39 | 101.30 | 6.35 | 69.70 | 3.36 | 94.56 | 1.24 |

Values are normalized to values obtained from mock-transfected cells (0 pM miRNA).
NC, negative control miRNA;
% SD, standard deviation.

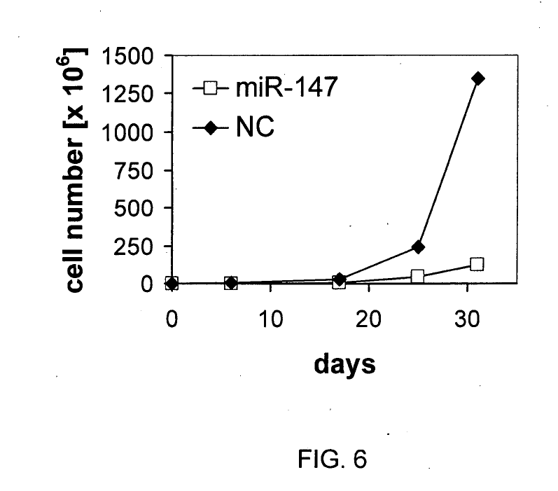

To evaluate the therapeutic activity of hsa-miR-147 over an extended period of time, the inventors conducted growth curve experiments in the presence of miRNA for up to 31 days in H226 lung cancer cells. Since in vitro transfections of naked interfering RNAs, such as synthetic miRNA, are transient by nature and compromised by the dilution of the oligo during ongoing cell divisions, miRNA was administered at multiple time points (Bartlett et al., 2006; Bartlett et al., 2007). To accommodate miRNA delivery into a large quantity of cells, hsa-miR-147 or negative control miRNA were delivered by the electroporation method. Briefly, 1×10⁶ H226 were electroporated in triplicate with 1.6 μM hsa-miR-147 or negative control using the BioRad Gene Pulser Xcell™ instrument (BioRad Laboratories Inc., Hercules, Calif., USA), seeded and propagated in regular growth medium. When the control cells reached confluence (days 6, 17 and 25), cells were harvested, counted and electroporated again with the respective miRNAs. To ensure similar treatment of both conditions as well as to accommodate exponential growth, the cell numbers used for the second and third electroporation were titrated down to the lowest count. The population doubling was calculated from these electroporation events using the formula PD=ln(Nf/N0)/ln2 and adjusting for the fact that approximately 72% of newly seeded cells adhere to the plate. Cell counts were extrapolated and plotted on a linear scale (FIG. 6). Arrows represent electroporation days. Standard deviations are included in the graphs.

Repeated administration of hsa-miR-147 robustly inhibited proliferation of human lung cancer cells (FIG. 6). In contrast, cells treated with negative control miRNA showed normal exponential growth. hsa-miR-147 treatment resulted in 90.9% inhibition of H226 cell growth on day 31 (9.1% remaining cells) relative to the proliferation of control cells (100%).

The data suggest that hsa-miR-147 provides a useful therapeutic tool in the treatment of human lung cancer cells and potentially other diseases.

### Example 8

### hsa-miR-147 in Combination with hsa-miR-124a, hsa-miR-126, hsa-let-7b, hsa-let-7c or hsa-let-7g Synergistically Inhibits Proliferation of Human Lung Cancer Cell Lines

miRNAs function in multiple pathways controlling multiple cellular processes. Cancer cells frequently show aberrations in several different pathways which determine their oncogenic properties. Therefore, combinations of multiple miRNAs may provide a better therapeutic benefit rather than a single miRNA. The inventors assessed the efficacy of pair-wise miRNA combinations, administering hsa-miR-147 concurrently with hsa-miR-124a, hsa-miR-126, hsa-let7b, hsa-let-7c or hsa-let7g. H460 lung cancer cells were transiently reverse transfected in triplicates with each miRNA at a final concentration of 300 pM, totaling in 600 pM of oligonucleotide. As a negative control, 600 pM of negative control miRNA (pre-miR NC#2, Ambion) was used. To correlate the effect of various combinations with the effect of the sole miRNA, each miRNA at 300 pM was also combined with 300 pM negative control miRNA. Reverse transfection was carried using the following parameters: 7000 cells per 96 well, 0.15 μl lipofectamine2000 (Invitrogen) in 20 μl OptiMEM (Invitrogen), 100 μl total transfection volume. As an internal control for the potency of miRNA, etoposide was added at 10 μM and 50 μM to mock-transfected cells 24 hours after transfection for the following 48 hours. Cells were harvested 72 hours after transfection and subjected to Alamar Blue assays (Invitrogen). Alamar Blue values were normalized to the ones obtained from cells treated with 600 pM negative control miRNA. Data are expressed as % proliferation relative to negative control miRNA-treated cells.

TABLE 9
Cellular proliferation of H460 lung cancer cells in the presence of
pair-wise miR-147 miRNA combinations. Values are normalized to
values obtained from cells transfected with 600 pM negative control
(NC) miRNA.

| | % | % | |
|---|---|---|---|
| miRNA [300 pM] + miRNA [300 pM] | Proliferation | SD | Effect |
| NC + NC | 100.00 | 1.45 | |
| NC + miR-124a | 69.43 | 1.38 | |
| NC + miR-126 | 89.46 | 2.27 | |
| NC + miR-147 | 76.97 | 1.46 | |
| NC + let-7b | 74.92 | 3.38 | |
| NC + let-7c | 86.74 | 2.28 | |
| NC + let-7g | 91.41 | 3.26 | |
| miR-147 + miR-124a | 42.81 | 1.73 | S |
| miR-147 + miR-126 | 62.64 | 3.79 | S |
| miR-147 + let-7b | 56.55 | 3.85 | A |
| miR-147 + let-7c | 60.74 | 0.60 | A |
| miR-147 + let-7g | 56.19 | 2.95 | S |
| Etoposide (10 μM) | 20.19 | 1.89 | |
| Etoposide (50 μM) | 14.94 | 0.31 | |

SD, standard deviation;
S, synergistic effect;
A, additive effect.

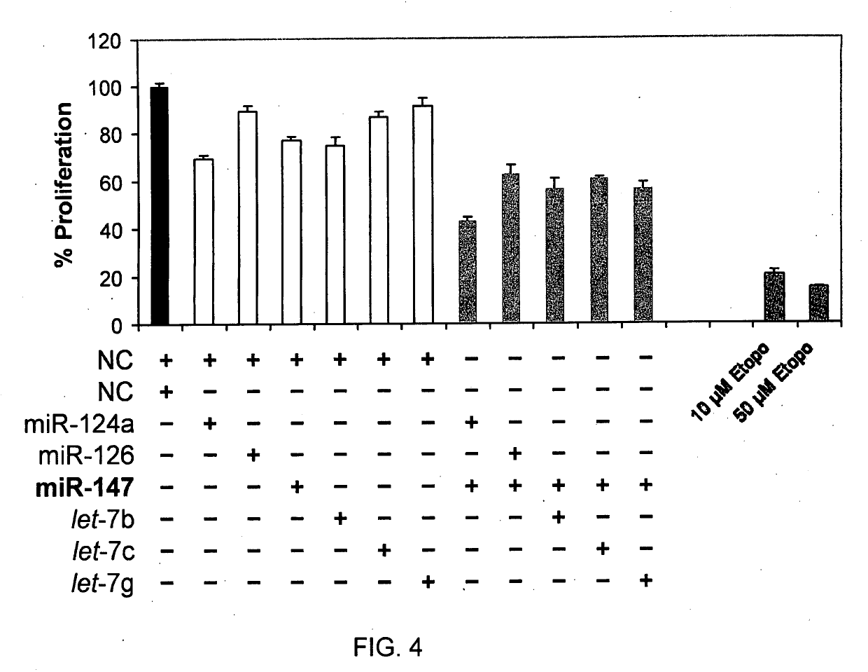

As shown in FIG. 4 and Table 9, transfection of 300 pM hsa-miR-147 reduces proliferation of H460 cells by 23%. Maximal activity of singly administered miRNAs was observed with hsa-miR-124a, diminished cellular proliferation by 30.6%. Additive activity of pair-wise combinations (e.g., hsa-miR-147 plus hsa-miR-124a) is defined as an activity that is greater than the sole activity of each miRNA (e.g., activity of hsa-miR-147 plus hsa-miR-124a>hsa-miR-147 plus NC AND activity of hsa-miR-147 plus hsa-miR-124a>hsa-miR-124a plus NC). Synergistic activity of pair-wise combinations is defined as an activity that is greater than the sum of the sole activity of each miRNA (e.g., activity of hsa-miR-147 plus hsa-miR-124a>SUM [activity of hsa-miR-147 plus NC AND activity of hsa-miR-124a plus NC]). The data suggest that hsa-miR-147 combined with hsa-let-7b or hsa-let-7c provides an additive effect; combinations of hsa-miR-147 with hsa-miR124a, hsa-miR-126 or hsa-let-7g results in synergistic activity (FIG. 4, Table 9). In summary, all pair-wise combinations of hsa-miR-147 induce a better therapeutic response in H460 lung cancer cells relative to the administration of the single miRNA.

The combinatorial use of miRNAs represents a potentially useful therapy for cancer and other diseases.

### Example 9

### Delivery of Synthetic hsa-miR-147 Inhibits Tumor Growth of Human Lung Cancer Cells in Mice

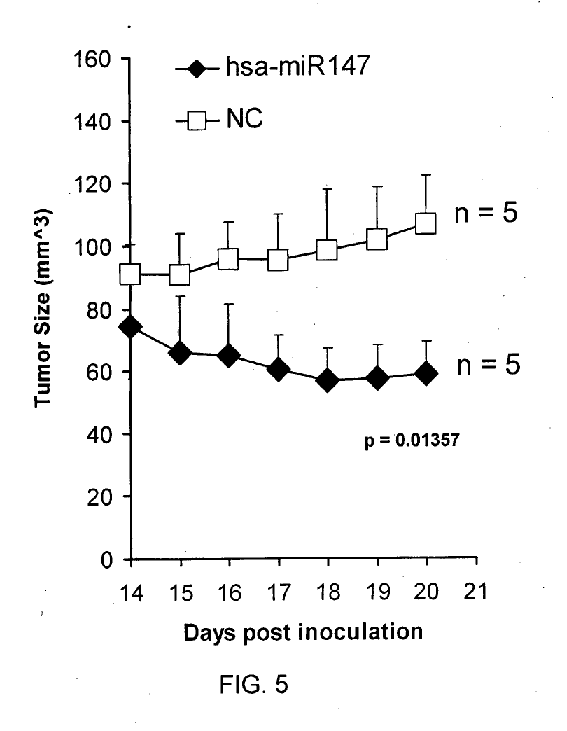

The inventors assessed the growth-inhibitory activity of hsa-miR-147 in a human lung cancer xenograft grown in immunodeficient mice. Hsa-miR-147 was delivered into A549 lung cancer cells via electroporation using the BioRad GenePulserXcell™ instrument with the following settings: 15×10⁶ cells with 5 μg miRNA in 200 μl OptiMEM, square wave pulse at 150 V for 10 ms. A total of 30×10⁶ A549 cells was used to 5×10⁶ electroporated cells were mixed with matrigel in a 1:1 ratio and injected subcutaneously into the flank of NOD/SCID mice. As a negative control, A549 cells were electroporated with negative control miRNA (pre-miR-NC#2, Ambion) as describe above. NC miRNA-treated cells were injected into the opposite flank of the same animal to control for animal-to-animal variability. A total of 30×10⁶ A549 cells per hsa-miR-147 and NC was used to accommodate 5 injections into 5 animals. Size measurements of tumors started 14 days after injection once tumors have reached a measurable size. Length and width of tumors were determined every day for the following 6 days. Tumor volumes were calculated using the formula V=length×width²/2 in which the length is greater than the width. Tumor volumes derived from NC-treated cells and hsa-miR-147-treated cells were averaged and plotted over time (FIG. 5). Standard deviations are shown in the graph. The p value, indicating statistical significance, is shown for values obtained on day 20.

Administration of hsa-miR-147 into the A549 lung cancer xenograft inhibited tumor growth in vivo (FIG. 5). Cancer cells that received negative control miRNA developed tumors more rapidly than cells treated with hsa-miR147. Administration of hsa-miR-147 A549 induced tumor regression and prevented further tumor growth. Data points obtained on day 20 are statistically significant (p=0.01357).

Delivery of hsa-miR-147 into human lung cancer cells prior to implantation into the animal inhibited the formation of lung tumor xenografts. These results demonstrate the anti-oncogenic activity of hsa-miR-147 and suggest that hsa-miR-147 may also provide a powerful therapeutic tool to treat established lung tumors. To explore this possibility, 3×10⁶ human H460 non-small cell lung cancer cells were mixed with BD Matrigel™, (BD Biosciences; San Jose, Calif., USA; cat. no. 356237) in a 1:1 ratio and subcutaneously injected into the lower back of each of 23 NOD/SCID mice (Charles River Laboratories, Inc.; Wilmington, Mass., USA). Once animals developed palpable tumors (day 11 post xenograft implantation), each animal in a group of six received intratumoral injections of 6.25 μg hsa-miR-147 (Dharmacon, Lafayette, Colo.) formulated with the lipid-based siPORT™ amine delivery agent (Ambion, Austin, Tex.; cat. no. AM4502) on days 11, 14 and 17. A control group of six animals each received intratumoral injections of 6.25 μg negative control miRNA (NC; Dharmacon, Lafayette, Colo.), following the same injection schedule that was used for hsa-miR-147. Given an average mouse weight of 20 g, this dose equals 0.3125 mg/kg. In addition, a group of six H460 tumor-bearing mice received intratumoral injections of the siPORT™ amine delivery formulation lacking any oligonucleotide, and a group of five animals received intratumoral injections of phosphate-buffered saline (PBS). Caliper measurements of tumors were taken every 1-2 days, and tumor volumes were calculated using the formula, Volume=length×width×width/2, in which the length is greater than the width.

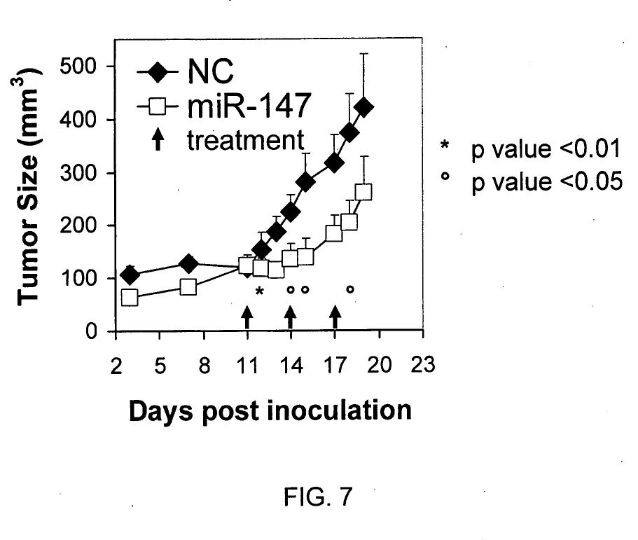

As shown in FIG. 7, three doses of hsa-miR-147 robustly inhibited growth of established H460 lung tumors and yielded tumors with an average size of 260 mm³ on day 19. In contrast, tumors treated with negative control miRNA grew at a steady pace and yielded tumors with an average size of 420 mm³ on day 19. Negative control tumors developed as quickly as tumors treated with either PBS or the siPORT amine-only control, indicating that the therapeutic activity of hsa-miR-147 is specific.

The data suggest that hsa-miR-147 represents a particularly useful candidate in the treatment of patients with lung cancer and potentially other diseases. The therapeutic activity of hsa-miR-147 is highlighted by the fact that hsa-miR-147 inhibits tumor growth of tumors that had developed prior to treatment.

In addition, the data demonstrate the therapeutic utility of hsa-miR-147 in a lipid-based formulation.

### Example 10

### Delivery of Synthetic hsa-miR-147 Inhibits Proliferation of Human Prostate Cancer Cells

The inventors assessed the therapeutic effect of hsa-miR-147 for prostate cancer by using 4 individual human prostate cancer cell lines. To measure cellular proliferation of prostate cancer cells, the following prostate cancer cell lines were used: PPC-1 and PC3, derived from a bone metastasis; Du145, derived from a brain metastasis; RWPE2, derived from prostate cells immortalized by human papillomavirus 18 and transformed by the K-RAS oncogene (Bello et al., 1997; Stone et al., 1978; Brothman et al., 1991). PC3, PPC-1, and Du145 cells lack expression of the prostate-specific antigen (PSA) and are independent of androgen receptor (AR) signaling. In contrast, RWPE2 cells test positive for PSA and AR.

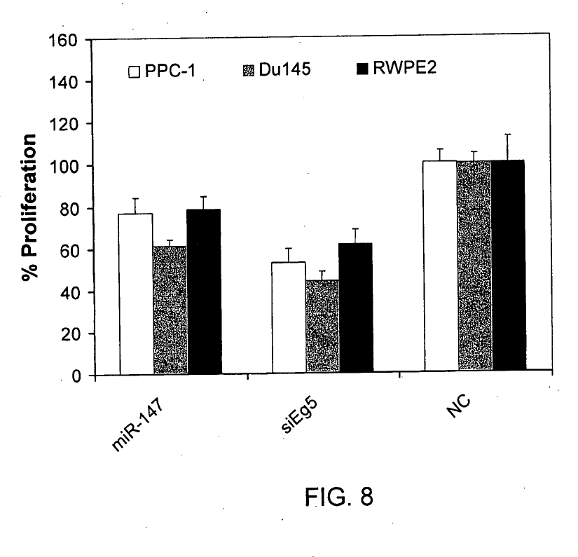

PPC-1, Du145 and RWPE2 cells were transfected with synthetic hsa-miR-147 (Pre-miR™-hsa-miR-147, Ambion cat. no. AM17100) or negative control miRNA (NC; Pre-miR™ microRNA Precursor Molecule-Negative Control #2; Ambion cat. no. AM17111) in a 96-well format using a lipid-based transfection reagent. Lipid-based reverse transfections were carried out in triplicate according to a published protocol (Ovcharenko et al., 2005) and the following parameters: Cells (6,000-7,000 per 96 well), 0.1-0.2 μl Lipofectamine™ 2000 (cat. no. 11668-019, Invitrogen Corp., Carlsbad, Calif., USA) in 20 μl OptiMEM (Invitrogen), 30 nM final concentration of miRNA in 100 μl. Proliferation was assessed 4-7 days post-transfection using Alamar Blue™ (Invitrogen) following the manufacturer's instructions. As a control for inhibition of cellular proliferation, siRNA against the motor protein kinesin 11, also known as Eg5, was used. Eg5 is essential for cellular survival of most eukaryotic cells and a lack thereof leads to reduced cell proliferation and cell death (Weil et al., 2002). siEg5 was used in lipid-based transfection following the same experimental parameters that apply to miRNA. Fluorescent light units (FLU) were measured after 3 hours, normalized to the control, and plotted as percent change in proliferation. Percent proliferation of hsa-miR-147 treated cells relative to cells treated with negative control miRNA (100%) is shown in Table 10 and in FIG. 8.

TABLE 10
Percent (%) proliferation of human prostate cancer cell lines treated
with hsa-miR-147, Eg5-specific siRNA (siEg5), or negative control
miRNA (NC).

| | hsa-miR-147 | | | | | |
|---|---|---|---|---|---|---|
| | (30 nM) | | siEg5 (30 nM) | | NC (30 nM) | |
| | % | | % | | % | |
| Cells | proliferation | % SD | proliferation | % SD | proliferation | % SD |
| PPC-1 | 76.98 | 7.37 | 52.90 | 6.97 | 100.00 | 5.82 |
| Du145 | 61.50 | 2.78 | 44.47 | 4.23 | 100.00 | 4.12 |
| RWPE2 | 79.08 | 5.59 | 61.87 | 6.56 | 100.00 | 12.28 |

Values are normalized to values obtained from cells transfected with negative control miRNA (100% proliferation).
NC, negative control miRNA;
siEg5, Eg5-specific siRNA;
SD, standard deviation.

Delivery of hsa-miR-147 inhibits cellular proliferation of human prostate cancer cells PPC-1, Du145 and RWPE2 (Table 10 and FIG. 8). On average, hsa-miR-147 inhibits cellular proliferation by 27.48%. The growth-inhibitory activity of hsa-miR-147 is comparable to that of Eg5-directed siRNA. Since hsa-miR-147 induces a therapeutic response in prostate cancer cells independent of PSA or AR status, hsa-miR-147 may provide therapeutic benefit to a broad range of patients with prostate cancer and other malignancies.

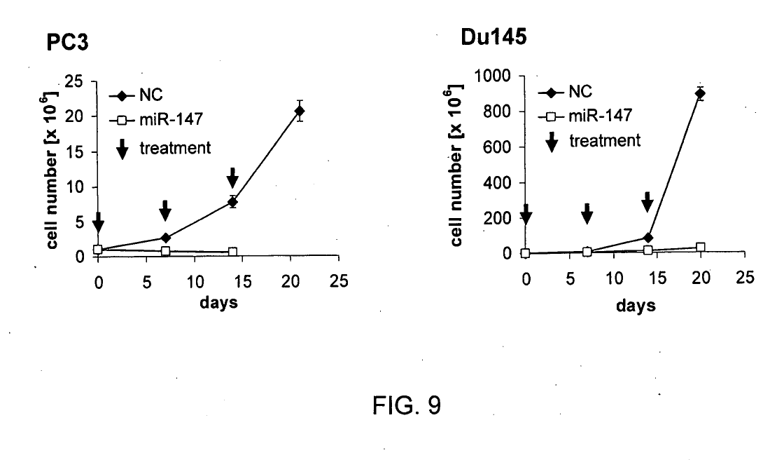

To evaluate the therapeutic activity of hsa-miR-147 over an extended period of time, the inventors conducted growth curve experiments in the presence of miRNA for up to 21 days. Since in vitro transfections of naked interfering RNAs, such as synthetic miRNA, are transient by nature and compromised by the dilution of the oligo during ongoing cell divisions, miRNA was administered at multiple time points (Bartlett et al., 2006; Bartlett et al., 2007). To accommodate miRNA delivery into a large quantity of cells, the inventors employed the electroporation method to delivery hsa-miR-147 or negative control miRNA into PC3 and Du145 human prostate cancer cells. Briefly, 1×10⁶ PC3 cells and 0.5×10⁶ Du145 cells were electroporated with 1.6 μM hsa-miR-147 or negative control using the BioRad Gene Pulser Xcell™ instrument (BioRad Laboratories Inc., Hercules, Calif., USA), seeded and propagated in regular growth medium. Experiments with PC3 and Du145 cells were carried out in triplicates. When the control cells reached confluence (days 7 and 14), cells were harvested, counted and electroporated again with the respective miRNAs. To ensure similar treatment of both conditions as well as to accommodate exponential growth, the cell numbers used for the second and third electroporation were titrated down to the lowest count. The population doubling was calculated from these electroporation events using the formula PD=ln(Nf/N0)/ln2 and adjusting for the fact that approximately 72% of newly seeded cells adhere to the plate. Cell counts were extrapolated and plotted on a linear scale (FIG. 9). Arrows represent electroporation days. Standard deviations are included in the graphs.

Repeated administration of hsa-miR-147 robustly inhibited proliferation of human prostate cancer cells (FIG. 9, white squares). In contrast, cells treated with negative control miRNA showed normal exponential growth (FIG. 9, black diamonds). hsa-miR-147 treatment resulted in 97.1% inhibition of Du145 cell growth on day 20 (2.90% cells relative to cells electroporated with negative control miRNA) relative to the proliferation of control cells (100%). All PC3 cells electroporated with hsa-miR-147 were eliminated by day 21.

The data suggest that hsa-miR-147 provides a useful therapeutic tool in the treatment of human patients with prostate cancer.

### Example 11

### Genes, Gene Pathways, and Cancer-Related Genes with Altered Expression Following Transfection with hsa-miR-188

As mentioned above in previous examples, the regulatory effects of miRNAs are revealed through changes in global gene expression profiles following miRNA expression or inhibition of miRNA expression. Microarray gene expression analyses were performed to identify genes that are mis-regulated by hsa-miR-188 expression. Synthetic pre-miR-188 (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1F.

Manipulation of the expression levels of the genes listed in Table 1F represents a potentially useful therapy for cancer and other diseases in which increased or reduced expression of hsa-miR-188 has a role in the disease.

The mis-regulation of gene expression by hsa-miR-188 (Table 1F) affects many cellular pathways that represent potential therapeutic targets for the control of cancer and other diseases and disorders. The inventors determined the identity and nature of the cellular genetic pathways affected by the regulatory cascade induced by hsa-miR-188 expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-188 in A549 cells are shown in Table 2F.

These data demonstrate that hsa-miR-188 directly or indirectly affects the expression of numerous cellular proliferation-, development-, and cell growth-related genes and thus primarily affects functional pathways related to cellular growth and cellular development. Those cellular processes have integral roles in the development and progression of various cancers. Manipulation of the expression levels of genes in the cellular pathways shown in Table 2F represents a potentially useful therapy for cancer and other diseases in which increased or reduced expression of hsa-miR-188 has a role in the disease.

Gene targets for binding of and regulation by hsa-miR-188 were predicted using the proprietary algorithm miRNATarget™ (Asuragen), which is an implementation of the method proposed by Krek et al., (2005). The predicted gene targets that exhibited altered mRNA expression levels in human cancer cells, following transfection with pre-miR hsa-miR-188, are shown in Table 3F below.

The verified gene targets of hsa-miR-188 in Table 3F represent particularly useful candidates for cancer therapy and therapy of other diseases through manipulation of their expression levels.

Cell proliferation and survival pathways are commonly altered in tumors (Hanahan and Weinberg, 2000). The inventors have shown that hsa-miR-188 directly or indirectly regulates the transcripts of proteins that are critical in the regulation of these pathways. Many of these targets have inherent oncogenic or tumor suppressor activity and are frequently deregulated in human cancer. Hsa-miR-188 targets that have prognostic and/or therapeutic value for the treatment of various malignancies are shown in Table 4D. Based on this review of the genes and related pathways that are regulated by miR-188, introduction of hsa-miR-188 or an anti-hsa-miR-188 into a variety of cancer cell types would likely result in a therapeutic response.

### Example 12

### Genes, Gene Pathways, and Cancer-Related Genes with Altered Expression Following Transfection with hsa-miR-215

Microarray gene expression analyses were performed to identify genes that are mis-regulated by hsa-miR-215 expression. Synthetic pre-miR-215 (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

As mentioned above in previous examples, the regulatory effects of miRNAs are revealed through changes in global gene expression profiles following miRNA expression or inhibition of miRNA expression. mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1G.

Manipulation of the expression levels of the genes listed in Table 1G represents a potentially useful therapy for cancer and other diseases in which increased or reduced expression of hsa-miR-215 has a role in the disease.

The mis-regulation of gene expression by hsa-miR-215 (Table 1G) affects many cellular pathways that represent potential therapeutic targets for the control of cancer and other diseases and disorders. The inventors determined the identity and nature of the cellular genetic pathways affected by the regulatory cascade induced by hsa-miR-215 expression. Cellular pathway analyses were performed using Ingenuity Pathways Analysis (Version 4.0, Ingenuity®Systems, Redwood City, Calif.). Alteration of a given pathway was determined by Fisher's Exact test (Fisher, 1922). The most significantly affected pathways following over-expression of hsa-miR-215 in A549 cells are shown in Table 2G.

These data demonstrate that hsa-miR-215 directly or indirectly affects the expression of numerous cellular proliferation-, development-, cell growth, and cancer-related genes and thus primarily affects functional pathways related to cellular growth and cellular development. Those cellular processes have integral roles in the development and progression of various cancers. Manipulation of the expression levels of genes in the cellular pathways shown in Table 2G represents a potentially useful therapy for cancer and other diseases in which increased or reduced expression of hsa-miR-215 has a role in the disease.

Gene targets for binding of and regulation by hsa-miR-215 were predicted using the proprietary algorithm miRNATarget™ (Asuragen), which is an implementation of the method proposed by Krek et al., (2005). The predicted gene targets that exhibited altered mRNA expression levels in human cancer cells, following transfection with pre-miR hsa-miR-215, are shown in Table 3G.

The verified gene targets of hsa-miR-215 in Table 3G represent particularly useful candidates for cancer therapy and therapy of other diseases through manipulation of their expression levels.

Cell proliferation and survival pathways are commonly altered in tumors (Hanahan and Weinberg, 2000). The inventors have shown that hsa-miR-215 directly or indirectly regulates the transcripts of proteins that are critical in the regulation of these pathways. Many of these targets have inherent oncogenic or tumor suppressor activity and are frequently deregulated in human cancer. Hsa-miR-215 targets that have prognostic and/or therapeutic value for the treatment of various malignancies are shown in Table 4E. Based on this review of the genes and related pathways that are regulated by miR-215, introduction of hsa-miR-215 or an anti-hsa-miR-215 into a variety of cancer cell types would likely result in a therapeutic response.

### Example 13

### Genes, Gene Pathways, and Cancer-Related Genes with Altered Expression Following Transfection with hsa-miR-216

As mentioned above in previous examples, the regulatory effects of miRNAs are revealed through changes in global gene expression profiles following miRNA expression or inhibition of miRNA expression. Microarray gene expression analyses were performed to identify genes that are mis-regulated by hsa-miR-216 expression. Synthetic pre-miR-216 (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1H.

Manipulation of the expression levels of the genes listed in Table 1H represents a potentially useful therapy for cancer and other diseases in which increased or reduced expression of hsa-miR-216 has a role in the disease.

The mis-regulation of gene expression by hsa-miR-216 (Table 1H) affects many cellular pathways that represent potential therapeutic targets for the control of cancer and other diseases and disorders. The inventors determined the identity and nature of the cellular genetic pathways affected by the regulatory cascade induced by hsa-miR-216 expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-216 in A549 cells are shown in Table 2H.

These data demonstrate that hsa-miR-216 directly or indirectly affects the expression of numerous cellular proliferation-, cellular development-, cell growth-, and cancer-related genes and thus primarily affects functional pathways related to cellular growth and cellular development. Those cellular processes have integral roles in the development and progression of various cancers. Manipulation of the expression levels of genes in the cellular pathways shown in Table 2H represents a potentially useful therapy for cancer and other diseases in which increased or reduced expression of hsa-miR-216 has a role in the disease.

Gene targets for binding of and regulation by hsa-miR-216 were predicted using the proprietary algorithm miRNATarget™ (Asuragen), which is an implementation of the method proposed by Krek et al., (2005). The predicted gene targets that exhibited altered mRNA expression levels in human cancer cells, following transfection with pre-miR hsa-miR-216, are shown in Table 3H.

The verified gene targets of hsa-miR-216 in Table 3H represent particularly useful candidates for cancer therapy and therapy of other diseases through manipulation of their expression levels.

Cell proliferation and survival pathways are commonly altered in tumors (Hanahan and Weinberg, 2000). The inventors have shown that hsa-miR-216 directly or indirectly regulates the transcripts of proteins that are critical in the regulation of these pathways. Many of these targets have inherent oncogenic or tumor suppressor activity and are frequently deregulated in human cancer. Hsa-miR-216 targets that have prognostic and/or therapeutic value for the treatment of various malignancies are shown in Table 4F. Based on this review of the genes and related pathways that are regulated by miR-216, introduction of hsa-miR216 or an anti-hsa-miR-216 into a variety of cancer cell types would likely result in a therapeutic response.

### Example 14

### Genes, Gene Pathways, and Cancer-Related Genes with Altered Expression Following Transfection with hsa-miR-331

As mentioned above in previous examples, the regulatory effects of miRNAs are revealed through changes in global gene expression profiles following miRNA expression or inhibition of miRNA expression. Microarray gene expression analyses were performed to identify genes that are mis-regulated by hsa-miR-331 expression. Synthetic pre-miR-331 (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 11.

Manipulation of the expression levels of the genes listed in Table 11 represents a potentially useful therapy for cancer and other diseases in which increased or reduced expression of hsa-miR-331 has a role in the disease.

The mis-regulation of gene expression by hsa-miR-331 (Table 11) affects many cellular pathways that represent potential therapeutic targets for the control of cancer and other diseases and disorders. The inventors determined the identity and nature of the cellular genetic pathways affected by the regulatory cascade induced by hsa-miR-331 expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-331 in A549 cells are shown in Table 21.

These data demonstrate that hsa-miR-331 directly or indirectly affects the expression of numerous cellular development-, and cancer-related genes and thus primarily affects functional pathways related to cancer and cellular development. Manipulation of the expression levels of genes in the cellular pathways shown in Table 21 represents a potentially useful therapy for cancer and other diseases in which increased or reduced expression of hsa-miR-331 has a role in the disease.

Gene targets for binding of and regulation by hsa-miR-331 were predicted using the proprietary algorithm miRNATarget™ (Asuragen), which is an implementation of the method proposed by Krek et al., (2005). The predicted gene targets that exhibited altered mRNA expression levels in human cancer cells, following transfection with pre-miR hsa-miR-331, are shown in Table 31.

The verified gene targets of hsa-miR-331 in Table 31 represent particularly useful candidates for cancer therapy and therapy of other diseases through manipulation of their expression levels.

Cell proliferation and survival pathways are commonly altered in tumors (Hanahan and Weinberg, 2000). The inventors have shown that hsa-miR-331 directly or indirectly regulates the transcripts of proteins that are critical in the regulation of these pathways. Many of these targets have inherent oncogenic or tumor suppressor activity and are frequently deregulated in human cancer. Hsa-miR-331 targets that have prognostic and/or therapeutic value for the treatment of various malignancies are shown in Table 4G. Based on this review of the genes and related pathways that are regulated by miR-331, introduction of hsa-miR-331 or an anti-hsa-miR-331 into a variety of cancer cell types would likely result in a therapeutic response.

### Example 15

### Genes, Gene Pathways, and Cancer-Related Genes with Altered Expression Following Transfection with mmu-miR-292-3p

As mentioned above in previous examples, the regulatory effects of miRNAs are revealed through changes in global gene expression profiles following miRNA expression or inhibition of miRNA expression. Microarray gene expression analyses were performed to identify genes that are mis-regulated by mmu-miR-292-3p expression in human cancer cells. Synthetic pre-miR-292-3p (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1J.

The mis-regulation of gene expression in human cancer cells by mmu-miR-292-3p (Table 1J) affects many cellular pathways that represent potential therapeutic targets for the control of cancer and other diseases and disorders. The inventors determined the identity and nature of the cellular genetic pathways affected by the regulatory cascade induced by mmu-miR-292-3p expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of mmu-miR-292-3p in A549 cells are shown in Table 2J.

These data demonstrate that mmu-miR-292-3p directly or indirectly affects the expression of numerous cellular proliferation-, cell development-, cell growth-, and cancer-related genes and thus primarily affects functional pathways, in human cancer cells, that are related to cellular growth and cellular development. Those cellular processes have integral roles in the development and progression of various cancers. Manipulation of the expression levels of genes in the cellular pathways shown in Table 2J represents a potentially useful therapy for cancer and other diseases.

Human gene targets for binding of and regulation by mmu-miR-292-3p were predicted using the proprietary algorithm miRNATarget™ (Asuragen), which is an implementation of the method proposed by Krek et al., (2005). The predicted gene targets that exhibited altered mRNA expression levels in human cancer cells, following transfection with pre-miR mmu-miR-292-3p, are shown in Table 3J.

The verified gene targets of mmu-miR-292-3p in Table 3J represent particularly useful candidates for cancer therapy and therapy of other diseases through manipulation of their expression levels.

Cell proliferation and survival pathways are commonly altered in tumors (Hanahan and Weinberg, 2000). The inventors have shown that mmu-miR-292-3p directly or indirectly regulates the transcripts of proteins that are critical in the regulation of these pathways. Many of these targets have inherent oncogenic or tumor suppressor activity and are frequently deregulated in human cancer. Human gene targets of mmu-miR-292-3p that have prognostic and/or therapeutic value for the treatment of various malignancies are shown in Table 4H. Based on this review of the genes and related pathways that are regulated by miR-292-3p, introduction of miR-292-3p or an anti-miR-292-3p into a variety of cancer cell types would likely result in a therapeutic response.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,870,287
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,399,363
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,468
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,525,464
U.S. Pat. No. 5,527,681
U.S. Pat. No. 5,529,756
U.S. Pat. No. 5,532,128
U.S. Pat. No. 5,543,158
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,289
U.S. Pat. No. 5,610,287
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,641,515
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,739,169
U.S. Pat. No. 5,744,305
U.S. Pat. No. 5,760,395
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,846,225
U.S. Pat. No. 5,846,945
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. application Ser. No. 09/545,207
U.S. application Ser. No. 10/667,126
U.S. application Ser. No. 11/141,707
U.S. application Ser. No. 11/141,707
U.S. application Ser. No. 11/141,707
U.S. application Ser. No. 11/273,640
U.S. application Ser. No. 11/273,640,
U.S. application Ser. No. 11/273,640,
U.S. application Ser. No. 11/349,727
U.S. Prov. Appln. Ser. No. 60/575,743
U.S. Prov. Appln. Ser. No. 60/649,584
U.S. Prov. Appln. Ser. No. 60/650,807
Aaboe et al., Biochim Biophys Acta, 1638(1):72-82, 2003.
Abuharbeid et al., Int. J. Biochem. Cell Biol., 38(9):1463-1468, 2006.
Adams et al., Cancer Lett, 220(2):137-144, 2005.
Adelaide et al., Genes Chromosomes Cancer, 37(4):333-345, 2003.
Akiba et al., Int. J. Oncol., 18(2):257-264, 2001.
Akino et al., Gastroenterology, 129(1):156-169, 2005.
Alevizos et al., Oncogene, 20(43):6196-6204, 2001.
Ambros, Cell, 107(7):823-826, 2001.
Arap et al., Cancer Res., 55(6):1351-1354, 1995.
Aspland et al., Oncogene, 20(40):5708-5717, 2001.
Austin-Ward and Villaseca, Revista Medica de Chile, 126(7):838-845, 1998.
Baba et al., Cancer Res, 60(24):6886-6889, 2000.
Bae et al., J. Biol. Chem., 275(33):25255-25261, 2000.
Bagga et al., Cell, 122(4):553-563, 2005.
Bai et al., Histol Histopathol, 18(2):449-457, 2003.
Bandyopadhyay et al., Oncogene, 21(22):3541-3551, 2002.
Bangoura et al., World J Gastroenterol, 10(4):525-530, 2004.
Barclay et al., Bmj. 305(6859):953; author reply 953-4, 1992.
Bartlett and Davis, Biotechnol Bioeng. 99(4):975-85, 2008.
Bartlett and Davis, Nucleic Acids Res. 34(1):322-33, 2006.
Barton et al., Clin Cancer Res, 3(9):1579-1586, 1997.
Bartsch and Tschesche, FEBS Lett., 357(3):255-259, 1995.
Beisner et al., Cancer Res, 66(15):7554-7561, 2006.
Bello et al., Carcinogenesis. 18(6):1215-23, 1997.
Bellovin et al., Oncogene, 25(52):6959-6967, 2006.
Biswas et al., Cancer Res, 64(14):4687-4692, 2004.
Blanc et al., Cancer Lett, 228(1-2): 117-123, 2005.
Bodner-Adler et al., Anticancer Res, 21(1B):809-812, 2001.
Boise et al., Cell, 74(4):597-608, 1993.
Bostwick et al., Prostate, 58(2):164-168, 2004.
Boultwood et al., Br J Haematol, 126(4):508-511, 2004.
Boutros and Byrne, Exp Cell Res, 310(1):152-165, 2005.
Boutros et al., Biochem Biophys Res Commun, 325(4):1115-1121, 2004.
Bradham et al., J. Cell Biol., 114(6):1285-1294, 1991.
Brennecke et al., Cell, 113(1):25-36, 2003.
Brothman et al., J. Urol. 145(5):1088-91, 1991.
Budhu et al., Cancer Cell, 10(2):99-111, 2006.
Bui et al., Br J Cancer, 77(2):319-324, 1998.
Bukowski et al., Clinical Cancer Res., 4(10):2337-2347, 1998.
Butler et al., Cancer Res, 62(14):4089-4094, 2002.
Cahill et al., Nature, 392(6673):300-303, 1998.
Caldas et al., Cancer Res., 54:3568-3573, 1994.
Calin and Croce, Nat. Rev. Cancer, 6(11):857-866, 2006.
Calin et al., Proc. Natl. Acad. Sci. USA, 99(24):15524-15529, 2002.
Cao et al., Cell, 107(6):763-775, 2001.
Carbone et al., Blood, 91(3):747-755, 1998.
Carrano et al., Nat Cell Biol, 1(4):193-199, 1999.
Carreiras et al., Gynecol Oncol, 62(2):260-267, 1996.
Carreiras et al., Gynecol Oncol, 72(3):312-322, 1999.
Carrington and Ambros, Science, 301(5631):336-338, 2003.
Castillo et al., Cancer Res., 66(12):6129-6138, 2006.
Chan et al., Oncogene, 22(44):6946-6953, 2003.
Chandler et al., Int. J. Cancer, 81(3):451-458, 1999.
Chen et al., J Pathol, 200(5):640-646, 2003.
Chen, Cytogenet Cell Genet. 62(2-3):183-4, 1993.
Cheng et al., Cancer Res., 54(21):5547-5551, 1994.
Choi et al., Oncogene, 23(42):7095-7103, 2004.
Choi et al., Oncogene. 23(42):7095-7103, 2004.
Cho-Vega et al., Hum Pathol. 35(9):1095-1100, 2004.
Christodoulides et al., Microbiology, 144(Pt 11):3027-3037, 1998.
Ciocca et al., J Natl Cancer Inst, 85(7):570-574, 1993.
Claudio et al., Clin. Cancer Res., 8(6):1808-1815, 2002.
Croci et al., Cancer Res., 64(5):1730-1736, 2004.
Cully et al., Cancer Res, 65(22):10363-10370, 2005.
Cummins et al., In: IRT: Nucleosides and nucleosides, La Jolla Calif., 72, 1996.
D'Antonio et al., Int. J. Oncol., 21(5):941-948, 2002.
Davalos et al., Oncogene, 26(2):308-311, 2007.
Davidson et al., J. Immunother., 21(5):389-398, 1998.
de Candia et al., Hum Pathol, 37(8):1032-1041, 2006.
de Nigris et al., Cancer Res, 61(5):2267-2275, 2001.
Denli et al., Trends Biochem. Sci., 28:196, 2003.
Didenko, Biotechniques, 31(5):1106-1116, 1118, 1120-1121, 2001.
Dillman, Cancer Biother. Radiopharm., 14(1):5-10, 1999.
Dong et al., Crit. Rev. Oncol. Hematol., 54(2):85-93, 2005.
Dong et al., Mol. Endocrinol., 20(10):2315-2325, 2006.
Donnellan and Chetty, Mol Pathol, 51(1):1-7, 1998.
Dyer and Bremner, Nat Rev Cancer, 5(2):91-101, 2005.
Ebert et al., Cancer Res., 54(15):3959-3962, 1994.
Eferl et al., Cell, 112(2):181-192, 2003.
Einama et al., Pancreas, 32(4):376-381, 2006.
Emptage et al., Neuron, 29(1):197-208, 2001.
Endoh et al., Br J Cancer, 93(12):1395-1399, 2005.
EP 266,032
EP 373 203
EP 785 280
EP 799 897
Esquela-Kerscher and Slack, Nat Rev Cancer, 6(4):259-269, 2006.
Eustace et al., Nat Cell Biol, 6(6):507-514, 2004.
Ezzat et al., Clin. Cancer Res., 11(3): 1336-1341, 2005.
Faried et al., Eur J Cancer, 42(10):1455-1465, 2006.
Feldman and Feldman, Nat Rev Cancer, 1(1):34-45, 2001.
Fernandez et al., Clin. Cancer Res., 11 (15):5390-5395, 2005.
Fesik, Nat Rev Cancer, 5(11):876-885, 2005.
Filipits et al., Clin Cancer Res, 8(3):729-733, 2002.
Firth and Baxter, Endocr. Rev., 23(6):824-854, 2002.
Fisher, J. Royal Statistical. Soc. 85(1):87-94, 1922.
Fleischer et al., Int J Oncol. 28(1):25-32, 2006.
Florenes et al., Clin Cancer Res, 6(9):3614-3620, 2000.
Fodor et al., Biochemistry, 30(33):8102-8108, 1991.
Froehler et al., Nucleic Acids Res., 14(13):5399-5407, 1986.
Fujiwara et al., Oncogene, 10(5):891-895, 1995.
Fujiwara et al., Oncogene. 10(5):891-895, 1995.
Furutani et al., Cancer Lett., 122(1-2):209-214, 1998.
Gao et al., Oncol Rep, 17(1):123-128, 2007.
Golay et al., Blood, 87(5):1900-1911, 1996.
Gottardo et al., Br J Haematol. 137(4):319-328, 2007.
Grabsch et al., J Pathol, 200(1):16-22, 2003.
Graff and Zimmer, Clin Exp Metastasis, 20(3):265-273, 2003.
Grandori et al., Annu Rev Cell Dev Biol, 16:653-699, 2000.
Gratas et al., Cancer Res., 58(10):2057-2062, 1998.
Griffey et al., J. Mass Spectrom, 32(3):305-13, 1997.
Gstaiger et al, Proc Natl Acad Sci USA, 98(9):5043-5048, 2001.
Guanti et al., Hum Mol Genet, 9(2):283-287, 2000.
Guo et al., Carcinogenesis, 27(3):454-464, 2006.
Hamamura et al., Proc Natl Acad Sci USA, 102(31):11041-11046, 2005.
Han et al., Oncogene, 23(7):1333-1341, 2004.
Hanahan and Weinberg, Cell, 100(1):57-70, 2000.
Hanibuchi et al., Int. J. Cancer, 78(4):480-485, 1998.
Hannigan et al., Nat Rev Cancer, 5(1):51-63, 2005.
Hartmann et al., Cancer Res, 59(7):1578-1583, 1999.
He et al., Nature, 435(7043):828-833, 2005a.
He et al., Proc. Natl. Acad. Sci. USA, 102(52):19075-19080, 2005b.
Hellstrand et al., Acta Oncologica, 37(4):347-353, 1998.
Hishikawa et al., J. Biol. Chem., 274(52):37461-37466, 1999.
Hooi et al., Oncogene, 25(28):3924-3933, 2006.
Hoshiko et al., Agents Actions Suppl. 34:323-333, 1991.
Houston and O'Connell, Curr. Opin. Pharmacol., 4(4):321-326, 2004.
Huang et al., Clin Cancer Res, 12(2):487-498, 2006.
Huang et al., J Clin Oncol, 23(34):8765-8773, 2005.
Huguet et al., Cancer Res, 54(10):2615-2621, 1994.
Hui and Hashimoto, Infection Immun., 66(11):5329-5336, 1998.
Hussussian et al., Nat. Genet., 8(1):15-21, 1994.
Huusko et al., Nat Genet, 36(9):979-983, 2004.
Hynes and Lane, Nat. Rev. Cancer, 5(5):341-354, 2005.
Iolascon et al., Hepatology, 27(4):989-995, 1998.
Ireton and Chen, Curr Cancer Drug Targets, 5(3):149-157, 2005.
Ishikawa et al., Cancer Res, 65(20):9176-9184, 2005.
Ishikawa et al., Cancer Res., 65(20):9176-9184, 2005.
Itakura and Riggs, Science, 209:1401-1405, 1980.
Ito et al., Anticancer Res, 21(2A):1043-1048, 2001.
Ito et al., Anticancer Res, 22(3):1581-1584, 2002.
Ito et al., Anticancer Res, 23(3B):2335-2338, 2003b.
Ito et al., Anticancer Res, 25(5):3419-3423, 2005.
Ito et al., Anticancer Res., 23(5A):3819-3824, 2003.
Jaakkola et al., Int. J. Cancer, 54(3):378-382, 1993.
Jansen et al., Mol Cancer Ther, 3(2):103-110, 2004.
Jiang et al., Cancer Res, 64(16):5787-5794, 2004.
Jonson et al., Int J Oncol, 19(1):71-81, 2001.
Jonsson et al., Cancer Res, 62(2):409-416, 2002.
Ju et al., Gene Ther., 7(19):1672-1679, 2000.
Jubb et al., Clin Cancer Res, 11(14):5181-5187, 2005.
Kaighn et al., Invest Urol. 17(1):16-23, 1979.
Kamata et al., J Cancer Res Clin Oncol, 131(9):591-596, 2005.
Kamb et al., Science, 2674:436-440, 1994.
Kaufmann et al., Blood, 91(3):991-1000, 1998.
Kirikoshi et al., Int J Oncol, 19(1):111-115, 2001.
Kitada et al., Blood, 91(9):3379-3389, 1998.
Kitadai et al., Jpn. J. Cancer Res., 84(8):879-884, 1993.
Kleer et al., Clin Cancer Res, 12(15):4485-4490, 2006.
Klostermeier and Millar, Biopolymers, 61(3):159-79, 2001-2002.
Koivunen et al., Cancer Lett, 235(1):1-10, 2006.
Kokko et al., BMC Cancer, 6:145, 2006.
Koliopanos et al., World J. Surg., 26(4):420-427, 2002.
Komiya et al., Jpn J Cancer Res, 88(4):389-393, 1997.
Krajewska et al., Am J Pathol. 148(5):1567-1576, 1996.
Krasagakis et al., Br J Cancer, 77(9):1492-1494, 1998.
Krek et al., Nature Genet. 37:495-500, 2005.
Kulkarni et al., Leukemia, 16(1):127-134, 2002.
Lagos-Quintana et al., Science, 294(5543):853-858, 2001.
Lahn and Sundell, Melanoma Res, 14(2):85-89, 2004.
Lambros et al., J Pathol, 205(1):29-40, 2005.
Landen et al., Expert Opin Ther Targets, 9(6):1179-1187, 2005.
Lau et al., Science, 294(5543):858-862, 2001.
Lauffart et al., BMC Womens Health, 5:8, 2005.
Lazaris et al., Breast Cancer Res Treat, 43(1):43-51, 1997.
Lazaris et al., Dis Colon Rectum, 38(7):739-745, 1995.
Lee and Ambros, Science, 294(5543):862-864, 2001.
Lee et al., Cell Struct Funct, 23(4):193-199, 1998.
Lee et al., Int. J. Cancer, 118(10):2490-2497, 2006.
Lee et al., Oncogene, 23(39):6672-6676, 2004.
Leprince et al., Nature, 306(5941):395-397, 1983.
Leris et al., Anticancer Res, 25(2A):731-734, 2005.
L'Hote and Knowles, Exp Cell Res, 304(2):417-431, 2005.
Li et al., World J Gastroenterol, 9(2):205-208, 2003.
Liby et al., Folia Biol (Praha), 52(1-2):21-33, 2006.
Lim et al., Nature, 433(7027):769-773, 2005
Lin et al., Gastroenterology, 128(1):9-23, 2005.
Liu and Matsuura, Cell Cycle, 4(1):63-66, 2005.
Liu et al., Cancer Res., 66(2):653-658, 2006.
Lo Vasco et al., Leukemia, 18(6):1122-1126, 2004.
Lopez-Beltran et al., J Pathol, 209(1):106-113, 2006.
Lu et al., Nature, 435(7043):834-838, 2005.
Lucke et al., Cancer Res, 61(2):482-485, 2001.
Mahtouk et al., Oncogene, 24(21):3512-3524, 2005.
Maki et al., Proc Natl Acad Sci USA, 84(9):2848-2852, 1987.
Malumbres and Barbacid, Nat Rev Cancer, 1(3):222-231, 2001.
Manion and Hockenbery, Cancer Biol Ther. 2(4 Suppl 1):S105-114, 2003.
Markowitz et al., Science, 268(5215):1336-1338, 1995.
Markowitz, Biochim Biophys Acta, 1470(1):M13-20, 2000.
Marks, Semin. Cancer Biol., 16(6):436-443, 2006.
Marsit et al., Int. J. Cancer, 119(8):1761-1766, 2006.
Marsters et al., Recent Prog. Horm. Res., 54:225-234, 1999.
Martinez-Lorenzo et al., Int. J. Cancer, 75(3):473-481, 1998.
Massague et al., Cell, 103(2):295-309, 2000.
Matsumoto et al., Leukemia, 14(10):1757-1765, 2000.
McGary et al., Cancer Biol Ther, 1(5):459-465, 2002.
Meng et al., Gastroenterology, 130:2113-2129, 2006.
Merle et al., Gastroenterology, 127(4):1110-1122, 2004.
Miyake et al., Cancer, 86(2):316-324, 1999.
Mizunuma et al., Br J Cancer, 88(10):1543-1548, 2003.
Moll and Slade, Mol Cancer Res, 2(7):371-386, 2004.
Moller et al., Int J Cancer, 57(3):371-377, 1994.
Momand et al., Nucleic Acids Res, 26(15):3453-3459, 1998.
Montero et al., Clin Cancer Res, 4(9):2161-2168, 1998.
Mori et al., Cancer Res., 54(13):3396-3397, 1994.
Mori et al., Gastroenterology, 131(3):797-808, 2006.
Morishita et al., Hepatology, 40(3):677-686, 2004.
Mossink et al., Oncogene, 22(47):7458-7467, 2003.
Nakada et al., Cancer Res, 64(9):3179-3185, 2004.
Nakagawa et al., Oncogene, 23(44):7366-7377, 2004.
Nakayama et al., Cancer, 92(12):3037-3044, 2001.
Nesbit et al., Oncogene, 18(19):3004-3016, 1999.
Nobri et al., Nature (London), 368:753-756, 1995.
Nupponen et al., Am J Pathol, 154(6):1777-1783, 1999.
Nupponen et al., Genes Chromosomes Cancer, 28(2):203-210, 2000.
O'Donnell et al., Nature, 435:839-843, 2005.
Ohsaki et al., Cancer Res, 1992. 52(13): p. 3534-8.
Okamoto et al., Hepatology, 38(5):1242-1249, 2003.
Okamoto et al., Proc. Natl. Acad. Sci. USA, 91(23):11045-11049, 1994.
Okino et al., Oncol Rep, 13(6):1069-1074, 2005.
Olsen et al., Dev. Biol., 216:671, 1999.
Orlow et al., Cancer Res, 54(11):2848-2851, 1994.
Ovcharenko et al., Rna, 2005. 11(6): p. 985-93.
Pan et al., Neurol. Res., 24(7):677-683, 2002.
Parekh et al., Biochem Pharmacol, 63(6):1149-1158, 2002.
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
Pennica et al., Proc Natl Acad Sci USA, 95(25):14717-14722, 1998.
Petit et al., Genomics, 57(3):438-441, 1999.
Pietras et al., Oncogene, 17(17):2235-2249, 1998.
Prentice et al., Oncogene, 24(49):7281-7289, 2005.
Pruitt et al., Nucleic Acids Res., 33(1):D501-D504, 2005.
Pruneri et al., Clin Cancer Res, 11(1):242-248, 2005.
Qian et al., Proc Natl Acad Sci USA, 99(23):14925-14930, 2002.
Qin et al., Proc. Natl. Acad. Sci. USA, 95(24):14411-14416, 1998.
Ree et al., Cancer Res, 59(18):4675-4680, 1999.
Reimer et al., J. Biol. Chem., 274(16):11022-11029, 1999.
Reinhart et al., Nature, 403(6772):901-906, 2000.
Remington's Pharmaceutical Sciences” 15th Edition, pages 1035-1038 and 1570-1580, 1990.
Ritch et al., J Biol Chem, 278(23):20971-20978, 2003.
Romieu-Mourez et al., Cancer Res, 61(9):3810-3818, 2001.
Rossi et al., Cancer Genet Cytogenet, 161(2):97-103, 2005.
Ru et al., Oncogene, 21(30):4673-4679, 2002.
Rubin and Gutmann, Nat Rev Cancer, 5(7):557-564, 2005.
Rust et al., J Clin Pathol. 58(5):520-524, 2005.
Ruth et al., J Invest Dermatol, 126(4):862-868, 2006.
Sacchi et al., Science, 231(4736):379-382, 1986.
Saigusa et al., Cancer Sci, 96(10):676-683, 2005.
Saitoh et al., Int J Mol Med, 9(5):515-519, 2002.
Salgia et al., Oncogene, 18(1):67-77, 1999.
Sambrook and Russell, Molecular Cloning: A Laboratory Manual 3rd Ed., Cold Spring Harbor Laboratory Press, 2001.
Sambrook et al., In: DNA microarrays: a molecular cloning manual, Cold Spring Harbor Laboratory Press, Cold Spring Harbor, N.Y., 2003.
Sanchez-Aguilera et al., Blood, 103(6):2351-2357, 2004.
Sano et al., Histopathology, 46(5):532-539, 2005.
Saxena et al., Mol Cell Biochem, 228(1-2):99-104, 2001.
Schulze-Bergkamen et al., BMC Cancer, 6:232, 2006.
Seggerson et al., Dev. Biol., 243:215, 2002.
Sementchenko et al., Oncogene, 17(22):2883-2888, 1998.
Serrano et al., Nature, 366:704-707, 1993.
Serrano et al., Science, 267(5195):249-252, 1995.
Shah et al., Oncogene, 21(54):8251-8261, 2002.
Shelly et al., J Biol Chem, 273(17):10496-10505, 1998.
Sherr and McCormick, Cancer Cell, 2(2):103-112, 2002.
Shibahara et al., Anticancer Res, 25(3B):1881-1888, 2005.
Shigeishi et al., Oncol Rep, 15(4):933-938, 2006.
Shigemasa et al., Jpn. J. Cancer Res, 93(5):542-550, 2002.
Shimo et al., Cancer Lett., 174(1):57-64, 2001.
Shimoyama et al., Clin Cancer Res, 5(5):1125-1130, 1999.
Shin et al., Cancer Lett, 174(2):189-194, 2001.
Shinoura et al., Cancer Gene Ther., 7(2):224-232, 2000.
Sieghart et al., J Hepatol. 44(1):151-157, 2006.
Sieghart et al., J. Hepatol., 44(1):151-157, 2006.
Simpson and Parsons, Exp Cell Res, 264(1):29-41, 2001.
Simpson et al., Oncogene, 14(18):2149-2157, 1997.
Skotzko et al., Cancer Res., 55(23):5493-5498, 1995.
Solic and Davies, Exp. Cell Res., 234(2):465-476, 1997.
Soufla et al., Cancer Lett, 221(1):105-118, 2005.
Sparmann and Bar-Sagi, Cancer Cell, 6(5):447-458, 2004.
Stone et al., Int. J. Cancer 21(3):274-281, 1978.
Su et al., Clin Cancer Res, 7(5):1320-1324, 2001.
Sui et al., Oncol Rep, 15(4):765-771, 2006.
Takanami, Oncol Rep, 13(4):727-731, 2005.
Takashima et al., Proteomics, 3(12):2487-2493, 2003.
Takimoto et al., Biochem. Biophys. Res. Commun., 251(1):264-268, 1998.
Tan et al., Leuk Res, 27(2):125-131, 2003.
Tanaka et al., Proc Natl Acad Sci USA, 95(17):10164-10169, 1998.
Tanami et al., Lab Invest, 85(9):1118-1129, 2005.
Taniwaki et al., Int J Oncol, 29(3):567-575, 2006.
Tassi et al., Cancer Res., 66(2):1191-1198, 2006.
Tassi et al., J. Biol. Chem., 276(43):40247-40253, 2001.
Thogersen et al., Cancer Res, 61(16):6227-6233, 2001.
Thome, Nat Rev Immunol, 4(5):348-359, 2004.
Tomasini-Johansson et al., Exp Cell Res, 214(1):303-312, 1994.
Torring et al., Anticancer Res, 20(1A):91-95, 2000.
Toyoda et al., Biochem J, 326 (Pt 1):69-75, 1997.
Traub et al., Breast Cancer Res Treat, 99(2):185-191, 2006.
Troncone et al., J Clin Pathol, 2006.
Tsai et al., J Natl Cancer Inst, 1993. 85(11): p. 897-901.
Tseng et al., Mol Pharmacol, 70(5):1534-1541, 2006.
Uemura et al., Int J Mol Med, 18(2):365-373, 2006.
Uhm et al., Clin Cancer Res, 5(6):1587-1594, 1999.
UK Appln. 8 803 000
UK Patent 1,529,202
Venkatasubbarao et al., Anticancer Res, 20(1A):43-51, 2000.
Viard-Leveugle et al., J. Pathol., 201(2):268-277, 2003.
Visvader et al., Proc Natl Acad Sci USA, 98(25):14452-14457, 2001.
Vivanco and Sawyers, Nat Rev Cancer, 2(7):489-501, 2002.
Vogt et al., Cell Cycle, 5(9):946-949, 2006.
Volinia et al., Proc Natl Acad Sci USA, 103(7):2257-2261, 2006.
Walker-Daniels et al., Am J Pathol, 162(4):1037-1042, 2003.
Wang et al., World J Gastroenterol, 11(3):336-339, 2005.
Weeraratna et al., Cancer Cell, 1(3):279-288, 2002.
Weichert et al., Int J Oncol, 23(3):633-639, 2003.
Weil et al., Biotechniques, 2002. 33(6): p. 1244-8.
Weiss and Bohmann, Cell Cycle, 3(2):111-113, 2004.
Wheeler and Ridley, Exp Cell Res, 301(1):43-49, 2004.
Wikman et al., Oncogene, 21(37):5804-5813, 2002.
Wilson et al., J Biol Chem, 281(19):13548-13558, 2006.
Wooster and Weber, N Engl J Med, 348(23):2339-2347, 2003.
Woszczyk et al., Med Sci Monit, 10(1):CR33-37, 2004.
Wu et al., Breast Cancer Res Treat, 84(1):3-12, 2004b.
Wu et al., Eur J Cancer, 38(14):1838-1848, 2002.
Wu et al., Eur J Cancer, 42(4):557-565, 2006a.
Wu et al., Eur. J. Cancer, 38(14):1838-1848, 2002.
Wu et al., Gynecol Oncol, 102(1):15-21, 2006b.
Wu et al., Int J Gynecol Cancer, 16(4):1668-1672, 2006b.
Wu et al., Pathol Oncol Res, 10(1):26-33, 2004a.
Wuilleme-Toumi et al., Leukemia, 19(7):1248-1252, 2005.
Xi et al., Clin. Cancer Res., 12(8):2484-2491, 2006a.
Xi et al., Clin. Chem., 52(3):520-523, 2006b.
Xia et al., Cancer Res, 61(14):5644-5651, 2001b.
Xia et al., Cancer, 91(8):1429-1436, 2001a.
Xia et al., Urology, 59(5):774-778, 2002.
Xie et al., BMC Cancer, 6:77, 2006.
Xu et al., Curr. Biol., 13(9):790-795, 2003.
Yamagata et al., Cancer Res, 65(1):157-165, 2005.
Yang et al., Cancer Cell, 9(6):445-457, 2006.
Yang et al., Cancer Res., 65(19):8887-8895, 2005.
Yang et al., J Androl, 22(3):471-480, 2001.
Yao et al., Oncogene, 25(16):2285-2296, 2006.
Yoshida et al., Ann Oncol. 15(2):252-256, 2004.
Yoshioka et al., Proc Natl Acad Sci USA, 100(12):7247-7252, 2003.
Yu et al., Nat Genet, 37(3):265-274, 2005.
Zangemeister-Wittke and Huwiler, Cancer Biol. Ther., 5(10):1355-1356, 2006.
Zeng et al., Cancer Res, 62(12):3538-3543, 2002.
Zhang et al., Oncogene, 23(12):2241-2249, 2004.
Zhang et al., Oncogene, 25(45):6101-6112, 2006.
Zhao et al., Mol. Cancer. Res., 1(3):195-206, 2003.
Zhu et al., Biochem Biophys Res Commun, 273(3):1019-1024, 2000.
Zhu et al., Cell, 94(6):703-714, 1998.

1. A method of modulating gene expression in a cell comprising administering to the cell an amount of an isolated nucleic acid comprising a miR-15, miR-26, miR-31, miR-145, miR-147, miR-188, miR-215, miR-216, miR-331, or miR-292-3p nucleic acid sequence in an amount sufficient to modulate the expression of one or more genes identified in Table 1, 3, or 4, wherein
(a) miR-15 modulated genes are selected from Table 1A, 3A, or 4A;
(b) miR-26 modulated genes are selected from Table 1B, 3B, or 4B;
(c) miR-31 modulated genes are selected from Table 1C, or 3C;
(d) miR-145 modulated genes are selected from Table 1D, or 3D;
(e) miR-147 modulated genes are selected from Table 1E, 3E, or 4C;
(f) miR-188 modulated genes are selected from Table 1F, 3F, or 4D;
(g) miR-215 modulated genes are selected from Table 1G, 3G, or 4E;
(h) miR-216 modulated genes are selected from Table 1H, 3H, or 4F;
(i) miR-331 modulated genes are selected from Table 11, 31, or 4G; and
(j) miR-292-3p modulated genes are selected from Table 1J, 3J, or 4H.

2. The method of claim 1, wherein the cell is in a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

3. (canceled)

4. The method of claim 2, wherein the cancerous condition is one or more of acute lymphoblastic leukemia; acute myeloid leukemia; anaplastic large cell lymphoma; angiosarcoma; astrocytoma; B-cell lymphoma; bladder carcinoma; breast carcinoma; Burkitt's lymphoma; carcinoma of the head and neck; cervical carcinoma; chronic lymphoblastic leukemia; chronic myeloid leukemia; colorectal carcinoma; endometrial carcinoma; esophageal carcinoma; esophageal squamous cell carcinoma; Ewing's sarcoma; fibrosarcoma; gastric carcinoma; glioblastoma; glioma; hepatoblastoma; hepatocellular carcinoma; high-grade non-Hodgkin lymphoma; Hodgkin lymphoma; Kaposi's sarcoma; laryngeal squamous cell carcinoma; larynx carcinoma; leiomyosarcoma; leukemia; lipoma; liposarcoma; lung carcinoma; mantle cell lymphoma; medulloblastoma; melanoma; mesothelioma; mucosa-associated lymphoid tissue B-cell lymphoma; multiple myeloma; myeloid leukemia; myeloma; myxofibrosarcoma; nasopharyngeal carcinoma; neuroblastoma; neurofibroma; non-Hodgkin lymphoma; non-small cell lung carcinoma; oligodendroglioma; osteosarcoma; ovarian carcinoma; pancreatic carcinoma; pheochromocytoma; prostate carcinoma; renal cell carcinoma; retinoblastoma; rhabdomyosarcoma; salivary gland tumor; schwannoma; small cell lung cancer; squamous cell carcinoma of the head and neck; testicular tumor; thyroid carcinoma; urothelial carcinoma; or Wilm's tumor wherein the modulation of one or more gene is sufficient for a therapeutic response.

5. The method of claims 2, wherein the nucleic acid comprises a miR-15 sequence and the cancerous condition is prostate carcinoma.

6. The method of claims 2, wherein the nucleic acid comprises a miR-147 sequence and the cancerous condition is lung carcinoma.

7. The method of claim 6, wherein lung carcinoma is non-small cell lung cancer.

8. The method of claims 2, wherein the nucleic acid comprises a miR-147 sequence and the cancerous condition is prostate carcinoma.

9. (canceled)

10. (canceled)

11. The method of claim 1, wherein the expression of a gene is down-regulated.

12. The method of claim 1, wherein the expression of a gene is up-regulated.

13. (canceled)

14. (canceled)

15. The method of claim 1, wherein the cell is a cancer cell.

16. The method of claim 15, wherein the cancer cell is a neuronal, glial, lung, liver, brain, breast, bladder, blood, cardiovascular, leukemic, glandular, lymphoid, adrenal, colon, colorectal, endometrial, epithelial, intestinal, meninges, mesothelial, oligodendrocyte, stomach, skin, ovarian, uterine, testicular, splenic, fat, bone, cervical, esophageal, pancreatic, prostate, kidney, retinal, connective tissue, salivary gland, smooth muscle, cardiac muscle, striated muscle, or thyroid cell.

17. The method of claim 1, wherein the isolated miR-15, miR-26, miR-31, miR-145, miR-147, miR-188, miR-215, miR-216, miR-331, or miR-292-3p nucleic acid is a recombinant nucleic acid.

18. (canceled)

19. The method of claim 17, wherein the recombinant nucleic acid is DNA.

20-22. (canceled)

23. The method of claim 1, wherein the miR-15, miR-26, miR-31, miR-145, miR-147, miR-188, miR-215, miR-216, miR-331, or miR-292-3p nucleic acid is a synthetic nucleic acid.

24. (canceled)

25. (canceled)

26. The method of claim 1, wherein the nucleic acid is administered enterally or parenterally.

27. (canceled)

28. (canceled)

29. The method of claim 1, wherein the nucleic acid is comprised in a pharmaceutical formulation.

30. The method of claim 29, wherein the pharmaceutical formulation is a lipid composition or a nanoparticle composition.

31. (canceled)

32. The method of claim 29, wherein the pharmaceutical formulation consists of biocompatible and/or biodegradable molecules.

33-49. (canceled)

50. A method of treating a patient diagnosed with or suspected of having or suspected of developing a pathological condition or disease related to a gene modulated by a miRNA comprising the steps of:
(a) administering to the patient an amount of an isolated nucleic acid comprising a miR-15, miR-26, miR-31, miR-145, miR-147, miR-188, miR-215, miR-216, miR-331, or miR-292-3p nucleic acid sequence in an amount sufficient to modulate a cellular pathway or a physiologic pathway; and
(b) administering a second therapy, wherein the modulation of the cellular pathway or physiologic pathway sensitizes the patient to the second therapy.

51. (canceled)

52. A method of selecting a miRNA to be administered to a subject with, suspected of having, or having a propensity for developing a pathological condition or disease comprising:
(a) determining an expression profile of one or more genes selected from Table 1, 3, or 4;
(b) assessing the sensitivity of the subject to miRNA therapy based on the expression profile; and
(c) selecting one or more miRNA based on the assessed sensitivity.

53-57. (canceled)
